SEQUENCE LISTING (349 sequences)

SEQ ID NO: 1 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCHFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 2 - protein, 323 aa, Synthetic construct
QNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 3 - protein, 160 aa, Homo sapiens
QNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAENLYFQ

SEQ ID NO: 4-9 - skipped (6 entries)

SEQ ID NO: 10 - protein, 16 aa, Synthetic construct
  VARIANT 1: note = K or R
  VARIANT 11: note = A, E, G, or T
  VARIANT 12: note = K or N
XSSQSIVHYNXXTYLE

SEQ ID NO: 11 - protein, 7 aa, Synthetic construct
  VARIANT 4: note = N or S
  VARIANT 6: note = A or F
KVSXRXS

SEQ ID NO: 12 - protein, 9 aa, Synthetic construct
FQGSHFPLT

SEQ ID NO: 13-14 - skipped (2 entries)

SEQ ID NO: 15 - protein, 5 aa, Synthetic construct
DYYMA

SEQ ID NO: 16 - protein, 17 aa, Synthetic construct
  VARIANT 5: note = D, E, or Y
  VARIANT 13: note = A, D, or E
NINYXGSRTDLLXSLKS

SEQ ID NO: 17 - protein, 10 aa, Synthetic construct
DTSRGSYFDV

SEQ ID NO: 18-19 - skipped (2 entries)

SEQ ID NO: 20 - protein, 214 aa, Synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 450 aa, Synthetic construct
EGQLVQSGGGLVHPGGSLRLSCAGSGFTFSSYGMHWVRQAPGKGLEWVSGIGTGGGTYSTDSVKGRFTISRDNAKNSLYLQMNSLRAEDMAVYYCARGDYYGSGSFFDCWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 215 aa, Synthetic construct
ETVVTQEPSLTVSPGGTVTLTCRSSIGAVTTSNYANWVQQKPGQAFRGLIGGTSNRAPWTPARFSGSLLGGKAALTLSGVQPEDEAEYYCALWYSTHYVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 23 - protein, 444 aa, Synthetic construct
EVTLKESGPVLVKPTETLTLTCTVSGFSLSTFGVHWIRQPPGKGLEWLGVIWRRGGTDYNAAFMSRLTISKDTSKSQVVFTMTNMDPVDTATYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 213 aa, Synthetic construct
EIVLTQSPATLSLSPGERATLSCSASSSVSYMHWFQQKPGQSPKLWIYSTSNLASGVPARFSGSGSGTDYTLTISSLEPEDFAVYYCQQRSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 445 aa, Synthetic construct
EVQLVESGGGLVQPGGSLRLSCAASGFSFNTYAMNWVRQAPGKGLEWVARIRTKSNNYATYYADSVKDRFTISRDDSESSLYLQMNSLKTEDTAVYYCVETGFAYWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 188 aa, Synthetic construct
QDSTSDLIPAPPLSKVPLQQNFQDNQFQGKWYVVGVAGNAILRPDKQPAKMFATIYELKEDKSYDVTSVRFEAKSCKYTIKTFVPGSQPGEFTSGQIKSAPGQTSTLVRVVSTNYNQHAMVFFKHVSQNREIFVITLYGRTKELTSELKENFIRFSKSLGLPENHIVFPVPIDQCIDGHHHHHHHHHH

SEQ ID NO: 27-99 - skipped (73 entries)

SEQ ID NO: 100 - protein, 16 aa, Synthetic construct
KSSQSIVHYNEKTYLE

SEQ ID NO: 101 - protein, 7 aa, Synthetic construct
KVSSRFS

SEQ ID NO: 102 - protein, 9 aa, Synthetic construct
FQGSHFPLT

SEQ ID NO: 103 - protein, 111 aa, Synthetic construct
VVMTQTPLSLSVSLGDQASISCKSSQSIVHYNEKTYLEWYLQKPGQSPQLLIYKVSSRFSGVPDRFSGSGSGTDFTLKISRVEAEDLGVYYCFQGSHFPLTFGQGTKLEIK

SEQ ID NO: 104 - protein, 218 aa, Synthetic construct
VVMTQTPLSLSVSLGDQASISCKSSQSIVHYNEKTYLEWYLQKPGQSPQLLIYKVSSRFSGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 5 aa, Synthetic construct
DYYMA

SEQ ID NO: 106 - protein, 17 aa, Synthetic construct
NINYEGSRTDLLESLKS

SEQ ID NO: 107 - protein, 10 aa, Synthetic construct
DTSRGSYFDV

SEQ ID NO: 108 - protein, 119 aa, Synthetic construct
EVQLVESGGGLVQPGGSLRLSCAASGFTFSDYYMAWVRQAPGKGLEWVANINYEGSRTDLLESLKSRFTISRDNAKNSVYLQMNSLRAEDTAVYYCARDTSRGSYFDVWGAGTTVTVSS

SEQ ID NO: 109 - protein, 448 aa, Synthetic construct
EVQLVESGGGLVQPGGSLRLSCAASGFTFSDYYMAWVRQAPGKGLEWVANINYEGSRTDLLESLKSRFTISRDNAKNSVYLQMNSLRAEDTAVYYCARDTSRGSYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 110 - protein, 16 aa, Synthetic construct
KSSQSIVHYNEKTYLE

SEQ ID NO: 111 - protein, 7 aa, Synthetic construct
KVSSRAS

SEQ ID NO: 112 - protein, 9 aa, Synthetic construct
FQGSHFPLT

SEQ ID NO: 113 - protein, 111 aa, Synthetic construct
VVMTQTPLSLSVSLGDQASISCKSSQSIVHYNEKTYLEWYLQKPGQSPQLLIYKVSSRASGVPDRFSGSGSGTDFTLKISRVEAEDLGVYYCFQGSHFPLTFGQGTKLEIK

SEQ ID NO: 114 - protein, 218 aa, Synthetic construct
VVMTQTPLSLSVSLGDQASISCKSSQSIVHYNEKTYLEWYLQKPGQSPQLLIYKVSSRASGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 5 aa, Synthetic construct
DYYMA

SEQ ID NO: 116 - protein, 17 aa, Synthetic construct
NINYYGSRTDLLESLKS

SEQ ID NO: 117 - protein, 10 aa, Synthetic construct
DTSRGSYFDV

SEQ ID NO: 118 - protein, 119 aa, Synthetic construct
EVQLVESGGGLVQPGGSLRLSCAASGFTFSDYYMAWVRQAPGKGLEWVANINYYGSRTDLLESLKSRFTISRDNAKNSVYLQMNSLRAEDTAVYYCARDTSRGSYFDVWGAGTTVTVSS

SEQ ID NO: 119 - protein, 448 aa, Synthetic construct
EVQLVESGGGLVQPGGSLRLSCAASGFTFSDYYMAWVRQAPGKGLEWVANINYYGSRTDLLESLKSRFTISRDNAKNSVYLQMNSLRAEDTAVYYCARDTSRGSYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 16 aa, Synthetic construct
KSSQSIVHYNGKTYLE

SEQ ID NO: 121 - protein, 7 aa, Synthetic construct
KVSSRFS

SEQ ID NO: 122 - protein, 9 aa, Synthetic construct
FQGSHFPLT

SEQ ID NO: 123 - protein, 111 aa, Synthetic construct
VVMTQTPLSLSVSLGDQASISCKSSQSIVHYNGKTYLEWYLQKPGQSPQLLIYKVSSRFSGVPDRFSGSGSGTDFTLKISRVEAEDLGVYYCFQGSHFPLTFGQGTKLEIK

SEQ ID NO: 124 - protein, 218 aa, Synthetic construct
VVMTQTPLSLSVSLGDQASISCKSSQSIVHYNGKTYLEWYLQKPGQSPQLLIYKVSSRFSGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 5 aa, Synthetic construct
DYYMA

SEQ ID NO: 126 - protein, 17 aa, Synthetic construct
NINYEGSRTDLLASLKS

SEQ ID NO: 127 - protein, 10 aa, Synthetic construct
DTSRGSYFDV

SEQ ID NO: 128 - protein, 119 aa, Synthetic construct
EVQLVESGGGLVQPGGSLRLSCAASGFTFSDYYMAWVRQAPGKGLEWVANINYEGSRTDLLASLKSRFTISRDNAKNSVYLQMNSLRAEDTAVYYCARDTSRGSYFDVWGAGTTVTVSS

SEQ ID NO: 129 - protein, 448 aa, Synthetic construct
EVQLVESGGGLVQPGGSLRLSCAASGFTFSDYYMAWVRQAPGKGLEWVANINYEGSRTDLLASLKSRFTISRDNAKNSVYLQMNSLRAEDTAVYYCARDTSRGSYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - protein, 16 aa, Synthetic construct
KSSQSIVHYNGKTYLE

SEQ ID NO: 131 - protein, 7 aa, Synthetic construct
KVSSRAS

SEQ ID NO: 132 - protein, 9 aa, Synthetic construct
FQGSHFPLT

SEQ ID NO: 133 - protein, 111 aa, Synthetic construct
VVMTQTPLSLSVSLGDQASISCKSSQSIVHYNGKTYLEWYLQKPGQSPQLLIYKVSSRASGVPDRFSGSGSGTDFTLKISRVEAEDLGVYYCFQGSHFPLTFGQGTKLEIK

SEQ ID NO: 134 - protein, 218 aa, Synthetic construct
VVMTQTPLSLSVSLGDQASISCKSSQSIVHYNGKTYLEWYLQKPGQSPQLLIYKVSSRASGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 5 aa, Synthetic construct
DYYMA

SEQ ID NO: 136 - protein, 17 aa, Synthetic construct
NINYYGSRTDLLASLKS

SEQ ID NO: 137 - protein, 10 aa, Synthetic construct
DTSRGSYFDV

SEQ ID NO: 138 - protein, 119 aa, Synthetic construct
EVQLVESGGGLVQPGGSLRLSCAASGFTFSDYYMAWVRQAPGKGLEWVANINYYGSRTDLLASLKSRFTISRDNAKNSVYLQMNSLRAEDTAVYYCARDTSRGSYFDVWGAGTTVTVSS

SEQ ID NO: 139 - protein, 448 aa, Synthetic construct
EVQLVESGGGLVQPGGSLRLSCAASGFTFSDYYMAWVRQAPGKGLEWVANINYYGSRTDLLASLKSRFTISRDNAKNSVYLQMNSLRAEDTAVYYCARDTSRGSYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 140 - protein, 16 aa, Synthetic construct
KSSQSIVHYNENTYLE

SEQ ID NO: 141 - protein, 7 aa, Synthetic construct
KVSSRFS

SEQ ID NO: 142 - protein, 9 aa, Synthetic construct
FQGSHFPLT

SEQ ID NO: 143 - protein, 111 aa, Synthetic construct
VVMTQTPLSLSVSLGDQASISCKSSQSIVHYNENTYLEWYLQKPGQSPQLLIYKVSSRFSGVPDRFSGSGSGTDFTLKISRVEAEDLGVYYCFQGSHFPLTFGQGTKLEIK

SEQ ID NO: 144 - protein, 218 aa, Synthetic construct
VVMTQTPLSLSVSLGDQASISCKSSQSIVHYNENTYLEWYLQKPGQSPQLLIYKVSSRFSGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 5 aa, Synthetic construct
DYYMA

SEQ ID NO: 146 - protein, 17 aa, Synthetic construct
NINYDGSRTDLLDSLKS

SEQ ID NO: 147 - protein, 10 aa, Synthetic construct
DTSRGSYFDV

SEQ ID NO: 148 - protein, 119 aa, Synthetic construct
EVQLVESGGGLVQPGGSLRLSCAASGFTFSDYYMAWVRQAPGKGLEWVANINYDGSRTDLLDSLKSRFTISRDNAKNSVYLQMNSLRAEDTAVYYCARDTSRGSYFDVWGAGTTVTVSS

SEQ ID NO: 149 - protein, 448 aa, Synthetic construct
EVQLVESGGGLVQPGGSLRLSCAASGFTFSDYYMAWVRQAPGKGLEWVANINYDGSRTDLLDSLKSRFTISRDNAKNSVYLQMNSLRAEDTAVYYCARDTSRGSYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 150 - protein, 16 aa, Synthetic construct
KSSQSIVHYNENTYLE

SEQ ID NO: 151 - protein, 7 aa, Synthetic construct
KVSSRAS

SEQ ID NO: 152 - protein, 9 aa, Synthetic construct
FQGSHFPLT

SEQ ID NO: 153 - protein, 111 aa, Synthetic construct
VVMTQTPLSLSVSLGDQASISCKSSQSIVHYNENTYLEWYLQKPGQSPQLLIYKVSSRASGVPDRFSGSGSGTDFTLKISRVEAEDLGVYYCFQGSHFPLTFGQGTKLEIK

SEQ ID NO: 154 - protein, 218 aa, Synthetic construct
VVMTQTPLSLSVSLGDQASISCKSSQSIVHYNENTYLEWYLQKPGQSPQLLIYKVSSRASGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 5 aa, Synthetic construct
DYYMA

SEQ ID NO: 156 - protein, 17 aa, Synthetic construct
NINYEGSRTDLLDSLKS

SEQ ID NO: 157 - protein, 10 aa, Synthetic construct
DTSRGSYFDV

SEQ ID NO: 158 - protein, 119 aa, Synthetic construct
EVQLVESGGGLVQPGGSLRLSCAASGFTFSDYYMAWVRQAPGKGLEWVANINYEGSRTDLLDSLKSRFTISRDNAKNSVYLQMNSLRAEDTAVYYCARDTSRGSYFDVWGAGTTVTVSS

SEQ ID NO: 159 - protein, 448 aa, Synthetic construct
EVQLVESGGGLVQPGGSLRLSCAASGFTFSDYYMAWVRQAPGKGLEWVANINYEGSRTDLLDSLKSRFTISRDNAKNSVYLQMNSLRAEDTAVYYCARDTSRGSYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 160 - protein, 16 aa, Synthetic construct
RSSQSIVHYNENTYLE

SEQ ID NO: 161 - protein, 7 aa, Synthetic construct
KVSNRFS

SEQ ID NO: 162 - protein, 9 aa, Synthetic construct
FQGSHFPLT

SEQ ID NO: 163 - protein, 111 aa, Synthetic construct
VVMTQTPLSLPVSLGDQASISCRSSQSIVHYNENTYLEWYLQKPGQSPQLLIYKVSNRFSGVPDRFSGSGSGTDFTLKISRVEAEDLGVYYCFQGSHFPLTFGQGTKLEIK

SEQ ID NO: 164 - protein, 218 aa, Synthetic construct
VVMTQTPLSLPVSLGDQASISCRSSQSIVHYNENTYLEWYLQKPGQSPQLLIYKVSNRFSGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 5 aa, Synthetic construct
DYYMA

SEQ ID NO: 166 - protein, 17 aa, Synthetic construct
NINYDGSRTDLLESLKS

SEQ ID NO: 167 - protein, 10 aa, Synthetic construct
DTSRGSYFDV

SEQ ID NO: 168 - protein, 119 aa, Synthetic construct
EVQLVESGGGLVQPGGSLRLSCAASGFTFSDYYMAWVRQAPGKGLEWVANINYDGSRTDLLESLKSRFTISRDNAKNSVYLQMNSLRAEDTAVYYCARDTSRGSYFDVWGAGTTVTVSS

SEQ ID NO: 169 - protein, 448 aa, Synthetic construct
EVQLVESGGGLVQPGGSLRLSCAASGFTFSDYYMAWVRQAPGKGLEWVANINYDGSRTDLLESLKSRFTISRDNAKNSVYLQMNSLRAEDTAVYYCARDTSRGSYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 170 - protein, 16 aa, Synthetic construct
RSSQSIVHYNANTYLE

SEQ ID NO: 171 - protein, 7 aa, Synthetic construct
KVSNRFS

SEQ ID NO: 172 - protein, 9 aa, Synthetic construct
FQGSHFPLT

SEQ ID NO: 173 - protein, 111 aa, Synthetic construct
VVMTQTPLSLPVSLGDQASISCRSSQSIVHYNANTYLEWYLQKPGQSPQLLIYKVSNRFSGVPDRFSGSGSGTDFTLKISRVEAEDLGVYYCFQGSHFPLTFGQGTKLEIK

SEQ ID NO: 174 - protein, 218 aa, Synthetic construct
VVMTQTPLSLPVSLGDQASISCRSSQSIVHYNANTYLEWYLQKPGQSPQLLIYKVSNRFSGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 5 aa, Synthetic construct
DYYMA

SEQ ID NO: 176 - protein, 17 aa, Synthetic construct
NINYDGSRTDLLDSLKS

SEQ ID NO: 177 - protein, 10 aa, Synthetic construct
DTSRGSYFDV

SEQ ID NO: 178 - protein, 119 aa, Synthetic construct
EVKLVESGGGLVQPGGSLRLSCAASGFTFSDYYMAWVRQAPGKGLEWVANINYDGSRTDLLDSLKSRFTISRDNAKNSVYLQMNSLRAEDTAVYYCARDTSRGSYFDVWGAGTTVTVSS

SEQ ID NO: 179 - protein, 448 aa, Synthetic construct
EVKLVESGGGLVQPGGSLRLSCAASGFTFSDYYMAWVRQAPGKGLEWVANINYDGSRTDLLDSLKSRFTISRDNAKNSVYLQMNSLRAEDTAVYYCARDTSRGSYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 180 - protein, 16 aa, Synthetic construct
RSSQSIVHYNTNTYLE

SEQ ID NO: 181 - protein, 7 aa, Synthetic construct
KVSNRFS

SEQ ID NO: 182 - protein, 9 aa, Synthetic construct
FQGSHFPLT

SEQ ID NO: 183 - protein, 111 aa, Synthetic construct
VVMTQTPLSLPVSLGDQASISCRSSQSIVHYNTNTYLEWYLQKPGQSPQLLIYKVSNRFSGVPDRFSGSGSGTDFTLKISRVEAEDLGVYYCFQGSHFPLTFGQGTKLEIK

SEQ ID NO: 184 - protein, 218 aa, Synthetic construct
VVMTQTPLSLPVSLGDQASISCRSSQSIVHYNTNTYLEWYLQKPGQSPQLLIYKVSNRFSGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 185-189 - skipped (5 entries)

SEQ ID NO: 190 - protein, 16 aa, Synthetic construct
RSSQSIVHYNGKTYLE

SEQ ID NO: 191 - protein, 7 aa, Synthetic construct
KVSNRFS

SEQ ID NO: 192 - protein, 9 aa, Synthetic construct
FQGSHFPLT

SEQ ID NO: 193 - protein, 111 aa, Synthetic construct
VVMTQTPLSLPVSLGDQASISCRSSQSIVHYNGKTYLEWYLQKPGQSPQLLIYKVSNRFSGVPDRFSGSGSGTDFTLKISRVEAEDLGVYYCFQGSHFPLTFGQGTKLEIK

SEQ ID NO: 194 - protein, 218 aa, Synthetic construct
VVMTQTPLSLPVSLGDQASISCRSSQSIVHYNGKTYLEWYLQKPGQSPQLLIYKVSNRFSGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195-199 - skipped (5 entries)

SEQ ID NO: 200 - protein, 16 aa, Synthetic construct
RSSQSIVHYNEKTYLE

SEQ ID NO: 201 - protein, 7 aa, Synthetic construct
KVSNRFS

SEQ ID NO: 202 - protein, 9 aa, Synthetic construct
FQGSHFPLT

SEQ ID NO: 203 - protein, 111 aa, Synthetic construct
VVMTQTPLSLPVSLGDQASISCRSSQSIVHYNEKTYLEWYLQKPGQSPQLLIYKVSNRFSGVPDRFSGSGSGTDFTLKISRVEAEDLGVYYCFQGSHFPLTFGQGTKLEIK

SEQ ID NO: 204 - protein, 218 aa, Synthetic construct
VVMTQTPLSLPVSLGDQASISCRSSQSIVHYNEKTYLEWYLQKPGQSPQLLIYKVSNRFSGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205-209 - skipped (5 entries)

SEQ ID NO: 210 - protein, 16 aa, Synthetic construct
KSSQSIVHYNGNTYLE

SEQ ID NO: 211 - protein, 7 aa, Synthetic construct
KVSSRFS

SEQ ID NO: 212 - protein, 9 aa, Synthetic construct
FQGSHFPLT

SEQ ID NO: 213 - protein, 111 aa, Synthetic construct
VVMTQTPLSLSVSLGDQASISCKSSQSIVHYNGNTYLEWYLQKPGQSPQLLIYKVSSRFSGVPDRFSGSGSGTDFTLKISRVEAEDLGVYYCFQGSHFPLTFGQGTKLEIK

SEQ ID NO: 214 - protein, 218 aa, Synthetic construct
VVMTQTPLSLSVSLGDQASISCKSSQSIVHYNGNTYLEWYLQKPGQSPQLLIYKVSSRFSGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215-219 - skipped (5 entries)

SEQ ID NO: 220 - protein, 16 aa, Synthetic construct
KSSQSIVHYNGNTYLE

SEQ ID NO: 221 - protein, 7 aa, Synthetic construct
KVSSRAS

SEQ ID NO: 222 - protein, 9 aa, Synthetic construct
FQGSHFPLT

SEQ ID NO: 223 - protein, 111 aa, Synthetic construct
VVMTQTPLSLSVSLGDQASISCKSSQSIVHYNGNTYLEWYLQKPGQSPQLLIYKVSSRASGVPDRFSGSGSGTDFTLKISRVEAEDLGVYYCFQGSHFPLTFGQGTKLEIK

SEQ ID NO: 224 - protein, 218 aa, Synthetic construct
VVMTQTPLSLSVSLGDQASISCKSSQSIVHYNGNTYLEWYLQKPGQSPQLLIYKVSSRASGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 225-229 - skipped (5 entries)

SEQ ID NO: 230 - protein, 16 aa, Synthetic construct
KSSQSIVHYNGNTYLE

SEQ ID NO: 231 - protein, 7 aa, Synthetic construct
KVSSRAS

SEQ ID NO: 232 - protein, 9 aa, Synthetic construct
FQGSHFPLT

SEQ ID NO: 233 - protein, 112 aa, Synthetic construct
DIVMTQTPLSLSVSLGDQASISCKSSQSIVHYNGNTYLEWYLQKPGQSPQLLIYKVSSRASGVPDRFSGSGSGTDFTLKISRVEAEDLGVYYCFQGSHFPLTFGQGTKLEIK

SEQ ID NO: 234 - protein, 219 aa, Synthetic construct
DIVMTQTPLSLSVSLGDQASISCKSSQSIVHYNGNTYLEWYLQKPGQSPQLLIYKVSSRASGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235-239 - skipped (5 entries)

SEQ ID NO: 240 - protein, 16 aa, Synthetic construct
KSSQSIVHYNEKTYLE

SEQ ID NO: 241 - protein, 7 aa, Synthetic construct
KVSSRAS

SEQ ID NO: 242 - protein, 9 aa, Synthetic construct
FQGSHFPLT

SEQ ID NO: 243 - protein, 112 aa, Synthetic construct
DIVMTQTPLSLSVSLGDQASISCKSSQSIVHYNEKTYLEWYLQKPGQSPQLLIYKVSSRASGVPDRFSGSGSGTDFTLKISRVEAEDLGVYYCFQGSHFPLTFGQGTKLEIK

SEQ ID NO: 244 - protein, 219 aa, Synthetic construct
DIVMTQTPLSLSVSLGDQASISCKSSQSIVHYNEKTYLEWYLQKPGQSPQLLIYKVSSRASGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245-249 - skipped (5 entries)

SEQ ID NO: 250 - protein, 16 aa, Synthetic construct
RSSQSIVHYNGNTYLE

SEQ ID NO: 251 - protein, 7 aa, Synthetic construct
KVSNRDS

SEQ ID NO: 252 - protein, 9 aa, Synthetic construct
FQGSHFPLT

SEQ ID NO: 253 - protein, 111 aa, Synthetic construct
VVMTQTPLSLPVSLGDQASISCRSSQSIVHYNGNTYLEWYLQKPGQSPQLLIYKVSNRDSGVPDRFSGSGSGTDFTLKISRVEAEDLGVYYCFQGSHFPLTFGQGTKLEIK

SEQ ID NO: 254 - protein, 218 aa, Synthetic construct
VVMTQTPLSLPVSLGDQASISCRSSQSIVHYNGNTYLEWYLQKPGQSPQLLIYKVSNRDSGVPDRFSGSGSGTDFTLKISRVEAEDLGVYYCFQGSHFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 255-299 - skipped (45 entries)

SEQ ID NO: 300 - protein, 16 aa, Synthetic construct
KSSQSIVHYNEKTYLE

SEQ ID NO: 301 - protein, 16 aa, Synthetic construct
KSSQSIVHYNGKTYLE

SEQ ID NO: 302 - protein, 16 aa, Synthetic construct
KSSQSIVHYNENTYLE

SEQ ID NO: 303 - protein, 16 aa, Synthetic construct
RSSQSIVHYNENTYLE

SEQ ID NO: 304 - protein, 16 aa, Synthetic construct
RSSQSIVHYNANTYLE

SEQ ID NO: 305 - protein, 16 aa, Synthetic construct
RSSQSIVHYNTNTYLE

SEQ ID NO: 306 - protein, 16 aa, Synthetic construct
RSSQSIVHYNGKTYLE

SEQ ID NO: 307 - protein, 16 aa, Synthetic construct
RSSQSIVHYNEKTYLE

SEQ ID NO: 308 - protein, 16 aa, Synthetic construct
KSSQSIVHYNGNTYLE

SEQ ID NO: 309 - protein, 16 aa, Synthetic construct
RSSQSIVHYNGNTYLE

SEQ ID NO: 310 - protein, 11 aa, Synthetic construct
QSIVHYNEKTY

SEQ ID NO: 311 - protein, 11 aa, Synthetic construct
QSIVHYNGKTY

SEQ ID NO: 312 - protein, 11 aa, Synthetic construct
QSIVHYNENTY

SEQ ID NO: 313 - protein, 11 aa, Synthetic construct
QSIVHYNANTY

SEQ ID NO: 314 - protein, 11 aa, Synthetic construct
QSIVHYNTNTY

SEQ ID NO: 315 - protein, 11 aa, Synthetic construct
QSIVHYNGNTY

SEQ ID NO: 316 - protein, 7 aa, Synthetic construct
KVSSRFS

SEQ ID NO: 317 - protein, 7 aa, Synthetic construct
KVSSRAS

SEQ ID NO: 318 - protein, 7 aa, Synthetic construct
KVSNRFS

SEQ ID NO: 319 - protein, 7 aa, Synthetic construct
KVSNRDS

SEQ ID NO: 320 - skipped

SEQ ID NO: 321 - protein, 9 aa, Synthetic construct
FQGSHFPLT

SEQ ID NO: 322-329 - skipped (8 entries)

SEQ ID NO: 330 - protein, 5 aa, Synthetic construct
DYYMA

SEQ ID NO: 331 - protein, 8 aa, Synthetic construct
GFTFSDYY

SEQ ID NO: 332 - protein, 10 aa, Synthetic construct
GFTFSDYYMA

SEQ ID NO: 333 - protein, 7 aa, Synthetic construct
GFTFSDY

SEQ ID NO: 334 - protein, 17 aa, Synthetic construct
NINYEGSRTDLLESLKS

SEQ ID NO: 335 - protein, 17 aa, Synthetic construct
NINYYGSRTDLLESLKS

SEQ ID NO: 336 - protein, 17 aa, Synthetic construct
NINYEGSRTDLLASLKS

SEQ ID NO: 337 - protein, 17 aa, Synthetic construct
NINYYGSRTDLLASLKS

SEQ ID NO: 338 - protein, 17 aa, Synthetic construct
NINYDGSRTDLLDSLKS

SEQ ID NO: 339 - protein, 17 aa, Synthetic construct
NINYEGSRTDLLDSLKS

SEQ ID NO: 340 - protein, 17 aa, Synthetic construct
NINYDGSRTDLLESLKS

SEQ ID NO: 341 - protein, 8 aa, Synthetic construct
INYEGSRT

SEQ ID NO: 342 - protein, 8 aa, Synthetic construct
INYYGSRT

SEQ ID NO: 343 - protein, 8 aa, Synthetic construct
INYDGSRT

SEQ ID NO: 344 - protein, 6 aa, Synthetic construct
NYEGSR

SEQ ID NO: 345 - protein, 6 aa, Synthetic construct
NYYGSR

SEQ ID NO: 346 - protein, 6 aa, Synthetic construct
NYDGSR

SEQ ID NO: 347 - protein, 10 aa, Synthetic construct
DTSRGSYFDV

SEQ ID NO: 348 - protein, 12 aa, Synthetic construct
ARDTSRGSYFDV

SEQ ID NO: 349 - protein, 8 aa, Synthetic construct
TSRGSYFD

(19) United States
(12) Patent Application Publication
(10) Pub. No.: US 2026/0347454 A1
(43) Pub. Date: Nov. 5, 2026

(54) ANTIBODIES TO CONNECTIVE TISSUE GROWTH FACTOR (CTGF) AND USES THEREOF

(71) Applicant: Boehringer Ingelheim International GmbH, Ingelheim am Rhein (DE)

(72) Inventors: Andreas-David BRUNNER, Mittelbiberach (DE); Lars DITTUS, Mittelbiberach (DE); Priyanka GUPTA, Newtown, CT (US); Muriel LIZE, Ulm (DE); Maria Bonatsakis MYZITHRAS, Sandy Hook, CT (US); Irina RYBINA, Sleepy Hollow, NY (US); Julia SAUER, Biberach (DE); Wioletta Anna SKRONSKA-WASEK, Biberach (DE); Heiko Friedrich STAHL, Biberach (DE)

(21) Appl. No.: 19/560,471

(22) Filed: Mar. 9, 2026

Related U.S. Application Data
(62) Division of application No. 19/065,094, filed on Feb. 27, 2025, now Pat. No. 12,600,770.
(60) Provisional application No. 63/723,664, filed on Nov. 22, 2024.

(30) Foreign Application Priority Data
Mar. 1, 2024 (EP) 24160771.2

(51) Int. Cl. C07K 16/22

(52) U.S. Cl. CPC C07K 16/22; C07K 2317/24; C07K 2317/76; C07K 2317/92

(57) ABSTRACT

The present disclosure generally relates to anti-CTGF (connective tissue growth factor) antibodies or antigen-binding fragments thereof. Exemplary anti-CTGF antibodies disclosed herein address the need for treatments of conditions modulated by CTGF signaling. In some aspects, the anti-CTGF antibodies or antigen-binding fragments thereof are for diagnostic and/or therapeutic use, for example in a subject in need thereof, such as a human.

### CROSS-REFERENCE TO RELATED APPLICATIONS

This application is a divisional of U.S. patent application Ser. No. 19/065,094, filed Feb. 27, 2025, which claims priority to European Patent Application No. EP24160771.2, filed Mar. 1, 2024, and U.S. Provisional Patent Application Ser. No. 63/723,664, filed Nov. 22, 2024, each of which is hereby incorporated by reference in its entirety.

### SEQUENCE LISTING

This application includes, as part of its disclosure, a “Sequence Listing XML” pursuant to 37 C.F.R. § 1.831 (a) which is submitted in XML file format in a file named “09-0753-US-3.xml”, created on Mar. 6, 2026, and having a size of 203,519 bytes, which is hereby incorporated by reference herein in its entirety.

### FIELD

This disclosure generally relates to anti-CTGF (connective tissue growth factor) antibodies. More specifically, the disclosure relates to anti-CTGF antibodies or antigen-binding fragments thereof and methods of their use for the treatment of various diseases or disorders, for example, respiratory disease, fibrosis, or cancer. Pharmaceutical compositions and kits comprising the anti-CTGF antibodies or antigen-binding fragments thereof are also disclosed.

### BACKGROUND

Idiopathic pulmonary fibrosis (IPF) is a chronic, fibrosing interstitial pneumonia of unknown etiology. It is characterized by progressive worsening of dyspnea and lung function, and has a poor prognosis. Standard of care (nintedanib, pirfenidone) can reduce the decline trajectory, nevertheless, life expectancy after diagnosis remains strongly shortened.

Progressive pulmonary fibrosis (PPF), of which IPF is a prototype, is a fatal condition that can develop in a variety of diseases, including inflammatory diseases such as rheumatoid arthritis or scleroderma.

CTGF is a secreted growth factor that modulates many profibrotic processes and is strongly upregulated in IPF and PPF. CTGF is composed of two N-terminal domains (together CTGF-NTF) and two C-terminal domains (together CTGF-CTF) bound by a hinge peptide containing cleavage sequences for matrix metalloproteases (MMP). CTGF is secreted as a full length protein of 38 kDa, and can be rapidly cleaved into CTGF-NTF and CTGF-CTF when exposed to activated matrix metalloproteases. Those proteases are known to be increased in disease.

CTGF has been proposed as prognostic biomarkers of progression in IPF. However, the precise mechanisms by which CTGF or the CTGF fragments may contribute to the pathobiology of pulmonary fibrosis remains unclear.

Current treatment methods in IPF and PPF (nintedanib and pirfenidone) slow progression of pulmonary fibrosis. However, the median treatment duration with both antifibrotics is not much longer than one year. Thus, there is an unmet need for additional therapies for IPF and PPF.

### BRIEF SUMMARY

The present disclosure addresses the above need and others by providing agents, in particular antibodies or antigen-binding fragments thereof, which specifically bind to CTGF. In particular, the antibodies or antigen-binding fragments thereof as disclosed herein specifically bind to human CTGF, such as full length CTGF (e.g., having the amino acid sequence of SEQ ID NO: 1 or SEQ ID NO: 2) or CTGF N-terminal fragment (CTGF-NTF) (e.g., having the amino acid sequence of SEQ ID NO: 3). In some aspects, the antibodies or antigen-binding fragments thereof block proteolytic cleavage of CTGF, e.g., by matrix metalloprotease 7 (MMP7). In some aspects, the antibodies or antigen-binding fragments thereof, block the interaction of CTGF with cells that express CTGF receptors, such as alveolar type II cells, fibroblasts and A549 cells. In some aspects, the antibodies or antigen-binding fragments thereof, block the interaction of CTGF-NTF with cells that express CTGF receptors, such as alveolar type II cells, fibroblasts and A549 cells.

The cleavage of CTGF by proteases, such as MMP7, activates the CTGF pathway since the fragments are biologically active. Exemplary anti-CTGF antibodies of the present disclosure bind to CTGF and inhibit this cleavage. They may, thus, reduce the occurrence of fragments. Exemplary anti-CTGF antibodies of the present disclosure additionally specifically bind CTGF-NTF. This inhibition of cleavage and binding of full length CTGF and CTGF-NTF thus reduce fibrogenesis and fibrillar collagen production. Exemplary anti-CTGF antibodies of the present disclosure can reduce levels of the human fibrosis biomarkers such as ProC3.

Macrophages engulfing CTGF fragments show reduced efferocytosis potential. Consequently, dead/senescent cells accumulate and perpetuate profibrotic signaling. Exemplary anti-CTGF antibodies of the present disclosure inhibit the generation of CTGF fragments. Thereby, macrophage homeostasis is promoted.

Alveolar cells undergo epithelial-mesenchymal transition in pulmonary fibrosis leading to a failure of the progenitor cells to repair the alveolus. Exemplary anti-CTGF antibodies of the present disclosure inhibit this epithelial-mesenchymal transition of alveolar cells. Thereby, the maintenance and functionality of the alveolar stem cells and thus repair of the epithelium is promoted.

In some aspects, the anti-CTGF antibody or antigen-binding fragment thereof comprises a light chain variable region comprising a light chain complementarity determining region 1 (L-CDR1) having the amino acid sequence of Y1-SSQSIVHYN-Y2-Y3-TYLE where Y1 is Kor R, Y2 is A, E, G, or T and Y3 is K or N (SEQ ID NO: 10), a light chain complementarity determining region 2 (L-CDR2) having the amino acid sequence of KVS-Z1-R-Z2-S where Z1 is N or S and Z2 is A or F (SEQ ID NO: 11), and a light chain complementarity determining region 3 (L-CDR3) having the amino acid sequence of FQGSHFPLT (SEQ ID NO: 12); and a heavy chain variable region comprising a heavy chain complementarity determining region 1 (H-CDR1) having the amino acid sequence of DYYMA (SEQ ID NO: 15), a heavy chain complementarity determining region 2 (H-CDR2) having the amino acid sequence of NINY-X1-GSRTDLL-X2-SLKS where X1 is D, E, or Y and X2 is A, D, or E (SEQ ID NO: 16), and a heavy chain complementarity determining region 3 (H-CDR3) having the amino acid sequence of DTSRGSYFDV (SEQ ID NO: 17).

In some aspects, the anti-CTGF antibody or antigen-binding fragment thereof comprises the six CDRs (L-CDR1, L-CDR2, L-CDR3, H-CDR1, H-CDR2 and H-CDR3) of any one of antibodies A1-A127 as specified in Table 13 (according to Kabat). Preferably, said antibody comprises the six CDRs (L-CDR1, L-CDR2, L-CDR3, H-CDR1, H-CDR2 and H-CDR3) of any one of antibodies A1-A25, A41-A44, A46, A48-A85, or A87-A127 as specified in Table 13 (according to Kabat). Most preferably, said antibody comprises the six CDRs (L-CDR1, L-CDR2, L-CDR3, H-CDR1, H-CDR2 and H-CDR3) of antibody A97 as specified in Table 13 (according to Kabat). While the specific CDR sequences mentioned in Table 13 are according to Kabat, it is understood that the CDR sequences of antibodies A1-A127 of Table 13 may also be determined according to another numbering scheme (e.g., as described herein) based on the sequences of the variable regions or the full-length sequences of the heavy and light chains as outlined for antibodies A1-A127 in Table 13, e.g., by reference to the CDR sequences as shown in Tables 3-8 and the light and heavy chain names corresponding to each antibody as shown in Tables 9-12.

In some aspects, the anti-CTGF antibody or antigen-binding fragment thereof comprises a light chain variable region and a heavy chain variable region each having at least 95% identity to the light chain variable region and the corresponding heavy chain variable region, respectively, of any one of antibodies A1-A127 as specified in Table 13. Preferably, said antibody comprises a light chain variable region and a heavy chain variable region each having at least 95% identity to the light chain variable region and the corresponding heavy chain variable region, respectively, of any one of antibodies A1-A25, A41-A44, A46, A48-A85, or A87-A127 as specified in Table 13. Most preferably, said antibody comprises a light chain variable region and a heavy chain variable region each having at least 95% identity to the light chain variable region and the corresponding heavy chain variable region, respectively, of antibody A97 as specified in Table 13.

In some aspects, the anti-CTGF antibody or antigen-binding fragment thereof comprises the light chain variable region and the corresponding heavy chain variable region of any one of antibodies A1-A127 as specified in Table 13. Preferably, said antibody comprises the light chain variable region and the corresponding heavy chain variable region of any one of antibodies A1-A25, A41-A44, A46, A48-A85, or A87-A127 as specified in Table 13. Most preferably, said antibody comprises the light chain variable region and the corresponding heavy chain variable region of antibody A97 as specified in Table 13.

In some aspects, the anti-CTGF antibody or antigen-binding fragment thereof comprises a light chain and a heavy chain each having at least 95% identity to the light chain and the corresponding heavy chain, respectively, of any one of antibodies A1-A127 as specified in Table 13. Preferably, said antibody comprises a light chain and a heavy chain each having at least 95% identity to the light chain and the corresponding heavy chain, respectively, of any one of antibodies A1-A25, A41-A44, A46, A48-A85, or A87-A127 as specified in Table 13. Most preferably, said antibody comprises a light chain and a heavy chain each having at least 95% identity to the light chain and the corresponding heavy chain, respectively, of antibody A97 as specified in Table 13.

In some aspects, the anti-CTGF antibody or antigen-binding fragment thereof comprises the light chain and the corresponding heavy chain of any one of antibodies A1-A127 as specified in Table 13. Preferably, said antibody comprises the light chain and the corresponding heavy chain of any one of antibodies A1-A25, A41-A44, A46, A48-A85, or A87-A127 as specified in Table 13. Most preferably, said antibody comprises the light chain and the corresponding heavy chain of antibody A97 as specified in Table 13.

In some aspects, the anti-CTGF antibody or antigen-binding fragment thereof as described herein comprises a heavy chain constant region selected from IgG₁, IgG₂, IgG₃, IgG₄, IgM, IgA and IgE or a variant thereof. Preferably, the anti-CTGF antibody or antigen-binding fragment thereof as described herein comprises an IgG₁ heavy chain constant region.

In some aspects, the anti-CTGF antibody or antigen-binding fragment thereof comprises a heavy chain lacking a C-terminal lysine residue.

In some aspects, the anti-CTGF antibody or antigen-binding fragment thereof comprises a heavy chain constant region wherein the heavy chain constant region is of an IgG₁ with L234A and L235A substitutions.

In some aspects, the anti-CTGF antibody or antigen-binding fragment thereof comprises a light chain constant region selected from the group consisting of a kappa and a lambda light chain.

In some aspects, the anti-CTGF antibody or antigen-binding fragment thereof is a monoclonal antibody.

In some aspects, the anti-CTGF antibody or antigen-binding fragment thereof is a monospecific antibody.

In some aspects, the anti-CTGF antibody or antigen-binding fragment thereof binds the full length human CTGF of SEQ ID NO: 2 with a binding affinity of 50 pM or lower, 25 pM or lower, 15 pM or lower, 10 pM or lower, 5 pM or lower, or 2 pM or lower.

In some aspects, the anti-CTGF antibody or antigen-binding fragment thereof binds the human CTGF-NTF of SEQ ID NO: 3 with a binding affinity of 10 nM or lower, 5 nM or lower, 2.5 nM or lower, 1 nM or lower, 0.5 nM or lower, or 0.2 nM or lower.

In some aspects, the anti-CTGF antibody or antigen-binding fragment thereof binds the human CTGF of SEQ ID NO: 2 with a K_{D} of 5 pM or lower.

In some aspects, the anti-CTGF antibody or antigen-binding fragment thereof binds the N-terminal fragment of human CTGF of SEQ ID NO: 3 with a K_{D} of 0.5 nM or lower.

In some aspects, the anti-CTGF antibody or antigen-binding fragment thereof decreases or blocks human CTGF binding to primary human fibroblast cells.

In some aspects, the anti-CTGF antibody or antigen-binding fragment thereof decreases or blocks cleavage of human CTGF by MMP7.

In some aspects, the anti-CTGF antibody or antigen-binding fragment thereof blocks or inhibits human TGFbeta-induced increase in COL1A1 expression in lung epithelial cells.

In some aspects, the anti-CTGF antibody or antigen-binding fragment thereof blocks or inhibits CTGF interaction with alveolar type II cells, fibroblasts and/or A549 cells.

In some aspects, the anti-CTGF antibody or antigen-binding fragment thereof blocks or inhibits CTGF-NTF interaction with alveolar type II cells, fibroblasts and/or A549 cells.

In some aspects, the disclosure provides a pharmaceutical composition comprising an anti-CTGF antibody or antigen-binding fragment thereof as disclosed herein, and a pharmaceutically acceptable carrier.

In some aspects, the disclosure provides an anti-CTGF antibody or antigen-binding fragment thereof as disclosed herein for use as a medicament.

In some aspects, the disclosure provides an anti-CTGF antibody or antigen-binding fragment thereof as disclosed herein for use for treating or preventing a CTGF-associated disorder, a fibrotic disease, hypertension, diabetes, myocardial infarction, arthritis, CTGF-related cell proliferative disease, atherosclerosis, glaucoma, or a cancer. Said fibrotic disease may be idiopathic pulmonary fibrosis (IPF), progressive pulmonary fibrosis (PPF), pulmonary fibrosis with interstitial lung disease (PF-ILD), systemic scleroderma (SSc), diabetic nephropathy, diabetic retinopathy, osteoarthritis, scleroderma, chronic heart failure, liver cirrhosis or renal fibrosis. Said cancer may be acute lymphoblastic leukemia, dermatofibroma, breast cancer, angiolipoma, angioleiomyoma, connective tissue-generating cancer, prostate cancer, ovarian cancer, colorectal cancer, pancreatic cancer, gastrointestinal cancer or liver cancer. Said anti-CTGF antibody or antigen-binding fragment thereof may be for parenteral or inhalation administration.

In some aspects, the disclosure provides a method of treating or preventing a CTGF-associated disorder, a fibrotic disease, hypertension, diabetes, myocardial infarction, arthritis, CTGF-related cell proliferative disease, atherosclerosis, glaucoma, or a cancer, comprising administering an effective amount of an anti-CTGF antibody or antigen-binding fragment thereof as disclosed herein to a patient in need thereof. Said fibrotic disease may be idiopathic pulmonary fibrosis (IPF), progressive pulmonary fibrosis (PPF), pulmonary fibrosis with interstitial lung disease (PF-ILD), systemic scleroderma (SSc), diabetic nephropathy, diabetic retinopathy, osteoarthritis, scleroderma, chronic heart failure, liver cirrhosis or renal fibrosis. Said cancer may be acute lymphoblastic leukemia, dermatofibroma, breast cancer, angiolipoma, angioleiomyoma, connective tissue-generating cancer, prostate cancer, ovarian cancer, colorectal cancer, pancreatic cancer, gastrointestinal cancer or liver cancer. Said antibody or antigen-binding fragment thereof may be administered by parenteral or inhalation administration.

In some aspects, the disclosure provides the use of an anti-CTGF antibody or antigen-binding fragment thereof as disclosed herein for the manufacture of a medicament for treating or preventing CTGF-associated disorder, a fibrotic disease, hypertension, diabetes, myocardial infarction, arthritis, CTGF-related cell proliferative disease, atherosclerosis, glaucoma, or a cancer. Said fibrotic disease may be idiopathic pulmonary fibrosis (IPF), progressive pulmonary fibrosis (PPF), pulmonary fibrosis with interstitial lung disease (PF-ILD), systemic scleroderma (SSc), diabetic nephropathy, diabetic retinopathy, osteoarthritis, scleroderma, chronic heart failure, liver cirrhosis or renal fibrosis. Said cancer may be acute lymphoblastic leukemia, dermatofibroma, breast cancer, angiolipoma, angioleiomyoma, connective tissue-generating cancer, prostate cancer, ovarian cancer, colorectal cancer, pancreatic cancer, gastrointestinal cancer or liver cancer. Said anti-CTGF antibody or antigen-binding fragment thereof may be administered by parenteral or inhalation administration.

In some aspects, the disclosure provides an isolated polynucleotide or a plurality of isolated nucleotides that encodes a sequence comprising the heavy chain variable region and/or light chain variable region of an anti-CTGF antibody or antigen-binding fragment thereof as disclosed herein.

In some aspects, the disclosure provides a vector or plurality of vectors comprising a polynucleotide or a plurality of polynucleotides that encode the light chain and/or the heavy chain of an anti-CTGF antibody or antigen-binding fragment thereof as disclosed herein. Said vector may comprise an expression vector or plurality of expression vectors. Said polynucleotide or plurality of polynucleotides may be in functional association with one or more expression control sequences.

In some aspects, the disclosure provides a host cell comprising a polynucleotide or plurality of nucleotides, or a vector or plurality of vectors, that encode the light chain and/or the heavy chain of an anti-CTGF antibody or antigen-binding fragment thereof as disclosed herein.

In some aspects, the disclosure provides a method for the production of an anti-CTGF antibody or antigen-binding fragment thereof as disclosed herein, comprising the steps: (a) cultivating a host cell comprising a polynucleotide or plurality of nucleotides, or a vector or plurality of vectors, that encode the light chain and the heavy chain of said anti-CTGF antibody or antigen-binding fragment thereof, under conditions allowing the expression of said anti-CTGF antibody or antigen-binding fragment thereof; and (b) recovering said anti-CTGF antibody or antigen-binding fragment thereof.

In some aspects, the disclosure provides a method of inhibiting the interaction between human CTGF and a cell, comprising contacting said human CTGF with (an effective amount of) the anti-CTGF antibody or antigen-binding fragment thereof as disclosed herein.

In some aspects, the disclosure provides a method of inhibiting the interaction between human CTGF-NTF and a cell, comprising contacting said human CTGF-NTF with (an effective amount of) the anti-CTGF antibody or antigen-binding fragment thereof as disclosed herein.

### BRIEF DESCRIPTION OF THE DRAWINGS

FIG. 1. Normalized full length intact CTGF signal compared to each reaction with addition of MMP7 and MMP7+antibodies. Antibody 97 compared to isotype control: (p<0.005). Antibody F1 compared to isotype control: (p<0.01).

FIG. 2A-2B. Measurement of N-terminal CTGF (FIG. 2A) and full length CTGF (FIG. 2B) in samples from healthy volunteers (“H/V”) and IPF patients (“IPF”). N-terminal CTGF concentration was significantly elevated in IPF patients (p≤0.05).

FIG. 3. Measurement of MMP7 in samples from healthy volunteers (“H/V”) and IPF patients (“IPF”). MMP7 concentration was significantly elevated in IPF patients (p≤0.0001).

FIG. 4A and FIG. 4B. MSD-ELISA analysis of smooth muscle actin (a-SMA) in the IPF small airway epithelial cell (SAEC) air liquid interface (ALI) co-culture system. Fold change expression values are shown, normalized to untreated control fibroblast lysate samples. Note elevated levels of profibrotic marker a-SMA in the IPF cocktail-treated co-culture after 72 h. The anti-CTGF Antibodies and isotype controls were added 24 hours after IPF cocktail stimulation. Antibody A97 (FIG. 4A) and antibody A13 (FIG. 4B) each show a dose-dependent reduction of a-SMA expression, whereas antibody F1 and corresponding isotype control antibodies does not show significant effects. (n=3 biological replicates performed in n=3 individual experiments). Each assay point represents a technical co-culture well. (***p<0.001, **p<0.01, *p<0.05; one-way ANOVA).

FIG. 5. Vimentin staining intensity per nucleus in 2D cultured alveolar EMT assay following AT2 cell culture treatment with TGFβ1. (*p<0.05)

FIG. 6. COL1A1 expression levels in 2D cultured AT2 EMT assay. MSD-ELISA analysis of Collagen1a1 in 2D cultured AT2 EMT system. Fold change expression values shown, normalized to untreated control AT2 cell lysate samples. TGFB1 treatment induces increased COL1A1 expression. Antibody A97 significantly inhibits collagen production compared to corresponding isotype control. (n=2 donors measured as 3 individual replicates. Each assay point represents a lysate sample of a technical well (***p<0.001; one-way ANOVA; ns: not significant).

FIG. 7. Results of collagen III (ProC3) production inhibition in the supernatant of an air-liquid-interface cell culture, shown in arbitrary units. Fibrosis and collagen III production is stimulated by IPF-cocktail. Anti-CTGF antibodies A97 or F1 were used in concentrations of 1 uM, 500 nM, or 100 nM as shown. (***p<0.001; **p<0.01; ns: not significant).

FIG. 8. Global protein expression correlation analysis of air-liquid-interface supernatant. Zooming into the protein cluster containing CTGF (Gene name: CCN2) shows a strong correlation to collagen proteins and other extracellular markers. All proteins highlighted in this cluster have a very high protein expression profile correlation with each other with >0.8 according to Pearson. Note that the black coloring on the diagonal only reflects that each protein has a Pearson correlation of 1 with itself.

FIG. 9A and FIG. 9B. Free CTGF concentrations determined in samples from an IPF SAEC ALI coculture assay using a ligand binding assay on the Gyrolab platform. CTGF levels are determined using a biotinylated derivative of F1 for immobilization of CTGF and an Alexa-Fluor conjugate of A97 binding to a complementary epitope. Comparing A97 (FIG. 9A) and A13 (FIG. 9B), respectively, with F1 at equal concentrations shows significantly stronger binding of CTGF by the A97 and A13 in this assay. The bars show mean values of up to 54 individual datapoints and error bars represent the standard deviation. Samples shown on the x-axis (y-value=0) have values at or below the lower quantification limit.

FIG. 10. Primary human lung fibroblasts (N=2-6; different shapes indicate distinct replicates) are exposed to either labeled CTGF or a mixture of labeled CTGF pre-incubated with isotype control or anti-CTGF antibodies as indicated on the X-axis (blank label indicates a no antibody control). After 1 hour incubation cells are washed 3 times with PBS and scanned with Incucyte for RPE signal. Results show the level of labeled CTGF binding to cells, normalized to cell density, and shown relatively to the control. Means were compared using One-way ANOVA; **p≤0.01.

FIG. 11. Primary human alveolar type II cells (N=2-3; different shapes indicate distinct replicates) are exposed to either labeled CTGF or mixture of labeled CTGF pre-incubated with isotype control or anti-CTGF antibodies as indicated on the X-axis (blank label indicates a no antibody control). After 4 hours incubation cells are washed 3 times with PBS and scanned with Incucyte for RPE signal. Results show the level of labeled CTGF binding to cells, normalized to cell density, and shown relatively to the control. Means were compared using One-way ANOVA; *p≤0.05.

FIG. 12. A549 cells (n=2-10; different shapes indicate distinct replicates) are exposed to either labeled CTGF or mixture of labeled CTGF pre-incubated with isotype control or anti-CTGF antibodies as indicated on the X-axis (blank label indicates a no antibody control). After 1 hour incubation cells are washed 3 times with PBS and scanned with Incucyte for RPE signal. Results show the level of labeled CTGF binding to cells, normalized to cell density, and shown relatively to the control. Means were compared using One-way ANOVA; *p≤0.05, ****p≤0.0001

FIG. 13. QPCT gene expression in homeostatic (IL4/13) stimulated IDMs. Without fragments (−), FL CTGF and NTF CTGF fragment.

FIG. 14A-14B. Efferocytosis IDM (n=4, FIG. 14A) and MDM (n=2, FIG. 14B). One-way ANOVA with Tukey's multiple comparison test **p≤0.01.

### NUMBERED STATEMENTS

The following numbered paragraphs (paras) describe particular aspects and embodiments of the present disclosure:

1. An anti-CTGF antibody or an antigen-binding fragment thereof comprising:
a light chain variable region comprising a light chain complementarity determining region 1 (L-CDR1) having the amino acid sequence of Y1-SSQSIVHYN-Y2-Y3-TYLE where Y1 is K or R, Y2 is A, E, G, or T and Y3 is K or N (SEQ ID NO: 10), a light chain complementarity determining region 2 (L-CDR2) having the amino acid sequence of KVS-Z1-R-Z2-S where Z1 is N or S and Z2 is A or F (SEQ ID NO: 11), and a light chain complementarity determining region 3 (L-CDR3) having the amino acid sequence of FQGSHFPLT (SEQ ID NO: 12); and a heavy chain variable region comprising a heavy chain complementarity determining region 1 (H-CDR1) having the amino acid sequence of DYYMA (SEQ ID NO: 15), a heavy chain complementarity determining region 2 (H-CDR2) having the amino acid sequence of NINY-X1-GSRTDLL-X2-SLKS where X1 is D, E, or Y and X2 is A, D, or E (SEQ ID NO: 16), and a heavy chain complementarity determining region 3 (H-CDR3) having the amino acid sequence of DTSRGSYFDV (SEQ ID NO: 17).

2. The anti-CTGF antibody or an antigen-binding fragment thereof according to para 1, wherein the anti-CTGF antibody or an antigen-binding fragment thereof comprises:
i. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain 105, an H-CDR2 having the amino acid sequence of SEQ ID NO: 106, and an H-CDR3 having the amino acid sequence of SEQ ID NO: 107; or
ii. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
iii. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
iv. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
v. a light chain variable region comprising an L-CDR1 having the amino acid sequence of SEQ ID NO: 130, an L-CDR2 having the amino acid sequence of SEQ ID NO: 131, and an L-CDR3 having the amino acid sequence of SEQ ID NO: 132; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
vi. a light chain variable region comprising an L-CDR1 having the amino acid sequence of SEQ ID NO: 140, an L-CDR2 having the amino acid sequence of SEQ ID NO: 141, and an L-CDR3 having the amino acid sequence of SEQ ID NO: 142; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
vii. a light chain variable region comprising an L-CDR1 having the amino acid sequence of SEQ ID NO: 150, an L-CDR2 having the amino acid sequence of SEQ ID NO: 151, and an L-CDR3 having the amino acid sequence of SEQ ID NO: 152; and a heavy chain 105, an H-CDR2 having the amino acid sequence of SEQ ID NO: 106, and an H-CDR3 having the amino acid sequence of SEQ ID NO: 107; or
viii. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
ix. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
x. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
xi. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
xii. a light chain variable region comprising an L-CDR1 having the amino acid sequence of SEQ ID NO: 200, an L-CDR2 having the amino acid sequence of SEQ ID NO: 201, and an L-CDR3 having the amino acid sequence of SEQ ID NO: 202; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
xiii. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain 105, an H-CDR2 having the amino acid sequence of SEQ ID NO: 106, and an H-CDR3 having the amino acid sequence of SEQ ID NO: 107; or
xiv. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
xv. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
xvi. a light chain variable region comprising an L-CDR1 having the amino acid sequence of SEQ ID NO: 250, an L-CDR2 having the amino acid sequence of SEQ ID NO: 251, and an L-CDR3 having the amino acid sequence of SEQ ID NO: 252; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
xvii. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xviii. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xix. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain having the amino acid sequence of SEQ ID NO: 117; or
xx. a light chain variable region comprising an L-CDR1 having the amino acid sequence of SEQ ID NO: 130, an L-CDR2 having the amino acid sequence of SEQ ID NO: 131, and an L-CDR3 having the amino acid sequence of SEQ ID NO: 132; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxi. a light chain variable region comprising an L-CDR1 having the amino acid sequence of SEQ ID NO: 140, an L-CDR2 having the amino acid sequence of SEQ ID NO: 141, and an L-CDR3 having the amino acid sequence of SEQ ID NO: 142; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxii. a light chain variable region comprising an L-CDR1 having the amino acid sequence of SEQ ID NO: 150, an L-CDR2 having the amino acid sequence of SEQ ID NO: 151, and an L-CDR3 having the amino acid sequence of SEQ ID NO: 152; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxiii. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxiv. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxv. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain having the amino acid sequence of SEQ ID NO: 117; or
xxvi. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxvii. a light chain variable region comprising an L-CDR1 having the amino acid sequence of SEQ ID NO: 200, an L-CDR2 having the amino acid sequence of SEQ ID NO: 201, and an L-CDR3 having the amino acid sequence of SEQ ID NO: 202; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxviii. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxix. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxx. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxxi. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain having the amino acid sequence of SEQ ID NO: 117; or
xxxii. a light chain variable region comprising an L-CDR1 having the amino acid sequence of SEQ ID NO: 250, an L-CDR2 having the amino acid sequence of SEQ ID NO: 251, and an L-CDR3 having the amino acid sequence of SEQ ID NO: 252; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxxiii. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xxxiv. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xxxv. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xxxvi. a light chain variable region comprising an L-CDR1 having the amino acid sequence of SEQ ID NO: 130, an L-CDR2 having the amino acid sequence of SEQ ID NO: 131, and an L-CDR3 having the amino acid sequence of SEQ ID NO: 132; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xxxvii. a light chain variable region comprising an L-CDR1 having the amino acid sequence of SEQ ID NO: 140, an L-CDR2 having the amino acid sequence of SEQ ID NO: 141, and an L-CDR3 having the amino acid sequence of SEQ ID NO: 142; and a heavy chain 125, an H-CDR2 having the amino acid sequence of SEQ ID NO: 126, and an H-CDR3 having the amino acid sequence of SEQ ID NO: 127; or
xxxviii. a light chain variable region comprising an L-CDR1 having the amino acid sequence of SEQ ID NO: 150, an L-CDR2 having the amino acid sequence of SEQ ID NO: 151, and an L-CDR3 having the amino acid sequence of SEQ ID NO: 152; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xxxix. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xl. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xli. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xlii. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xliii. a light chain variable region comprising an L-CDR1 having the amino acid sequence of SEQ ID NO: 200, an L-CDR2 having the amino acid sequence of SEQ ID NO: 201, and an L-CDR3 having the amino acid sequence of SEQ ID NO: 202; and a heavy chain 125, an H-CDR2 having the amino acid sequence of SEQ ID NO: 126, and an H-CDR3 having the amino acid sequence of SEQ ID NO: 127; or
xliv. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xlv. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xlvi. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xlvii. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xlviii. a light chain variable region comprising an L-CDR1 having the amino acid sequence of SEQ ID NO: 250, an L-CDR2 having the amino acid sequence of SEQ ID NO: 251, and an L-CDR3 having the amino acid sequence of SEQ ID NO: 252; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xlix. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain having the amino acid sequence of SEQ ID NO: 137; or
l. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
li. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
lii. a light chain variable region comprising an L-CDR1 having the amino acid sequence of SEQ ID NO: 130, an L-CDR2 having the amino acid sequence of SEQ ID NO: 131, and an L-CDR3 having the amino acid sequence of SEQ ID NO: 132; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
liii. a light chain variable region comprising an L-CDR1 having the amino acid sequence of SEQ ID NO: 140, an L-CDR2 having the amino acid sequence of SEQ ID NO: 141, and an L-CDR3 having the amino acid sequence of SEQ ID NO: 142; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
liv. a light chain variable region comprising an L-CDR1 having the amino acid sequence of SEQ ID NO: 150, an L-CDR2 having the amino acid sequence of SEQ ID NO: 151, and an L-CDR3 having the amino acid sequence of SEQ ID NO: 152; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
lv. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain having the amino acid sequence of SEQ ID NO: 137; or
lvi. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
lvii. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
lviii. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
lix. a light chain variable region comprising an L-CDR1 having the amino acid sequence of SEQ ID NO: 200, an L-CDR2 having the amino acid sequence of SEQ ID NO: 201, and an L-CDR3 having the amino acid sequence of SEQ ID NO: 202; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
lx. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
lxi. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain having the amino acid sequence of SEQ ID NO: 137; or
lxii. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
lxiii. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
lxiv. a light chain variable region comprising an L-CDR1 having the amino acid sequence of SEQ ID NO: 250, an L-CDR2 having the amino acid sequence of SEQ ID NO: 251, and an L-CDR3 having the amino acid sequence of SEQ ID NO: 252; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
lxv. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxvi. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxvii. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain having the amino acid sequence of SEQ ID NO: 147; or
lxviii. a light chain variable region comprising an L-CDR1 having the amino acid sequence of SEQ ID NO: 130, an L-CDR2 having the amino acid sequence of SEQ ID NO: 131, and an L-CDR3 having the amino acid sequence of SEQ ID NO: 132; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxix. a light chain variable region comprising an L-CDR1 having the amino acid sequence of SEQ ID NO: 140, an L-CDR2 having the amino acid sequence of SEQ ID NO: 141, and an L-CDR3 having the amino acid sequence of SEQ ID NO: 142; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxx. a light chain variable region comprising an L-CDR1 having the amino acid sequence of SEQ ID NO: 150, an L-CDR2 having the amino acid sequence of SEQ ID NO: 151, and an L-CDR3 having the amino acid sequence of SEQ ID NO: 152; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxxi. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxxii. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxxiii. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain having the amino acid sequence of SEQ ID NO: 147; or
lxxiv. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxxv. a light chain variable region comprising an L-CDR1 having the amino acid sequence of SEQ ID NO: 200, an L-CDR2 having the amino acid sequence of SEQ ID NO: 201, and an L-CDR3 having the amino acid sequence of SEQ ID NO: 202; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxxvi. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxxvii. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxxviii. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxxix. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain having the amino acid sequence of SEQ ID NO: 147; or
lxxx. a light chain variable region comprising an L-CDR1 having the amino acid sequence of SEQ ID NO: 250, an L-CDR2 having the amino acid sequence of SEQ ID NO: 251, and an L-CDR3 having the amino acid sequence of SEQ ID NO: 252; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxxxi. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxxii. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxxiii. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxxiv. a light chain variable region comprising an L-CDR1 having the amino acid sequence of SEQ ID NO: 130, an L-CDR2 having the amino acid sequence of SEQ ID NO: 131, and an L-CDR3 having the amino acid sequence of SEQ ID NO: 132; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxxv. a light chain variable region comprising an L-CDR1 having the amino acid sequence of SEQ ID NO: 140, an L-CDR2 having the amino acid sequence of SEQ ID NO: 141, and an L-CDR3 having the amino acid sequence of SEQ ID NO: 142; and a heavy chain 155, an H-CDR2 having the amino acid sequence of SEQ ID NO: 156, and an H-CDR3 having the amino acid sequence of SEQ ID NO: 157; or
lxxxvi. a light chain variable region comprising an L-CDR1 having the amino acid sequence of SEQ ID NO: 150, an L-CDR2 having the amino acid sequence of SEQ ID NO: 151, and an L-CDR3 having the amino acid sequence of SEQ ID NO: 152; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxxvii. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxxviii. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxxix. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
xc. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
xci. a light chain variable region comprising an L-CDR1 having the amino acid sequence of SEQ ID NO: 200, an L-CDR2 having the amino acid sequence of SEQ ID NO: 201, and an L-CDR3 having the amino acid sequence of SEQ ID NO: 202; and a heavy chain 155, an H-CDR2 having the amino acid sequence of SEQ ID NO: 156, and an H-CDR3 having the amino acid sequence of SEQ ID NO: 157; or
xcii. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
xciii. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
xciv. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
xcv. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
xcvi. a light chain variable region comprising an L-CDR1 having the amino acid sequence of SEQ ID NO: 250, an L-CDR2 having the amino acid sequence of SEQ ID NO: 251, and an L-CDR3 having the amino acid sequence of SEQ ID NO: 252; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
xcvii. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain having the amino acid sequence of SEQ ID NO: 167; or
xcviii. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
xcix. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
c. a light chain variable region comprising an L-CDR1 having the amino acid sequence of SEQ ID NO: 130, an L-CDR2 having the amino acid sequence of SEQ ID NO: 131, and an L-CDR3 having the amino acid sequence of SEQ ID NO: 132; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
ci. a light chain variable region comprising an L-CDR1 having the amino acid sequence of SEQ ID NO: 140, an L-CDR2 having the amino acid sequence of SEQ ID NO: 141, and an L-CDR3 having the amino acid sequence of SEQ ID NO: 142; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
cii. a light chain variable region comprising an L-CDR1 having the amino acid sequence of SEQ ID NO: 150, an L-CDR2 having the amino acid sequence of SEQ ID NO: 151, and an L-CDR3 having the amino acid sequence of SEQ ID NO: 152; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
ciii. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain having the amino acid sequence of SEQ ID NO: 167; or
civ. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
cv. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
cvi. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
cvii. a light chain variable region comprising an L-CDR1 having the amino acid sequence of SEQ ID NO: 200, an L-CDR2 having the amino acid sequence of SEQ ID NO: 201, and an L-CDR3 having the amino acid sequence of SEQ ID NO: 202; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
cviii. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
cix. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
cx. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
cxi. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
cxii. a light chain variable region comprising an L-CDR1 having the amino acid sequence of SEQ ID NO: 250, an L-CDR2 having the amino acid sequence of SEQ ID NO: 251, and an L-CDR3 having the amino acid sequence of SEQ ID NO: 252; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
cxiii. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cxiv. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cxv. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain having the amino acid sequence of SEQ ID NO: 177; or
cxvi. a light chain variable region comprising an L-CDR1 having the amino acid sequence of SEQ ID NO: 130, an L-CDR2 having the amino acid sequence of SEQ ID NO: 131, and an L-CDR3 having the amino acid sequence of SEQ ID NO: 132; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cxvii. a light chain variable region comprising an L-CDR1 having the amino acid sequence of SEQ ID NO: 140, an L-CDR2 having the amino acid sequence of SEQ ID NO: 141, and an L-CDR3 having the amino acid sequence of SEQ ID NO: 142; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cxviii. a light chain variable region comprising an L-CDR1 having the amino acid sequence of SEQ ID NO: 150, an L-CDR2 having the amino acid sequence of SEQ ID NO: 151, and an L-CDR3 having the amino acid sequence of SEQ ID NO: 152; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cxix. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cxx. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cxxi. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain having the amino acid sequence of SEQ ID NO: 177; or
cxxii. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cxxiii. a light chain variable region comprising an L-CDR1 having the amino acid sequence of SEQ ID NO: 200, an L-CDR2 having the amino acid sequence of SEQ ID NO: 201, and an L-CDR3 having the amino acid sequence of SEQ ID NO: 202; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cxxiv. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cxxv. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cxxvi. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cxxvii. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain having the amino acid sequence of SEQ ID NO: 177.

3. The anti-CTGF antibody or an antigen-binding fragment thereof according to any one of the foregoing paras, wherein the anti-CTGF antibody or an antigen-binding fragment thereof comprises:
i. a light chain variable region comprising an amino acid sequence at least 95% identical to the amino acid sequence of SEQ ID NO: 223, and a heavy chain variable region comprising an amino acid sequence at least 95% identical to the amino acid sequence of SEQ ID NO: 108; or
ii. a light chain variable region comprising an amino acid sequence at least 95% identical to the amino acid sequence of SEQ ID NO: 103, and a heavy chain variable region comprising an amino acid sequence at least 95% identical to the amino acid sequence of SEQ ID NO: 108; or
iii. a light chain variable region comprising an amino acid sequence at least 95% identical to the amino acid sequence of SEQ ID NO: 113, and a heavy chain variable region comprising an amino acid sequence at least 95% identical to the amino acid sequence of SEQ ID NO: 108; or
iv. a light chain variable region comprising an amino acid sequence at least 95% identical to the amino acid sequence of SEQ ID NO: 123, and a heavy chain variable region comprising an amino acid sequence at least 95% identical to the amino acid sequence of SEQ ID NO: 108; or
v. a light chain variable region comprising an amino acid sequence at least 95% identical to the amino acid sequence of SEQ ID NO: 133, and a heavy chain variable region comprising an amino acid sequence at least 95% identical to the amino acid sequence of SEQ ID NO: 108; or
vi. a light chain variable region comprising an amino acid sequence at least 95% identical to the amino acid sequence of SEQ ID NO: 143, and a heavy chain variable region comprising an amino acid sequence at least 95% identical to the amino acid sequence of SEQ ID NO: 108; or
vii. a light chain variable region comprising an amino acid sequence at least 95% identical to the amino acid sequence of SEQ ID NO: 153, and a heavy chain variable region comprising an amino acid sequence at least 95% identical to the amino acid sequence of SEQ ID NO: 108; or
viii. a light chain variable region comprising an amino acid sequence at least 95% identical to the amino acid sequence of SEQ ID NO: 163, and a heavy chain variable region
ix. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 108; or
x. a light chain variable region comprising an amino acid sequence at least 95% identical to the amino acid sequence of SEQ ID NO: 183, and a heavy chain variable region comprising an amino acid sequence at least 95% identical to the amino acid sequence of SEQ ID NO: 108; or
xi. a light chain variable region comprising an amino acid sequence at least 95% identical to the amino acid sequence of SEQ ID NO: 193, and a heavy chain variable region comprising an amino acid sequence at least 95% identical to the amino acid sequence of SEQ ID NO: 108; or
xii. a light chain variable region comprising an amino acid sequence at least 95% identical to the amino acid sequence of SEQ ID NO: 203, and a heavy chain variable region comprising an amino acid sequence at least 95% identical to the amino acid sequence of SEQ ID NO: 108; or
xiii. a light chain variable region comprising an amino acid sequence at least 95% identical to the amino acid sequence of SEQ ID NO: 213, and a heavy chain variable region comprising an amino acid sequence at least 95% identical to the amino acid sequence of SEQ ID NO: 108; or
xiv. a light chain variable region comprising an amino acid sequence at least 95% identical to the amino acid sequence of SEQ ID NO: 233, and a heavy chain variable region comprising an amino acid sequence at least 95% identical to the amino acid sequence of SEQ ID NO: 108; or
xv. a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 108; or
xvi. a light chain variable region comprising an amino acid sequence at least 95% identical to the amino acid sequence of SEQ ID NO: 253, and a heavy chain variable region comprising an amino acid sequence at least 95% identical to the amino acid sequence of SEQ ID NO: 108; or
xvii. a light chain variable region comprising an amino acid sequence at least 95% identical to the amino acid sequence of SEQ ID NO: 103, and a heavy chain variable region
xviii. a light chain variable region comprising an amino acid sequence at least 95% identical to the amino acid sequence of SEQ ID NO: 113, and a heavy chain variable region comprising an amino acid sequence at least 95% identical to the amino acid sequence of SEQ ID NO: 118; or
xix. a light chain variable region comprising an amino acid sequence at least 95% identical to the amino acid sequence of SEQ ID NO: 123, and a heavy chain variable region comprising an amino acid sequence at least 95% identical to the amino acid sequence of SEQ ID NO: 118; or
xx. a light chain variable region comprising an amino acid sequence at least 95% identical to the amino acid sequence of SEQ ID NO: 133, and a heavy chain variable region comprising an amino acid sequence at least 95% identical to the amino acid sequence of SEQ ID NO: 118; or
xxi. a light chain variable region comprising an amino acid sequence at least 95% identical to the amino acid sequence of SEQ ID NO: 143, and a heavy chain variable region comprising an amino acid sequence at least 95% identical to the amino acid sequence of SEQ ID NO: 118; or
xxii. a light chain variable region comprising an amino acid sequence at least 95% identical to the amino acid sequence of SEQ ID NO: 153, and a heavy chain variable region comprising an amino acid sequence at least 95% identical to the amino acid sequence of SEQ ID NO: 118; or
xxiii. a light chain variable region comprising an amino acid sequence at least 95% identical to the amino acid sequence of SEQ ID NO: 163, and a heavy chain variable region comprising an amino acid sequence at least 95% identical to the amino acid sequence of SEQ ID NO: 118; or
xxiv. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 118; or
xxv. a light chain variable region comprising an amino acid sequence at least 95% identical to the amino acid sequence of SEQ ID NO: 183, and a heavy chain variable region comprising an amino acid sequence at least 95% identical to the amino acid sequence of SEQ ID NO: 118; or
xxvi. a light chain variable region comprising an amino acid sequence at least 95% identical to the amino acid sequence of SEQ ID NO: 193, and a heavy chain variable region
xxvii. a light chain variable region comprising an amino acid sequence at least 95% identical to the amino acid sequence of SEQ ID NO: 203, and a heavy chain variable region comprising an amino acid sequence at least 95% identical to the amino acid sequence of SEQ ID NO: 118; or
xxviii. a light chain variable region comprising an amino acid sequence at least 95% identical to the amino acid sequence of SEQ ID NO: 213, and a heavy chain variable region comprising an amino acid sequence at least 95% identical to the amino acid sequence of SEQ ID NO: 118; or
xxix. a light chain variable region comprising an amino acid sequence at least 95% identical to the amino acid sequence of SEQ ID NO: 223, and a heavy chain variable region comprising an amino acid sequence at least 95% identical to the amino acid sequence of SEQ ID NO: 118; or
xxx. a light chain variable region comprising an amino acid sequence at least 95% identical to the amino acid sequence of SEQ ID NO: 233, and a heavy chain variable region comprising an amino acid sequence at least 95% identical to the amino acid sequence of SEQ ID NO: 118; or
xxxi. a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 118; or
xxxii. a light chain variable region comprising an amino acid sequence at least 95% identical to the amino acid sequence of SEQ ID NO: 253, and a heavy chain variable region comprising an amino acid sequence at least 95% identical to the amino acid sequence of SEQ ID NO: 118; or
xxxiii. a light chain variable region comprising an amino acid sequence at least 95% identical to the amino acid sequence of SEQ ID NO: 103, and a heavy chain variable region comprising an amino acid sequence at least 95% identical to the amino acid sequence of SEQ ID NO: 128; or
xxxiv. a light chain variable region comprising an amino acid sequence at least 95% identical to the amino acid sequence of SEQ ID NO: 113, and a heavy chain variable region comprising an amino acid sequence at least 95% identical to the amino acid sequence of SEQ ID NO: 128; or
xxxv. a light chain variable region comprising an amino acid sequence at least 95% identical to the amino acid sequence of SEQ ID NO: 123, and a heavy chain variable region
xxxvi. a light chain variable region comprising an amino acid sequence at least 95% identical to the amino acid sequence of SEQ ID NO: 133, and a heavy chain variable region comprising an amino acid sequence at least 95% identical to the amino acid sequence of SEQ ID NO: 128; or
xxxvii. a light chain variable region comprising an amino acid sequence at least 95% identical to the amino acid sequence of SEQ ID NO: 143, and a heavy chain variable region comprising an amino acid sequence at least 95% identical to the amino acid sequence of SEQ ID NO: 128; or
xxxviii. a light chain variable region comprising an amino acid sequence at least 95% identical to the amino acid sequence of SEQ ID NO: 153, and a heavy chain variable region comprising an amino acid sequence at least 95% identical to the amino acid sequence of SEQ ID NO: 128; or
xxxix. a light chain variable region comprising an amino acid sequence at least 95% identical to the amino acid sequence of SEQ ID NO: 163, and a heavy chain variable region comprising an amino acid sequence at least 95% identical to the amino acid sequence of SEQ ID NO: 128; or
xl. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 128; or
xli. a light chain variable region comprising an amino acid sequence at least 95% identical to the amino acid sequence of SEQ ID NO: 183, and a heavy chain variable region comprising an amino acid sequence at least 95% identical to the amino acid sequence of SEQ ID NO: 128; or
xlii. a light chain variable region comprising an amino acid sequence at least 95% identical to the amino acid sequence of SEQ ID NO: 193, and a heavy chain variable region comprising an amino acid sequence at least 95% identical to the amino acid sequence of SEQ ID NO: 128; or
xliii. a light chain variable region comprising an amino acid sequence at least 95% identical to the amino acid sequence of SEQ ID NO: 203, and a heavy chain variable region comprising an amino acid sequence at least 95% identical to the amino acid sequence of SEQ ID NO: 128; or
xliv. a light chain variable region comprising an amino acid sequence at least 95% identical to the amino acid sequence of SEQ ID NO: 213, and a heavy chain variable region
xlv. a light chain variable region comprising an amino acid sequence at least 95% identical to the amino acid sequence of SEQ ID NO: 223, and a heavy chain variable region comprising an amino acid sequence at least 95% identical to the amino acid sequence of SEQ ID NO: 128; or
xlvi. a light chain variable region comprising an amino acid sequence at least 95% identical to the amino acid sequence of SEQ ID NO: 233, and a heavy chain variable region comprising an amino acid sequence at least 95% identical to the amino acid sequence of SEQ ID NO: 128; or
xlvii. a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 128; or
xlviii. a light chain variable region comprising an amino acid sequence at least 95% identical to the amino acid sequence of SEQ ID NO: 253, and a heavy chain variable region comprising an amino acid sequence at least 95% identical to the amino acid sequence of SEQ ID NO: 128; or
xlix. a light chain variable region comprising an amino acid sequence at least 95% identical to the amino acid sequence of SEQ ID NO: 103, and a heavy chain variable region comprising an amino acid sequence at least 95% identical to the amino acid sequence of SEQ ID NO: 138; or
l. a light chain variable region comprising an amino acid sequence at least 95% identical to the amino acid sequence of SEQ ID NO: 113, and a heavy chain variable region comprising an amino acid sequence at least 95% identical to the amino acid sequence of SEQ ID NO: 138; or
li. a light chain variable region comprising an amino acid sequence at least 95% identical to the amino acid sequence of SEQ ID NO: 123, and a heavy chain variable region comprising an amino acid sequence at least 95% identical to the amino acid sequence of SEQ ID NO: 138; or
lii. a light chain variable region comprising an amino acid sequence at least 95% identical to the amino acid sequence of SEQ ID NO: 133, and a heavy chain variable region comprising an amino acid sequence at least 95% identical to the amino acid sequence of SEQ ID NO: 138; or
liii. a light chain variable region comprising an amino acid sequence at least 95% identical to the amino acid sequence of SEQ ID NO: 143, and a heavy chain variable region
liv. a light chain variable region comprising an amino acid sequence at least 95% identical to the amino acid sequence of SEQ ID NO: 153, and a heavy chain variable region comprising an amino acid sequence at least 95% identical to the amino acid sequence of SEQ ID NO: 138; or
lv. a light chain variable region comprising an amino acid sequence at least 95% identical to the amino acid sequence of SEQ ID NO: 163, and a heavy chain variable region comprising an amino acid sequence at least 95% identical to the amino acid sequence of SEQ ID NO: 138; or
lvi. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 138; or
lvii. a light chain variable region comprising an amino acid sequence at least 95% identical to the amino acid sequence of SEQ ID NO: 183, and a heavy chain variable region comprising an amino acid sequence at least 95% identical to the amino acid sequence of SEQ ID NO: 138; or
lviii. a light chain variable region comprising an amino acid sequence at least 95% identical to the amino acid sequence of SEQ ID NO: 193, and a heavy chain variable region comprising an amino acid sequence at least 95% identical to the amino acid sequence of SEQ ID NO: 138; or
lix. a light chain variable region comprising an amino acid sequence at least 95% identical to the amino acid sequence of SEQ ID NO: 203, and a heavy chain variable region comprising an amino acid sequence at least 95% identical to the amino acid sequence of SEQ ID NO: 138; or
lx. a light chain variable region comprising an amino acid sequence at least 95% identical to the amino acid sequence of SEQ ID NO: 213, and a heavy chain variable region comprising an amino acid sequence at least 95% identical to the amino acid sequence of SEQ ID NO: 138; or
lxi. a light chain variable region comprising an amino acid sequence at least 95% identical to the amino acid sequence of SEQ ID NO: 223, and a heavy chain variable region comprising an amino acid sequence at least 95% identical to the amino acid sequence of SEQ ID NO: 138; or
lxii. a light chain variable region comprising an amino acid sequence at least 95% identical to the amino acid sequence of SEQ ID NO: 233, and a heavy chain variable region
lxiii. a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 138; or
lxiv. a light chain variable region comprising an amino acid sequence at least 95% identical to the amino acid sequence of SEQ ID NO: 253, and a heavy chain variable region comprising an amino acid sequence at least 95% identical to the amino acid sequence of SEQ ID NO: 138; or
lxv. a light chain variable region comprising an amino acid sequence at least 95% identical to the amino acid sequence of SEQ ID NO: 103, and a heavy chain variable region comprising an amino acid sequence at least 95% identical to the amino acid sequence of SEQ ID NO: 148; or
lxvi. a light chain variable region comprising an amino acid sequence at least 95% identical to the amino acid sequence of SEQ ID NO: 113, and a heavy chain variable region comprising an amino acid sequence at least 95% identical to the amino acid sequence of SEQ ID NO: 148; or
lxvii. a light chain variable region comprising an amino acid sequence at least 95% identical to the amino acid sequence of SEQ ID NO: 123, and a heavy chain variable region comprising an amino acid sequence at least 95% identical to the amino acid sequence of SEQ ID NO: 148; or
lxviii. a light chain variable region comprising an amino acid sequence at least 95% identical to the amino acid sequence of SEQ ID NO: 133, and a heavy chain variable region comprising an amino acid sequence at least 95% identical to the amino acid sequence of SEQ ID NO: 148; or
lxix. a light chain variable region comprising an amino acid sequence at least 95% identical to the amino acid sequence of SEQ ID NO: 143, and a heavy chain variable region comprising an amino acid sequence at least 95% identical to the amino acid sequence of SEQ ID NO: 148; or
lxx. a light chain variable region comprising an amino acid sequence at least 95% identical to the amino acid sequence of SEQ ID NO: 153, and a heavy chain variable region comprising an amino acid sequence at least 95% identical to the amino acid sequence of SEQ ID NO: 148; or
lxxi. a light chain variable region comprising an amino acid sequence at least 95% identical to the amino acid sequence of SEQ ID NO: 163, and a heavy chain variable region
lxxii. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 148; or
lxxiii. a light chain variable region comprising an amino acid sequence at least 95% identical to the amino acid sequence of SEQ ID NO: 183, and a heavy chain variable region comprising an amino acid sequence at least 95% identical to the amino acid sequence of SEQ ID NO: 148; or
lxxiv. a light chain variable region comprising an amino acid sequence at least 95% identical to the amino acid sequence of SEQ ID NO: 193, and a heavy chain variable region comprising an amino acid sequence at least 95% identical to the amino acid sequence of SEQ ID NO: 148; or
lxxv. a light chain variable region comprising an amino acid sequence at least 95% identical to the amino acid sequence of SEQ ID NO: 203, and a heavy chain variable region comprising an amino acid sequence at least 95% identical to the amino acid sequence of SEQ ID NO: 148; or
lxxvi. a light chain variable region comprising an amino acid sequence at least 95% identical to the amino acid sequence of SEQ ID NO: 213, and a heavy chain variable region comprising an amino acid sequence at least 95% identical to the amino acid sequence of SEQ ID NO: 148; or
lxxvii. a light chain variable region comprising an amino acid sequence at least 95% identical to the amino acid sequence of SEQ ID NO: 223, and a heavy chain variable region comprising an amino acid sequence at least 95% identical to the amino acid sequence of SEQ ID NO: 148; or
lxxviii. a light chain variable region comprising an amino acid sequence at least 95% identical to the amino acid sequence of SEQ ID NO: 233, and a heavy chain variable region comprising an amino acid sequence at least 95% identical to the amino acid sequence of SEQ ID NO: 148; or
lxxix. a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 148; or
lxxx. a light chain variable region comprising an amino acid sequence at least 95% identical to the amino acid sequence of SEQ ID NO: 253, and a heavy chain variable region
lxxxi. a light chain variable region comprising an amino acid sequence at least 95% identical to the amino acid sequence of SEQ ID NO: 103, and a heavy chain variable region comprising an amino acid sequence at least 95% identical to the amino acid sequence of SEQ ID NO: 158; or
lxxxii. a light chain variable region comprising an amino acid sequence at least 95% identical to the amino acid sequence of SEQ ID NO: 113, and a heavy chain variable region comprising an amino acid sequence at least 95% identical to the amino acid sequence of SEQ ID NO: 158; or
lxxxiii. a light chain variable region comprising an amino acid sequence at least 95% identical to the amino acid sequence of SEQ ID NO: 123, and a heavy chain variable region comprising an amino acid sequence at least 95% identical to the amino acid sequence of SEQ ID NO: 158; or
lxxxiv. a light chain variable region comprising an amino acid sequence at least 95% identical to the amino acid sequence of SEQ ID NO: 133, and a heavy chain variable region comprising an amino acid sequence at least 95% identical to the amino acid sequence of SEQ ID NO: 158; or
lxxxv. a light chain variable region comprising an amino acid sequence at least 95% identical to the amino acid sequence of SEQ ID NO: 143, and a heavy chain variable region comprising an amino acid sequence at least 95% identical to the amino acid sequence of SEQ ID NO: 158; or
lxxxvi. a light chain variable region comprising an amino acid sequence at least 95% identical to the amino acid sequence of SEQ ID NO: 153, and a heavy chain variable region comprising an amino acid sequence at least 95% identical to the amino acid sequence of SEQ ID NO: 158; or
lxxxvii. a light chain variable region comprising an amino acid sequence at least 95% identical to the amino acid sequence of SEQ ID NO: 163, and a heavy chain variable region comprising an amino acid sequence at least 95% identical to the amino acid sequence of SEQ ID NO: 158; or
lxxxviii. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 158; or
lxxxix. a light chain variable region comprising an amino acid sequence at least 95% identical to the amino acid sequence of SEQ ID NO: 183, and a heavy chain variable region
xc. a light chain variable region comprising an amino acid sequence at least 95% identical to the amino acid sequence of SEQ ID NO: 193, and a heavy chain variable region comprising an amino acid sequence at least 95% identical to the amino acid sequence of SEQ ID NO: 158; or
xci. a light chain variable region comprising an amino acid sequence at least 95% identical to the amino acid sequence of SEQ ID NO: 203, and a heavy chain variable region comprising an amino acid sequence at least 95% identical to the amino acid sequence of SEQ ID NO: 158; or
xcii. a light chain variable region comprising an amino acid sequence at least 95% identical to the amino acid sequence of SEQ ID NO: 213, and a heavy chain variable region comprising an amino acid sequence at least 95% identical to the amino acid sequence of SEQ ID NO: 158; or
xciii. a light chain variable region comprising an amino acid sequence at least 95% identical to the amino acid sequence of SEQ ID NO: 223, and a heavy chain variable region comprising an amino acid sequence at least 95% identical to the amino acid sequence of SEQ ID NO: 158; or
xciv. a light chain variable region comprising an amino acid sequence at least 95% identical to the amino acid sequence of SEQ ID NO: 233, and a heavy chain variable region comprising an amino acid sequence at least 95% identical to the amino acid sequence of SEQ ID NO: 158; or
xcv. a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 158; or
xcvi. a light chain variable region comprising an amino acid sequence at least 95% identical to the amino acid sequence of SEQ ID NO: 253, and a heavy chain variable region comprising an amino acid sequence at least 95% identical to the amino acid sequence of SEQ ID NO: 158; or
xcvii. a light chain variable region comprising an amino acid sequence at least 95% identical to the amino acid sequence of SEQ ID NO: 103, and a heavy chain variable region comprising an amino acid sequence at least 95% identical to the amino acid sequence of SEQ ID NO: 168; or
xcviii. a light chain variable region comprising an amino acid sequence at least 95% identical to the amino acid sequence of SEQ ID NO: 113, and a heavy chain variable region
xcix. a light chain variable region comprising an amino acid sequence at least 95% identical to the amino acid sequence of SEQ ID NO: 123, and a heavy chain variable region comprising an amino acid sequence at least 95% identical to the amino acid sequence of SEQ ID NO: 168; or
c. a light chain variable region comprising an amino acid sequence at least 95% identical to the amino acid sequence of SEQ ID NO: 133, and a heavy chain variable region comprising an amino acid sequence at least 95% identical to the amino acid sequence of SEQ ID NO: 168; or
ci. a light chain variable region comprising an amino acid sequence at least 95% identical to the amino acid sequence of SEQ ID NO: 143, and a heavy chain variable region comprising an amino acid sequence at least 95% identical to the amino acid sequence of SEQ ID NO: 168; or
cii. a light chain variable region comprising an amino acid sequence at least 95% identical to the amino acid sequence of SEQ ID NO: 153, and a heavy chain variable region comprising an amino acid sequence at least 95% identical to the amino acid sequence of SEQ ID NO: 168; or
ciii. a light chain variable region comprising an amino acid sequence at least 95% identical to the amino acid sequence of SEQ ID NO: 163, and a heavy chain variable region comprising an amino acid sequence at least 95% identical to the amino acid sequence of SEQ ID NO: 168; or
civ. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 168; or
cv. a light chain variable region comprising an amino acid sequence at least 95% identical to the amino acid sequence of SEQ ID NO: 183, and a heavy chain variable region comprising an amino acid sequence at least 95% identical to the amino acid sequence of SEQ ID NO: 168; or
cvi. a light chain variable region comprising an amino acid sequence at least 95% identical to the amino acid sequence of SEQ ID NO: 193, and a heavy chain variable region comprising an amino acid sequence at least 95% identical to the amino acid sequence of SEQ ID NO: 168; or
cvii. a light chain variable region comprising an amino acid sequence at least 95% identical to the amino acid sequence of SEQ ID NO: 203, and a heavy chain variable region
cviii. a light chain variable region comprising an amino acid sequence at least 95% identical to the amino acid sequence of SEQ ID NO: 213, and a heavy chain variable region comprising an amino acid sequence at least 95% identical to the amino acid sequence of SEQ ID NO: 168; or
cix. a light chain variable region comprising an amino acid sequence at least 95% identical to the amino acid sequence of SEQ ID NO: 223, and a heavy chain variable region comprising an amino acid sequence at least 95% identical to the amino acid sequence of SEQ ID NO: 168; or
cx. a light chain variable region comprising an amino acid sequence at least 95% identical to the amino acid sequence of SEQ ID NO: 233, and a heavy chain variable region comprising an amino acid sequence at least 95% identical to the amino acid sequence of SEQ ID NO: 168; or
cxi. a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 168; or
cxii. a light chain variable region comprising an amino acid sequence at least 95% identical to the amino acid sequence of SEQ ID NO: 253, and a heavy chain variable region comprising an amino acid sequence at least 95% identical to the amino acid sequence of SEQ ID NO: 168; or
cxiii. a light chain variable region comprising an amino acid sequence at least 95% identical to the amino acid sequence of SEQ ID NO: 103, and a heavy chain variable region comprising an amino acid sequence at least 95% identical to the amino acid sequence of SEQ ID NO: 178; or
cxiv. a light chain variable region comprising an amino acid sequence at least 95% identical to the amino acid sequence of SEQ ID NO: 113, and a heavy chain variable region comprising an amino acid sequence at least 95% identical to the amino acid sequence of SEQ ID NO: 178; or
cxv. a light chain variable region comprising an amino acid sequence at least 95% identical to the amino acid sequence of SEQ ID NO: 123, and a heavy chain variable region comprising an amino acid sequence at least 95% identical to the amino acid sequence of SEQ ID NO: 178; or
cxvi. a light chain variable region comprising an amino acid sequence at least 95% identical to the amino acid sequence of SEQ ID NO: 133, and a heavy chain variable region
cxvii. a light chain variable region comprising an amino acid sequence at least 95% identical to the amino acid sequence of SEQ ID NO: 143, and a heavy chain variable region comprising an amino acid sequence at least 95% identical to the amino acid sequence of SEQ ID NO: 178; or
cxviii. a light chain variable region comprising an amino acid sequence at least 95% identical to the amino acid sequence of SEQ ID NO: 153, and a heavy chain variable region comprising an amino acid sequence at least 95% identical to the amino acid sequence of SEQ ID NO: 178; or
cxix. a light chain variable region comprising an amino acid sequence at least 95% identical to the amino acid sequence of SEQ ID NO: 163, and a heavy chain variable region comprising an amino acid sequence at least 95% identical to the amino acid sequence of SEQ ID NO: 178; or
cxx. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 178; or
cxxi. a light chain variable region comprising an amino acid sequence at least 95% identical to the amino acid sequence of SEQ ID NO: 183, and a heavy chain variable region comprising an amino acid sequence at least 95% identical to the amino acid sequence of SEQ ID NO: 178; or
cxxii. a light chain variable region comprising an amino acid sequence at least 95% identical to the amino acid sequence of SEQ ID NO: 193, and a heavy chain variable region comprising an amino acid sequence at least 95% identical to the amino acid sequence of SEQ ID NO: 178; or
cxxiii. a light chain variable region comprising an amino acid sequence at least 95% identical to the amino acid sequence of SEQ ID NO: 203, and a heavy chain variable region comprising an amino acid sequence at least 95% identical to the amino acid sequence of SEQ ID NO: 178; or
cxxiv. a light chain variable region comprising an amino acid sequence at least 95% identical to the amino acid sequence of SEQ ID NO: 213, and a heavy chain variable region comprising an amino acid sequence at least 95% identical to the amino acid sequence of SEQ ID NO: 178; or
cxxv. a light chain variable region comprising an amino acid sequence at least 95% identical to the amino acid sequence of SEQ ID NO: 223, and a heavy chain variable region
cxxvi. a light chain variable region comprising an amino acid sequence at least 95% identical to the amino acid sequence of SEQ ID NO: 233, and a heavy chain variable region comprising an amino acid sequence at least 95% identical to the amino acid sequence of SEQ ID NO: 178; or
cxxvii. a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 178.

4. The anti-CTGF antibody or an antigen-binding fragment thereof according to any one of the foregoing paras, wherein the anti-CTGF antibody or an antigen-binding fragment thereof comprises:
i. a light chain variable region comprising the amino acid sequence of SEQ ID NO: 223, and a heavy chain variable region comprising the amino acid sequence of SEQ ID NO: 108; or
ii. a light chain variable region comprising the amino acid sequence of SEQ ID NO: 103, and a heavy chain variable region comprising the amino acid sequence of SEQ ID NO: 108; or
iii. a light chain variable region comprising the amino acid sequence of SEQ ID NO: 113, and a heavy chain variable region comprising the amino acid sequence of SEQ ID NO: 108; or
iv. a light chain variable region comprising the amino acid sequence of SEQ ID NO: 123, and a heavy chain variable region comprising the amino acid sequence of SEQ ID NO: 108; or
v. a light chain variable region comprising the amino acid sequence of SEQ ID NO: 133, and a heavy chain variable region comprising the amino acid sequence of SEQ ID NO: 108; or
vi. a light chain variable region comprising the amino acid sequence of SEQ ID NO: 143, and a heavy chain variable region comprising the amino acid sequence of SEQ ID NO: 108; or
vii. a light chain variable region comprising the amino acid sequence of SEQ ID NO: 153, and a heavy chain variable region comprising the amino acid sequence of SEQ ID NO: 108; or
viii. a light chain variable region comprising the amino acid sequence of SEQ ID NO: 163, and a heavy chain variable region comprising the amino acid sequence of SEQ ID NO: 108; or
ix. a light chain variable region comprising the amino acid sequence of SEQ ID NO: 173, and a heavy chain variable region comprising the amino acid sequence of SEQ ID NO: 108; or
x. a light chain variable region comprising the amino acid sequence of SEQ ID NO: 183, and a heavy chain variable region comprising the amino acid sequence of SEQ ID NO: 108; or
xi. a light chain variable region comprising the amino acid sequence of SEQ ID NO: 193, and a heavy chain variable region comprising the amino acid sequence of SEQ ID NO: 108; or
xii. a light chain variable region comprising the amino acid sequence of SEQ ID NO: 203, and a heavy chain variable region comprising the amino acid sequence of SEQ ID NO: 108; or
xiii. a light chain variable region comprising the amino acid sequence of SEQ ID NO: 213, and a heavy chain variable region comprising the amino acid sequence of SEQ ID NO: 108; or
xiv. a light chain variable region comprising the amino acid sequence of SEQ ID NO: 233, and a heavy chain variable region comprising the amino acid sequence of SEQ ID NO: 108; or
xv. a light chain variable region comprising the amino acid sequence of SEQ ID NO: 243, and a heavy chain variable region comprising the amino acid sequence of SEQ ID NO: 108; or
xvi. a light chain variable region comprising the amino acid sequence of SEQ ID NO: 253, and a heavy chain variable region comprising the amino acid sequence of SEQ ID NO: 108; or
xvii. a light chain variable region comprising the amino acid sequence of SEQ ID NO: 103, and a heavy chain variable region comprising the amino acid sequence of SEQ ID NO: 118; or
xviii. a light chain variable region comprising the amino acid sequence of SEQ ID NO: 113, and a heavy chain variable region comprising the amino acid sequence of SEQ ID NO: 118; or
xix. a light chain variable region comprising the amino acid sequence of SEQ ID NO: 123, and a heavy chain variable region comprising the amino acid sequence of SEQ ID NO: 118; or
xx. a light chain variable region comprising the amino acid sequence of SEQ ID NO: 133, and a heavy chain variable region comprising the amino acid sequence of SEQ ID NO: 118; or
xxi. a light chain variable region comprising the amino acid sequence of SEQ ID NO: 143, and a heavy chain variable region comprising the amino acid sequence of SEQ ID NO: 118; or
xxii. a light chain variable region comprising the amino acid sequence of SEQ ID NO: 153, and a heavy chain variable region comprising the amino acid sequence of SEQ ID NO: 118; or
xxiii. a light chain variable region comprising the amino acid sequence of SEQ ID NO: 163, and a heavy chain variable region comprising the amino acid sequence of SEQ ID NO: 118; or
xxiv. a light chain variable region comprising the amino acid sequence of SEQ ID NO: 173, and a heavy chain variable region comprising the amino acid sequence of SEQ ID NO: 118; or
xxv. a light chain variable region comprising the amino acid sequence of SEQ ID NO: 183, and a heavy chain variable region comprising the amino acid sequence of SEQ ID NO: 118; or
xxvi. a light chain variable region comprising the amino acid sequence of SEQ ID NO: 193, and a heavy chain variable region comprising the amino acid sequence of SEQ ID NO: 118; or
xxvii. a light chain variable region comprising the amino acid sequence of SEQ ID NO: 203, and a heavy chain variable region comprising the amino acid sequence of SEQ ID NO: 118; or
xxviii. a light chain variable region comprising the amino acid sequence of SEQ ID NO: 213, and a heavy chain variable region comprising the amino acid sequence of SEQ ID NO: 118; or
xxix. a light chain variable region comprising the amino acid sequence of SEQ ID NO: 223, and a heavy chain variable region comprising the amino acid sequence of SEQ ID NO: 118; or
xxx. a light chain variable region comprising the amino acid sequence of SEQ ID NO: 233, and a heavy chain variable region comprising the amino acid sequence of SEQ ID NO: 118; or
xxxi. a light chain variable region comprising the amino acid sequence of SEQ ID NO: 243, and a heavy chain variable region comprising the amino acid sequence of SEQ ID NO: 118; or
xxxii. a light chain variable region comprising the amino acid sequence of SEQ ID NO: 253, and a heavy chain variable region comprising the amino acid sequence of SEQ ID NO: 118; or
xxxiii. a light chain variable region comprising the amino acid sequence of SEQ ID NO: 103, and a heavy chain variable region comprising the amino acid sequence of SEQ ID NO: 128; or
xxxiv. a light chain variable region comprising the amino acid sequence of SEQ ID NO: 113, and a heavy chain variable region comprising the amino acid sequence of SEQ ID NO: 128; or
xxxv. a light chain variable region comprising the amino acid sequence of SEQ ID NO: 123, and a heavy chain variable region comprising the amino acid sequence of SEQ ID NO: 128; or
xxxvi. a light chain variable region comprising the amino acid sequence of SEQ ID NO: 133, and a heavy chain variable region comprising the amino acid sequence of SEQ ID NO: 128; or
xxxvii. a light chain variable region comprising the amino acid sequence of SEQ ID NO: 143, and a heavy chain variable region comprising the amino acid sequence of SEQ ID NO: 128; or
xxxviii. a light chain variable region comprising the amino acid sequence of SEQ ID NO: 153, and a heavy chain variable region comprising the amino acid sequence of SEQ ID NO: 128; or
xxxix. a light chain variable region comprising the amino acid sequence of SEQ ID NO: 163, and a heavy chain variable region comprising the amino acid sequence of SEQ ID NO: 128; or
xl. a light chain variable region comprising the amino acid sequence of SEQ ID NO: 173, and a heavy chain variable region comprising the amino acid sequence of SEQ ID NO: 128; or
xli. a light chain variable region comprising the amino acid sequence of SEQ ID NO: 183, and a heavy chain variable region comprising the amino acid sequence of SEQ ID NO: 128; or
xlii. a light chain variable region comprising the amino acid sequence of SEQ ID NO: 193, and a heavy chain variable region comprising the amino acid sequence of SEQ ID NO: 128; or
xliii. a light chain variable region comprising the amino acid sequence of SEQ ID NO: 203, and a heavy chain variable region comprising the amino acid sequence of SEQ ID NO: 128; or
xliv. a light chain variable region comprising the amino acid sequence of SEQ ID NO: 213, and a heavy chain variable region comprising the amino acid sequence of SEQ ID NO: 128; or
xlv. a light chain variable region comprising the amino acid sequence of SEQ ID NO: 223, and a heavy chain variable region comprising the amino acid sequence of SEQ ID NO: 128; or
xlvi. a light chain variable region comprising the amino acid sequence of SEQ ID NO: 233, and a heavy chain variable region comprising the amino acid sequence of SEQ ID NO: 128; or
xlvii. a light chain variable region comprising the amino acid sequence of SEQ ID NO: 243, and a heavy chain variable region comprising the amino acid sequence of SEQ ID NO: 128; or
xlviii. a light chain variable region comprising the amino acid sequence of SEQ ID NO: 253, and a heavy chain variable region comprising the amino acid sequence of SEQ ID NO: 128; or
xlix. a light chain variable region comprising the amino acid sequence of SEQ ID NO: 103, and a heavy chain variable region comprising the amino acid sequence of SEQ ID NO: 138; or
l. a light chain variable region comprising the amino acid sequence of SEQ ID NO: 113, and a heavy chain variable region comprising the amino acid sequence of SEQ ID NO: 138; or
li. a light chain variable region comprising the amino acid sequence of SEQ ID NO: 123, and a heavy chain variable region comprising the amino acid sequence of SEQ ID NO: 138; or
lii. a light chain variable region comprising the amino acid sequence of SEQ ID NO: 133, and a heavy chain variable region comprising the amino acid sequence of SEQ ID NO: 138; or
liii. a light chain variable region comprising the amino acid sequence of SEQ ID NO: 143, and a heavy chain variable region comprising the amino acid sequence of SEQ ID NO: 138; or
liv. a light chain variable region comprising the amino acid sequence of SEQ ID NO: 153, and a heavy chain variable region comprising the amino acid sequence of SEQ ID NO: 138; or
lv. a light chain variable region comprising the amino acid sequence of SEQ ID NO: 163, and a heavy chain variable region comprising the amino acid sequence of SEQ ID NO: 138; or
lvi. a light chain variable region comprising the amino acid sequence of SEQ ID NO: 173, and a heavy chain variable region comprising the amino acid sequence of SEQ ID NO: 138; or
lvii. a light chain variable region comprising the amino acid sequence of SEQ ID NO: 183, and a heavy chain variable region comprising the amino acid sequence of SEQ ID NO: 138; or
lviii. a light chain variable region comprising the amino acid sequence of SEQ ID NO: 193, and a heavy chain variable region comprising the amino acid sequence of SEQ ID NO: 138; or
lix. a light chain variable region comprising the amino acid sequence of SEQ ID NO: 203, and a heavy chain variable region comprising the amino acid sequence of SEQ ID NO: 138; or
lx. a light chain variable region comprising the amino acid sequence of SEQ ID NO: 213, and a heavy chain variable region comprising the amino acid sequence of SEQ ID NO: 138; or
lxi. a light chain variable region comprising the amino acid sequence of SEQ ID NO: 223, and a heavy chain variable region comprising the amino acid sequence of SEQ ID NO: 138; or
lxii. a light chain variable region comprising the amino acid sequence of SEQ ID NO: 233, and a heavy chain variable region comprising the amino acid sequence of SEQ ID NO: 138; or
lxiii. a light chain variable region comprising the amino acid sequence of SEQ ID NO: 243, and a heavy chain variable region comprising the amino acid sequence of SEQ ID NO: 138; or
lxiv. a light chain variable region comprising the amino acid sequence of SEQ ID NO: 253, and a heavy chain variable region comprising the amino acid sequence of SEQ ID NO: 138; or
lxv. a light chain variable region comprising the amino acid sequence of SEQ ID NO: 103, and a heavy chain variable region comprising the amino acid sequence of SEQ ID NO: 148; or
lxvi a light chain variable region comprising the amino acid sequence of SEQ ID NO: 113, and a heavy chain variable region comprising the amino acid sequence of SEQ ID NO: 148; or
lxvii. a light chain variable region comprising the amino acid sequence of SEQ ID NO: 123, and a heavy chain variable region comprising the amino acid sequence of SEQ ID NO: 148; or
lxviii. a light chain variable region comprising the amino acid sequence of SEQ ID NO: 133, and a heavy chain variable region comprising the amino acid sequence of SEQ ID NO: 148; or
lxix. a light chain variable region comprising the amino acid sequence of SEQ ID NO: 143, and a heavy chain variable region comprising the amino acid sequence of SEQ ID NO: 148; or
lxx. a light chain variable region comprising the amino acid sequence of SEQ ID NO: 153, and a heavy chain variable region comprising the amino acid sequence of SEQ ID NO: 148; or
lxxi. a light chain variable region comprising the amino acid sequence of SEQ ID NO: 163, and a heavy chain variable region comprising the amino acid sequence of SEQ ID NO: 148; or
lxxii. a light chain variable region comprising the amino acid sequence of SEQ ID NO: 173, and a heavy chain variable region comprising the amino acid sequence of SEQ ID NO: 148; or
lxxiii. a light chain variable region comprising the amino acid sequence of SEQ ID NO: 183, and a heavy chain variable region comprising the amino acid sequence of SEQ ID NO: 148; or
lxxiv. a light chain variable region comprising the amino acid sequence of SEQ ID NO: 193, and a heavy chain variable region comprising the amino acid sequence of SEQ ID NO: 148; or
lxxv. a light chain variable region comprising the amino acid sequence of SEQ ID NO: 203, and a heavy chain variable region comprising the amino acid sequence of SEQ ID NO: 148; or
lxxvi. a light chain variable region comprising the amino acid sequence of SEQ ID NO: 213, and a heavy chain variable region comprising the amino acid sequence of SEQ ID NO: 148; or
lxxvii. a light chain variable region comprising the amino acid sequence of SEQ ID NO: 223, and a heavy chain variable region comprising the amino acid sequence of SEQ ID NO: 148; or
lxxviii. a light chain variable region comprising the amino acid sequence of SEQ ID NO: 233, and a heavy chain variable region comprising the amino acid sequence of SEQ ID NO: 148; or
lxxix. a light chain variable region comprising the amino acid sequence of SEQ ID NO: 243, and a heavy chain variable region comprising the amino acid sequence of SEQ ID NO: 148; or
lxxx. a light chain variable region comprising the amino acid sequence of SEQ ID NO: 253, and a heavy chain variable region comprising the amino acid sequence of SEQ ID NO: 148; or
lxxxi. a light chain variable region comprising the amino acid sequence of SEQ ID NO: 103, and a heavy chain variable region comprising the amino acid sequence of SEQ ID NO: 158; or
lxxxii. a light chain variable region comprising the amino acid sequence of SEQ ID NO: 113, and a heavy chain variable region comprising the amino acid sequence of SEQ ID NO: 158; or
lxxxiii. a light chain variable region comprising the amino acid sequence of SEQ ID NO: 123, and a heavy chain variable region comprising the amino acid sequence of SEQ ID NO: 158; or
lxxxiv. a light chain variable region comprising the amino acid sequence of SEQ ID NO: 133, and a heavy chain variable region comprising the amino acid sequence of SEQ ID NO: 158; or
lxxxv. a light chain variable region comprising the amino acid sequence of SEQ ID NO: 143, and a heavy chain variable region comprising the amino acid sequence of SEQ ID NO: 158; or
lxxxvi. a light chain variable region comprising the amino acid sequence of SEQ ID NO: 153, and a heavy chain variable region comprising the amino acid sequence of SEQ ID NO: 158; or
lxxxvii. a light chain variable region comprising the amino acid sequence of SEQ ID NO: 163, and a heavy chain variable region comprising the amino acid sequence of SEQ ID NO: 158; or
lxxxviii. a light chain variable region comprising the amino acid sequence of SEQ ID NO: 173, and a heavy chain variable region comprising the amino acid sequence of SEQ ID NO: 158; or
lxxxix. a light chain variable region comprising the amino acid sequence of SEQ ID NO: 183, and a heavy chain variable region comprising the amino acid sequence of SEQ ID NO: 158; or
xc. a light chain variable region comprising the amino acid sequence of SEQ ID NO: 193, and a heavy chain variable region comprising the amino acid sequence of SEQ ID NO: 158; or
xci. a light chain variable region comprising the amino acid sequence of SEQ ID NO: 203, and a heavy chain variable region comprising the amino acid sequence of SEQ ID NO: 158; or
xcii. a light chain variable region comprising the amino acid sequence of SEQ ID NO: 213, and a heavy chain variable region comprising the amino acid sequence of SEQ ID NO: 158; or
xciii. a light chain variable region comprising the amino acid sequence of SEQ ID NO: 223, and a heavy chain variable region comprising the amino acid sequence of SEQ ID NO: 158; or
xciv. a light chain variable region comprising the amino acid sequence of SEQ ID NO: 233, and a heavy chain variable region comprising the amino acid sequence of SEQ ID NO: 158; or
xcv. a light chain variable region comprising the amino acid sequence of SEQ ID NO: 243, and a heavy chain variable region comprising the amino acid sequence of SEQ ID NO: 158; or
xcvi. a light chain variable region comprising the amino acid sequence of SEQ ID NO: 253, and a heavy chain variable region comprising the amino acid sequence of SEQ ID NO: 158; or
xcvii. a light chain variable region comprising the amino acid sequence of SEQ ID NO: 103, and a heavy chain variable region comprising the amino acid sequence of SEQ ID NO: 168; or
xcviii. a light chain variable region comprising the amino acid sequence of SEQ ID NO: 113, and a heavy chain variable region comprising the amino acid sequence of SEQ ID NO: 168; or
xcix. a light chain variable region comprising the amino acid sequence of SEQ ID NO: 123, and a heavy chain variable region comprising the amino acid sequence of SEQ ID NO: 168; or
c. a light chain variable region comprising the amino acid sequence of SEQ ID NO: 133, and a heavy chain variable region comprising the amino acid sequence of SEQ ID NO: 168; or
ci. a light chain variable region comprising the amino acid sequence of SEQ ID NO: 143, and a heavy chain variable region comprising the amino acid sequence of SEQ ID NO: 168; or
cii. a light chain variable region comprising the amino acid sequence of SEQ ID NO: 153, and a heavy chain variable region comprising the amino acid sequence of SEQ ID NO: 168; or
ciii. a light chain variable region comprising the amino acid sequence of SEQ ID NO: 163, and a heavy chain variable region comprising the amino acid sequence of SEQ ID NO: 168; or
civ. a light chain variable region comprising the amino acid sequence of SEQ ID NO: 173, and a heavy chain variable region comprising the amino acid sequence of SEQ ID NO: 168; or
cv. a light chain variable region comprising the amino acid sequence of SEQ ID NO: 183, and a heavy chain variable region comprising the amino acid sequence of SEQ ID NO: 168; or
cvi. a light chain variable region comprising the amino acid sequence of SEQ ID NO: 193, and a heavy chain variable region comprising the amino acid sequence of SEQ ID NO: 168; or
cvii. a light chain variable region comprising the amino acid sequence of SEQ ID NO: 203, and a heavy chain variable region comprising the amino acid sequence of SEQ ID NO: 168; or
cviii. a light chain variable region comprising the amino acid sequence of SEQ ID NO: 213, and a heavy chain variable region comprising the amino acid sequence of SEQ ID NO: 168; or
cix. a light chain variable region comprising the amino acid sequence of SEQ ID NO: 223, and a heavy chain variable region comprising the amino acid sequence of SEQ ID NO: 168; or
cx. a light chain variable region comprising the amino acid sequence of SEQ ID NO: 233, and a heavy chain variable region comprising the amino acid sequence of SEQ ID NO: 168; or
cxi. a light chain variable region comprising the amino acid sequence of SEQ ID NO: 243, and a heavy chain variable region comprising the amino acid sequence of SEQ ID NO: 168; or
cxii. a light chain variable region comprising the amino acid sequence of SEQ ID NO: 253, and a heavy chain variable region comprising the amino acid sequence of SEQ ID NO: 168; or
cxiii. a light chain variable region comprising the amino acid sequence of SEQ ID NO: 103, and a heavy chain variable region comprising the amino acid sequence of SEQ ID NO: 178; or
cxiv. a light chain variable region comprising the amino acid sequence of SEQ ID NO: 113, and a heavy chain variable region comprising the amino acid sequence of SEQ ID NO: 178; or
cxv. a light chain variable region comprising the amino acid sequence of SEQ ID NO: 123, and a heavy chain variable region comprising the amino acid sequence of SEQ ID NO: 178; or
cxvi. a light chain variable region comprising the amino acid sequence of SEQ ID NO: 133, and a heavy chain variable region comprising the amino acid sequence of SEQ ID NO: 178; or
cxvii. a light chain variable region comprising the amino acid sequence of SEQ ID NO: 143, and a heavy chain variable region comprising the amino acid sequence of SEQ ID NO: 178; or
cxviii. a light chain variable region comprising the amino acid sequence of SEQ ID NO: 153, and a heavy chain variable region comprising the amino acid sequence of SEQ ID NO: 178; or
cxix. a light chain variable region comprising the amino acid sequence of SEQ ID NO: 163, and a heavy chain variable region comprising the amino acid sequence of SEQ ID NO: 178; or
cxx. a light chain variable region comprising the amino acid sequence of SEQ ID NO: 173, and a heavy chain variable region comprising the amino acid sequence of SEQ ID NO: 178; or
cxxi. a light chain variable region comprising the amino acid sequence of SEQ ID NO: 183, and a heavy chain variable region comprising the amino acid sequence of SEQ ID NO: 178; or
cxxii. a light chain variable region comprising the amino acid sequence of SEQ ID NO: 193, and a heavy chain variable region comprising the amino acid sequence of SEQ ID NO: 178; or
cxxiii. a light chain variable region comprising the amino acid sequence of SEQ ID NO: 203, and a heavy chain variable region comprising the amino acid sequence of SEQ ID NO: 178; or
cxxiv. a light chain variable region comprising the amino acid sequence of SEQ ID NO: 213, and a heavy chain variable region comprising the amino acid sequence of SEQ ID NO: 178; or
cxxv. a light chain variable region comprising the amino acid sequence of SEQ ID NO: 223, and a heavy chain variable region comprising the amino acid sequence of SEQ ID NO: 178; or
cxxvi. a light chain variable region comprising the amino acid sequence of SEQ ID NO: 233, and a heavy chain variable region comprising the amino acid sequence of SEQ ID NO: 178; or
cxxvii. a light chain variable region comprising the amino acid sequence of SEQ ID NO: 243, and a heavy chain variable region comprising the amino acid sequence of SEQ ID NO: 178.

5. The anti-CTGF antibody or antigen-binding fragment thereof according to any one of the foregoing paras, comprising:
i. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 109; or
ii. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 109; or
iii. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 109; or
iv. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 109; or
v. a light chain comprising an amino acid sequence at least 95% identical to the amino acid sequence of SEQ ID NO: 134, and a heavy chain comprising an amino acid sequence at least 95% identical to the amino acid sequence of SEQ ID NO: 109; or
vi. a light chain comprising an amino acid sequence at least 95% identical to the amino acid sequence of SEQ ID NO: 144, and a heavy chain comprising an amino acid sequence at least 95% identical to the amino acid sequence of SEQ ID NO: 109; or
vii. a light chain comprising an amino acid sequence at least 95% identical to the amino acid sequence of SEQ ID NO: 154, and a heavy chain comprising an amino acid sequence at least 95% identical to the amino acid sequence of SEQ ID NO: 109; or
viii. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 109; or
ix. a light chain comprising an amino acid sequence at least 95% identical to the amino acid sequence of SEQ ID NO: 174, and a heavy chain comprising an amino acid sequence at least 95% identical to the amino acid sequence of SEQ ID NO: 109; or
x. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 109; or
xi. a light chain comprising an amino acid sequence at least 95% identical to the amino acid sequence of SEQ ID NO: 194, and a heavy chain comprising an amino acid sequence at least 95% identical to the amino acid sequence of SEQ ID NO: 109; or
xii. a light chain comprising an amino acid sequence at least 95% identical to the amino acid sequence of SEQ ID NO: 204, and a heavy chain comprising an amino acid sequence at least 95% identical to the amino acid sequence of SEQ ID NO: 109; or
xiii. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 109; or
xiv. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 109; or
xv. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 109; or
xvi. a light chain comprising an amino acid sequence at least 95% identical to the amino acid sequence of SEQ ID NO: 254, and a heavy chain comprising an amino acid sequence at least 95% identical to the amino acid sequence of SEQ ID NO: 109; or
xvii. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 119; or
xviii. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 119; or
xix. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 119; or
xx. a light chain comprising an amino acid sequence at least 95% identical to the amino acid sequence of SEQ ID NO: 134, and a heavy chain comprising an amino acid sequence at least 95% identical to the amino acid sequence of SEQ ID NO: 119; or
xxi. a light chain comprising an amino acid sequence at least 95% identical to the amino acid sequence of SEQ ID NO: 144, and a heavy chain comprising an amino acid sequence at least 95% identical to the amino acid sequence of SEQ ID NO: 119; or
xxii. a light chain comprising an amino acid sequence at least 95% identical to the amino acid sequence of SEQ ID NO: 154, and a heavy chain comprising an amino acid sequence at least 95% identical to the amino acid sequence of SEQ ID NO: 119; or
xxiii. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 119; or
xxiv. a light chain comprising an amino acid sequence at least 95% identical to the amino sequence at least 95% identical to the amino acid sequence of SEQ ID NO: 119; or
xxv. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 119; or
xxvi. a light chain comprising an amino acid sequence at least 95% identical to the amino acid sequence of SEQ ID NO: 194, and a heavy chain comprising an amino acid sequence at least 95% identical to the amino acid sequence of SEQ ID NO: 119; or
xxvii. a light chain comprising an amino acid sequence at least 95% identical to the amino acid sequence of SEQ ID NO: 204, and a heavy chain comprising an amino acid sequence at least 95% identical to the amino acid sequence of SEQ ID NO: 119; or
xxviii. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 119; or
xxix. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 119; or
xxx. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 119; or
xxxi. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 119; or
xxxii. a light chain comprising an amino acid sequence at least 95% identical to the amino acid sequence of SEQ ID NO: 254, and a heavy chain comprising an amino acid sequence at least 95% identical to the amino acid sequence of SEQ ID NO: 119; or
xxxiii. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 129; or
xxxiv. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 129; or
xxxv. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 129; or
xxxvi. a light chain comprising an amino acid sequence at least 95% identical to the amino acid sequence of SEQ ID NO: 134, and a heavy chain comprising an amino acid sequence at least 95% identical to the amino acid sequence of SEQ ID NO: 129; or
xxxvii. a light chain comprising an amino acid sequence at least 95% identical to the amino acid sequence of SEQ ID NO: 144, and a heavy chain comprising an amino acid sequence at least 95% identical to the amino acid sequence of SEQ ID NO: 129; or
xxxviii. a light chain comprising an amino acid sequence at least 95% identical to the amino acid sequence of SEQ ID NO: 154, and a heavy chain comprising an amino acid sequence at least 95% identical to the amino acid sequence of SEQ ID NO: 129; or
xxxix. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 129; or
xl. a light chain comprising an amino acid sequence at least 95% identical to the amino acid sequence of SEQ ID NO: 174, and a heavy chain comprising an amino acid sequence at least 95% identical to the amino acid sequence of SEQ ID NO: 129; or
xli. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 129; or
xlii. a light chain comprising an amino acid sequence at least 95% identical to the amino acid sequence of SEQ ID NO: 194, and a heavy chain comprising an amino acid sequence at least 95% identical to the amino acid sequence of SEQ ID NO: 129; or
xliii. a light chain comprising an amino acid sequence at least 95% identical to the amino acid sequence of SEQ ID NO: 204, and a heavy chain comprising an amino acid sequence at least 95% identical to the amino acid sequence of SEQ ID NO: 129; or
xliv. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 129; or
xlv. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 129; or
xlvi. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 129; or
xlvii. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 129; or
xlviii. a light chain comprising an amino acid sequence at least 95% identical to the amino acid sequence of SEQ ID NO: 254, and a heavy chain comprising an amino acid sequence at least 95% identical to the amino acid sequence of SEQ ID NO: 129; or
xlix. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 139; or
l. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 139; or
li. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 139; or
lii. a light chain comprising an amino acid sequence at least 95% identical to the amino acid sequence of SEQ ID NO: 134, and a heavy chain comprising an amino acid sequence at least 95% identical to the amino acid sequence of SEQ ID NO: 139; or
liii. a light chain comprising an amino acid sequence at least 95% identical to the amino acid sequence of SEQ ID NO: 144, and a heavy chain comprising an amino acid sequence at least 95% identical to the amino acid sequence of SEQ ID NO: 139; or
liv. a light chain comprising an amino acid sequence at least 95% identical to the amino acid sequence of SEQ ID NO: 154, and a heavy chain comprising an amino acid sequence at least 95% identical to the amino acid sequence of SEQ ID NO: 139; or
lv. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 139; or
lvi. a light chain comprising an amino acid sequence at least 95% identical to the amino acid sequence of SEQ ID NO: 174, and a heavy chain comprising an amino acid sequence at least 95% identical to the amino acid sequence of SEQ ID NO: 139; or
lvii. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 139; or
lviii a light chain comprising an amino acid sequence at least 95% identical to the amino acid sequence of SEQ ID NO: 194, and a heavy chain comprising an amino acid sequence at least 95% identical to the amino acid sequence of SEQ ID NO: 139; or
lix. a light chain comprising an amino acid sequence at least 95% identical to the amino acid sequence of SEQ ID NO: 204, and a heavy chain comprising an amino acid sequence at least 95% identical to the amino acid sequence of SEQ ID NO: 139; or
lx. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 139; or
lxi. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 139; or
lxii. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 139; or
lxiii. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 139; or
lxiv. a light chain comprising an amino acid sequence at least 95% identical to the amino acid sequence of SEQ ID NO: 254, and a heavy chain comprising an amino acid sequence at least 95% identical to the amino acid sequence of SEQ ID NO: 139; or
lxv. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 149; or
lxvi. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 149; or
lxvii. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 149; or
lxviii. a light chain comprising an amino acid sequence at least 95% identical to the amino acid sequence of SEQ ID NO: 134, and a heavy chain comprising an amino acid sequence at least 95% identical to the amino acid sequence of SEQ ID NO: 149; or
lxix. a light chain comprising an amino acid sequence at least 95% identical to the amino acid sequence of SEQ ID NO: 144, and a heavy chain comprising an amino acid sequence at least 95% identical to the amino acid sequence of SEQ ID NO: 149; or
lxx. a light chain comprising an amino acid sequence at least 95% identical to the amino acid sequence of SEQ ID NO: 154, and a heavy chain comprising an amino acid sequence at least 95% identical to the amino acid sequence of SEQ ID NO: 149; or
lxxi. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 149; or
lxxii. a light chain comprising an amino acid sequence at least 95% identical to the amino sequence at least 95% identical to the amino acid sequence of SEQ ID NO: 149; or
lxxiii. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 149; or
lxxiv. a light chain comprising an amino acid sequence at least 95% identical to the amino acid sequence of SEQ ID NO: 194, and a heavy chain comprising an amino acid sequence at least 95% identical to the amino acid sequence of SEQ ID NO: 149; or
lxxv. a light chain comprising an amino acid sequence at least 95% identical to the amino acid sequence of SEQ ID NO: 204, and a heavy chain comprising an amino acid sequence at least 95% identical to the amino acid sequence of SEQ ID NO: 149; or
lxxvi. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 149; or
lxxvii. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 149; or
lxxviii. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 149; or
lxxix. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 149; or
lxxx. a light chain comprising an amino acid sequence at least 95% identical to the amino acid sequence of SEQ ID NO: 254, and a heavy chain comprising an amino acid sequence at least 95% identical to the amino acid sequence of SEQ ID NO: 149; or
lxxxi. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 159; or
lxxxii. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 159; or
lxxxiii. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 159; or
lxxxiv. a light chain comprising an amino acid sequence at least 95% identical to the amino acid sequence of SEQ ID NO: 134, and a heavy chain comprising an amino acid sequence at least 95% identical to the amino acid sequence of SEQ ID NO: 159; or
lxxxv. a light chain comprising an amino acid sequence at least 95% identical to the amino acid sequence of SEQ ID NO: 144, and a heavy chain comprising an amino acid sequence at least 95% identical to the amino acid sequence of SEQ ID NO: 159; or
lxxxvi. a light chain comprising an amino acid sequence at least 95% identical to the amino acid sequence of SEQ ID NO: 154, and a heavy chain comprising an amino acid sequence at least 95% identical to the amino acid sequence of SEQ ID NO: 159; or
lxxxvii. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 159; or
lxxxviii. a light chain comprising an amino acid sequence at least 95% identical to the amino acid sequence of SEQ ID NO: 174, and a heavy chain comprising an amino acid sequence at least 95% identical to the amino acid sequence of SEQ ID NO: 159; or
lxxxix. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 159; or
xc. a light chain comprising an amino acid sequence at least 95% identical to the amino acid sequence of SEQ ID NO: 194, and a heavy chain comprising an amino acid sequence at least 95% identical to the amino acid sequence of SEQ ID NO: 159; or
xci. a light chain comprising an amino acid sequence at least 95% identical to the amino acid sequence of SEQ ID NO: 204, and a heavy chain comprising an amino acid sequence at least 95% identical to the amino acid sequence of SEQ ID NO: 159; or
xcii. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 159; or
xciii. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 159; or
xciv. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 159; or
xcv. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 159; or
xcvi. a light chain comprising an amino acid sequence at least 95% identical to the amino acid sequence of SEQ ID NO: 254, and a heavy chain comprising an amino acid sequence at least 95% identical to the amino acid sequence of SEQ ID NO: 159; or
xcvii. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 169; or
xcviii. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 169; or
xcix. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 169; or
c. a light chain comprising an amino acid sequence at least 95% identical to the amino acid sequence of SEQ ID NO: 134, and a heavy chain comprising an amino acid sequence at least 95% identical to the amino acid sequence of SEQ ID NO: 169; or
ci. a light chain comprising an amino acid sequence at least 95% identical to the amino acid sequence of SEQ ID NO: 144, and a heavy chain comprising an amino acid sequence at least 95% identical to the amino acid sequence of SEQ ID NO: 169; or
cii. a light chain comprising an amino acid sequence at least 95% identical to the amino acid sequence of SEQ ID NO: 154, and a heavy chain comprising an amino acid sequence at least 95% identical to the amino acid sequence of SEQ ID NO: 169; or
ciii. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 169; or
civ. a light chain comprising an amino acid sequence at least 95% identical to the amino acid sequence of SEQ ID NO: 174, and a heavy chain comprising an amino acid sequence at least 95% identical to the amino acid sequence of SEQ ID NO: 169; or
cv. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 169; or
cvi. a light chain comprising an amino acid sequence at least 95% identical to the amino acid sequence of SEQ ID NO: 194, and a heavy chain comprising an amino acid sequence at least 95% identical to the amino acid sequence of SEQ ID NO: 169; or
cvii. a light chain comprising an amino acid sequence at least 95% identical to the amino acid sequence of SEQ ID NO: 204, and a heavy chain comprising an amino acid sequence at least 95% identical to the amino acid sequence of SEQ ID NO: 169; or
cviii. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 169; or
cix. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 169; or
cx. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 169; or
cxi. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 169; or
cxii. a light chain comprising an amino acid sequence at least 95% identical to the amino acid sequence of SEQ ID NO: 254, and a heavy chain comprising an amino acid sequence at least 95% identical to the amino acid sequence of SEQ ID NO: 169; or
cxiii. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 179; or
cxiv. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 179; or
cxv. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 179; or
cxvi. a light chain comprising an amino acid sequence at least 95% identical to the amino acid sequence of SEQ ID NO: 134, and a heavy chain comprising an amino acid sequence at least 95% identical to the amino acid sequence of SEQ ID NO: 179; or
cxvii. a light chain comprising an amino acid sequence at least 95% identical to the amino acid sequence of SEQ ID NO: 144, and a heavy chain comprising an amino acid sequence at least 95% identical to the amino acid sequence of SEQ ID NO: 179; or
cxviii. a light chain comprising an amino acid sequence at least 95% identical to the amino acid sequence of SEQ ID NO: 154, and a heavy chain comprising an amino acid sequence at least 95% identical to the amino acid sequence of SEQ ID NO: 179; or
cxix. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 179; or
cxx. a light chain comprising an amino acid sequence at least 95% identical to the amino sequence at least 95% identical to the amino acid sequence of SEQ ID NO: 179; or
cxxi. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 179; or
cxxii. a light chain comprising an amino acid sequence at least 95% identical to the amino acid sequence of SEQ ID NO: 194, and a heavy chain comprising an amino acid sequence at least 95% identical to the amino acid sequence of SEQ ID NO: 179; or
cxxiii. a light chain comprising an amino acid sequence at least 95% identical to the amino acid sequence of SEQ ID NO: 204, and a heavy chain comprising an amino acid sequence at least 95% identical to the amino acid sequence of SEQ ID NO: 179; or
cxxiv. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 179; or
cxxv. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 179; or
cxxvi. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 179; or
cxxvii. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 179.

6. The anti-CTGF antibody or antigen-binding fragment thereof according to any one of the foregoing paras, comprising:
i. a light chain comprising the amino acid sequence of SEQ ID NO: 224, and a heavy chain comprising the amino acid sequence of SEQ ID NO: 109; or
ii. a light chain comprising the amino acid sequence of SEQ ID NO: 104, and a heavy chain comprising the amino acid sequence of SEQ ID NO: 109; or
iii. a light chain comprising the amino acid sequence of SEQ ID NO: 114, and a heavy chain comprising the amino acid sequence of SEQ ID NO: 109; or
iv. a light chain comprising the amino acid sequence of SEQ ID NO: 124, and a heavy chain comprising the amino acid sequence of SEQ ID NO: 109; or
v. a light chain comprising the amino acid sequence of SEQ ID NO: 134, and a heavy chain comprising the amino acid sequence of SEQ ID NO: 109; or
vi. a light chain comprising the amino acid sequence of SEQ ID NO: 144, and a heavy chain comprising the amino acid sequence of SEQ ID NO: 109; or
vii. a light chain comprising the amino acid sequence of SEQ ID NO: 154, and a heavy chain comprising the amino acid sequence of SEQ ID NO: 109; or
viii. a light chain comprising the amino acid sequence of SEQ ID NO: 164, and a heavy chain comprising the amino acid sequence of SEQ ID NO: 109; or
ix. a light chain comprising the amino acid sequence of SEQ ID NO: 174, and a heavy chain comprising the amino acid sequence of SEQ ID NO: 109; or
x. a light chain comprising the amino acid sequence of SEQ ID NO: 184, and a heavy chain comprising the amino acid sequence of SEQ ID NO: 109; or
xi. a light chain comprising the amino acid sequence of SEQ ID NO: 194, and a heavy chain comprising the amino acid sequence of SEQ ID NO: 109; or
xii. a light chain comprising the amino acid sequence of SEQ ID NO: 204, and a heavy chain comprising the amino acid sequence of SEQ ID NO: 109; or
xiii a light chain comprising the amino acid sequence of SEQ ID NO: 214, and a heavy chain comprising the amino acid sequence of SEQ ID NO: 109; or
xiv. a light chain comprising the amino acid sequence of SEQ ID NO: 234, and a heavy chain comprising the amino acid sequence of SEQ ID NO: 109; or
xv. a light chain comprising the amino acid sequence of SEQ ID NO: 244, and a heavy chain comprising the amino acid sequence of SEQ ID NO: 109; or
xvi. a light chain comprising the amino acid sequence of SEQ ID NO: 254, and a heavy chain comprising the amino acid sequence of SEQ ID NO: 109; or
xvii. a light chain comprising the amino acid sequence of SEQ ID NO: 104, and a heavy chain comprising the amino acid sequence of SEQ ID NO: 119; or
xviii. a light chain comprising the amino acid sequence of SEQ ID NO: 114, and a heavy chain comprising the amino acid sequence of SEQ ID NO: 119; or
xix. a light chain comprising the amino acid sequence of SEQ ID NO: 124, and a heavy chain comprising the amino acid sequence of SEQ ID NO: 119; or
xx. a light chain comprising the amino acid sequence of SEQ ID NO: 134, and a heavy chain comprising the amino acid sequence of SEQ ID NO: 119; or
xxi. a light chain comprising the amino acid sequence of SEQ ID NO: 144, and a heavy chain comprising the amino acid sequence of SEQ ID NO: 119; or
xxii. a light chain comprising the amino acid sequence of SEQ ID NO: 154, and a heavy chain comprising the amino acid sequence of SEQ ID NO: 119; or
xxiii. a light chain comprising the amino acid sequence of SEQ ID NO: 164, and a heavy chain comprising the amino acid sequence of SEQ ID NO: 119; or
xxiv. a light chain comprising the amino acid sequence of SEQ ID NO: 174, and a heavy chain comprising the amino acid sequence of SEQ ID NO: 119; or
xxv. a light chain comprising the amino acid sequence of SEQ ID NO: 184, and a heavy chain comprising the amino acid sequence of SEQ ID NO: 119; or
xxvi. a light chain comprising the amino acid sequence of SEQ ID NO: 194, and a heavy chain comprising the amino acid sequence of SEQ ID NO: 119; or
xxvii. a light chain comprising the amino acid sequence of SEQ ID NO: 204, and a heavy chain comprising the amino acid sequence of SEQ ID NO: 119; or
xxviii. a light chain comprising the amino acid sequence of SEQ ID NO: 214, and a heavy chain comprising the amino acid sequence of SEQ ID NO: 119; or
xxix. a light chain comprising the amino acid sequence of SEQ ID NO: 224, and a heavy chain comprising the amino acid sequence of SEQ ID NO: 119; or
xxx. a light chain comprising the amino acid sequence of SEQ ID NO: 234, and a heavy chain comprising the amino acid sequence of SEQ ID NO: 119; or
xxxi. a light chain comprising the amino acid sequence of SEQ ID NO: 244, and a heavy chain comprising the amino acid sequence of SEQ ID NO: 119; or
xxxii. a light chain comprising the amino acid sequence of SEQ ID NO: 254, and a heavy chain comprising the amino acid sequence of SEQ ID NO: 119; or
xxxiii. a light chain comprising the amino acid sequence of SEQ ID NO: 104, and a heavy chain comprising the amino acid sequence of SEQ ID NO: 129; or
xxxiv. a light chain comprising the amino acid sequence of SEQ ID NO: 114, and a heavy chain comprising the amino acid sequence of SEQ ID NO: 129; or
xxxv. a light chain comprising the amino acid sequence of SEQ ID NO: 124, and a heavy chain comprising the amino acid sequence of SEQ ID NO: 129; or
xxxvi. a light chain comprising the amino acid sequence of SEQ ID NO: 134, and a heavy chain comprising the amino acid sequence of SEQ ID NO: 129; or
xxxvii. a light chain comprising the amino acid sequence of SEQ ID NO: 144, and a heavy chain comprising the amino acid sequence of SEQ ID NO: 129; or
xxxviii. a light chain comprising the amino acid sequence of SEQ ID NO: 154, and a heavy chain comprising the amino acid sequence of SEQ ID NO: 129; or
xxxix. a light chain comprising the amino acid sequence of SEQ ID NO: 164, and a heavy chain comprising the amino acid sequence of SEQ ID NO: 129; or
xl. a light chain comprising the amino acid sequence of SEQ ID NO: 174, and a heavy chain comprising the amino acid sequence of SEQ ID NO: 129; or
xli. a light chain comprising the amino acid sequence of SEQ ID NO: 184, and a heavy chain comprising the amino acid sequence of SEQ ID NO: 129; or
xlii. a light chain comprising the amino acid sequence of SEQ ID NO: 194, and a heavy chain comprising the amino acid sequence of SEQ ID NO: 129; or
xliii. a light chain comprising the amino acid sequence of SEQ ID NO: 204, and a heavy chain comprising the amino acid sequence of SEQ ID NO: 129; or
xliv. a light chain comprising the amino acid sequence of SEQ ID NO: 214, and a heavy chain comprising the amino acid sequence of SEQ ID NO: 129; or
xlv. a light chain comprising the amino acid sequence of SEQ ID NO: 224, and a heavy chain comprising the amino acid sequence of SEQ ID NO: 129; or
xlvi. a light chain comprising the amino acid sequence of SEQ ID NO: 234, and a heavy chain comprising the amino acid sequence of SEQ ID NO: 129; or
xlvii. a light chain comprising the amino acid sequence of SEQ ID NO: 244, and a heavy chain comprising the amino acid sequence of SEQ ID NO: 129; or
xlviii. a light chain comprising the amino acid sequence of SEQ ID NO: 254, and a heavy chain comprising the amino acid sequence of SEQ ID NO: 129; or
xlix. a light chain comprising the amino acid sequence of SEQ ID NO: 104, and a heavy chain comprising the amino acid sequence of SEQ ID NO: 139; or
l. a light chain comprising the amino acid sequence of SEQ ID NO: 114, and a heavy chain comprising the amino acid sequence of SEQ ID NO: 139; or
li. a light chain comprising the amino acid sequence of SEQ ID NO: 124, and a heavy chain comprising the amino acid sequence of SEQ ID NO: 139; or
lii. a light chain comprising the amino acid sequence of SEQ ID NO: 134, and a heavy chain comprising the amino acid sequence of SEQ ID NO: 139; or
liii. a light chain comprising the amino acid sequence of SEQ ID NO: 144, and a heavy chain comprising the amino acid sequence of SEQ ID NO: 139; or
liv. a light chain comprising the amino acid sequence of SEQ ID NO: 154, and a heavy chain comprising the amino acid sequence of SEQ ID NO: 139; or
lv. a light chain comprising the amino acid sequence of SEQ ID NO: 164, and a heavy chain comprising the amino acid sequence of SEQ ID NO: 139; or
lvi. a light chain comprising the amino acid sequence of SEQ ID NO: 174, and a heavy chain comprising the amino acid sequence of SEQ ID NO: 139; or
lvii. a light chain comprising the amino acid sequence of SEQ ID NO: 184, and a heavy chain comprising the amino acid sequence of SEQ ID NO: 139; or
lviii. a light chain comprising the amino acid sequence of SEQ ID NO: 194, and a heavy chain comprising the amino acid sequence of SEQ ID NO: 139; or
lix. a light chain comprising the amino acid sequence of SEQ ID NO: 204, and a heavy chain comprising the amino acid sequence of SEQ ID NO: 139; or
lx. a light chain comprising the amino acid sequence of SEQ ID NO: 214, and a heavy chain comprising the amino acid sequence of SEQ ID NO: 139; or
lxi. a light chain comprising the amino acid sequence of SEQ ID NO: 224, and a heavy chain comprising the amino acid sequence of SEQ ID NO: 139; or
lxii. a light chain comprising the amino acid sequence of SEQ ID NO: 234, and a heavy chain comprising the amino acid sequence of SEQ ID NO: 139; or
lxiii. a light chain comprising the amino acid sequence of SEQ ID NO: 244, and a heavy chain comprising the amino acid sequence of SEQ ID NO: 139; or
lxiv. a light chain comprising the amino acid sequence of SEQ ID NO: 254, and a heavy chain comprising the amino acid sequence of SEQ ID NO: 139; or
lxv. a light chain comprising the amino acid sequence of SEQ ID NO: 104, and a heavy chain comprising the amino acid sequence of SEQ ID NO: 149; or
lxvi. a light chain comprising the amino acid sequence of SEQ ID NO: 114, and a heavy chain comprising the amino acid sequence of SEQ ID NO: 149; or
lxvii. a light chain comprising the amino acid sequence of SEQ ID NO: 124, and a heavy chain comprising the amino acid sequence of SEQ ID NO: 149; or
lxviii. a light chain comprising the amino acid sequence of SEQ ID NO: 134, and a heavy chain comprising the amino acid sequence of SEQ ID NO: 149; or
lxix. a light chain comprising the amino acid sequence of SEQ ID NO: 144, and a heavy chain comprising the amino acid sequence of SEQ ID NO: 149; or
lxx. a light chain comprising the amino acid sequence of SEQ ID NO: 154, and a heavy chain comprising the amino acid sequence of SEQ ID NO: 149; or
lxxi. a light chain comprising the amino acid sequence of SEQ ID NO: 164, and a heavy chain comprising the amino acid sequence of SEQ ID NO: 149; or
lxxii. a light chain comprising the amino acid sequence of SEQ ID NO: 174, and a heavy chain comprising the amino acid sequence of SEQ ID NO: 149; or
lxxiii. a light chain comprising the amino acid sequence of SEQ ID NO: 184, and a heavy chain comprising the amino acid sequence of SEQ ID NO: 149; or
lxxiv. a light chain comprising the amino acid sequence of SEQ ID NO: 194, and a heavy chain comprising the amino acid sequence of SEQ ID NO: 149; or
lxxv. a light chain comprising the amino acid sequence of SEQ ID NO: 204, and a heavy chain comprising the amino acid sequence of SEQ ID NO: 149; or
lxxvi. a light chain comprising the amino acid sequence of SEQ ID NO: 214, and a heavy chain comprising the amino acid sequence of SEQ ID NO: 149; or
lxxvii. a light chain comprising the amino acid sequence of SEQ ID NO: 224, and a heavy chain comprising the amino acid sequence of SEQ ID NO: 149; or
lxxviii. a light chain comprising the amino acid sequence of SEQ ID NO: 234, and a heavy chain comprising the amino acid sequence of SEQ ID NO: 149; or
lxxix. a light chain comprising the amino acid sequence of SEQ ID NO: 244, and a heavy chain comprising the amino acid sequence of SEQ ID NO: 149; or
lxxx. a light chain comprising the amino acid sequence of SEQ ID NO: 254, and a heavy chain comprising the amino acid sequence of SEQ ID NO: 149; or
lxxxi. a light chain comprising the amino acid sequence of SEQ ID NO: 104, and a heavy chain comprising the amino acid sequence of SEQ ID NO: 159; or
lxxxii. a light chain comprising the amino acid sequence of SEQ ID NO: 114, and a heavy chain comprising the amino acid sequence of SEQ ID NO: 159; or
lxxxiii. a light chain comprising the amino acid sequence of SEQ ID NO: 124, and a heavy chain comprising the amino acid sequence of SEQ ID NO: 159; or
lxxxiv. a light chain comprising the amino acid sequence of SEQ ID NO: 134, and a heavy chain comprising the amino acid sequence of SEQ ID NO: 159; or
lxxxv. a light chain comprising the amino acid sequence of SEQ ID NO: 144, and a heavy chain comprising the amino acid sequence of SEQ ID NO: 159; or
lxxxvi. a light chain comprising the amino acid sequence of SEQ ID NO: 154, and a heavy chain comprising the amino acid sequence of SEQ ID NO: 159; or
lxxxvii. a light chain comprising the amino acid sequence of SEQ ID NO: 164, and a heavy chain comprising the amino acid sequence of SEQ ID NO: 159; or
lxxxviii. a light chain comprising the amino acid sequence of SEQ ID NO: 174, and a heavy chain comprising the amino acid sequence of SEQ ID NO: 159; or
lxxxix. a light chain comprising the amino acid sequence of SEQ ID NO: 184, and a heavy chain comprising the amino acid sequence of SEQ ID NO: 159; or
xc. a light chain comprising the amino acid sequence of SEQ ID NO: 194, and a heavy chain comprising the amino acid sequence of SEQ ID NO: 159; or
xci. a light chain comprising the amino acid sequence of SEQ ID NO: 204, and a heavy chain comprising the amino acid sequence of SEQ ID NO: 159; or
xcii. a light chain comprising the amino acid sequence of SEQ ID NO: 214, and a heavy chain comprising the amino acid sequence of SEQ ID NO: 159; or
xciii. a light chain comprising the amino acid sequence of SEQ ID NO: 224, and a heavy chain comprising the amino acid sequence of SEQ ID NO: 159; or
xciv. a light chain comprising the amino acid sequence of SEQ ID NO: 234, and a heavy chain comprising the amino acid sequence of SEQ ID NO: 159; or
xcv. a light chain comprising the amino acid sequence of SEQ ID NO: 244, and a heavy chain comprising the amino acid sequence of SEQ ID NO: 159; or
xcvi. a light chain comprising the amino acid sequence of SEQ ID NO: 254, and a heavy chain comprising the amino acid sequence of SEQ ID NO: 159; or
xcvii. a light chain comprising the amino acid sequence of SEQ ID NO: 104, and a heavy chain comprising the amino acid sequence of SEQ ID NO: 169; or
xcviii. a light chain comprising the amino acid sequence of SEQ ID NO: 114, and a heavy chain comprising the amino acid sequence of SEQ ID NO: 169; or
xcix. a light chain comprising the amino acid sequence of SEQ ID NO: 124, and a heavy chain comprising the amino acid sequence of SEQ ID NO: 169; or
c. a light chain comprising the amino acid sequence of SEQ ID NO: 134, and a heavy chain comprising the amino acid sequence of SEQ ID NO: 169; or
ci. a light chain comprising the amino acid sequence of SEQ ID NO: 144, and a heavy chain comprising the amino acid sequence of SEQ ID NO: 169; or
cii. a light chain comprising the amino acid sequence of SEQ ID NO: 154, and a heavy chain comprising the amino acid sequence of SEQ ID NO: 169; or
ciii. a light chain comprising the amino acid sequence of SEQ ID NO: 164, and a heavy chain comprising the amino acid sequence of SEQ ID NO: 169; or
civ. a light chain comprising the amino acid sequence of SEQ ID NO: 174, and a heavy chain comprising the amino acid sequence of SEQ ID NO: 169; or
cv. a light chain comprising the amino acid sequence of SEQ ID NO: 184, and a heavy chain comprising the amino acid sequence of SEQ ID NO: 169; or
cvi. a light chain comprising the amino acid sequence of SEQ ID NO: 194, and a heavy chain comprising the amino acid sequence of SEQ ID NO: 169; or
cvii. a light chain comprising the amino acid sequence of SEQ ID NO: 204, and a heavy chain comprising the amino acid sequence of SEQ ID NO: 169; or
cviii. a light chain comprising the amino acid sequence of SEQ ID NO: 214, and a heavy chain comprising the amino acid sequence of SEQ ID NO: 169; or
cix. a light chain comprising the amino acid sequence of SEQ ID NO: 224, and a heavy chain comprising the amino acid sequence of SEQ ID NO: 169; or
cx. a light chain comprising the amino acid sequence of SEQ ID NO: 234, and a heavy chain comprising the amino acid sequence of SEQ ID NO: 169; or
cxi. a light chain comprising the amino acid sequence of SEQ ID NO: 244, and a heavy chain comprising the amino acid sequence of SEQ ID NO: 169; or
cxii. a light chain comprising the amino acid sequence of SEQ ID NO: 254, and a heavy chain comprising the amino acid sequence of SEQ ID NO: 169; or
cxiii. a light chain comprising the amino acid sequence of SEQ ID NO: 104, and a heavy chain comprising the amino acid sequence of SEQ ID NO: 179; or
cxiv. a light chain comprising the amino acid sequence of SEQ ID NO: 114, and a heavy chain comprising the amino acid sequence of SEQ ID NO: 179; or
cxv. a light chain comprising the amino acid sequence of SEQ ID NO: 124, and a heavy chain comprising the amino acid sequence of SEQ ID NO: 179; or
cxvi. a light chain comprising the amino acid sequence of SEQ ID NO: 134, and a heavy chain comprising the amino acid sequence of SEQ ID NO: 179; or
cxvii. a light chain comprising the amino acid sequence of SEQ ID NO: 144, and a heavy chain comprising the amino acid sequence of SEQ ID NO: 179; or
cxviii. a light chain comprising the amino acid sequence of SEQ ID NO: 154, and a heavy chain comprising the amino acid sequence of SEQ ID NO: 179; or
cxix. a light chain comprising the amino acid sequence of SEQ ID NO: 164, and a heavy chain comprising the amino acid sequence of SEQ ID NO: 179; or
cxx. a light chain comprising the amino acid sequence of SEQ ID NO: 174, and a heavy chain comprising the amino acid sequence of SEQ ID NO: 179; or
cxxi. a light chain comprising the amino acid sequence of SEQ ID NO: 184, and a heavy chain comprising the amino acid sequence of SEQ ID NO: 179; or
cxxii. a light chain comprising the amino acid sequence of SEQ ID NO: 194, and a heavy chain comprising the amino acid sequence of SEQ ID NO: 179; or
cxxiii. a light chain comprising the amino acid sequence of SEQ ID NO: 204, and a heavy chain comprising the amino acid sequence of SEQ ID NO: 179; or
cxxiv. a light chain comprising the amino acid sequence of SEQ ID NO: 214, and a heavy chain comprising the amino acid sequence of SEQ ID NO: 179; or
cxxv. a light chain comprising the amino acid sequence of SEQ ID NO: 224, and a heavy chain comprising the amino acid sequence of SEQ ID NO: 179; or
cxxvi. a light chain comprising the amino acid sequence of SEQ ID NO: 234, and a heavy chain comprising the amino acid sequence of SEQ ID NO: 179; or
cxxvii. a light chain comprising the amino acid sequence of SEQ ID NO: 244, and a heavy chain comprising the amino acid sequence of SEQ ID NO: 179.

7. The anti-CTGF antibody or an antigen-binding fragment thereof according to any one of the foregoing paras, comprising a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107.

8. The anti-CTGF antibody or an antigen-binding fragment thereof according to any one of the foregoing paras, comprising a light chain variable region comprising the amino acid sequence of SEQ ID NO: 223, and a heavy chain variable region comprising the amino acid sequence of SEQ ID NO: 108.

9. The anti-CTGF antibody or antigen-binding fragment thereof according to any one of the foregoing paras, comprising a light chain comprising the amino acid sequence of SEQ ID NO: 224, and a heavy chain comprising the amino acid sequence of SEQ ID NO: 109.

10. The anti-CTGF antibody of any one of the foregoing paras, wherein (a) said antibody comprises a heavy chain constant region selected from IgG₁, IgG₂, IgG₃, IgG₄, IgM, IgA and IgE or a variant thereof; (b) the antibody is a monoclonal antibody; and/or (c) the antibody is a human, humanized, or chimeric antibody, preferably a humanized antibody.

11. The anti-CTGF antibody of any one of the foregoing paras, wherein said antibody (a) binds human CTGF with a K_{D} of 5 pM or lower; (b) binds the N-terminal fragment of human CTGF with a K_{D} of 0.5 nM or lower; (c) decreases or blocks human CTGF binding to primary human fibroblast cells; (d) decreases or blocks cleavage of human CTGF by MMP7; (e) blocks or inhibits human TGFbeta-induced increase in COL1A1 expression in lung epithelial cells; (f) blocks or inhibits CTGF interaction with alveolar type II cells, fibroblasts and A549 cells; and/or (g) blocks or inhibits CTGF-NTF interaction with alveolar type II cells, fibroblasts and A549 cells.

12. A pharmaceutical composition comprising the antibody or an antigen-binding fragment thereof according to any of the foregoing paras and a pharmaceutically acceptable carrier.

13. The antibody or an antigen-binding fragment thereof according to any one of paras 1 to 11 or the composition according to para 12 for use as a medicament.

14. The antibody or an antigen-binding fragment thereof according to any one of paras 1 to 11 or the composition according to para 12 for use in treating or preventing a CTGF-associated disorder, a fibrotic disease, hypertension, diabetes, myocardial infarction, arthritis, CTGF-related cell proliferative disease, atherosclerosis, glaucoma, or a cancer.

15. The antibody, or an antigen-binding fragment thereof, or the composition for use according to para 13 or 14, wherein said fibrotic disease is idiopathic pulmonary fibrosis (IPF), progressive pulmonary fibrosis (PPF), pulmonary fibrosis with interstitial lung disease (PF-ILD), systemic scleroderma (SSc), Duchenne muscular dystrophy, diabetic nephropathy, diabetic retinopathy, osteoarthritis, scleroderma, chronic heart failure, liver cirrhosis or renal fibrosis, or wherein said cancer is acute lymphoblastic leukemia, dermatofibroma, breast cancer, angiolipoma, angioleiomyoma, connective tissue-generating cancer, prostate cancer, ovarian cancer, colorectal cancer, pancreatic cancer, gastrointestinal cancer, or liver cancer.

16. An isolated polynucleotide or a plurality of isolated polynucleotides that encodes a sequence comprising the heavy chain variable region and/or light chain variable region of the antibody or an antigen-binding fragment thereof according to any one of paras 1 to 11.

17. A vector or plurality of vectors comprising the polynucleotide or the plurality of polynucleotides according to para 16, preferably an expression vector or plurality of expression vectors, more preferably a vector or plurality of vectors comprising said polynucleotide or plurality of polynucleotides in functional association with one or more expression control sequences.

18. A host cell comprising the polynucleotide or plurality of nucleotides according to para 17.

19. A method for the production of an anti-CTGF antibody or antigen-binding fragment thereof according to any one of paras 1 to 11, comprising the steps:
(a) cultivating a host cell according to para 18 under conditions allowing the expression of the anti-CTGF antibody or antigen-binding fragment thereof; and
(b) recovering said anti-CTGF antibody or antigen-binding fragment thereof.

20. A method of inhibiting the interaction between human CTGF or human CTGF-NTF and a cell, comprising contacting said human CTGF with an effective amount of the anti-CTGF antibody or antigen-binding fragment thereof according to any one of paras 1 to 11.

### DETAILED DESCRIPTION

This disclosure generally relates to anti-CTGF antibodies or antigen-binding fragments thereof. Exemplary anti-CTGF antibodies disclosed herein address the need for treatments of conditions modulated by CTGF signaling. In some aspects, the anti-CTGF antibodies or antigen-binding fragments thereof are for diagnostic and/or therapeutic use, for example in a subject in need thereof, such as a human.

In some aspects, the present disclosure provides an anti-CTGF antibody, in particular, a monoclonal anti-CTGF antibody, for example a human or humanized monoclonal anti-CTGF antibody, having one or more of the properties described herein.

In some aspects, the anti-CTGF-antibody or antigen-binding fragment thereof decreases or prevents the cleavage of full length CTGF by a protease, such as MMP7.

In some aspects, the anti-CTGF-antibody or antigen-binding fragment thereof decreases or blocks the interaction between CTGF and cells that express one or more receptors for CTGF, such as alveolar type II cells, fibroblasts and A549 cells.

In some aspects, the anti-CTGF-antibody or antigen-binding fragment thereof decreases or prevents the inhibition of macrophage efferocytosis by CTGF-NTF.

In some aspects, the anti-CTGF-antibody or antigen-binding fragment thereof decreases or prevents fibrotic progression in human small airway cells raised at an air-liquid interface and exposed to pro-fibrotic cytokines, such as the IPF cytokine cocktail (IPF-RC) described in Example 5 herein.

In some aspects, the anti-CTGF-antibody or antigen-binding fragment thereof exhibits a binding affinity (K_{D}) for full length human CTGF of lower than 50 pM, lower than 25 pM, lower than 15 pM, lower than 10 pM, lower than 5 pM, or lower than 2 pM, preferably when measured by surface plasmon resonance at 25 °C.

In some aspects, the anti-CTGF-antibody or antigen-binding fragment thereof exhibits a binding affinity (K_{D}) for human CTGF-NTF of lower than 10 nM, lower than 5 nM, lower than 2.5 nM, lower than 1 nM, lower than 0.5 nM, or lower than 0.2 nM, preferably when measured by surface plasmon resonance at 25 °C.

In some aspects, the anti-CTGF antibody or antigen-binding fragment thereof of the present disclosure has favorable pharmacokinetic properties. In some aspects, the anti-CTGF antibody of the present disclosure has favorable biophysical properties, for example yield, quality, stability, or solubility.

In the present detailed description, numerous specific details are set forth to provide a full understanding of the subject technology. It will be apparent, however, to one of ordinary skill in the art that the subject technology may be practiced without some of these specific details. In other instances, well-known structures and techniques have not been shown in detail so as not to obscure the subject technology. Preferences and options for a given aspect, feature, embodiment, or parameter should, unless the context indicates otherwise, be regarded as having been disclosed in combination with any and all preferences and options for all other aspects, features, embodiments and parameters of the invention.

### Antibodies

The generalized structure of antibodies or immunoglobulin is well known to those of skill in the art, these molecules are heterotetrametric glycoproteins, typically of about 150,000 daltons, composed of two identical light (L) chains and two identical heavy (H) chains. Each light chain is covalently linked to a heavy chain by one disulfide bond to form a heterodimer, and the heterotrimeric molecule is formed through a covalent disulfide linkage between the two identical heavy chains of the heterodimers. Although the light and heavy chains are linked together by one disulfide bond, the number of disulfide linkages between the two heavy chains varies by immunoglobulin isotype. Each heavy and light chain also has regularly spaced intrachain disulfide bridges. Each heavy chain has at the amino-terminus a variable domain (V_{H} is the variable heavy chain), followed by three or four constant domains (C_{H}1, C_{H}2, C_{H}3, and optionally C_{H}4), as well as a hinge region between C_{H}1 and C_{H}2. Each light chain has two domains, an amino-terminal variable domain (V_{L} is the variable light chain) and a carboxy-terminal constant domain (C_{L}). The V_{L} domain associates non-covalently with the V_{H} domain, whereas the C_{L} domain is commonly covalently linked to the C_{H}1 domain via a disulfide bond. Particular amino acid residues are believed to form an interface between the light and heavy chain variable domains (Chothia et al., 1985, J. Mol. Biol. 186:651-663, Vargas-Madrazo E, Paz-García E. J Mol Recognit. 2003; 16(3):b 113-120). The variable domains are also referred herein as variable regions, and the constant domains as constant regions.

Certain domains within the variable domains differ extensively between different antibodies i.e., are “hypervariable.” These hypervariable domains contain residues that are directly involved in the binding and specificity of each particular antibody for its specific antigenic determinant. Hypervariability, both in the light chain and the heavy chain variable domains, is concentrated in three segments known as complementarity determining regions (CDRs) or hypervariable loops (HVLs). CDRs are defined by sequence comparison in Kabat et al., 1991, In: Sequences of Proteins of Immunological Interest, 5th Ed. Public Health Service, National Institutes of Health, Bethesda, Md., whereas HVLs are structurally defined according to the three-dimensional structure of the variable domain, as described by Chothia and Lesk, 1987, J. Mol. Biol. 196:901-917. As defined by Kabat, CDR-L1 is positioned at about residues 24-34, CDR-L2, at about residues 50-56, and CDR-L3, at about residues 89-97 in the light chain variable domain; CDR-H1 is positioned at about residues 31-35, CDR-H2 at about residues 50-65, and CDR-H3 at about residues 95-102 in the heavy chain variable domain. Unless indicated otherwise, references to CDR sequences herein refer to the CDR sequences defined by Kabat. IMGT and NORTH provide alternative definitions of the CDRs (see, Lefranc MP. Unique database numbering system for immunogenetic analysis. Immunol Today (1997) 18:509; and North B, Lehmann A, Dunbrack RLJ. A new clustering of antibody CDR loop conformations. J Mol Biol. (2011) 406:228-56). Additionally, CDRs may be defined per the Chemical Computing Group (CCG) numbering (Almagro et al., Proteins 2011; 79:3050-3066 and Maier et al, Proteins 2014; 82:1599-1610). The CDR1, CDR2, CDR3 of the heavy and light chains therefore define the unique and functional properties specific for a given antibody. The skilled person is able to determine the CDR sequences according to aforementioned numbering schemes from a specified VH and/or VL sequences. Except where indicated otherwise, the present invention has been disclosed with reference to the CDRs defined according to Kabat, though it is to be understood that the invention could likewise be determined with reference to other CDR numbering schemes, such as those CDRs disclosed in Tables 3-8 herein (e.g., Chothia, CCG, IMGT, or Aho).

The three CDRs within each of the heavy and light chains are separated by framework regions (FR), which contain sequences that tend to be less variable. From the amino terminus to the carboxy terminus of the heavy and light chain variable domains, the FRs and CDRs are arranged in the order: FR1, CDR1, FR2, CDR2, FR3, CDR3, and FR4. The largely β-sheet configuration of the FRs brings the CDRs within each of the chains into close proximity to each other as well as to the CDRs from the other chain. The resulting conformation contributes to the antigen binding site (see Kabat et al., 1991, NIH Publ. No. 91-3242, Vol. I, pages 647-669), although not all CDR residues are necessarily directly involved in antigen binding. Those skilled in the art can routinely determine which residues comprise a particular CDR given the variable region amino acid sequence of the antibody. The CDR1, CDR2, CDR3 of the heavy and light chains therefore define the unique and functional properties specific for a given antibody.

FR residues and Ig constant domains are generally not directly involved in antigen binding but contribute to antigen binding and/or mediate antibody effector function. Some FR residues are thought to have a significant effect on antigen binding in at least three ways: by noncovalently binding directly to an epitope, by interacting with one or more CDR residues, and by affecting the interface between the heavy and light chains. The constant domains are not directly involved in antigen binding but mediate various Ig effector functions, such as participation of the antibody in antibody-dependent cellular cytotoxicity (ADCC), complement-dependent cytotoxicity (CDC) and antibody-dependent cellular phagocytosis (ADCP).

The light chains of vertebrate immunoglobulins are assigned to one of two clearly distinct classes, kappa (κ) and lambda (λ), based on the amino acid sequence of the constant domain. By comparison, the heavy chains of mammalian immunoglobulins are assigned to one of five major classes, according to the sequence of the constant domains: IgA, IgD, IgE, IgG, and IgM. IgG and IgA are further divided into subclasses (isotypes), e.g., IgG₁, IgG₂, IgG₃, IgG₄, IgA₁, and IgA₂, respectively. The antibodies, as described herein, may be of any one these classes or subclasses. Preferably, the antibody is of the IgG-type, more preferably IgG₁. The heavy chain constant domains that correspond to the different classes of immunoglobulins are called α, δ, ε, γ, and μ, respectively. The subunit structures and three-dimensional configurations of the classes of native immunoglobulins are well known.

### DEFINITIONS

The terms, “antibody”, and “anti-CTGF antibody”, are used herein interchangeably and encompass monoclonal antibodies (including full length monoclonal antibodies), multispecific antibodies (e.g., bispecific antibodies), antibodies with minor modifications such as N-or C-terminal truncations and antibody fragments such as variable domains and other portions of antibodies that exhibit a desired biological activity, e.g., CTGF binding.

The term “monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation that may be present. Monoclonal antibodies typically bind one antigenic epitope. A bispecific monoclonal antibody binds two distinct antigenic epitopes. A monoclonal antibody may be monospecific or multispecific such as bispecific, monovalent, bivalent or multivalent. It should be understood that monoclonal antibodies can be made by any technique or methodology known in the art; including e.g., the hybridoma method (Kohler et al., 1975, Nature 256:495), or recombinant DNA methods known in the art (see, e.g., U.S. Pat. No. 4,816,567), or methods of isolation of monoclonal recombinantly produced using phage antibody libraries, using techniques described in Clackson et al., 1991, Nature 352:624-628, and Marks et al., 1991, J. Mol. Biol. 222:581-597.

Chimeric antibodies consist of the heavy and light chain variable regions of an antibody from one species (e.g., a non-human mammal such as a mouse) and the heavy and light chain constant regions of another species (e.g., human) antibody and can be obtained by linking the DNA sequences encoding the variable regions of the antibody from the first species (e.g., mouse) to the DNA sequences for the constant regions of the antibody from the second (e.g. human) species and transforming a host with an expression vector containing the linked sequences to allow it to produce a chimeric antibody. Alternatively, the chimeric antibody also could be one in which one or more regions or domains of the heavy and/or light chain is identical with, homologous to, or a variant of the corresponding sequence in a monoclonal antibody from another immunoglobulin class or isotype, or from a consensus or germline sequence. Chimeric antibodies can include fragments of such antibodies, provided that the antibody fragment exhibits the desired biological activity of its parent antibody, for example binding to the same epitope (see, e.g., U.S. Pat. No. 4,816,567; and Morrison et al., 1984, Proc. Natl. Acad. Sci. USA 81:6851-6855).

The terms “antibody fragment”, “antigen-binding fragment”, “anti-CTGF antibody fragment”, “anti-CTGF antibody fragment”, “engineered anti-CTGF antibody fragment” refer to a portion of a full length anti-CTGF antibody, in which a variable region or a functional capability is retained, for example, CTGF binding. Examples of antibody fragments include, but are not limited to, a Fab, Fab′, F(ab′)₂, Fd, Fv, scFv and scFv-Fc fragment, a diabody, a linear antibody, a single-chain antibody, a minibody, a diabody formed from antibody fragments, and multispecific antibodies formed from antibody fragments.

Antibody fragments can be obtained for example by treating full length antibodies treated with enzymes such as papain or pepsin to generate useful antibody fragments. Papain digestion is used to produce two identical antigen-binding antibody fragments called “Fab” fragments, each with a single antigen-binding site, and a residual “Fc” fragment. The Fab fragment also contains the constant domain of the light chain and the C_{H}1 domain of the heavy chain. Pepsin treatment yields a F(ab′)₂ fragment that has two antigen-binding sites and is still capable of cross-linking antigen.

An example of antibody fragments according to the present disclosure are Fab′ fragments. Fab′ fragments differ from Fab fragments by the presence of additional residues including one or more cysteines from the antibody hinge region at the C-terminus of the C_{H}1 domain. F(ab′)₂ antibody fragments are pairs of Fab′ fragments linked by cysteine residues in the hinge region. Other chemical couplings of antibody fragments are also known.

A “Fv” fragment contains a complete antigen-recognition and binding site consisting of a dimer of one heavy and one light chain variable domain in tight, non-covalent association. In this configuration, the three CDRs of each variable domain interact to define an antigen-biding site on the surface of the V_{H}-V_{L} dimer. Collectively, the six CDRs confer antigen-binding specificity to the antibody.

Antibody fragments may also include “single-chain Fv” or “scFv” fragments. A “single-chain Fv” or “scFv” antibody fragment is a single chain Fv variant comprising the V_{H} and V_{L} domains of an antibody where the domains are present in a single polypeptide chain. The single chain Fv is capable of recognizing and binding antigen. The scFv polypeptide may optionally also contain a polypeptide linker positioned between the V_{H} and V_{L} domains in order to facilitate formation of a desired three-dimensional structure for antigen binding by the scFv (see, e.g., Pluckthun, 1994, In The Pharmacology of monoclonal Antibodies, Vol. 113, Rosenburg and Moore eds., Springer-Verlag, New York, pp. 269-315).

Antibody fragments may also form tandem Fd segments, which comprise a pair of tandem Fd segments (V_{H}-C_{H}1-V_{H}-C_{H}1) to form a pair of antigen binding regions. These “linear antibodies” can be bispecific or monospecific as described in, for example, Zapata et al. 1995, Protein Eng. 8(10):1057-1062.

The term “human antibody” as used herein includes antibodies or fragments thereof derived from human germline immunoglobulin sequences. The term “human antibody” is not intended to include antibodies in which CDR sequences derived from the germline of another (mammalian) species, such as a mouse, rat or rabbit, have been grafted onto human framework sequences. Thus, as used herein, the term “human antibody” refers to an antibody or fragment thereof in which every part of the protein (e.g., CDR, framework, C_{L}, C_{H} domains (e.g., C_{H}1, C_{H}2, C_{H}3), hinge, V_{L}, V_{H}) is substantially non-immunogenic in humans, with only minor sequence changes or variations as further described herein below.

Technologies for creating such a “human antibody” have been described and include without being limiting phage display or use of transgenic animals (www.Ablexis.com/technology-alivamab.php; WO 90/05144; D. Marks, H. R. Hoogenboom, T. P. Bonnert, J. McCafferty, A. D. Griffiths and G. Winter (1991) “By-passing immunisation. Human antibodies from V-gene libraries displayed on phage.” J.Mol.Biol., 222, 581-597; Knappik et al., J. Mol. Biol. 296:57-86, 2000; S. Carmen and L. Jermutus, “Concepts in antibody phage display”. Briefings in Functional Genomics and Proteomics 2002 1(2): 189-203; Lonberg N, Huszar D. “Human antibodies from transgenic mice”. Int Rev Immunol. 1995; 13(1):65-93; Brüggemann M, Taussig MJ. “Production of human antibody repertoires in transgenic mice”. Curr Opin Biotechnol. 1997 August; 8(4):455-8).

Thus, a human antibody is distinct from e.g., a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes.

In some aspects, an anti-CTGF antibody is a humanized antibody or antibody fragment thereof. A humanized antibody or a humanized antibody fragment is a specific type of chimeric antibody which includes an immunoglobulin amino acid sequence variant, or fragment thereof, which is capable of binding to a predetermined antigen and which, comprises one or more FRs having substantially the amino acid sequence of a human immunoglobulin and one or more CDRs having substantially the amino acid sequence of a non-human immunoglobulin. This non-human amino acid sequence often referred to as an “import” sequence is typically taken from an “import” antibody domain, particularly a variable domain. In general, a humanized antibody includes at least the CDRs or HVLs of a non-human antibody, inserted between the FRs of a human heavy or light chain variable domain. Methods of humanization of antibodies are for example described by Almagro et al., (2008) Frontiers in Bioscience 13, 1619-1633, or in WO12092374 A2.

The chimeric, humanized or human antibodies or antigen-binding fragments thereof of the present disclosure may further be engineered. Such engineering includes without limitation the removal or exchange of undesired amino acids, for example to reduce immunogenicity in humans, or to avoid deamidation, undesirable charges or lipophilicity or non-specific binding. Such removal or exchange of undesired amino acids can, for example, be introduced by random or site-specific mutagenesis in vitro or by somatic mutation in vivo. Moreover, in connection with chimeric or humanized antibodies, it will be understood that certain mouse FR residues may be retained in an antibody or fragment thereof.

In some aspects, an anti-CTGF antibody comprises substantially all of at least one, and typically two, variable domains (such as contained, for example, in Fab, Fab′, F(ab′)₂, ScFv, and Fv fragments). In some aspects, the anti-CTGF antibody also includes at least a portion of an immunoglobulin Fc region, typically that of a human immunoglobulin. Ordinarily, the antibody will contain both the light chain as well as at least the variable domain of a heavy chain. The antibody also may include one or more of the C_{H}1, hinge, C_{H}2, C_{H}3, and/or C_{H}4 regions of the heavy chain, as appropriate.

In some aspects, an anti-CTGF antibody can be selected from any class of immunoglobulins, including IgM, IgG, IgD, IgA and IgE, and any isotype, including IgG₁, IgG₂, IgG₃, IgG₄, IgA₁ and IgA₂. An alternative anti-CTGF antibody can comprise sequences from more than one immunoglobulin class or isotype, and selecting particular modified or unmodified constant domains to optimize desired effector functions is within the ordinary skill in the art.

For example, the Fc region of an antibody mediates its serum half-life and effector functions, such as complement-dependent cytotoxicity (CDC), antibody-dependent cellular cytotoxicity (ADCC) and antibody-dependent cell phagocytosis (ADCP). Fc engineering can be employed to optimize antibody properties suited to the pharmacology activity required of them. Where such cytotoxic activity is not desirable, such as targeting an immune cell in the treatment of cancer, the constant domain may be of isotype with reduced effector function, such as IgG₄, and/or be modified with known modifications that reduce effector function. Where such cytotoxic activity is desirable, such as for destruction of a targeted tumor cell, the constant domain may be of isotype with increased effector function and/or be modified with known modifications to increase effector function. Several mutations are known to either reduce or increase effector function. See, e.g., “The future of antibodies as cancer drugs” Janice M Reichert, Eugen Dhimolea, Drug Discov Today (2012) September; 17(17-18):954-63; PMID: 22561895, “Antibody Drug Discovery” (Volume 4 of Molecular medicine and medicinal chemistry) Clive R. Wood, World Scientific, 2012 ISBN 1848166281, 9781848166288; “FcγR requirements leading to successful immunotherapy” Immunol Rev. (2015) November; 268(1):104-22; PMID: 26497516.

In some aspects, the constant domain of an antibody of the present disclosure is human IgG₁ having two modifications in the constant region, Leu234Ala and Leu235Ala. Said modifications in the constant region reduce effector function.

The FRs and CDRs, or HVLs, of an engineered anti-CTGF antibody or antigen-binding fragment thereof need not correspond precisely to the parental sequences, i.e., the sequences of the antibody as isolated prior to engineering. For example, a parental sequence may be altered (e.g., mutagenized) by substitution, insertion or deletion such that the resulting amino acid residue is no longer identical to the original residue in the corresponding position in either parental sequence but the antibody nevertheless retains the function of binding to CTGF. Such alteration typically will not be extensive. Such alterations typically will be conservative alterations. Usually, at least 75% of the engineered antibody residues will correspond to those of the parental sequences, more often at least 90%, and most frequently greater than 95%, or greater than 98% or greater than 99%.

Immunoglobulin residues that affect the interface between heavy and light chain variable regions (“the V_{L}-V_{H} interface”) are those that affect the proximity or orientation of the two chains with respect to one another. Certain residues that may be involved in interchain interactions include V_{L} residues 34, 36, 38, 44, 46, 87, 89, 91, 96, and 98 and V_{H} residues 35, 37, 39, 45, 47, 91, 93, 95, 100, and 103 (utilizing the numbering system set forth in Kabat et al., Sequences of Proteins of Immunological Interest (National Institutes of Health, Bethesda, Md., 1987)). U.S. Pat. No. 6,407,213 also discusses that residues such as V_{L} residues 43 and 85, and V_{H} residues 43 and 60 also may be involved in this interaction. While these residues are indicated for human IgG only, they are applicable across species. Important antibody residues that are reasonably expected to be involved in interchain interactions are selected for substitution into the consensus sequence.

The terms “consensus sequence” and “consensus antibody” refer to an amino acid sequence which comprises the most frequently occurring amino acid residue at each location in all immunoglobulins of any particular class, isotype, or subunit structure, e.g., a human immunoglobulin variable domain. The consensus sequence may be based on immunoglobulins of a particular species or of many species. A “consensus” sequence, structure, or antibody is understood to encompass a consensus human sequence as described in certain embodiments, and to refer to an amino acid sequence which comprises the most frequently occurring amino acid residues at each location in all human immunoglobulins of any particular class, isotype, or subunit structure. Thus, the consensus sequence contains an amino acid sequence having at each position an amino acid that is present in one or more known immunoglobulins, but which may not exactly duplicate the entire amino acid sequence of any single immunoglobulin. The variable region consensus sequence is not obtained from any naturally produced antibody or immunoglobulin. Kabat et al., 1991, Sequences of Proteins of Immunological Interest, 5th Ed. Public Health Service, National Institutes of Health, Bethesda, Md., and variants thereof. The FRs of heavy and light chain consensus sequences, and variants thereof, provide useful sequences for the preparation of human or humanized anti-CTGF antibodies. See, for example, U.S. Pat. Nos. 6,037,454 and 6,054,297.

An “isolated” antibody is one that has been identified and separated and/or recovered from a component of its natural environment or from a cell culture from which it was expressed. An isolated antibody or antibody fragment may have one or more co- or post-translational modifications that arise during production, purification, and/or storage of the antibody or antibody fragment. Contaminant components of the antibody's natural environment are those materials that may interfere with diagnostic or therapeutic uses of the antibody, and can be enzymes, hormones, or other proteinaceous or non-proteinaceous solutes. In some aspects, the antibody will be purified to at least greater than 95% isolation by weight of antibody, for example purified to at least greater than 95%, 96%, 97%, 98%, or 99%.

An isolated antibody includes an antibody in situ within recombinant cells in which it is produced, since at least one component of the antibody's natural environment will not be present. Ordinarily however, an isolated antibody will be prepared by at least one purification step in which the recombinant cellular material is removed.

“Multispecific” refers to a protein, such as an antibody, that specifically binds two or more distinct antigens or two or more distinct epitopes within the same antigen.

“Bispecific” refers to a protein, such as an antibody, that specifically binds two distinct antigens or two distinct epitopes within the same antigen.

In some embodiments, the antibody that specifically binds CTGF or the antigen-binding fragment thereof is a monospecific antibody. In some embodiments, the antibody that specifically binds CTGF or the antigen-binding fragment thereof is a bispecific antibody. In some embodiments, the antibody or the antigen-binding fragment thereof is a multispecific antibody. The monospecific antibodies that specifically bind CTGF provided herein may be engineered into bispecific antibodies, which are also encompassed within the scope of the present disclosure.

Full length bispecific antibodies may be generated for example using Fab arm exchange (e.g., half-molecule exchange, exchanging one heavy chain-light chain pair) between two monospecific bivalent antibodies by introducing substitutions at the heavy chain C_{H}3 interface in each half molecule to favor heterodimer formation of two antibody half molecules having distinct specificity either in vitro in cell-free environment or using co-expression. The Fab arm exchange reaction is the result of a disulfide-bond.

Bispecific antibodies may also be generated using designs such as the Triomab/Quadroma (Trion Pharma/Fresenius Biotech), Knob-in-Hole (Genentech), CrossMAbs (Roche) and the electrostatically-induced C_{H}3 interaction (Chugai, Amgen, NovoNordisk, Oncomed), the LUZ-Y (Genentech), the Strand Exchange Engineered Domain body (SEEDbody) (EMD Serono), the Biclonic (Merus) and as DuoBody® Products (Genmab A/S).

As used herein, the terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=#of identical positions/total #of positions (e.g., overlapping positions)×100). In some embodiments, the two sequences that are compared are the same length after gaps are introduced within the sequences, as appropriate (e.g., excluding additional sequence extending beyond the sequences being compared). For example, when variable region sequences are compared, the leader and/or constant domain sequences are not considered. For sequence comparisons between two sequences, a “corresponding” CDR refers to a CDR in the same location in both sequences (e.g., CDR-H1 of each sequence).

The determination of percent identity or percent similarity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (ld.). When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. Alternatively, protein sequence alignment may be carried out using the CLUSTAL W algorithm, as described by Higgins et al., 1996, Methods Enzymol. 266:383-402.

For diagnostic as well as therapeutic monitoring purposes, the antibodies or antigen-binding fragment thereof of the invention also may be conjugated to a label, either a label alone or a label and an additional second agent (prodrug, chemotherapeutic agent and the like). A label, as distinguished from the other second agents refers to an agent that is a detectable compound or composition and it may be conjugated directly or indirectly to an anti-CTGF antibody or antigen-binding fragment thereof of the present invention. The label may itself be detectable (e.g., radioisotope labels or fluorescent labels) or, in the case of an enzymatic label, may catalyze chemical alteration of a substrate compound or composition that is detectable. Labeled anti-CTGF antibodies or antigen-binding fragments thereof can be prepared and used in various applications including in vitro and in vivo diagnostics.

In various aspects of the present invention one or more domains of the anti-CTGF antibodies or antigen-binding fragments thereof will be recombinantly expressed. Such recombinant expression may employ one or more control sequences, i.e., polynucleotide sequences necessary for expression of an operably linked coding sequence in a particular host organism. The control sequences suitable for use in prokaryotic cells include, for example, promoter, operator, and ribosome binding site sequences. Eukaryotic control sequences include, but are not limited to, promoters, polyadenylation signals, and enhancers. These control sequences can be utilized for expression and production of anti-CTGF antibodies or antigen-binding fragments thereof in prokaryotic and eukaryotic host cells.

A nucleic acid sequence is “operably linked” when it is placed into a functional relationship with another nucleic acid sequence. For example, a nucleic acid presequence or secretory leader is operably linked to a nucleic acid encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers are optionally contiguous. Linking can be accomplished by ligation at convenient restriction sites. If such sites do not exist, synthetic oligonucleotide adaptors or linkers can be used.

As used herein, the expressions “cell”, “cell line”, and “cell culture” are used interchangeably and all such designations include the progeny thereof. Thus, “transformants” and “transformed cells” include the primary subject cell and cultures derived therefrom without regard for the number of transfers, which may for example have been transfected with one or more expression vectors encoding one or more amino acids sequences of an antibody or antigen-binding fragment thereof of the present invention.

The term “mammal” for purposes of treatment according to the invention refers to any animal classified as a mammal, including humans, domesticated and farm animals, and zoo, sports, or companion animals, such as dogs, horses, cats, cows, and the like. Preferably, the mammal is a human.

A “disorder”, as used herein, is any condition that would benefit from treatment with an anti-CTGF antibody or antigen-binding fragment thereof described herein. This includes chronic and acute disorders or diseases including those pathological conditions that predispose the mammal to the disorder in question. Non-limiting examples or disorders to be treated herein include CTGF-associated disorders.

As used herein, the term “CTGF-associated disorder” or “CTGF-associated disease” refers to a condition, which can be alleviated by inhibiting the CTGF cleavage and/or by inhibiting CTGF signaling, e.g., CTGF-NTF signaling. The terms “CTGF-associated disorder” or “CTGF-associated disease” include inflammatory disease, respiratory disease, or fibrotic disorders. CTGF-associated disorders include progressive pulmonary fibrosis, idiopathic pulmonary fibrosis, arthritis, atherosclerosis, cardiac fibrosis, connective tissue fibrosis, Duchenne muscular dystrophy, diabetic nephropathy, diabetic retinopathy, diffuse scleroderma, endothelial fibrosis, epithelial fibrosis, fibrotic disorders, glaucoma, joint fibrosis, kidney fibrosis, limited scleroderma, liver fibrosis, macular degeneration, ocular fibrosis, osteoarthritis, proliferative vitreoretinopathy, pulmonary fibrosis, and systemic fibrosis. CTGF-associated disorders include cancers including acute lymphoblastic leukemia, dermatofibromas, breast cancer, breast carcinoma, glioma and glioblastoma, rhabdomyosarcoma and fibrosarcoma, desmoplasia, angiolipoma, angioleiomyoma, desmoplastic cancers, and prostate, ovarian, colorectal, pancreatic, gastrointestinal, and liver cancer and other tumor growth and metastases.

The terms “specifically binds” or “specific binding” in the context of a binding agent, e.g., an antibody or antigen-binding fragment thereof, refers to a binding agent that associates more frequently, more rapidly, with greater duration, with greater affinity, with greater avidity or with some combination of the above, to an antigen or an epitope within the antigen than with an unrelated antigen. In certain embodiments, an antibody or antigen-binding fragment thereof specifically binds to an antigen or epitope within an antigen with a K_{D} of about 0.1 mM or less, preferably less than about 1 pM. Because of the sequence identity between homologous proteins in different species, or variants of a protein within a single species, specific binding can include an antibody or antigen-binding fragment thereof that recognizes a protein in more than one species. It is understood that, in certain embodiments, an antibody or antigen-binding fragment thereof that specifically binds a first protein may or may not specifically bind a second protein. As such, “specific binding” does not necessarily require (although it can include) exclusive binding, i.e. binding to a single protein. Thus, an antibody or antigen-binding fragment thereof may, in certain embodiments, specifically bind more than one protein.

Methods for determining whether two molecules specifically bind a protein are described herein or a known in the art and include, for example, equilibrium dialysis, surface plasmon resonance, and the like. In one embodiment, specific binding is characterized by a K_{D} of about 1×10⁻⁷ M (100 nM) or less according to the affinity binding method described in the Examples section herein. In some embodiments, specific binding is characterized by a K_{D} of about 5×10⁻⁸ M (50 nM) or less according to the affinity binding method described in the Examples section herein. In some embodiments, specific binding is characterized by a K_{D} of about 1×10⁻⁸ M (10 nM) or less according to the affinity binding method described in the Examples section herein. In some embodiments, specific binding is characterized by a K_{D} of about 5×10⁻⁹ M (5 nM) or less according to the affinity binding method described in the Examples section herein.

The term “subcutaneous administration” refers to introduction of a drug, for example an anti-CTGF antibody or antigen-binding fragment thereof of the invention, under the skin of a subject such as an animal or human patient. Subcutaneous administration may be performed within a pocket between the skin and underlying tissue. Subcutaneous administration may be performed by relatively slow, sustained delivery from a drug receptacle. Pinching or drawing the skin up and away from underlying tissue may create the pocket.

The term “subcutaneous infusion” refers to introduction of a drug, for example an anti-CTGF antibody or antigen-binding fragment thereof of the invention, under the skin of a subject, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the subject, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of a subject, where bolus drug delivery is less than approximately 15 minutes; in some aspects, less than 5 minutes, and some aspects aspect, less than 60 seconds. In some aspects, administration is within a pocket between the skin and underlying tissue, where the pocket may be created by pinching or drawing the skin up and away from underlying tissue. For example, “subcutaneous bolus” refers to the administration of an anti-CTGF antibody or antigen-binding fragment thereof of the invention to a subject in less than approximately 15 minutes; in some aspects, less than 5 minutes, and in some aspects, less than 60 seconds

The term “therapeutically effective amount” is used to refer to an amount of an anti-CTGF antibody or antigen-binding fragment thereof that relieves or ameliorates one or more of the symptoms of the disorder being treated. In doing so, it is that amount that has a beneficial patient outcome. Efficacy can be measured in conventional ways, depending on the condition to be treated.

The terms “treatment” and “therapy” and the like, as used herein, are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an anti-CTGF antibody or antigen-binding fragment thereof prior to or following the onset of a symptom of a disease or disorder thereby preventing or removing one or more signs of the disease or disorder. As an example, the term includes the administration of an anti-CTGF antibody or antigen-binding fragment thereof after clinical manifestation of the disease to combat the symptoms of the disease. Further, administration of an anti-CTGF antibody or antigen-binding fragment thereof after onset and after clinical symptoms have developed where administration affects clinical parameters of the disease or disorder, such as the degree of tissue injury or the amount or extent of metastasis, whether or not the treatment leads to amelioration of the disease, comprises “treatment” or “therapy” as used herein. Moreover, as long as the compositions of the invention either alone or in combination with another therapeutic agent alleviate or ameliorate at least one symptom of a disorder being treated as compared to that symptom in the absence of use of the anti-CTGF antibody or antigen-binding fragment thereof composition or antigen-binding fragment thereof, the result should be considered an effective treatment of the underlying disorder regardless of whether all the symptoms of the disorder are alleviated or not.

To “prevent” refers to prophylactic or preventative measures that prevent and/or slow the development of a targeted pathologic condition or disorder. Thus, those in need of prevention include those prone to have or susceptible to the disorder. In some embodiments, a disease or disorder (preferably a CTGF-associated disorder) is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the invention.

An “effective amount” of an agent, e.g., a pharmaceutical composition, refers to an amount effective, at dosages and for periods of time necessary, to achieve the desired therapeutic or prophylactic result.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present disclosure, the term “comprise” encompasses the term “consist of”. The term “comprising” thus encompasses “including” as well as “consisting” e.g., a composition “comprising” X may consist exclusively of X or may include something additional e.g., X+Y. For example, unless the context requires otherwise, an antibody chain disclosed as “comprising” a specified amino acid sequence encompasses an antibody chain “consisting of” that amino acid sequence. Optionally, said antibody chain may be glycosylated at one or more sites and/or may have one or more post-translational modifications.

The term “about” in relation to a numerical value x means x plus or minus (±) 10%, for example, x±1%, or x±2%, or x±3%, or x±4%, or x±5%, or x±6%, or x±7%, or x±8%, or x±9%, or x±10%.

### Antibody Preparation

In further aspects, the present invention provides a method of producing anti-CTGF antibodies of the invention and antigen-binding fragments thereof, comprising culturing a recombinant host cell expressing the heavy and light chains and isolating the antibody or antigen-binding fragment thereof produced by the cell.

Preferably, the method for producing an antibody or antigen-binding fragment thereof comprises (a) culturing the host cell and (b) isolating the antibody or antigen-binding fragment thereof expressed from the cell. In addition, the method may optionally contain the step (c) of purifying the antibody or antigen-binding fragment thereof.

An antibody of the invention can be produced by transfecting a host cell with one or more vectors comprising polynucleotides encoding the respective antibodies or fragments, culturing the host cell (in particular under conditions that allow synthesis of said antibody molecule); and recovering said antibody molecule from said culture. It is understood that the heavy and the light chain of an antibody may be encoded by separate polynucleotides. Accordingly, provided herein is also a plurality of polynucleotides encoding the (heavy and light chain of) the antibodies or antigen-binding fragments thereof as described herein (by separate polynucleotides). Such a plurality of polynucleotides may be comprised in a (single) vector or in a plurality of vectors.

The antibody or antigen-binding fragment thereof (e.g. as monoclonal antibodies) can be made using recombinant DNA methods as described in U.S. Pat. No. 4,816,567, which is incorporated by reference herein.

The (isolated) polynucleotides encoding the heavy and light chains may be transfected into host cells such as E. coli cells, simian COS cells, Chinese hamster ovary (CHO) cells, or myeloma cells that do not otherwise produce immunoglobulin protein, monoclonal antibodies are generated by the host cells. For example, isolated polynucleotides encoding the heavy and light chains may be stably transfected into a suitable host cell, e.g., a CHO cell, for antibody production.

Various techniques are known for the production of antibody fragments. Traditionally, these fragments are derived via proteolytic digestion of intact antibodies, as described, for example, by Morimoto et al., J. Biochem. Biophys. Meth. 24:107-117 (1993) and Brennan et al., Science 229:81 (1985). In some embodiments, anti-CTGF antibody fragments are produced recombinantly. Fab, Fv, and scFv antibody fragments can all be expressed in and secreted from E. coli or other host cells, thus allowing the production of large amounts of these fragments. Other techniques for the production of antibody fragments will be apparent to the skilled practitioner. For example, antibody fragments can be produced by techniques known in the art including, but not limited to: F(ab′)₂ fragment produced by pepsin digestion of an antibody molecule; Fab fragment generated by reducing the disulfide bridges of an F(ab′)₂ fragment; Fab fragment generated by the treatment of the antibody molecule with papain and a reducing agent. According to the present invention, techniques can be adapted for the production of single-chain antibodies specific to CTGF (see, e.g., U.S. Pat. No. 4,946,778).

### Exemplary Anti-CTGF Antibodies of the Disclosure

Described and disclosed herein are anti-CTGF antibodies, in particular human anti-CTGF antibodies, as well as compositions and articles of manufacture comprising anti-CTGF antibodies of the present invention. Also described are antigen-binding fragments of an anti-CTGF antibody. The anti-CTGF antibodies and antigen-binding fragments thereof can be used in the treatment of a variety of diseases or disorders, in particular, CTGF-associated disorders. An anti-CTGF antibody and an antigen-binding fragment thereof each include at least a portion that specifically binds to CTGF, or a fragment thereof, e.g., CTGF-NTF, preferably human CTGF or human CTGF-NTF.

The generation of anti-CTGF antibodies and their characterization is described in the Examples. CDRs of representative anti-CTGF antibodies of the present invention are disclosed in Tables 1-8 below. Heavy Chain CDR-1, CDR-2, CDR3 (H-CDR1-3) and Light Chain CDR-1, CDR-2, CDR3 (L-CDR1-3) are provided according to the numbering systems according to Kabat, Aho, CCG, and Chothia.

TABLE 1
Light chain CDRs according to Kabat

| Anti- | | SEQ | | SEQ | | SEQ |
|---|---|---|---|---|---|---|
| body | | ID | | ID | | ID |
| Chain | CDR1 | NO: | CDR2 | NO: | CDR3 | NO: |
| L1 | KSSQSIVHYNEKTYLE | 100 | KVSSRFS | 101 | FQGSHFPLT | 102 |
| L2 | KSSQSIVHYNEKTYLE | 110 | KVSSRAS | 111 | FQGSHFPLT | 112 |
| L3 | KSSQSIVHYNGKTYLE | 120 | KVSSRFS | 121 | FQGSHFPLT | 122 |
| L4 | KSSQSIVHYNGKTYLE | 130 | KVSSRAS | 131 | FQGSHFPLT | 132 |
| L5 | KSSQSIVHYNENTYLE | 140 | KVSSRFS | 141 | FQGSHFPLT | 142 |
| L6 | KSSQSIVHYNENTYLE | 150 | KVSSRAS | 151 | FQGSHFPLT | 152 |
| L7 | RSSQSIVHYNENTYLE | 160 | KVSNRFS | 161 | FQGSHFPLT | 162 |
| L8 | RSSQSIVHYNANTYLE | 170 | KVSNRFS | 171 | FQGSHFPLT | 172 |
| L9 | RSSQSIVHYNTNTYLE | 180 | KVSNRFS | 181 | FQGSHFPLT | 182 |
| L10 | RSSQSIVHYNGKTYLE | 190 | KVSNRFS | 191 | FQGSHFPLT | 192 |
| L11 | RSSQSIVHYNEKTYLE | 200 | KVSNRFS | 201 | FQGSHFPLT | 202 |
| L12 | KSSQSIVHYNGNTYLE | 210 | KVSSRFS | 211 | FQGSHFPLT | 212 |
| L13 | KSSQSIVHYNGNTYLE | 220 | KVSSRAS | 221 | FQGSHFPLT | 222 |
| L14 | KSSQSIVHYNGNTYLE | 230 | KVSSRAS | 231 | FQGSHFPLT | 232 |
| L15 | KSSQSIVHYNEKTYLE | 240 | KVSSRAS | 241 | FQGSHFPLT | 242 |
| L16 | RSSQSIVHYNGNTYLE | 250 | KVSNRDS | 251 | FQGSHFPLT | 252 |

TABLE 2
Heavy chain CDRs according to Kabat

| Anti- | | SEQ | | SEQ | | SEQ |
|---|---|---|---|---|---|---|
| body | | ID | | ID | | ID |
| Chain | CDR1 | NO: | CDR2 | NO: | CDR3 | NO: |
| H1 | DYYMA | 105 | NINYEGSRTDLLESLKS | 106 | DTSRGSYFDV | 107 |
| H2 | DYYMA | 115 | NINYYGSRTDLLESLKS | 116 | DTSRGSYFDV | 117 |
| H3 | DYYMA | 125 | NINYEGSRTDLLASLKS | 126 | DTSRGSYFDV | 127 |
| H4 | DYYMA | 135 | NINYYGSRTDLLASLKS | 136 | DTSRGSYFDV | 137 |
| H5 | DYYMA | 145 | NINYDGSRTDLLDSLKS | 146 | DTSRGSYFDV | 147 |
| H6 | DYYMA | 155 | NINYEGSRTDLLDSLKS | 156 | DTSRGSYFDV | 157 |
| H7 | DYYMA | 165 | NINYDGSRTDLLESLKS | 166 | DTSRGSYFDV | 167 |
| H8 | DYYMA | 175 | NINYDGSRTDLLDSLKS | 176 | DTSRGSYFDV | 177 |

TABLE 3
Light chain CDRs according to Aho

| | | SEQ | | SEQ | | SEQ |
|---|---|---|---|---|---|---|
| Antibody | | ID | | ID | | ID |
| chain | CDR1 | NO: | CDR2 | NO: | CDR3 | NO: |
| L1 | QSIVHYNEKTY | 310 | KVS | 320 | FQGSHFPLT | 321 |
| L2 | QSIVHYNEKTY | 310 | KVS | 320 | FQGSHFPLT | 321 |
| L3 | QSIVHYNGKTY | 311 | KVS | 320 | FQGSHFPLT | 321 |
| L4 | QSIVHYNGKTY | 311 | KVS | 320 | FQGSHFPLT | 321 |
| L5 | QSIVHYNENTY | 312 | KVS | 320 | FQGSHFPLT | 321 |
| L6 | QSIVHYNENTY | 312 | KVS | 320 | FQGSHFPLT | 321 |
| L7 | QSIVHYNENTY | 312 | KVS | 320 | FQGSHFPLT | 321 |
| L8 | QSIVHYNANTY | 313 | KVS | 320 | FQGSHFPLT | 321 |
| L9 | QSIVHYNTNTY | 314 | KVS | 320 | FQGSHFPLT | 321 |
| L10 | QSIVHYNGKTY | 311 | KVS | 320 | FQGSHFPLT | 321 |
| L11 | QSIVHYNEKTY | 310 | KVS | 320 | FQGSHFPLT | 321 |
| L12 | QSIVHYNGNTY | 315 | KVS | 320 | FQGSHFPLT | 321 |
| L13 | QSIVHYNGNTY | 315 | KVS | 320 | FQGSHFPLT | 321 |
| L14 | QSIVHYNGNTY | 315 | KVS | 320 | FQGSHFPLT | 321 |
| L15 | QSIVHYNEKTY | 310 | KVS | 320 | FQGSHFPLT | 321 |
| L16 | QSIVHYNGNTY | 315 | KVS | 320 | FQGSHFPLT | 321 |

TABLE 4
Heavy chain CDRs according to Aho

| | | SEQ | | SEQ | | SEQ |
|---|---|---|---|---|---|---|
| Antibody | | ID | | ID | | ID |
| chain | CDR1 | NO: | CDR2 | NO: | CDR3 | NO: |
| H1 | GFTFSDYY | 331 | INYEGSRT | 341 | ARDTSRGSYFDV | 348 |
| H2 | GFTFSDYY | 331 | INYYGSRT | 342 | ARDTSRGSYFDV | 348 |
| H3 | GFTFSDYY | 331 | INYEGSRT | 341 | ARDTSRGSYFDV | 348 |
| H4 | GFTFSDYY | 331 | INYYGSRT | 342 | ARDTSRGSYFDV | 348 |
| H5 | GFTFSDYY | 331 | INYDGSRT | 343 | ARDTSRGSYFDV | 348 |
| H6 | GFTFSDYY | 331 | INYEGSRT | 341 | ARDTSRGSYFDV | 348 |
| H7 | GFTFSDYY | 331 | INYDGSRT | 343 | ARDTSRGSYFDV | 348 |
| H8 | GFTFSDYY | 331 | INYDGSRT | 343 | ARDTSRGSYFDV | 348 |

TABLE 5
Light chain CDRs according to CCG

| Anti- | | SEQ | | SEQ | | SEQ |
|---|---|---|---|---|---|---|
| body | | ID | | ID | | ID |
| chain | CDR1 | NO: | CDR2 | NO: | CDR3 | NO: |
| L1 | KSSQSIVHYNEKTYLE | 300 | KVSSRFS | 316 | FQGSHFPLT | 321 |
| L2 | KSSQSIVHYNEKTYLE | 300 | KVSSRAS | 317 | FQGSHFPLT | 321 |
| L3 | KSSQSIVHYNGKTYLE | 301 | KVSSRFS | 316 | FQGSHFPLT | 321 |
| L4 | KSSQSIVHYNGKTYLE | 301 | KVSSRAS | 317 | FQGSHFPLT | 321 |
| L5 | KSSQSIVHYNENTYLE | 302 | KVSSRFS | 316 | FQGSHFPLT | 321 |
| L6 | KSSQSIVHYNENTYLE | 302 | KVSSRAS | 317 | FQGSHFPLT | 321 |
| L7 | RSSQSIVHYNENTYLE | 303 | KVSNRFS | 318 | FQGSHFPLT | 321 |
| L8 | RSSQSIVHYNANTYLE | 304 | KVSNRFS | 318 | FQGSHFPLT | 321 |
| L9 | RSSQSIVHYNTNTYLE | 305 | KVSNRFS | 318 | FQGSHFPLT | 321 |
| L10 | RSSQSIVHYNGKTYLE | 306 | KVSNRFS | 318 | FQGSHFPLT | 321 |
| L11 | RSSQSIVHYNEKTYLE | 307 | KVSNRFS | 318 | FQGSHFPLT | 321 |
| L12 | KSSQSIVHYNGNTYLE | 308 | KVSSRFS | 316 | FQGSHFPLT | 321 |
| L13 | KSSQSIVHYNGNTYLE | 308 | KVSSRAS | 317 | FQGSHFPLT | 321 |
| L14 | KSSQSIVHYNGNTYLE | 308 | KVSSRAS | 317 | FQGSHFPLT | 321 |
| L15 | KSSQSIVHYNEKTYLE | 300 | KVSSRAS | 317 | FQGSHFPLT | 321 |
| L16 | RSSQSIVHYNGNTYLE | 309 | KVSNRDS | 319 | FQGSHFPLT | 321 |

TABLE 6
Heavy chain CDRs according to CCG

| Antibody | | SEQ | | SEQ | | SEQ |
|---|---|---|---|---|---|---|
| chain | CDR1 | ID NO: | CDR2 | ID NO: | CDR3 | ID NO: |
| H1 | GFTFSDYYMA | 332 | NINYEGSRTDLLESLKS | 334 | DTSRGSYFDV | 347 |
| H2 | GFTFSDYYMA | 332 | NINYYGSRTDLLESLKS | 335 | DTSRGSYFDV | 347 |
| H3 | GFTFSDYYMA | 332 | NINYEGSRTDLLASLKS | 336 | DTSRGSYFDV | 347 |
| H4 | GFTFSDYYMA | 332 | NINYYGSRTDLLASLKS | 337 | DTSRGSYFDV | 347 |
| H5 | GFTFSDYYMA | 332 | NINYDGSRTDLLDSLKS | 338 | DTSRGSYFDV | 347 |
| H6 | GFTFSDYYMA | 332 | NINYEGSRTDLLDSLKS | 339 | DTSRGSYFDV | 347 |
| H7 | GFTFSDYYMA | 332 | NINYDGSRTDLLESLKS | 340 | DTSRGSYFDV | 347 |
| H8 | GFTFSDYYMA | 332 | NINYDGSRTDLLDSLKS | 338 | DTSRGSYFDV | 347 |

TABLE 7
Light chain CDRs according to Chothia

| Anti- | | SEQ | | SEQ | | SEQ |
|---|---|---|---|---|---|---|
| body | | ID | | ID | | ID |
| chain | CDR1 | NO: | CDR2 | NO: | CDR3 | NO: |
| L1 | KSSQSIVHYNEKTYLE | 300 | KVSSRFS | 316 | FQGSHFPLT | 321 |
| L2 | KSSQSIVHYNEKTYLE | 300 | KVSSRAS | 317 | FQGSHFPLT | 321 |
| L3 | KSSQSIVHYNGKTYLE | 301 | KVSSRFS | 316 | FQGSHFPLT | 321 |
| L4 | KSSQSIVHYNGKTYLE | 301 | KVSSRAS | 317 | FQGSHFPLT | 321 |
| L5 | KSSQSIVHYNENTYLE | 302 | KVSSRFS | 316 | FQGSHFPLT | 321 |
| L6 | KSSQSIVHYNENTYLE | 302 | KVSSRAS | 317 | FQGSHFPLT | 321 |
| L7 | RSSQSIVHYNENTYLE | 303 | KVSNRFS | 318 | FQGSHFPLT | 321 |
| L8 | RSSQSIVHYNANTYLE | 304 | KVSNRFS | 318 | FQGSHFPLT | 321 |
| L9 | RSSQSIVHYNTNTYLE | 305 | KVSNRFS | 318 | FQGSHFPLT | 321 |
| L10 | RSSQSIVHYNGKTYLE | 306 | KVSNRFS | 318 | FQGSHFPLT | 321 |
| L11 | RSSQSIVHYNEKTYLE | 307 | KVSNRFS | 318 | FQGSHFPLT | 321 |
| L12 | KSSQSIVHYNGNTYLE | 308 | KVSSRFS | 316 | FQGSHFPLT | 321 |
| L13 | KSSQSIVHYNGNTYLE | 308 | KVSSRAS | 317 | FQGSHFPLT | 321 |
| L14 | KSSQSIVHYNGNTYLE | 308 | KVSSRAS | 317 | FQGSHFPLT | 321 |
| L15 | KSSQSIVHYNEKTYLE | 300 | KVSSRAS | 317 | FQGSHFPLT | 321 |
| L16 | RSSQSIVHYNGNTYLE | 309 | KVSNRDS | 319 | FQGSHFPLT | 321 |

TABLE 8
Heavy chain CDRs according to Chothia

| Antibody | | SEQ | | SEQ | | SEQ |
|---|---|---|---|---|---|---|
| chain | CDR1 | ID NO: | CDR2 | ID NO: | CDR3 | ID NO: |
| H1 | GFTFSDY | 333 | NYEGSR | 344 | TSRGSYFD | 349 |
| H2 | GFTFSDY | 333 | NYYGSR | 345 | TSRGSYFD | 349 |
| H3 | GFTFSDY | 333 | NYEGSR | 344 | TSRGSYFD | 349 |
| H4 | GFTFSDY | 333 | NYYGSR | 345 | TSRGSYFD | 349 |
| H5 | GFTFSDY | 333 | NYDGSR | 346 | TSRGSYFD | 349 |
| H6 | GFTFSDY | 333 | NYEGSR | 344 | TSRGSYFD | 349 |
| H7 | GFTFSDY | 333 | NYDGSR | 346 | TSRGSYFD | 349 |
| H8 | GFTFSDY | 333 | NYDGSR | 346 | TSRGSYFD | 349 |

### Amino Acid Sequence Variants

Variant anti-CTGF antibodies and antibody fragments thereof can be engineered based on a set of CDRs depicted in Tables 1-8. It is to be understood that in the variant anti-CTGF antibodies and antibody fragments the amino acid sequence of the CDRs remain unchanged or have minimal changes (e.g., 1-5 changes), but the surrounding regions, e.g., FR regions can be engineered. Amino acid sequence variants of the anti-CTGF antibody can be prepared by introducing appropriate nucleotide changes into the anti-CTGF antibody DNA, or by peptide synthesis. Such variants include, for example, deletions from, and/or insertions into and/or substitutions of, residues within the amino acid sequences of the anti-CTGF antibodies of the examples herein. Any combination of deletions, insertions, and substitutions is made to arrive at the final construct, provided that the final construct possesses the desired characteristics. The amino acid changes also may alter post-translational processes of the human or variant anti-CTGF antibody, such as changing the number or position of glycosylation sites.

In some embodiments, the present invention includes anti-CTGF antibodies or antibody fragments thereof having a variable heavy chain (also referred to as “heavy chain variable region”) and a variable light chain (also referred to as “light chain variable region”), wherein the variable heavy chain amino acid sequence and the variable light chain amino acid sequence are at least at least 90%, preferably at least 92.5%, more preferably at least 95%, still more preferably at least 98%, or even more preferably at least 99% identical to the amino acid sequences disclosed in Tables 9-10 provided that the antibody or fragments thereof retain binding to CTGF.

TABLE 9
Anti-CTGF antibody light chain variable region sequences

| Antibody | Light Chain Variable Region | SEQ | Antibodies That |
|---|---|---|---|
| Chain Name | Sequence | ID NO | Contain This Chain |
| L1 | VVMTQTPLSLSVSLGDQASISCKSSQSIVHYNEK | 103 | A1, A2, A3, A4, A5, A6, |
| | TYLEWYLQKPGQSPQLLIYKVSSRFSGVPDRFSG | | A7, A8 |
| | SGSGTDFTLKISRVEAEDLGVYYCFQGSHFPLTF | | |
| | GQGTKLEIK | | |
| L2 | VVMTQTPLSLSVSLGDQASISCKSSQSIVHYNEK | 113 | A9, A10, A11, A12, A13, |
| | TYLEWYLQKPGQSPQLLIYKVSSRASGVPDRFSG | | A14, A15, A16 |
| | SGSGTDFTLKISRVEAEDLGVYYCFQGSHFPLTF | | |
| | GQGTKLEIK | | |
| L3 | VVMTQTPLSLSVSLGDQASISCKSSQSIVHYNGK | 123 | A17, A18, A19, A20, |
| | TYLEWYLQKPGQSPQLLIYKVSSRFSGVPDRFSG | | A21, A22, A23, A24 |
| | SGSGTDFTLKISRVEAEDLGVYYCFQGSHFPLTF | | |
| | GQGTKLEIK | | |
| L4 | VVMTQTPLSLSVSLGDQASISCKSSQSIVHYNGK | 133 | A25, A26, A27, A28, |
| | TYLEWYLQKPGQSPQLLIYKVSSRASGVPDRFSG | | A29, A30, A31, A32 |
| | SGSGTDFTLKISRVEAEDLGVYYCFQGSHFPLTF | | |
| | GQGTKLEIK | | |
| L5 | VVMTQTPLSLSVSLGDQASISCKSSQSIVHYNEN | 143 | A33, A34, A35, A36, |
| | TYLEWYLQKPGQSPQLLIYKVSSRFSGVPDRFSG | | A37, A38, A39, A40 |
| | SGSGTDFTLKISRVEAEDLGVYYCFQGSHFPLTF | | |
| | GQGTKLEIK | | |
| L6 | VVMTQTPLSLSVSLGDQASISCKSSQSIVHYNEN | 153 | A41, A42, A43, A44, |
| | TYLEWYLQKPGQSPQLLIYKVSSRASGVPDRFSG | | A45, A46, A47, A48 |
| | SGSGTDFTLKISRVEAEDLGVYYCFQGSHFPLTF | | |
| | GQGTKLEIK | | |
| L7 | VVMTQTPLSLPVSLGDQASISCRSSQSIVHYNEN | 163 | A49, A50, A51, A52, |
| | TYLEWYLQKPGQSPQLLIYKVSNRFSGVPDRFSG | | A53, A54, A55, A56 |
| | SGSGTDFTLKISRVEAEDLGVYYCFQGSHFPLTF | | |
| | GQGTKLEIK | | |
| L8 | VVMTQTPLSLPVSLGDQASISCRSSQSIVHYNAN | 173 | A57, A58, A59, A60, |
| | TYLEWYLQKPGQSPQLLIYKVSNRFSGVPDRFSG | | A61, A62, A63, A64 |
| | SGSGTDFTLKISRVEAEDLGVYYCFQGSHFPLTF | | |
| | GQGTKLEIK | | |
| L9 | VVMTQTPLSLPVSLGDQASISCRSSQSIVHYNTN | 183 | A65, A66, A67, A68, |
| | TYLEWYLQKPGQSPQLLIYKVSNRFSGVPDRFSG | | A69, A70, A71, A72 |
| | SGSGTDFTLKISRVEAEDLGVYYCFQGSHFPLTF | | |
| | GQGTKLEIK | | |
| L10 | VVMTQTPLSLPVSLGDQASISCRSSQSIVHYNGK | 193 | A73, A74, A75, A76, |
| | TYLEWYLQKPGQSPQLLIYKVSNRFSGVPDRFSG | | A77, A78, A79, A80 |
| | SGSGTDFTLKISRVEAEDLGVYYCFQGSHFPLTF | | |
| | GQGTKLEIK | | |
| L11 | VVMTQTPLSLPVSLGDQASISCRSSQSIVHYNEK | 203 | A81, A82, A83, A84, |
| | TYLEWYLQKPGQSPQLLIYKVSNRFSGVPDRFSG | | A85, A86, A87, A88 |
| | SGSGTDFTLKISRVEAEDLGVYYCFQGSHFPLTF | | |
| | GQGTKLEIK | | |
| L12 | VVMTQTPLSLSVSLGDQASISCKSSQSIVHYNGN | 213 | A89, A90, A91, A92, |
| | TYLEWYLQKPGQSPQLLIYKVSSRFSGVPDRFSG | | A93, A94, A95, A96 |
| | SGSGTDFTLKISRVEAEDLGVYYCFQGSHFPLTF | | |
| | GQGTKLEIK | | |
| L13 | VVMTQTPLSLSVSLGDQASISCKSSQSIVHYNGN | 223 | A97, A98, A99, A100, |
| | TYLEWYLQKPGQSPQLLIYKVSSRASGVPDRFSG | | A101, A102, A103, |
| | SGSGTDFTLKISRVEAEDLGVYYCFQGSHFPLTF | | A104 |
| | GQGTKLEIK | | |
| L14 | DIVMTQTPLSLSVSLGDQASISCKSSQSIVHYNG | 233 | A105, A106, A107, |
| | NTYLEWYLQKPGQSPQLLIYKVSSRASGVPDRES | | A108, A109, A110, |
| | GSGSGTDFTLKISRVEAEDLGVYYCFQGSHFPLT | | A111, A112 |
| | FGQGTKLEIK | | |
| L15 | DIVMTQTPLSLSVSLGDQASISCKSSQSIVHYNE | 243 | A113, A114, A115, |
| | KTYLEWYLQKPGQSPQLLIYKVSSRASGVPDRES | | A116, A117, A118, |
| | GSGSGTDFTLKISRVEAEDLGVYYCFQGSHFPLT | | A119, A120 |
| | FGQGTKLEIK | | |
| L16 | VVMTQTPLSLPVSLGDQASISCRSSQSIVHYNGN | 253 | A121, A122, A123, |
| | TYLEWYLQKPGQSPQLLIYKVSNRDSGVPDRFSG | | A124, A125, A126, |
| | SGSGTDFTLKISRVEAEDLGVYYCFQGSHFPLTF | | A127, A128 |
| | GQGTKLEIK | | |

TABLE 10
Anti-CTGF antibody heavy chain variable region sequences

| Antibody | Heavy Chain Variable Region | SEQ | Antibodies That |
|---|---|---|---|
| Chain Name | Sequence | ID NO | Contain This Chain |
| H1 | EVQLVESGGGLVQPGGSLRLSCAASGFTFSDYYM | 108 | A1, A9, A17, A25, A33, |
| | AWVRQAPGKGLEWVANINYEGSRTDLLESLKSRF | | A41, A49, A57, A65, |
| | TISRDNAKNSVYLQMNSLRAEDTAVYYCARDTSR | | A73, A81, A89, A97, |
| | GSYFDVWGAGTTVTVSS | | A105, A113, A121 |
| H2 | EVQLVESGGGLVQPGGSLRLSCAASGFTFSDYYM | 118 | A2, A10, A18, A26, A34, |
| | AWVRQAPGKGLEWVANINYYGSRTDLLESLKSRF | | A42, A50, A58, A66, |
| | TISRDNAKNSVYLQMNSLRAEDTAVYYCARDTSR | | A74, A82, A90, A98, |
| | GSYFDVWGAGTTVTVSS | | A106, A114, A122 |
| H3 | EVQLVESGGGLVQPGGSLRLSCAASGFTFSDYYM | 128 | A3, A11, A19, A27, A35, |
| | AWVRQAPGKGLEWVANINYEGSRTDLLASLKSRF | | A43, A51, A59, A67, |
| | TISRDNAKNSVYLQMNSLRAEDTAVYYCARDTSR | | A75, A83, A91, A99, |
| | GSYFDVWGAGTTVTVSS | | A107, A115, A123 |
| H4 | EVQLVESGGGLVQPGGSLRLSCAASGFTFSDYYM | 138 | A4, A12, A20, A28, A36, |
| | AWVRQAPGKGLEWVANINYYGSRTDLLASLKSRF | | A44, A52, A60, A68, |
| | TISRDNAKNSVYLQMNSLRAEDTAVYYCARDTSR | | A76, A84, A92, A100, |
| | GSYFDVWGAGTTVTVSS | | A108, A116, A124 |
| H5 | EVQLVESGGGLVQPGGSLRLSCAASGFTFSDYYM | 148 | A5, A13, A21, A29, A37, |
| | AWVRQAPGKGLEWVANINYDGSRTDLLDSLKSRF | | A45, A53, A61, A69, |
| | TISRDNAKNSVYLQMNSLRAEDTAVYYCARDTSR | | A77, A85, A93, A101, |
| | GSYFDVWGAGTTVTVSS | | A109, A117, A125 |
| H6 | EVQLVESGGGLVQPGGSLRLSCAASGFTFSDYYM | 158 | A6, A14, A22, A30, A38, |
| | AWVRQAPGKGLEWVANINYEGSRTDLLDSLKSRF | | A46, A54, A62, A70, |
| | TISRDNAKNSVYLQMNSLRAEDTAVYYCARDTSR | | A78, A86, A94, A102, |
| | GSYFDVWGAGTTVTVSS | | A110, A118, A126 |
| H7 | EVQLVESGGGLVQPGGSLRLSCAASGFTFSDYYM | 168 | A7, A15, A23, A31, A39, |
| | AWVRQAPGKGLEWVANINYDGSRTDLLESLKSRF | | A47, A55, A63, A71, |
| | TISRDNAKNSVYLQMNSLRAEDTAVYYCARDTSR | | A79, A87, A95, A103, |
| | GSYFDVWGAGTTVTVSS | | A111, A119, A127 |
| H8 | EVKLVESGGGLVQPGGSLRLSCAASGFTFSDYYM | 178 | A8, A16, A24, A32, A40, |
| | AWVRQAPGKGLEWVANINYDGSRTDLLDSLKSRF | | A48, A56, A64, A72, |
| | TISRDNAKNSVYLQMNSLRAEDTAVYYCARDTSR | | A80, A88, A96, A104, |
| | GSYFDVWGAGTTVTVSS | | A112, A120, A128 |

TABLE 11
Anti-CTGF antibody light chain sequences

| | | | Antibodies |
|---|---|---|---|
| Antibody | | SEQ | That Contain |
| Chain Name | Light Chain Sequence | ID NO | This Chain |
| L1 | VVMTQTPLSLSVSLGDQASISCKSSQSIVHYNEKTYLEW | 104 | A1, A2, A3, A4, |
| | YLQKPGQSPQLLIYKVSSRFSGVPDRFSGSGSGTDFTLK | | A5, A6, A7, A8 |
| | ISRVEAEDLGVYYCFQGSHFPLTFGQGTKLEIKRTVAAP | | |
| | SVFIFPPSDEQLKSGTASVVCLLNNFYPREAKVQWKVDN | | |
| | ALQSGNSQESVTEQDSKDSTYSLSSTLTLSKADYEKHKV | | |
| | YACEVTHQGLSSPVTKSFNRGEC | | |
| L2 | VVMTQTPLSLSVSLGDQASISCKSSQSIVHYNEKTYLEW | 114 | A9, A10, A11, |
| | YLQKPGQSPQLLIYKVSSRASGVPDRFSGSGSGTDFTLK | | A12, A13, A14, |
| | ISRVEAEDLGVYYCFQGSHFPLTFGQGTKLEIKRTVAAP | | A15, A16 |
| | SVFIFPPSDEQLKSGTASVVCLLNNFYPREAKVQWKVDN | | |
| | ALQSGNSQESVTEQDSKDSTYSLSSTLTLSKADYEKHKV | | |
| | YACEVTHQGLSSPVTKSFNRGEC | | |
| L3 | VVMTQTPLSLSVSLGDQASISCKSSQSIVHYNGKTYLEW | 124 | A17, A18, A19, |
| | YLQKPGQSPQLLIYKVSSRFSGVPDRFSGSGSGTDFTLK | | A20, A21, A22, |
| | ISRVEAEDLGVYYCFQGSHFPLTFGQGTKLEIKRTVAAP | | A23, A24 |
| | SVFIFPPSDEQLKSGTASVVCLLNNFYPREAKVQWKVDN | | |
| | ALQSGNSQESVTEQDSKDSTYSLSSTLTLSKADYEKHKV | | |
| | YACEVTHQGLSSPVTKSFNRGEC | | |
| L4 | VVMTQTPLSLSVSLGDQASISCKSSQSIVHYNGKTYLEW | 134 | A25, A26, A27, |
| | YLQKPGQSPQLLIYKVSSRASGVPDRFSGSGSGTDFTLK | | A28, A29, A30, |
| | ISRVEAEDLGVYYCFQGSHFPLTFGQGTKLEIKRTVAAP | | A31, A32 |
| | SVFIFPPSDEQLKSGTASVVCLLNNFYPREAKVQWKVDN | | |
| | ALQSGNSQESVTEQDSKDSTYSLSSTLTLSKADYEKHKV | | |
| | YACEVTHQGLSSPVTKSFNRGEC | | |
| L5 | VVMTQTPLSLSVSLGDQASISCKSSQSIVHYNENTYLEW | 144 | A33, A34, A35, |
| | YLQKPGQSPQLLIYKVSSRFSGVPDRFSGSGSGTDFTLK | | A36, A37, A38, |
| | ISRVEAEDLGVYYCFQGSHFPLTFGQGTKLEIKRTVAAP | | A39, A40 |
| | SVFIFPPSDEQLKSGTASVVCLLNNFYPREAKVQWKVDN | | |
| | ALQSGNSQESVTEQDSKDSTYSLSSTLTLSKADYEKHKV | | |
| | YACEVTHQGLSSPVTKSFNRGEC | | |
| L6 | VVMTQTPLSLSVSLGDQASISCKSSQSIVHYNENTYLEW | 154 | A41, A42, A43, |
| | YLQKPGQSPQLLIYKVSSRASGVPDRFSGSGSGTDFTLK | | A44, A45, A46, |
| | ISRVEAEDLGVYYCFQGSHFPLTFGQGTKLEIKRTVAAP | | A47, A48 |
| | SVFIFPPSDEQLKSGTASVVCLLNNFYPREAKVQWKVDN | | |
| | ALQSGNSQESVTEQDSKDSTYSLSSTLTLSKADYEKHKV | | |
| | YACEVTHQGLSSPVTKSFNRGEC | | |
| L7 | VVMTQTPLSLPVSLGDQASISCRSSQSIVHYNENTYLEW | 164 | A49, A50, A51, |
| | YLQKPGQSPQLLIYKVSNRFSGVPDRFSGSGSGTDFTLK | | A52, A53, A54, |
| | ISRVEAEDLGVYYCFQGSHFPLTFGQGTKLEIKRTVAAP | | A55, A56 |
| | SVFIFPPSDEQLKSGTASVVCLLNNFYPREAKVQWKVDN | | |
| | ALQSGNSQESVTEQDSKDSTYSLSSTLTLSKADYEKHKV | | |
| | YACEVTHQGLSSPVTKSFNRGEC | | |
| L8 | VVMTQTPLSLPVSLGDQASISCRSSQSIVHYNANTYLEW | 174 | A57, A58, A59, |
| | YLQKPGQSPQLLIYKVSNRFSGVPDRFSGSGSGTDFTLK | | A60, A61, A62, |
| | ISRVEAEDLGVYYCFQGSHFPLTFGQGTKLEIKRTVAAP | | A63, A64 |
| | SVFIFPPSDEQLKSGTASVVCLLNNFYPREAKVQWKVDN | | |
| | ALQSGNSQESVTEQDSKDSTYSLSSTLTLSKADYEKHKV | | |
| | YACEVTHQGLSSPVTKSFNRGEC | | |
| L9 | VVMTQTPLSLPVSLGDQASISCRSSQSIVHYNTNTYLEW | 184 | A65, A66, A67, |
| | YLQKPGQSPQLLIYKVSNRFSGVPDRFSGSGSGTDFTLK | | A68, A69, A70, |
| | ISRVEAEDLGVYYCFQGSHFPLTFGQGTKLEIKRTVAAP | | A71, A72 |
| | SVFIFPPSDEQLKSGTASVVCLLNNFYPREAKVQWKVDN | | |
| | ALQSGNSQESVTEQDSKDSTYSLSSTLTLSKADYEKHKV | | |
| | YACEVTHQGLSSPVTKSFNRGEC | | |
| L10 | VVMTQTPLSLPVSLGDQASISCRSSQSIVHYNGKTYLEW | 194 | A73, A74, A75, |
| | YLQKPGQSPQLLIYKVSNRFSGVPDRFSGSGSGTDFTLK | | A76, A77, A78, |
| | ISRVEAEDLGVYYCFQGSHFPLTFGQGTKLEIKRTVAAP | | A79, A80 |
| | SVFIFPPSDEQLKSGTASVVCLLNNFYPREAKVQWKVDN | | |
| | ALQSGNSQESVTEQDSKDSTYSLSSTLTLSKADYEKHKV | | |
| | YACEVTHQGLSSPVTKSFNRGEC | | |
| L11 | VVMTQTPLSLPVSLGDQASISCRSSQSIVHYNEKTYLEW | 204 | A81, A82, A83, |
| | YLQKPGQSPQLLIYKVSNRFSGVPDRFSGSGSGTDFTLK | | A84, A85, A86, |
| | ISRVEAEDLGVYYCFQGSHFPLTFGQGTKLEIKRTVAAP | | A87, A88 |
| | SVFIFPPSDEQLKSGTASVVCLLNNFYPREAKVQWKVDN | | |
| | ALQSGNSQESVTEQDSKDSTYSLSSTLTLSKADYEKHKV | | |
| | YACEVTHQGLSSPVTKSFNRGEC | | |
| L12 | VVMTQTPLSLSVSLGDQASISCKSSQSIVHYNGNTYLEW | 214 | A89, A90, A91, |
| | YLQKPGQSPQLLIYKVSSRFSGVPDRFSGSGSGTDFTLK | | A92, A93, A94, |
| | ISRVEAEDLGVYYCFQGSHFPLTFGQGTKLEIKRTVAAP | | A95, A96 |
| | SVFIFPPSDEQLKSGTASVVCLLNNFYPREAKVQWKVDN | | |
| | ALQSGNSQESVTEQDSKDSTYSLSSTLTLSKADYEKHKV | | |
| | YACEVTHQGLSSPVTKSFNRGEC | | |
| L13 | VVMTQTPLSLSVSLGDQASISCKSSQSIVHYNGNTYLEW | 224 | A97, A98, A99, |
| | YLQKPGQSPQLLIYKVSSRASGVPDRFSGSGSGTDFTLK | | A100, A101, |
| | ISRVEAEDLGVYYCFQGSHFPLTFGQGTKLEIKRTVAAP | | A102, A103, |
| | SVFIFPPSDEQLKSGTASVVCLLNNFYPREAKVQWKVDN | | A104 |
| | ALQSGNSQESVTEQDSKDSTYSLSSTLTLSKADYEKHKV | | |
| | YACEVTHQGLSSPVTKSFNRGEC | | |
| L14 | DIVMTQTPLSLSVSLGDQASISCKSSQSIVHYNGNTYLE | 234 | A105, A106, |
| | WYLQKPGQSPQLLIYKVSSRASGVPDRFSGSGSGTDFTL | | A107, A108, |
| | KISRVEAEDLGVYYCFQGSHFPLTFGQGTKLEIKRTVAA | | A109, A110, |
| | PSVFIFPPSDEQLKSGTASVVCLLNNFYPREAKVQWKVD | | A111, A112 |
| | NALQSGNSQESVTEQDSKDSTYSLSSTLTLSKADYEKHK | | |
| | VYACEVTHQGLSSPVTKSFNRGEC | | |
| L15 | DIVMTQTPLSLSVSLGDQASISCKSSQSIVHYNEKTYLE | 244 | A113, A114, |
| | WYLQKPGQSPQLLIYKVSSRASGVPDRFSGSGSGTDFTL | | A115, A116, |
| | KISRVEAEDLGVYYCFQGSHFPLTFGQGTKLEIKRTVAA | | A117, A118, |
| | PSVFIFPPSDEQLKSGTASVVCLLNNFYPREAKVQWKVD | | A119, A120 |
| | NALQSGNSQESVTEQDSKDSTYSLSSTLTLSKADYEKHK | | |
| | VYACEVTHQGLSSPVTKSFNRGEC | | |
| L16 | VVMTQTPLSLPVSLGDQASISCRSSQSIVHYNGNTYLEW | 254 | A121, A122, |
| | YLQKPGQSPQLLIYKVSNRDSGVPDRFSGSGSGTDFTLK | | A123, A124, |
| | ISRVEAEDLGVYYCFQGSHFPLTFGQGTKLEIKRTVAAP | | A125, A126, |
| | SVFIFPPSDEQLKSGTASVVCLLNNFYPREAKVQWKVDN | | A127, A128 |
| | ALQSGNSQESVTEQDSKDSTYSLSSTLTLSKADYEKHKV | | |
| | YACEVTHQGLSSPVTKSFNRGEC | | |

TABLE 12
Anti-CTGF antibody heavy chain sequences

| | | | Antibodies |
|---|---|---|---|
| Antibody | | SEQ | That Contain |
| Chain Name | Heavy Chain Sequence | ID NO | This Chain |
| H1 | EVQLVESGGGLVQPGGSLRLSCAASGFTFSDYYMAWVRQ | 109 | A1, A9, A17, |
| | APGKGLEWVANINYEGSRTDLLESLKSRFTISRDNAKNS | | A25, A33, A41, |
| | VYLQMNSLRAEDTAVYYCARDTSRGSYFDVWGAGTTVTV | | A49, A57, A65, |
| | SSASTKGPSVFPLAPSSKSTSGGTAALGCLVKDYFPEPV | | A73, A81, A89, |
| | TVSWNSGALTSGVHTFPAVLQSSGLYSLSSVVTVPSSSL | | A97, A105, |
| | GTQTYICNVNHKPSNTKVDKRVEPKSCDKTHTCPPCPAP | | A113, A121 |
| | EAAGGPSVFLFPPKPKDTLMISRTPEVTCVVVDVSHEDP | | |
| | EVKENWYVDGVEVHNAKTKPREEQYNSTYRVVSVLTVLH | | |
| | QDWLNGKEYKCKVSNKALPAPIEKTISKAKGQPREPQVY | | |
| | TLPPSRFEMTKNQVSLTCLVKGFYPSDIAVEWESNGQPE | | |
| | NNYKTTPPVLDSDGSFFLYSKLTVDKSRWQQGNVESCSV | | |
| | MHEALHNHYTQKSLSLSPG | | |
| H2 | EVQLVESGGGLVQPGGSLRLSCAASGFTFSDYYMAWVRQ | 119 | A2, A10, A18, |
| | APGKGLEWVANINYYGSRTDLLESLKSRFTISRDNAKNS | | A26, A34, A42, |
| | VYLQMNSLRAEDTAVYYCARDTSRGSYFDVWGAGTTVTV | | A50, A58, A66, |
| | SSASTKGPSVFPLAPSSKSTSGGTAALGCLVKDYFPEPV | | A74, A82, A90, |
| | TVSWNSGALTSGVHTFPAVLQSSGLYSLSSVVTVPSSSL | | A98, A106, |
| | GTQTYICNVNHKPSNTKVDKRVEPKSCDKTHTCPPCPAP | | A114, A122 |
| | EAAGGPSVFLFPPKPKDTLMISRTPEVTCVVVDVSHEDP | | |
| | EVKFNWYVDGVEVHNAKTKPREEQYNSTYRVVSVLTVLH | | |
| | QDWLNGKEYKCKVSNKALPAPIEKTISKAKGQPREPQVY | | |
| | TLPPSRFEMTKNQVSLTCLVKGFYPSDIAVEWESNGQPE | | |
| | NNYKTTPPVLDSDGSFFLYSKLTVDKSRWQQGNVFSCSV | | |
| | MHEALHNHYTQKSLSLSPG | | |
| H3 | EVQLVESGGGLVQPGGSLRLSCAASGFTFSDYYMAWVRQ | 129 | A3, A11, A19, |
| | APGKGLEWVANINYEGSRTDLLASLKSRFTISRDNAKNS | | A27, A35, A43, |
| | VYLQMNSLRAEDTAVYYCARDTSRGSYFDVWGAGTTVTV | | A51, A59, A67, |
| | SSASTKGPSVFPLAPSSKSTSGGTAALGCLVKDYFPEPV | | A75, A83, A91, |
| | TVSWNSGALTSGVHTFPAVLQSSGLYSLSSVVTVPSSSL | | A99, A107, |
| | GTQTYICNVNHKPSNTKVDKRVEPKSCDKTHTCPPCPAP | | A115, A123 |
| | EAAGGPSVFLFPPKPKDTLMISRTPEVTCVVVDVSHEDP | | |
| | EVKENWYVDGVEVHNAKTKPREEQYNSTYRVVSVLTVLH | | |
| | QDWLNGKEYKCKVSNKALPAPIEKTISKAKGQPREPQVY | | |
| | TLPPSRFEMTKNQVSLTCLVKGFYPSDIAVEWESNGQPE | | |
| | NNYKTTPPVLDSDGSFFLYSKLTVDKSRWQQGNVFSCSV | | |
| | MHEALHNHYTQKSLSLSPG | | |
| H4 | EVQLVESGGGLVQPGGSLRLSCAASGFTFSDYYMAWVRQ | 139 | A4, A12, A20, |
| | APGKGLEWVANINYYGSRTDLLASLKSRFTISRDNAKNS | | A28, A36, A44, |
| | VYLQMNSLRAEDTAVYYCARDTSRGSYFDVWGAGTTVTV | | A52, A60, A68, |
| | SSASTKGPSVFPLAPSSKSTSGGTAALGCLVKDYFPEPV | | A76, A84, A92, |
| | TVSWNSGALTSGVHTFPAVLQSSGLYSLSSVVTVPSSSL | | A100, A108, |
| | GTQTYICNVNHKPSNTKVDKRVEPKSCDKTHTCPPCPAP | | A116, A124 |
| | EAAGGPSVFLFPPKPKDTLMISRTPEVTCVVVDVSHEDP | | |
| | EVKENWYVDGVEVHNAKTKPREEQYNSTYRVVSVLTVLH | | |
| | QDWLNGKEYKCKVSNKALPAPIEKTISKAKGQPREPQVY | | |
| | TLPPSRFEMTKNQVSLTCLVKGFYPSDIAVEWESNGQPE | | |
| | NNYKTTPPVLDSDGSFFLYSKLTVDKSRWQQGNVFSCSV | | |
| | MHEALHNHYTQKSLSLSPG | | |
| H5 | EVQLVESGGGLVQPGGSLRLSCAASGFTFSDYYMAWVRQ | 149 | A5, A13, A21, |
| | APGKGLEWVANINYDGSRTDLLDSLKSRFTISRDNAKNS | | A29, A37, A45, |
| | VYLQMNSLRAEDTAVYYCARDTSRGSYFDVWGAGTTVTV | | A53, A61, A69, |
| | SSASTKGPSVFPLAPSSKSTSGGTAALGCLVKDYFPEPV | | A77, A85, A93, |
| | TVSWNSGALTSGVHTFPAVLQSSGLYSLSSVVTVPSSSL | | A101, A109, |
| | GTQTYICNVNHKPSNTKVDKRVEPKSCDKTHTCPPCPAP | | A117, A125 |
| | EAAGGPSVFLFPPKPKDTLMISRTPEVTCVVVDVSHEDP | | |
| | EVKFNWYVDGVEVHNAKTKPREEQYNSTYRVVSVLTVLH | | |
| | QDWLNGKEYKCKVSNKALPAPIEKTISKAKGQPREPQVY | | |
| | TLPPSRFEMTKNQVSLTCLVKGFYPSDIAVEWESNGQPE | | |
| | NNYKTTPPVLDSDGSFFLYSKLTVDKSRWQQGNVESCSV | | |
| | MHEALHNHYTQKSLSLSPG | | |
| H6 | EVQLVESGGGLVQPGGSLRLSCAASGFTFSDYYMAWVRQ | 159 | A6, A14, A22, |
| | APGKGLEWVANINYEGSRTDLLDSLKSRFTISRDNAKNS | | A30, A38, A46, |
| | VYLQMNSLRAEDTAVYYCARDTSRGSYFDVWGAGTTVTV | | A54, A62, A70, |
| | SSASTKGPSVFPLAPSSKSTSGGTAALGCLVKDYFPEPV | | A78, A86, A94, |
| | TVSWNSGALTSGVHTFPAVLQSSGLYSLSSVVTVPSSSL | | A102, A110, |
| | GTQTYICNVNHKPSNTKVDKRVEPKSCDKTHTCPPCPAP | | A118, A126 |
| | EAAGGPSVFLFPPKPKDTLMISRTPEVTCVVVDVSHEDP | | |
| | EVKFNWYVDGVEVHNAKTKPREEQYNSTYRVVSVLTVLH | | |
| | QDWLNGKEYKCKVSNKALPAPIEKTISKAKGQPREPQVY | | |
| | TLPPSRFEMTKNQVSLTCLVKGFYPSDIAVEWESNGQPE | | |
| | NNYKTTPPVLDSDGSFFLYSKLTVDKSRWQQGNVFSCSV | | |
| | MHEALHNHYTQKSLSLSPG | | |
| H7 | EVQLVESGGGLVQPGGSLRLSCAASGFTFSDYYMAWVRQ | 169 | A7, A15, A23, |
| | APGKGLEWVANINYDGSRTDLLESLKSRFTISRDNAKNS | | A31, A39, A47, |
| | VYLQMNSLRAEDTAVYYCARDTSRGSYFDVWGAGTTVTV | | A55, A63, A71, |
| | SSASTKGPSVFPLAPSSKSTSGGTAALGCLVKDYFPEPV | | A79, A87, A95, |
| | TVSWNSGALTSGVHTFPAVLQSSGLYSLSSVVTVPSSSL | | A103, A111, |
| | GTQTYICNVNHKPSNTKVDKRVEPKSCDKTHTCPPCPAP | | A119, A127 |
| | EAAGGPSVELFPPKPKDTLMISRTPEVTCVVVDVSHEDP | | |
| | EVKFNWYVDGVEVHNAKTKPREEQYNSTYRVVSVLTVLH | | |
| | QDWLNGKEYKCKVSNKALPAPIEKTISKAKGQPREPQVY | | |
| | TLPPSRFEMTKNQVSLTCLVKGFYPSDIAVEWESNGQPE | | |
| | NNYKTTPPVLDSDGSFFLYSKLTVDKSRWQQGNVESCSV | | |
| | MHEALHNHYTQKSLSLSPG | | |
| H8 | EVKLVESGGGLVQPGGSLRLSCAASGFTFSDYYMAWVRQ | 179 | A8, A16, A24, |
| | APGKGLEWVANINYDGSRTDLLDSLKSRFTISRDNAKNS | | A32, A40, A48, |
| | VYLQMNSLRAEDTAVYYCARDTSRGSYFDVWGAGTTVTV | | A56, A64, A72, |
| | SSASTKGPSVFPLAPSSKSTSGGTAALGCLVKDYFPEPV | | A80, A88, A96, |
| | TVSWNSGALTSGVHTFPAVLQSSGLYSLSSVVTVPSSSL | | A104, A112, |
| | GTQTYICNVNHKPSNTKVDKRVEPKSCDKTHTCPPCPAP | | A120, A128 |
| | EAAGGPSVFLFPPKPKDTLMISRTPEVTCVVVDVSHEDP | | |
| | EVKFNWYVDGVEVHNAKTKPREEQYNSTYRVVSVLTVLH | | |
| | QDWLNGKEYKCKVSNKALPAPIEKTISKAKGQPREPQVY | | |
| | TLPPSRFEMTKNQVSLTCLVKGFYPSDIAVEWESNGQPE | | |
| | NNYKTTPPVLDSDGSFFLYSKLTVDKSRWQQGNVFSCSV | | |
| | MHEALHNHYTQKSLSLSPG | | |

Combinations of light chain variable regions and heavy chain variable regions shown in Tables 9 and 10, or of light chains and heavy chains shown in Tables 11 and 12, result in certain antibodies of the present disclosure shown in Table 13.

TABLE 13
SEQ ID NOs of exemplary antibodies according to the disclosure.

| | Light | Heavy | | | VL | VL | VL | VH | VH | VH |
|---|---|---|---|---|---|---|---|---|---|---|
| | Chain | Chain | VL | VH | CDR1 | CDR2 | CDR3 | CDR1 | CDR2 | CDR3 |
| Antibody | SEQ ID | SEQ ID | SEQ ID | SEQ ID | SEQ ID | SEQ ID | SEQ ID | SEQ ID | SEQ ID | SEQ ID |
| Name | NO: | NO: | NO: | NO: | NO: | NO: | NO: | NO: | NO: | NO: |
| A1 | 104 | 109 | 103 | 108 | 100 | 101 | 102 | 105 | 106 | 107 |
| A2 | 104 | 119 | 103 | 118 | 100 | 101 | 102 | 115 | 116 | 117 |
| A3 | 104 | 129 | 103 | 128 | 100 | 101 | 102 | 125 | 126 | 127 |
| A4 | 104 | 139 | 103 | 138 | 100 | 101 | 102 | 135 | 136 | 137 |
| A5 | 104 | 149 | 103 | 148 | 100 | 101 | 102 | 145 | 146 | 147 |
| A6 | 104 | 159 | 103 | 158 | 100 | 101 | 102 | 155 | 156 | 157 |
| A7 | 104 | 169 | 103 | 168 | 100 | 101 | 102 | 165 | 166 | 167 |
| A8 | 104 | 179 | 103 | 178 | 100 | 101 | 102 | 175 | 176 | 177 |
| A9 | 114 | 109 | 113 | 108 | 110 | 111 | 112 | 105 | 106 | 107 |
| A10 | 114 | 119 | 113 | 118 | 110 | 111 | 112 | 115 | 116 | 117 |
| A11 | 114 | 129 | 113 | 128 | 110 | 111 | 112 | 125 | 126 | 127 |
| A12 | 114 | 139 | 113 | 138 | 110 | 111 | 112 | 135 | 136 | 137 |
| A13 | 114 | 149 | 113 | 148 | 110 | 111 | 112 | 145 | 146 | 147 |
| A14 | 114 | 159 | 113 | 158 | 110 | 111 | 112 | 155 | 156 | 157 |
| A15 | 114 | 169 | 113 | 168 | 110 | 111 | 112 | 165 | 166 | 167 |
| A16 | 114 | 179 | 113 | 178 | 110 | 111 | 112 | 175 | 176 | 177 |
| A17 | 124 | 109 | 123 | 108 | 120 | 121 | 122 | 105 | 106 | 107 |
| A18 | 124 | 119 | 123 | 118 | 120 | 121 | 122 | 115 | 116 | 117 |
| A19 | 124 | 129 | 123 | 128 | 120 | 121 | 122 | 125 | 126 | 127 |
| A20 | 124 | 139 | 123 | 138 | 120 | 121 | 122 | 135 | 136 | 137 |
| A21 | 124 | 149 | 123 | 148 | 120 | 121 | 122 | 145 | 146 | 147 |
| A22 | 124 | 159 | 123 | 158 | 120 | 121 | 122 | 155 | 156 | 157 |
| A23 | 124 | 169 | 123 | 168 | 120 | 121 | 122 | 165 | 166 | 167 |
| A24 | 124 | 179 | 123 | 178 | 120 | 121 | 122 | 175 | 176 | 177 |
| A25 | 134 | 109 | 133 | 108 | 130 | 131 | 132 | 105 | 106 | 107 |
| A26 | 134 | 119 | 133 | 118 | 130 | 131 | 132 | 115 | 116 | 117 |
| A27 | 134 | 129 | 133 | 128 | 130 | 131 | 132 | 125 | 126 | 127 |
| A28 | 134 | 139 | 133 | 138 | 130 | 131 | 132 | 135 | 136 | 137 |
| A29 | 134 | 149 | 133 | 148 | 130 | 131 | 132 | 145 | 146 | 147 |
| A30 | 134 | 159 | 133 | 158 | 130 | 131 | 132 | 155 | 156 | 157 |
| A31 | 134 | 169 | 133 | 168 | 130 | 131 | 132 | 165 | 166 | 167 |
| A32 | 134 | 179 | 133 | 178 | 130 | 131 | 132 | 175 | 176 | 177 |
| A33 | 144 | 109 | 143 | 108 | 140 | 141 | 142 | 105 | 106 | 107 |
| A34 | 144 | 119 | 143 | 118 | 140 | 141 | 142 | 115 | 116 | 117 |
| A35 | 144 | 129 | 143 | 128 | 140 | 141 | 142 | 125 | 126 | 127 |
| A36 | 144 | 139 | 143 | 138 | 140 | 141 | 142 | 135 | 136 | 137 |
| A37 | 144 | 149 | 143 | 148 | 140 | 141 | 142 | 145 | 146 | 147 |
| A38 | 144 | 159 | 143 | 158 | 140 | 141 | 142 | 155 | 156 | 157 |
| A39 | 144 | 169 | 143 | 168 | 140 | 141 | 142 | 165 | 166 | 167 |
| A40 | 144 | 179 | 143 | 178 | 140 | 141 | 142 | 175 | 176 | 177 |
| A41 | 154 | 109 | 153 | 108 | 150 | 151 | 152 | 105 | 106 | 107 |
| A42 | 154 | 119 | 153 | 118 | 150 | 151 | 152 | 115 | 116 | 117 |
| A43 | 154 | 129 | 153 | 128 | 150 | 151 | 152 | 125 | 126 | 127 |
| A44 | 154 | 139 | 153 | 138 | 150 | 151 | 152 | 135 | 136 | 137 |
| A45 | 154 | 149 | 153 | 148 | 150 | 151 | 152 | 145 | 146 | 147 |
| A46 | 154 | 159 | 153 | 158 | 150 | 151 | 152 | 155 | 156 | 157 |
| A47 | 154 | 169 | 153 | 168 | 150 | 151 | 152 | 165 | 166 | 167 |
| A48 | 154 | 179 | 153 | 178 | 150 | 151 | 152 | 175 | 176 | 177 |
| A49 | 164 | 109 | 163 | 108 | 160 | 161 | 162 | 105 | 106 | 107 |
| A50 | 164 | 119 | 163 | 118 | 160 | 161 | 162 | 115 | 116 | 117 |
| A51 | 164 | 129 | 163 | 128 | 160 | 161 | 162 | 125 | 126 | 127 |
| A52 | 164 | 139 | 163 | 138 | 160 | 161 | 162 | 135 | 136 | 137 |
| A53 | 164 | 149 | 163 | 148 | 160 | 161 | 162 | 145 | 146 | 147 |
| A54 | 164 | 159 | 163 | 158 | 160 | 161 | 162 | 155 | 156 | 157 |
| A55 | 164 | 169 | 163 | 168 | 160 | 161 | 162 | 165 | 166 | 167 |
| A56 | 164 | 179 | 163 | 178 | 160 | 161 | 162 | 175 | 176 | 177 |
| A57 | 174 | 109 | 173 | 108 | 170 | 171 | 172 | 105 | 106 | 107 |
| A58 | 174 | 119 | 173 | 118 | 170 | 171 | 172 | 115 | 116 | 117 |
| A59 | 174 | 129 | 173 | 128 | 170 | 171 | 172 | 125 | 126 | 127 |
| A60 | 174 | 139 | 173 | 138 | 170 | 171 | 172 | 135 | 136 | 137 |
| A61 | 174 | 149 | 173 | 148 | 170 | 171 | 172 | 145 | 146 | 147 |
| A62 | 174 | 159 | 173 | 158 | 170 | 171 | 172 | 155 | 156 | 157 |
| A63 | 174 | 169 | 173 | 168 | 170 | 171 | 172 | 165 | 166 | 167 |
| A64 | 174 | 179 | 173 | 178 | 170 | 171 | 172 | 175 | 176 | 177 |
| A65 | 184 | 109 | 183 | 108 | 180 | 181 | 182 | 105 | 106 | 107 |
| A66 | 184 | 119 | 183 | 118 | 180 | 181 | 182 | 115 | 116 | 117 |
| A67 | 184 | 129 | 183 | 128 | 180 | 181 | 182 | 125 | 126 | 127 |
| A68 | 184 | 139 | 183 | 138 | 180 | 181 | 182 | 135 | 136 | 137 |
| A69 | 184 | 149 | 183 | 148 | 180 | 181 | 182 | 145 | 146 | 147 |
| A70 | 184 | 159 | 183 | 158 | 180 | 181 | 182 | 155 | 156 | 157 |
| A71 | 184 | 169 | 183 | 168 | 180 | 181 | 182 | 165 | 166 | 167 |
| A72 | 184 | 179 | 183 | 178 | 180 | 181 | 182 | 175 | 176 | 177 |
| A73 | 194 | 109 | 193 | 108 | 190 | 191 | 192 | 105 | 106 | 107 |
| A74 | 194 | 119 | 193 | 118 | 190 | 191 | 192 | 115 | 116 | 117 |
| A75 | 194 | 129 | 193 | 128 | 190 | 191 | 192 | 125 | 126 | 127 |
| A76 | 194 | 139 | 193 | 138 | 190 | 191 | 192 | 135 | 136 | 137 |
| A77 | 194 | 149 | 193 | 148 | 190 | 191 | 192 | 145 | 146 | 147 |
| A78 | 194 | 159 | 193 | 158 | 190 | 191 | 192 | 155 | 156 | 157 |
| A79 | 194 | 169 | 193 | 168 | 190 | 191 | 192 | 165 | 166 | 167 |
| A80 | 194 | 179 | 193 | 178 | 190 | 191 | 192 | 175 | 176 | 177 |
| A81 | 204 | 109 | 203 | 108 | 200 | 201 | 202 | 105 | 106 | 107 |
| A82 | 204 | 119 | 203 | 118 | 200 | 201 | 202 | 115 | 116 | 117 |
| A83 | 204 | 129 | 203 | 128 | 200 | 201 | 202 | 125 | 126 | 127 |
| A84 | 204 | 139 | 203 | 138 | 200 | 201 | 202 | 135 | 136 | 137 |
| A85 | 204 | 149 | 203 | 148 | 200 | 201 | 202 | 145 | 146 | 147 |
| A86 | 204 | 159 | 203 | 158 | 200 | 201 | 202 | 155 | 156 | 157 |
| A87 | 204 | 169 | 203 | 168 | 200 | 201 | 202 | 165 | 166 | 167 |
| A88 | 204 | 179 | 203 | 178 | 200 | 201 | 202 | 175 | 176 | 177 |
| A89 | 214 | 109 | 213 | 108 | 210 | 211 | 212 | 105 | 106 | 107 |
| A90 | 214 | 119 | 213 | 118 | 210 | 211 | 212 | 115 | 116 | 117 |
| A91 | 214 | 129 | 213 | 128 | 210 | 211 | 212 | 125 | 126 | 127 |
| A92 | 214 | 139 | 213 | 138 | 210 | 211 | 212 | 135 | 136 | 137 |
| A93 | 214 | 149 | 213 | 148 | 210 | 211 | 212 | 145 | 146 | 147 |
| A94 | 214 | 159 | 213 | 158 | 210 | 211 | 212 | 155 | 156 | 157 |
| A95 | 214 | 169 | 213 | 168 | 210 | 211 | 212 | 165 | 166 | 167 |
| A96 | 214 | 179 | 213 | 178 | 210 | 211 | 212 | 175 | 176 | 177 |
| A97 | 224 | 109 | 223 | 108 | 220 | 221 | 222 | 105 | 106 | 107 |
| A98 | 224 | 119 | 223 | 118 | 220 | 221 | 222 | 115 | 116 | 117 |
| A99 | 224 | 129 | 223 | 128 | 220 | 221 | 222 | 125 | 126 | 127 |
| A100 | 224 | 139 | 223 | 138 | 220 | 221 | 222 | 135 | 136 | 137 |
| A101 | 224 | 149 | 223 | 148 | 220 | 221 | 222 | 145 | 146 | 147 |
| A102 | 224 | 159 | 223 | 158 | 220 | 221 | 222 | 155 | 156 | 157 |
| A103 | 224 | 169 | 223 | 168 | 220 | 221 | 222 | 165 | 166 | 167 |
| A104 | 224 | 179 | 223 | 178 | 220 | 221 | 222 | 175 | 176 | 177 |
| A105 | 234 | 109 | 233 | 108 | 230 | 231 | 232 | 105 | 106 | 107 |
| A106 | 234 | 119 | 233 | 118 | 230 | 231 | 232 | 115 | 116 | 117 |
| A107 | 234 | 129 | 233 | 128 | 230 | 231 | 232 | 125 | 126 | 127 |
| A108 | 234 | 139 | 233 | 138 | 230 | 231 | 232 | 135 | 136 | 137 |
| A109 | 234 | 149 | 233 | 148 | 230 | 231 | 232 | 145 | 146 | 147 |
| A110 | 234 | 159 | 233 | 158 | 230 | 231 | 232 | 155 | 156 | 157 |
| A111 | 234 | 169 | 233 | 168 | 230 | 231 | 232 | 165 | 166 | 167 |
| A112 | 234 | 179 | 233 | 178 | 230 | 231 | 232 | 175 | 176 | 177 |
| A113 | 244 | 109 | 243 | 108 | 240 | 241 | 242 | 105 | 106 | 107 |
| A114 | 244 | 119 | 243 | 118 | 240 | 241 | 242 | 115 | 116 | 117 |
| A115 | 244 | 129 | 243 | 128 | 240 | 241 | 242 | 125 | 126 | 127 |
| A116 | 244 | 139 | 243 | 138 | 240 | 241 | 242 | 135 | 136 | 137 |
| A117 | 244 | 149 | 243 | 148 | 240 | 241 | 242 | 145 | 146 | 147 |
| A118 | 244 | 159 | 243 | 158 | 240 | 241 | 242 | 155 | 156 | 157 |
| A119 | 244 | 169 | 243 | 168 | 240 | 241 | 242 | 165 | 166 | 167 |
| A120 | 244 | 179 | 243 | 178 | 240 | 241 | 242 | 175 | 176 | 177 |
| A121 | 254 | 109 | 253 | 108 | 250 | 251 | 252 | 105 | 106 | 107 |
| A122 | 254 | 119 | 253 | 118 | 250 | 251 | 252 | 115 | 116 | 117 |
| A123 | 254 | 129 | 253 | 128 | 250 | 251 | 252 | 125 | 126 | 127 |
| A124 | 254 | 139 | 253 | 138 | 250 | 251 | 252 | 135 | 136 | 137 |
| A125 | 254 | 149 | 253 | 148 | 250 | 251 | 252 | 145 | 146 | 147 |
| A126 | 254 | 159 | 253 | 158 | 250 | 251 | 252 | 155 | 156 | 157 |
| A127 | 254 | 169 | 253 | 168 | 250 | 251 | 252 | 165 | 166 | 167 |
| A128 | 254 | 179 | 253 | 178 | 250 | 251 | 252 | 175 | 176 | 177 |

In some embodiments, the present invention includes anti-CTGF antibodies or antibody fragments thereof having a variable heavy chain and a variable light chain, wherein the variable heavy chain amino acid sequence are at least 80% or at least 85%, preferably at least 90%, more preferably at least 92.5%, still more preferably at least 95%, even more preferably at least 98%, or most preferably at least 99% identical to the amino acid sequences of SEQ ID NO: 108, 118, 128, 138, 148, 158, 168, or 178 and the variable light chain amino acid sequence are at least 80% or at least 85%, preferably at least 90%, more preferably at least 92.5%, still more preferably at least 95%, even more preferably at least 98%, or most preferably at least 99% identical to the amino acid sequences of SEQ ID NO: 103, 113, 123, 133, 143, 153, 163, 173, 183, 193, 203, 213, 223, 233, 243, or 253.

In some embodiments, the present invention includes anti-CTGF antibodies having a heavy chain and a light chain, wherein the heavy chain amino acid sequence and the light chain amino acid sequence are at least 95%, preferably at least 98%, or more preferably at least 99% identical to the amino acid sequences disclosed in Tables 11 and 12 provided that the antibody or fragments thereof retain binding to CTGF.

In some embodiments, the anti-CTGF antibodies or antibody fragments thereof comprise a variable heavy chain sequence that comprises an amino acid sequence with at least about 95%, preferably about 96%, more preferably about 97%, still more preferably about 98%, or even more preferably about 99%, sequence identity to the amino acid sequence set forth in SEQ ID NOs: 100, 101, 102, 103, 110, 111, 112, 113, 114, 115, 116, 117, 104, 118, 119, 120, 121, 122, 123, 124, or 221. Preferably, the anti-CTGF antibodies or antibody fragments thereof retains the binding and/or functional activity of an anti-CTGF antibody or antibody fragment thereof that comprises the variable heavy chain sequence of SEQ ID NOs: 100, 101, 102, 103, 110, 111, 112, 113, 114, 115, 116, 117, 104, 118, 119, 120, 121, 122, 123, 124, or 221. In some embodiments, the anti-CTGF antibodies or antibody fragments thereof comprise the variable heavy chain sequence of SEQ ID NOs: 108, 118, 128, 138, 148, 158, 168, or 178 and have one or more conservative amino acid substitutions, e.g., 1, 2, 3, 4, 5, 1-2, 1-3, 1-4 or 1-5 conservative amino acid substitutions in the heavy chain variable sequence. In some embodiments, the one or more conservative amino acid substitutions fall within one or more framework regions in SEQ ID NOs: 108, 118, 128, 138, 148, 158, 168, or 178 (based on the numbering system of Kabat).

In some embodiments, the anti-CTGF antibody or antibody fragment thereof comprises a variable heavy chain sequence with at least about 95%, preferably about 96%, more preferably about 97%, still more preferably about 98%, or even more preferably about 99% sequence identity to the anti-CTGF heavy chain variable region sequence set forth in 108, 118, 128, 138, 148, 158, 168, or 178 comprises one or more conservative amino acid substitutions in a framework region (based on the numbering system of Kabat), and retains the binding and/or functional activity of an anti-CTGF antibody or antibody fragment thereof that comprises a variable heavy chain sequence as set forth in SEQ ID NOs: 108, 118, 128, 138, 148, 158, 168, or 178 and a variable light chain sequence as set forth in SEQ ID NOs: 103, 113, 123, 133, 143, 153, 163, 173, 183, 193, 203, 213, 223, 233, 243, or 253.

In some embodiments, the anti-CTGF antibodies or antibody fragments thereof comprise a variable light chain sequence that comprises an amino acid sequence with at least about 95%, preferably about 96%, more preferably about 97%, still more preferably about 98%, or even more preferably about 99%, sequence identity to the amino acid sequence set forth in SEQ ID NOs: 103, 113, 123, 133, 143, 153, 163, 173, 183, 193, 203, 213, 223, 233, 243, or 253. Preferably, the anti-CTGF antibodies or antibody fragments thereof retains the binding and/or functional activity of an anti-CTGF antibody or antibody fragment thereof that comprises the variable light chain sequence of SEQ ID NOs: 103, 113, 123, 133, 143, 153, 163, 173, 183, 193, 203, 213, 223, 233, 243, or 253. In some embodiments, the anti-CTGF antibodies or antibody fragments thereof comprise the variable light chain sequence of SEQ ID NOs: 103, 113, 123, 133, 143, 153, 163, 173, 183, 193, 203, 213, 223, 233, 243, or 253 and have one or more conservative amino acid substitutions, e.g., 1, 2, 3, 4, 5, 1-2, 1-3, 1-4 or 1-5 conservative amino acid substitutions in the light chain variable sequence. In some embodiments, the one or more conservative amino acid substitutions fall within one or more framework regions in SEQ ID NOs: 103, 113, 123, 133, 143, 153, 163, 173, 183, 193, 203, 213, 223, 233, 243, or 253 (based on the numbering system of Kabat).

In some embodiments, the anti-CTGF antibody or antibody fragment thereof comprises a variable light chain sequence with at least about 95%, preferably about 96%, more preferably about 97%, still more preferably about 98%, or even more preferably about 99% sequence identity to the anti-CTGF light chain variable region sequence set forth in SEQ ID NOs: 103, 113, 123, 133, 143, 153, 163, 173, 183, 193, 203, 213, 223, 233, 243, or 253 comprises one or more conservative amino acid substitutions in a framework region (based on the numbering system of Kabat), and retains the binding and/or functional activity of an anti-CTGF antibody or antibody fragment thereof that comprises a variable heavy chain sequence as set forth in SEQ ID NOs: 108, 118, 128, 138, 148, 158, 168, or 178 and a variable light chain sequence as set forth in SEQ ID NOs: 103, 113, 123, 133, 143, 153, 163, 173, 183, 193, 203, 213, 223, 233, 243, or 253.

In some embodiments, the present invention includes anti-CTGF antibodies or antigen-binding fragments thereof having an amino acid substitution. These variants have at least one amino acid residue in the anti-CTGF antibody or antigen-binding fragment thereof removed and a different residue inserted in its place. The sites of greatest interest for substitutional mutagenesis include the hypervariable regions, but FR alterations are also contemplated. Conservative substitutions are shown in Table 14 under the heading of “preferred substitutions”. If such substitutions result in a change in biological activity, then more substantial changes, denominated “exemplary substitutions”, or as further described below in reference to amino acid classes, may be introduced and the products screened.

TABLE 14
Exemplary amino acid substitutions

| | | Preferred |
|---|---|---|
| Original Residue | Exemplary Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | arg; asn; gln; lys; | arg |
| Ile (I) | leu; val; met; ala; phe; norleucine | leu |
| Leu (L) | ile; norleucine; val; met; ala; phe | ile |
| Lys (K) | arg; gln; asn | arg |
| Met (M) | leu; phe; ile | leu |
| Phe (F) | tyr; leu; val; ile; ala; | tyr |
| Pro (P) | ala | ala |
| Ser (S) | thr | thr |
| Thr (T) | ser | ser |
| Trp (W) | tyr; phe | tyr |
| Tyr (Y) | phe;trp; thr; ser | phe |
| Val (V) | leu; ile; met; phe ala; norleucine; | leu |

Naturally occurring amino acid residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.

Conservative substitutions will entail exchanging a member of one of these classes for another member of the same class. Non-conservative substitutions will entail exchanging a member of one of these classes for another class.

Any cysteine residue not involved in maintaining the proper conformation of the anti-CTGF antibody or antigen-binding fragment thereof also may be substituted, generally with serine, to improve the oxidative stability of the molecule, prevent aberrant crosslinking, or provide for established points of conjugation to a cytotoxic or cytostatic compound. Conversely, cysteine bond(s) may be added to the antibody or antigen-binding fragment thereof to improve its stability (particularly where the antibody is an antibody fragment such as an Fv fragment).

Another type of variant of the antibody involves altering the original glycosylation pattern of the antibody. The term “altering” in this context means deleting one or more carbohydrate moieties found in the antibody, and/or adding one or more glycosylation sites that were not previously present in the antibody. For example, an antibody may comprise an amino acid substitution at position 297 of the human IgG₁ heavy chain to abrogate oligosaccharyltransferase enzyme complex-mediated glycosylation by replacing the asparagine 297 (e.g. N297A, N297G).

In some aspects, the present invention includes nucleic acid molecules that encode the amino acid sequence variants of the anti-CTGF antibodies or antigen-binding fragments thereof described herein. Nucleic acid molecules encoding amino acid sequence variants of an anti-CTGF antibody or antigen-binding fragment thereof are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-CTGF antibody or antigen-binding fragment thereof. For example, nucleic acid molecules according to the invention also encompass nucleic acid molecules which hybridize under stringent conditions to nucleic acid molecules as disclosed herein, whereby the term “stringent conditions” within the scope of the invention can include, e.g., hybridization in a buffer comprising 50% formamide, 5×SSC, and 1% SDS at 42 °C, or hybridization in a buffer comprising 5×SSC and 1% SDS at 65 °C, both with a wash of 0.2×SSC and 0.1% SDS at 65 °C. Exemplary stringent hybridization conditions can also include a hybridization in a buffer of 40% formamide, 1 M NaCl, and 1% SDS at 37 °C, and a wash in 1×SSC at 45 °C.

In certain embodiments, the anti-CTGF antibody is an antibody fragment. There are techniques that have been developed for the production of antibody fragments. Fragments can be derived via proteolytic digestion of intact antibodies (see, e.g., Morimoto et al., 1992, Journal of Biochemical and Biophysical Methods 24:107-117; and Brennan et al., 1985, Science 229:81). Alternatively, the fragments can be produced directly in recombinant host cells. For example, Fab′-SH fragments can be directly recovered from E. coli and chemically coupled to form F(ab′)₂ fragments (see, e.g., Carter et al., 1992, Bio/Technology 10:163-167). By another approach, F(ab′)₂ fragments can be isolated directly from recombinant host cell culture. Other techniques for the production of antibody fragments will be apparent to a skilled practitioner.

In some aspects, the anti-CTGF antibodies and antigen-binding fragments thereof can include modifications, such as glycosylation, oxidation, or deamidation.

In certain embodiments, it may be desirable to use an anti-CTGF antibody fragment, rather than an intact antibody. It may be desirable to modify the antibody fragment in order to increase its serum half-life. This can be achieved, for example, by incorporation of a salvage receptor binding epitope into the antibody fragment. In one method, the appropriate region of the antibody fragment can be altered (e.g., mutated), or the epitope can be incorporated into a peptide tag that is then fused to the antibody fragment at either end or in the middle, for example, by DNA or peptide synthesis (see, e.g., WO 96/32478). For example, antibody fragments of the invention may also be fused to human serum albumin to increase the serum half-life, if the use of a full length IgG scaffold is undesirable. Such fusion proteins of the antibody fragment with human serum albumin may be advantageous in situations in which two different antibody fragments need to be fused to increase avidity, or to generate a bispecific binding protein with extended serum half-life (see e.g. WO 05/077042 A2).

Removal of any carbohydrate moieties present on the antibody can be accomplished chemically or enzymatically. Chemical deglycosylation is described by Hakimuddin et al., 1987, Arch. Biochem. Biophys. 259:52 and by Edge et al., 1981, Anal. Biochem., 118:131. Enzymatic cleavage of carbohydrate moieties on antibodies can be achieved by the use of a variety of endo- and exo-glycosidases as described by Thotakura et al., 1987, Meth. Enzymol 138:350.

A type of useful modification comprises linking the antibody to one of a variety of nonproteinaceous polymers, e.g., polyethylene glycol, polypropylene glycol, or polyoxyalkylenes, in the manner set forth in one or more of U.S. Pat. Nos. 4,640,835, 4,496,689, 4,301,144, 4,670,417, 4,791,192 and 4,179,337.

The present invention also provides the anti-CTGF antibody or antigen-binding fragment thereof that competes for binding to CTGF with an anti-CTGF antibody according to the present invention. In one embodiment, the present invention provides an anti-CTGF antibody or antigen-binding fragment thereof that competes for binding to CTGF with antibodies A1-A128. Competition assays may be conducted for example as described in PLOS One. 2014; 9(3):e92451 using a biosensor, or PLOS One 2020 Mar. 5; 15(3):e0229206, or by a method disclosed herein.

### Therapeutic Uses

In one embodiment, the anti-CTGF antibodies of the invention or antigen-binding fragments thereof are useful for treating and/or preventing CTGF-associated disorders. In some embodiments, the anti-CTGF antibodies of the invention or antigen-binding fragments thereof are useful as a medicament.

The present invention provides antibodies for treatment (use in) of various diseases and disorders associated with CTGF. The antibodies of the invention reduce the deleterious effects of CTGF production or activity in several disorders. Further, the antibodies show favorable pharmacokinetics and/or pharmacodynamics, making them superior therapeutic agents for the treatment of disorders associated with CTGF.

The anti-CTGF antibodies of the present invention inhibit development of fibrosis. The present disclosure contemplates treating patients with a CTGF-associated disorder with an anti-CTGF antibody to improve or stabilize the pathology, restore organ function, improve the quality of life, and extend survival.

The antibodies of the invention are useful in therapeutic applications to prevent or treat CTGF-associated disorders involving fibrosis. In some aspects, the antibodies of the invention are administered to a subject to prevent or treat a CTGF-associated disorder including, but are not limited to, disorders exhibiting altered expression and deposition of extracellular matrix-associated proteins, e.g., fibrotic disorders. In various aspects, the fibrosis may be localized to a particular tissue, such as epithelial, endothelial, or connective tissue; or to an organ, such as kidney, lung, or liver. Fibrosis can also occur in the eye and joints. In other aspects, the fibrosis may be systemic and involve multiple organ and tissue systems. CTGF-associated disorders include, for example, atherosclerosis, arthritis, retinopathies such as diabetic retinopathy; nephropathies such as diabetic nephropathy; Duchenne muscular dystrophy, cardiac, pulmonary, liver, and kidney fibrosis, and diseases associated with chronic inflammation and/or infection.

Additionally, the antibodies of the invention are useful in therapeutic applications, to prevent or treat CTGF-associated disorders in a subject. Such disorders include, but are not limited to, disorders characterized by angiogenesis and other processes which play a central role in conditions such as atherosclerosis, glaucoma, etc.; and in cancer, including acute lymphoblastic leukemia, dermatofibromas, breast cancer, breast carcinoma, glioma and glioblastoma, rhabdomyosarcoma and fibrosarcoma, desmoplasia, angiolipoma, angioleiomyoma, desmoplastic cancers, and prostate, ovarian, colorectal, pancreatic, gastrointestinal, and liver cancer and other tumor growth and metastases.

In some aspects, the invention provides antibodies for reducing the incidence of or preventing a CTGF-associated disorder in a subject having a predisposition to develop such a disorder. A predisposition may include, e.g., hyperglycemia, hypertension, or obesity in the subject. Such disorders may occur, e.g., due to diabetes, obesity, etc., and include diabetic nephropathy, retinopathy, and cardiovascular disease. Additionally, a predisposition may be suspected due to an event, e.g., a myocardial infarction, surgery, peritoneal dialysis, chronic and acute transplant rejection, chemotherapy, radiation therapy, trauma, orthopedic or paralytic immobilization, congestive heart failure, pregnancy, or varicosities in the subject.

In particular embodiments, as exemplified herein, the antibodies of the present invention are administered to a subject to treat fibrosis of an organ, e.g., lung or kidney. In additional embodiments, the antibodies are administered to a subject to treat or prevent ocular disorders such as proliferative vitreoretinopathy, diabetic retinopathy, or macular degeneration. Because CTGF is implicated in a wide variety of disorders, the invention contemplates treating patients having a CTGF-associated disorder using an antibody of the invention to improve or stabilize pathology and organ function, improve the quality of life, and extend survival.

For therapeutic applications, the antibodies of the invention are administered to a mammal, preferably a human, in a pharmaceutically acceptable dosage form. The antibodies may be administered intravenously. The antibodies may be administered intravenously as a bolus or by continuous infusion over a period of time. The antibodies may be administered by intramuscular, subcutaneous, intra-articular, intrasynovial, intrathecal, intravitreal, intracranial, oral, topical, or inhalation routes. The antibodies may be administered subcutaneously. When the antibody possesses the suitable activity, intratumoral, peritumoral, intralesional, or perilesional routes of administration can also be utilized to exert local as well as systemic therapeutic effects.

Such dosage forms encompass pharmaceutically acceptable carriers that are nontoxic and nontherapeutic. When present in an aqueous dosage form, rather than being lyophilized, the antibody typically will be formulated at a concentration of about 0.1 mg/ml to 100 mg/ml, although wide variation outside of these ranges is permitted.

For the prevention or treatment of disease, the appropriate dosage of antibody will depend on the type of disease to be treated, as defined above, the severity and course of the disease, whether the antibodies are administered for preventive or therapeutic purposes, the course of previous therapy, the patient's clinical history and response to the antibody, and the discretion of the attending physician. The antibody is suitably administered to the patient at one time or over a series of treatments.

Depending on the type and severity of the disease, about 0.015 to 25 mg of antibody/kg of patient weight is an initial candidate dosage for administration to the patient, whether, for example, by one or more separate administrations, or by continuous infusion. For repeated administrations over several days or longer, depending on the condition, the treatment is repeated until a desired suppression of disease symptoms occurs. However, other dosage regimens may be useful and are not excluded from the present invention.

According to some embodiments of the invention, the effectiveness of the antibody in preventing or treating disease may be improved by administering the antibody serially or in combination with another agent that is effective for the same clinical objective, such as another antibody directed against a different epitope than the principal antibody, or one or more conventional therapeutic agents known for the intended therapeutic indication, e.g. prevention or treatment of conditions associated with excessive extracellular matrix production such as fibrosis or sclerosis, inhibition of tumor cell growth or metastasis, inhibition of neovascularization, or reduction of inflammation. Such agents may ameliorate symptoms or improve outcome via a similar mechanism of action, e.g., anti-TGFβ antibodies, or by a different mechanism, e.g., interferon-γ. Such agents may additionally ameliorate symptoms directly or indirectly associated with a CTGF-associated disorder or a predisposition to develop a CTGF-associated disorder, e.g., angiotensin-converting enzyme (ACE) inhibitors and angiotensin receptor blockers (Arbs).

### Non-Therapeutic Uses

The antibodies described herein are useful as affinity purification agents. In this process, the antibodies are immobilized on a solid phase such a Protein A resin, using methods well known in the art. The immobilized antibody is contacted with a sample containing the CTGF protein (or fragment thereof, such as CTGF-NTF) to be purified, and thereafter the support is washed with a suitable solvent that will remove substantially all the material in the sample except the CTGF protein, which is bound to the immobilized antibody. Finally, the support is washed with another suitable solvent that will release the CTGF protein from the antibody.

The CTGF antibodies and fragments thereof of the invention as disclosed herein are also useful in diagnostic assays to detect and/or quantify CTGF protein, for example, detecting CTGF expression in specific cells, tissues, or plasma.

It will be advantageous in some embodiments, for example, for diagnostic purposes to label the antibody with a detectable moiety. Numerous detectable labels are available, including radioisotopes, fluorescent labels, enzyme substrate labels, quantum dots and the like. The label may be indirectly conjugated with the antibody using various known techniques. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody can be conjugated with a small hapten (such as digoxin) and one of the different types of labels mentioned above is conjugated with an anti-hapten antibody (e.g., anti-digoxin antibody). Thus, indirect conjugation of the label with the antibody can be achieved.

### Diagnostic Kits

An anti-CTGF antibody or fragment thereof can be used in a diagnostic kit, i.e., a packaged combination of reagents in predetermined amounts with instructions for performing the diagnostic assay, such as an assay for the detection of full length CTGF and CTGF-NTF, e.g., in plasma. Accordingly, also provided herein is the use of the antibody or antigen-binding fragment thereof as described herein for diagnosis of a CTGF-associated disorder. Where the antibody is labeled with an enzyme, the kit may include substrates and cofactors required by the enzyme such as a substrate precursor that provides the detectable chromophore or fluorophore. In addition, other additives may be included such as stabilizers, buffers (for example a block buffer or lysis buffer), and the like. The relative amounts of the various reagents may be varied widely to provide for concentrations in solution of the reagents that substantially optimize the sensitivity of the assay. The reagents may be provided as dry powders, usually lyophilized, including excipients that on dissolution will provide a reagent solution having the appropriate concentration.

### Compositions, Combinations and Administration Thereof

A composition comprising an anti-CTGF antibody or an antigen-binding fragment thereof according to the invention can be administered to a subject having or at risk of the CTGF-associated diseases and/or disorders described herein. The invention provides for the use of an anti-CTGF antibody or an antigen-binding fragment thereof in the manufacture of a medicament for prevention or treatment of a CTGF-associated disease or disorder. The term “subject” as used herein means any mammalian patient to which an anti-CTGF antibody or an antigen-binding fragment thereof can be administered, including, e.g., humans and certain non-human mammals, such as domesticated animals (e.g., cows, sheep, pigs, cats, dogs, and horses), primates (e.g., humans and non-human primates such as monkeys), rabbits, and rodents (e.g., mice and rats). Subjects specifically intended for treatment using the methods described herein include humans.

An anti-CTGF antibody or an antigen-binding fragment thereof may be administered on their own or in combination with one or more additional therapeutic agents, such as state-of-the-art or standard-of-care compounds, such as e.g., anti-fibrotic agents, steroids, immune modulators, checkpoint inhibitors, cell proliferation inhibitors, anti-angiogenic substances, cytostatic or cytotoxic substances, and the like.

In some aspects, the present invention also provides pharmaceutical compositions administered as pharmaceutical compositions comprising a therapeutically effective amount of the anti-CTGF antibody or an antigen-binding fragment thereof, and optionally one or more additional therapeutic agents.

Some aspects of the invention provide a binding molecule of the invention for use in the therapy of a CTGF-associated disease, wherein said therapy comprises one or more pharmacologically active substances.

Some aspects of the invention provide the use of one or more active ingredients in the manufacture of a medicament for the therapy of CTGF-associated disease, wherein said medicament comprises the binding molecule of the invention.

In some aspects of the invention, the anti-CTGF antibody or antigen-binding fragment thereof is optionally administered in combination with an additional therapeutic agent. The additional therapeutic agent may be a phosphodiesterase 4 inhibitor, a tyrosine kinase inhibitor, a lysophosphatidic acid receptor 1 (LPA1) antagonist, an inhibitor of Src and Abl kinases, a prostacyclin vasodilator, an angiotensin II type 2 receptor agonist (ATRAG), a c-Jun N-terminal kinase inhibitor, an analgesic, an opioid, a P2X3 homotrimeric receptor antagonist, an N-methyl-D-aspartate (NMDA) receptor inhibitor, an additional CTGF inhibitor, an anxiolytic, a proton pump inhibitor, an antifibrotic agent, or any combination thereof. The additional therapeutic agent may be 9MW3811 (Mabwell Shanghai Bioscience Co., Ltd.), ANG-3070 (Angion Biomedica Corp), AP01 (Avalyn Pharma Inc.), autoantibody reductive therapy, autologous lung spheroid stem cells (University of North Carolina, Chapel Hill), AZD5055 (AstraZeneca), AZD8965 (AstraZeneca), BBT-877 (Bridge Biotherapeutics), belumosudil (K_{D}025, Kadmon), BG00011 (Biogen, STX-100), BI 1819479 (Boehringer Ingelheim), BI 1839100 (Boehringer Ingelheim), BLD-0409 (Blade Therapeutics), BLD-2660 (Blade), BLU-5937, BMS-986020 (Bristol Myers Squibb), BMS-986278 (Bristol-Myers Squibb), BNC 1021 (BONAC/Toray, TRK-250, PK-7010), bosentan, buloxibutid (Vicore Pharma AB, C21), buspirone, C106 (Vicore Pharma AB), CAL101 (Calluna Pharma AS), carlumab (Janssen, CNTO-888), CC-90001 (Celgene), clonazepam, CMR316 (Calibr), codeine, dasatinib, dectrekumab (Novartis, QAX576), desvenlafaxine, deupirfenidone (PureTech, LYT-100), DWN12088 (Daewoong Pharmaceutical Co. LTD.), Taladegib (Endeavor Biomedicines, Inc., ENV-101), epigallocatechin-3-gallate (University of California, San Francisco), fipaxalparant (Horizon Therapeutics, HZN-825), fluoxetine, gabapentin, garadacimab (CSL, CSL312), GB 2064 (Galecto, PAT-1251), GB0139 (Galecto), gefapixant, GLPG1690 (Galapagos), GSK3915393 (GlaxoSmithKline), HEC 68498 (HEC Pharm), HEC-866 (HEC Pharm), HSK44459 (Haisco Pharmaceutical Group Co., Ltd.), HuL001 (HuniLife Biotechnology, Inc.), human umbilical cord mesenchymal stem cell injection (Shanghai Life Science & Technology), ifenprodil, ifetroban (Cumberland Pharmaceuticals), imatinib, indolinone, ISM001-055 (InSilico Medicine Hong Kong Limited, INS018_055), IW001 (ImmuneWorks), lanalumab (Novartis), lebrikizumab, leramistat (Modern Biosciences Ltd), letemovir, lorazepam, LTI-03 (Lung Therapeutics, Inc), LTP001 (Novartis), macitentan, minocycline, N-acetylcysteine (NAC), nalbuphine (Trevi Therapeutics), ND-L02-50201 (Nitto Denko), nerandomilast (Boehringer Ingelheim, BI 1015550), nintedanib (Boehringer Ingelheim), NIP292 (CR Pharma, The National Institutes of Pharmaceutical R&D Co. Ltd, China), olokizumab (R-Pharm International, LLC), omeprazole, ORIN1001 (Fosun, Orinove), orvepitant, pamrevlumab (FibroGen), pantoprazole, pirfenidone, PLN-74809 (Pliant Therapeutics, bexotegrast), prednisone, pregabalin, PRM-151 (recombinant human pentraxin-2 protein, Genetech, Promedior), PRS-220 (Pieris Australia Pty Ltd), quercetin, rabeprazole, rituximab, RSN0402 (Shenzhen Resproly Biopharmaceutical Co., Ltd), saracatinib, SB17170 (SPARK Biopharma), setanaxib (Genkyotex, GKT137831), setogepram (ProMetic, PBI-4050), SHR-1906 (Hengrui Pharmaceuticals Co., Ltd), sildenafil, simtuzumab (Gilead, GS-6624), sirolimus, SM-04646 (Samumed), tanzisertib (Celgene, CC-930), thalidomide, tipelukast (MediciNova, MN-001), treprostinil (United Therapeutics), TRK-250 (Toray, BNC-1021), TTI-101 (Tvardi Therapeutics), valganciclovir, vixarelimab (Genentech), voxelotor (GBT446), zileuton, zinc and nicotinamide riboside (Cedars-Sinai Medical Center), or any combination thereof. The additional therapeutic agent is preferably nintedanib, nerandomilast, pirfenidone, or any combination thereof.

Various delivery systems are known and can be used to administer the anti-CTGF antibody or an antigen-binding fragment thereof. Methods of introduction include but are not limited to intravitreal, eye drops, intradermal, intramuscular, intraperitoneal, intravenous, subcutaneous, intranasal, epidural, and oral routes. The anti-CTGF antibody or an antigen-binding fragment thereof can be administered, for example by infusion, bolus or injection, and can be administered together with other biologically active agents. Administration can be systemic or local. Formulations for such injections may be prepared in, for example, prefilled syringes. As such in an aspect of the invention, pre-filled syringes are provided that include an anti-CTGF antibody or an antigen-binding fragment thereof.

To be used in therapy, the anti-CTGF antibody of the invention is formulated into pharmaceutical compositions appropriate to facilitate administration to animals or humans. Typical formulations of the binding molecule or antibody molecule described herein can be prepared by mixing the binding molecule or antibody molecule with physiologically acceptable carriers, excipients or stabilizers, in the form of lyophilized or otherwise dried formulations or aqueous solutions or aqueous or non-aqueous suspensions. Carriers, excipients, modifiers or stabilizers are nontoxic at the dosages and concentrations employed.

In typical embodiments, the pharmaceutical composition is formulated in accordance with routine procedures as a pharmaceutical composition adapted for intravenous or subcutaneous administration to a subject. Typically, compositions for administration by injection are solutions in sterile isotonic aqueous buffer. Where necessary, the pharmaceutical composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

Further, the pharmaceutical composition can be provided as a pharmaceutical kit comprising (a) a container containing an anti-CTGF antibody or an antigen-binding fragment thereof in lyophilized form and (b) a second container containing a pharmaceutically acceptable diluent (e.g., sterile water) for injection. The pharmaceutically acceptable diluent can be used for reconstitution or dilution of the lyophilized anti-CTGF antibody or antigen-binding fragment thereof. Optionally associated with such container(s) can be a notice in the form prescribed by a governmental agency regulating the manufacture, use or sale of pharmaceuticals or biological products, which notice reflects approval by the agency of manufacture, use or sale for human administration.

In some embodiments, antibodies of the invention can be formulated to doses, which include for example a dose of 1 mg/kg to 200 mg/kg. including, for example, 1 mg/kg to 25 mg/kg, 25 mg/kg to 50 mg/kg, 50 mg/kg to 75 mg/kg, 75 mg/kg to 100 mg/kg, 100 mg/kg to 125 mg/kg, 125 mg/kg to 150 mg/kg, 150 mg/kg to 175 mg/kg, or 175 mg/kg to 200 mg/kg. With respect to therapeutic regimens for combinatorial administration, in a specific embodiment, an anti-CTGF antibody or antigen-binding fragment thereof is administered concurrently with a second and/or third therapeutic agent. In some embodiments, the second and/or third therapeutic agent is administered prior or subsequent to administration of the anti-CTGF antibody or antigen-binding fragment thereof.

### Polynucleotides, Vectors, and Host Cells

The present invention relates to (isolated) polynucleotides that comprise a sequence encoding an anti-CTGF antibody or antigen-binding fragment thereof, vectors, and host cells comprising the polynucleotides, and recombinant techniques for production of the antibody. As described above, the antibody or antigen-binding fragment thereof, as described herein, may be encoded on a single or on a plurality of polynucleotides (e.g., with heavy and light chain encoded on separate polynucleotides). The (isolated) polynucleotides can encode any desired form of the anti-CTGF antibody including, for example, full length monoclonal antibodies, Fab, Fab′, F(ab′)₂, and Fv fragments, diabodies, linear antibodies, single-chain antibody molecules, and multispecific antibodies formed from antibody fragments.

The polynucleotide(s) that comprise a sequence encoding an anti-CTGF antibody or a fragment or chain thereof can be fused to one or more regulatory or control sequence, as known in the art, and can be contained in suitable expression vectors or host cell as known in the art. Each of the polynucleotide molecules encoding the heavy or light chain variable domains can be independently fused to a polynucleotide sequence encoding a constant domain, such as a human constant domain, enabling the production of intact antibodies. Alternatively, polynucleotides, or portions thereof, can be fused together, providing a template for production of a single chain antibody.

For recombinant production, a polynucleotide encoding the antibody is inserted into a replicable vector for cloning (amplification of the DNA) or for expression. Many suitable vectors for expressing the recombinant antibody are available. The vector components generally include, but are not limited to, one or more of the following: a signal sequence, an origin of replication, one or more marker genes, an enhancer element, a promoter, and a transcription termination sequence.

The anti-CTGF antibodies can also be produced as fusion polypeptides, in which the antibody is fused with a heterologous polypeptide, such as a signal sequence or other polypeptide having a specific cleavage site at the amino terminus of the mature protein or polypeptide. The heterologous signal sequence selected is typically one that is recognized and processed (e.g., cleaved by a signal peptidase) by the host cell. For prokaryotic host cells that do not recognize and process the anti-CTGF antibody signal sequence, the signal sequence can be substituted by a prokaryotic signal sequence. The signal sequence can be, for example, alkaline phosphatase, penicillinase, lipoprotein, heat-stable enterotoxin II leaders, and the like. For yeast secretion, the native signal sequence can be substituted, for example, with a leader sequence obtained from yeast invertase alpha-factor (including Saccharomyces and Kluyveromyces α-factor leaders), acid phosphatase, C. albicans glucoamylase, or the signal described in WO 90/13646. In mammalian cells, mammalian signal sequences as well as viral secretory leaders, for example, the herpes simplex gD signal, can be used. The DNA for such precursor region is ligated in reading frame to DNA encoding the anti-CTGF antibody.

Anti-CTGF antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, or from heat-shock promoters, provided such promoters are compatible with the host cell systems.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for anti-CTGF antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastors (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated anti-CTGF antibody are derived from multicellular organisms. Examples of invertebrate cells include plant and insect cells, including, e.g., numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori (silk worm). A variety of viral strains for transfection are publicly available, e.g., the L-1 variant of Autographa californica NPV and the Bm-5 strain of Bombyx mori NPV, and such viruses may be used, particularly for transfection of Spodoptera frugiperda cells.

Plant cell cultures of cotton, corn, potato, soybean, petunia, tomato, and tobacco can also be utilized as hosts.

The anti-CTGF antibodies or antigen-binding fragments thereof can also be incorporated in viral vectors, e.g. the polynucleotide encoding for the anti-CTGF antibody or antigen-binding fragment thereof is introduced into the viral vector and then expressed in the body of the subject after infection with the virus.

In some aspects, expression of the anti-CTGF antibody or antigen-binding fragment thereof is carried out in vertebrate cells. The propagation of vertebrate cells in culture (tissue culture) has become routine procedure and techniques are widely available. Examples of useful mammalian host cell lines are monkey kidney CV1 line transformed by SV40 (COS-7, ATCC CRL 1651), human embryonic kidney line (293 or 293 cells subcloned for growth in suspension culture, (Graham et al., 1977, J. Gen Virol. 36:59), baby hamster kidney cells (BHK, ATCC CCL 10), Chinese hamster ovary cells/-DHFR1 (CHO, Urlaub et al., 1980, Proc. Natl. Acad. Sci. USA 77:4216; e.g., DG44), mouse sertoli cells (TM4, Mather, 1980, Biol. Reprod. 23:243-251), monkey kidney cells (CV1 ATCC CCL 70), African green monkey kidney cells (VERO-76, ATCC CRL-1587), human cervical carcinoma cells (HELA, ATCC CCL 2), canine kidney cells (MDCK, ATCC CCL 34), buffalo rat liver cells (BRL 3A, ATCC CRL 1442), human lung cells (W138, ATCC CCL 75), human liver cells (Hep G2, HB 8065), mouse mammary tumor (MMT 060562, ATCC CCL51), TR1 cells (Mather et al., 1982, Annals N.Y. Acad. Sci. 383:44-68), MRC 5 cells, FS4 cells, and human hepatoma line (Hep G2).

Also included are nucleic acids that hybridize under low, moderate, and high stringency conditions, in particular under high stringency conditions, as defined herein, to all or a portion (e.g., the portion encoding the variable region) of the nucleotide sequence represented by isolated polynucleotide sequence(s) that encode an anti-CTGF antibody or antibody fragment. The hybridizing portion of the hybridizing nucleic acid is typically at least 15 (e.g., 20, 25, 30 or 50) nucleotides in length. The hybridizing portion of the hybridizing nucleic acid is at least 80%, e.g., at least 90%, at least 95%, at least 98%, or at least 99% identical to the sequence of a portion or all of a nucleic acid encoding an anti-CTGF polypeptide (e.g., a heavy chain or light chain variable region), or its complement. Hybridizing nucleic acids of the type described herein can be used, for example, as a cloning probe, a primer, e.g., a PCR primer, or a diagnostic probe. In some aspects, “high stringency conditions” means for probes of at least 100 nucleotides in length, prehybridization and hybridization at 42 °C in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65 °C.

In one embodiment, the present invention relates to an isolated polynucleotide comprising a nucleotide sequence encoding a heavy chain variable region comprising the amino acid sequence of any one of SEQ ID NOs: 108, 118, 128, 138, 148, 158, 168, or 178.

In one embodiment, the present invention relates to an isolated polynucleotide comprising a nucleotide sequence encoding a light chain variable region comprising the amino acid sequence of any one of SEQ ID NOs: 103, 113, 123, 133, 143, 153, 163, 173, 183, 193, 203, 213, 223, 233, 243, or 253.

In one embodiment, the present invention relates to an isolated polynucleotide comprising a nucleotide sequence encoding a heavy chain region comprising the amino acid sequence of any one of SEQ NO: 109, 119, 129, 139, 149, 159, 169, or 179.

In one embodiment, the present invention relates to an isolated polynucleotide comprising the nucleotide sequence encoding of a light chain region comprising the amino acid sequence of any one any one of SEQ NO: 104, 114, 124, 134, 144, 154, 164, 174, 184, 194, 204, 214, 224, 234, 244, or 254.

### Articles of Manufacture

In some aspects, an article of manufacture containing materials useful for the treatment of the disorders described above is included. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port. For example, the container may be an intravenous solution bag or a vial having a stopper pierceable by a hypodermic injection needle. The active agent in the composition is the anti-CTGF antibody or the antigen-binding fragment thereof. The label on or associated with the container indicates that the composition is used for treating the condition of choice. The article of manufacture may further comprise a second container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use.

### Additional Exemplary Anti-CTGF Antibody and Compound Sequences

The sequences of certain exemplary anti-CTGF antibodies and compounds used in the working examples herein are provided as follows.

Antibody F1 light chain:
(SEQ ID NO: 20)
DIQMTQSPSSLSASVGDRVTITCRASQGISSWLAWYQQKPEKAPKSLIYA
ASSLQSGVPSRFSGSGSGTDFTLTISSLQPEDFATYYCQQYNSYPPTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.
Antibody F1 heavy chain:
(SEQ ID NO: 21)
EGQLVQSGGGLVHPGGSLRLSCAGSGFTFSSYGMHWVRQAPGKGLEWVSG
IGTGGGTYSTDSVKGRFTISRDNAKNSLYLQMNSLRAEDMAVYYCARGDY
YGSGSFFDCWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRFEMTKNQVSLTCLVKGFYPSDIAVEWESNGQPENNYKTTPPV
LDSDGSFFLYSKLTVDKSRWQQGNVFSCSVMHEALHNHYTQKSLSLSPGK.
Antibody H1 light chain:
(SEQ ID NO: 22)
ETVVTQEPSLTVSPGGTVTLTCRSSIGAVTTSNYANWVQQKPGQAFRGLI
GGTSNRAPWTPARFSGSLLGGKAALTLSGVQPEDEAEYYCALWYSTHYVF
GGGTKLTVLGQPKANPTVTLFPPSSEELQANKATLVCLISDFYPGAVTVA
WKADGSPVKAGVETTKPSKQSNNKYAASSYLSLTPEQWKSHRSYSCQVTH
EGSTVEKTVAPTECS.
Antibody H1 heavy chain:
(SEQ ID NO: 23)
EVTLKESGPVLVKPTETLTLTCTVSGFSLSTFGVHWIRQPPGKGLEWLGV
IWRRGGTDYNAAFMSRLTISKDTSKSQVVFTMTNMDPVDTATYYCARDGG
FDYWGQGTTVTVSSASTKGPSVFPLAPSSKSTSGGTAALGCLVKDYFPEP
VTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLYI
TREPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK.
Antibody H2 light chain:
(SEQ ID NO: 24)
EIVLTQSPATLSLSPGERATLSCSASSSVSYMHWFQQKPGQSPKLWIYST
SNLASGVPARFSGSGSGTDYTLTISSLEPEDFAVYYCQQRSSYPLTFGQG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.
Antibody H2 heavy chain:
(SEQ ID NO: 25)
EVQLVESGGGLVQPGGSLRLSCAASGFSFNTYAMNWVRQAPGKGLEWVAR
IRTKSNNYATYYADSVKDRFTISRDDSESSLYLQMNSLKTEDTAVYYCVE
TGFAYWDQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPG.
Compound P1:
(SEQ ID NO: 26)
QDSTSDLIPAPPLSKVPLQQNFQDNQFQGKWYVVGVAGNAILRPDKQPAK
MFATIYELKEDKSYDVTSVRFEAKSCKYTIKTFVPGSQPGEFTSGQIKSA
PGQTSTLVRVVSTNYNQHAMVFFKHVSQNREIFVITLYGRTKELTSELKE
NFIRFSKSLGLPENHIVFPVPIDQCIDGHHHHHHHHHH.

### Human CTGF and CTGF-NTF Sequences

A wild-type human CTGF sequence is available as NCBI Accession No. NP_001892.2 having the following amino acid sequence (with the signal peptide included): MTAASMGPVRVAFVVLLALCSRPAVGQNCSGPCRCPDEPAPRCPAGVSL VLDGCGCCRVCAKQLGELCTERDPCDPHKGLFCHFGSPANRKIGVCTAKDGAPCIFGGTVYR SGESFQSSCKYQCTCLDGAVGCMPLCSMDVRLPSPDCPFPRRVKLPGKCCEEWVCDEPKDQ TVVGPALAAYRLEDTFGPDPTMIRANCLVQTTEWSACSKTCGMGISTRVTNDNASCRLEKQSR LCMVRPCEADLEENIKKGKKCIRTPKISKPIKFELSGCTSMKTYRAKFCGVCTDGRCCTPHRTT TLPVEFKCPDGEVMKKNMMFIKTCACHYNCPGDNDIFESLYYRKMYGDMA (SEQ ID NO: 1).

Full-length human CTGF used in the working examples herein has the following amino acid sequence, corresponding to NCBI Accession No. NP_001892.1 (with the signal peptide omitted): QNCSGPCRCPDEPAPRCPAGVSLVLDGCGCCRVCAKQLGELCTERDPCD PHKGLFCDFGSPANRKIGVCTAKDGAPCIFGGTVYRSGESFQSSCKYQCTCLDGAVGCMPLC SMDVRLPSPDCPFPRRVKLPGKCCEEWVCDEPKDQTVVGPALAAYRLEDTFGPDPTMIRANC LVQTTEWSACSKTCGMGISTRVTNDNASCRLEKQSRLCMVRPCEADLEENIKKGKKCIRTPKIS KPIKFELSGCTSMKTYRAKFCGVCTDGRCCTPHRTTTLPVEFKCPDGEVMKKNMMFIKTCACH YNCPGDNDIFESLYYRKMYGDMA (SEQ ID NO:2), which was purchased from R&D Systems (catalog no. 9190-CC).

The human N-terminal fragment of CTGF (CTGF-NTF) used in the working examples herein has the following amino acid sequence: QNCSGPCRCPDEPAPRCPAGVSLVLDGCGC CRVCAKQLGELCTERDPCDPHKGLFCDFGSPANRKIGVCTAKDGAPCIFGGTVYRSGESFQS SCKYQCTCLDGAVGCMPLCSMDVRLPSPDCPFPRRVKLPGKCCEEWVCDEPKDQTVVGPAL AENLYFQ (SEQ ID NO: 3), which was recombinantly expressed. The final 6 amino acids at the C-terminus of the CTGF-NTF sequence are a partial TEV protease cleavage site that remains following purification.

### EXAMPLES

### Example 1. Generation of Anti-CTGF Antibodies

### Methods

Mice are immunized with a human N-terminal fragment of CTGF (CTGF-NTF). B cells are harvested and used to produce hybridomas by fusion with PAI myeloma cells. The resulting antibodies are screened for binding to recombinant human, cynomolgus monkey, and mouse CTGF-NTF. Antibodies are purified from serum-free hybridoma supernatants using MabSelectSuRe resin (GE Healthcare). Binding of the purified antibodies human and cynomolgus CTGF-NTF are assayed on an Octet QK system (ForteBio) equipped with streptavidin (SA) biosensor tips (ForteBio). Variable gene sequences are recovered from hybridomas, and chimeric antibodies are generated containing these variable gene sequences.

### Results

The two clones were obtained having binding affinity to human CTGF-NTF of 15 pM and 40 pM respectively. These clones were observed to have similar sequences to one another. The antibody having binding affinity to human CTGF-NTF of 15 pM was selected for humanization, resulting in the humanized antibody A128, having the light chain variable region of SEQ ID NO: 253 and the heavy chain variable region of SEQ ID NO: 178.

### Example 2. Production and Characterization of Humanized Antibodies

This example describes production and testing of variants of humanized antibody A128 described in Example 1. In total, eight variants of the heavy chain variable region and sixteen variants of the light chain variable region were produced. Each heavy chain variable region variant is co-expressed with each light chain variable region variant in CHO cells, resulting in 128 different combinations.

Percentage humanness of the heavy- and light-chain variable regions, expression titer, and binding affinity for full length CTGF determined by surface plasmon resonance (SPR) were determined. Results are shown in Table 15 below.

TABLE 15

| | VL | VH | | | | |
|---|---|---|---|---|---|---|
| Antibody | SEQ ID | SEQ ID | VL | VH | Titer | K_{D} |
| Name | NO: | NO: | % Humanness | % Humanness | (mg/mL) | (pM) |
| A1 | 103 | 108 | 83.838 | 84.694 | 43.7 | 290 |
| A2 | 103 | 118 | 83.838 | 84.694 | 46.6 | 250 |
| A3 | 103 | 128 | 83.838 | 84.694 | 45.7 | 230 |
| A4 | 103 | 138 | 83.838 | 84.694 | 50.3 | 220 |
| A5 | 103 | 148 | 83.838 | 86.735 | 49.4 | 320 |
| A6 | 103 | 158 | 83.838 | 85.714 | 32.9 | 320 |
| A7 | 103 | 168 | 83.838 | 85.714 | 52.8 | 330 |
| A8 | 103 | 178 | 83.838 | 85.714 | 60.2 | 340 |
| A9 | 113 | 108 | 82.828 | 84.694 | 63.5 | 210 |
| A10 | 113 | 118 | 82.828 | 84.694 | 80.3 | 190 |
| A11 | 113 | 128 | 82.828 | 84.694 | 69.9 | 170 |
| A12 | 113 | 138 | 82.828 | 84.694 | 80.2 | 170 |
| A13 | 113 | 148 | 82.828 | 86.735 | 70.1 | 250 |
| A14 | 113 | 158 | 82.828 | 85.714 | 49.7 | 220 |
| A15 | 113 | 168 | 82.828 | 85.714 | 74.9 | 260 |
| A16 | 113 | 178 | 82.828 | 85.714 | 83.8 | 240 |
| A17 | 123 | 108 | 84.848 | 84.694 | 58.5 | 200 |
| A18 | 123 | 118 | 84.848 | 84.694 | 73.6 | 190 |
| A19 | 123 | 128 | 84.848 | 84.694 | 67.3 | 170 |
| A20 | 123 | 138 | 84.848 | 84.694 | 75.8 | 180 |
| A21 | 123 | 148 | 84.848 | 86.735 | 68.8 | 250 |
| A22 | 123 | 158 | 84.848 | 85.714 | 45.4 | 210 |
| A23 | 123 | 168 | 84.848 | 85.714 | 70.6 | 230 |
| A24 | 123 | 178 | 84.848 | 85.714 | 80.4 | 230 |
| A25 | 133 | 108 | 83.838 | 84.694 | 64 | 150 |
| A26 | 133 | 118 | 83.838 | 84.694 | Too Low | No capture |
| A27 | 133 | 128 | 83.838 | 84.694 | Too Low | No capture |
| A28 | 133 | 138 | 83.838 | 84.694 | Too Low | No capture |
| A29 | 133 | 148 | 83.838 | 86.735 | 1.26 | No capture |
| A30 | 133 | 158 | 83.838 | 85.714 | 0.7835 | No capture |
| A31 | 133 | 168 | 83.838 | 85.714 | 0.6562 | No capture |
| A32 | 133 | 178 | 83.838 | 85.714 | Too Low | No capture |
| A33 | 143 | 108 | 82.828 | 84.694 | 4.66 | No binding |
| A34 | 143 | 118 | 82.828 | 84.694 | 6.26 | No binding |
| A35 | 143 | 128 | 82.828 | 84.694 | 2.5 | No binding |
| A36 | 143 | 138 | 82.828 | 84.694 | 4.98 | No binding |
| A37 | 143 | 148 | 82.828 | 86.735 | 2.88 | No binding |
| A38 | 143 | 158 | 82.828 | 85.714 | 1.57 | No capture |
| A39 | 143 | 168 | 82.828 | 85.714 | 0.2674 | No capture |
| A40 | 143 | 178 | 82.828 | 85.714 | 2.59 | No capture |
| A41 | 153 | 108 | 81.818 | 84.694 | 37 | 190 |
| A42 | 153 | 118 | 81.818 | 84.694 | 43.9 | 150 |
| A43 | 153 | 128 | 81.818 | 84.694 | 42.6 | 150 |
| A44 | 153 | 138 | 81.818 | 84.694 | 45.8 | 160 |
| A45 | 153 | 148 | 81.818 | 86.735 | 11.4 | No binding |
| A46 | 153 | 158 | 81.818 | 85.714 | 28 | 200 |
| A47 | 153 | 168 | 81.818 | 85.714 | 7.1 | No binding |
| A48 | 153 | 178 | 81.818 | 85.714 | 46.4 | 230 |
| A49 | 163 | 108 | 80.808 | 84.694 | 16.7 | 300 |
| A50 | 163 | 118 | 80.808 | 84.694 | 20.7 | 260 |
| A51 | 163 | 128 | 80.808 | 84.694 | 21.3 | 250 |
| A52 | 163 | 138 | 80.808 | 84.694 | 22.5 | 270 |
| A53 | 163 | 148 | 80.808 | 86.735 | 21.9 | 360 |
| A54 | 163 | 158 | 80.808 | 85.714 | 14.8 | 310 |
| A55 | 163 | 168 | 80.808 | 85.714 | 23.1 | 350 |
| A56 | 163 | 178 | 80.808 | 85.714 | 19.4 | 310 |
| A57 | 173 | 108 | 80.808 | 84.694 | 51.7 | 350 |
| A58 | 173 | 118 | 80.808 | 84.694 | 57.9 | 300 |
| A59 | 173 | 128 | 80.808 | 84.694 | 61.3 | 270 |
| A60 | 173 | 138 | 80.808 | 84.694 | 66.1 | 300 |
| A61 | 173 | 148 | 80.808 | 86.735 | 61.2 | 350 |
| A62 | 173 | 158 | 80.808 | 85.714 | 36.9 | 350 |
| A63 | 173 | 168 | 80.808 | 85.714 | 63.1 | 340 |
| A64 | 173 | 178 | 80.808 | 85.714 | 78.3 | 320 |
| A65 | 183 | 108 | 80.808 | 84.694 | 42.7 | 270 |
| A66 | 183 | 118 | 80.808 | 84.694 | 56 | 250 |
| A67 | 183 | 128 | 80.808 | 84.694 | 53.9 | 240 |
| A68 | 183 | 138 | 80.808 | 84.694 | 62.5 | 150 |
| A69 | 183 | 148 | 80.808 | 86.735 | 56.2 | 280 |
| A70 | 183 | 158 | 80.808 | 85.714 | 36.1 | 270 |
| A71 | 183 | 168 | 80.808 | 85.714 | 55.5 | 280 |
| A72 | 183 | 178 | 80.808 | 85.714 | 73.6 | 270 |
| A73 | 193 | 108 | 81.818 | 84.694 | 73.7 | 580 |
| A74 | 193 | 118 | 81.818 | 84.694 | 95 | 200 |
| A75 | 193 | 128 | 81.818 | 84.694 | 83.3 | 170 |
| A76 | 193 | 138 | 81.818 | 84.694 | 103.9 | 180 |
| A77 | 193 | 148 | 81.818 | 86.735 | 80.9 | 230 |
| A78 | 193 | 158 | 81.818 | 85.714 | 57.1 | 230 |
| A79 | 193 | 168 | 81.818 | 85.714 | 87.7 | 160 |
| A80 | 193 | 178 | 81.818 | 85.714 | 98.8 | 250 |
| A81 | 203 | 108 | 80.808 | 84.694 | 25.1 | 290 |
| A82 | 203 | 118 | 80.808 | 84.694 | 29.1 | 360 |
| A83 | 203 | 128 | 80.808 | 84.694 | 30 | 260 |
| A84 | 203 | 138 | 80.808 | 84.694 | 32.4 | 290 |
| A85 | 203 | 148 | 80.808 | 86.735 | 31 | 280 |
| A86 | 203 | 158 | 80.808 | 85.714 | 0.2403 | No capture |
| A87 | 203 | 168 | 80.808 | 85.714 | 31 | 340 |
| A88 | 203 | 178 | 80.808 | 85.714 | 38.7 | 320 |
| A89 | 213 | 108 | 83.838 | 84.694 | 38.1 | 160 |
| A90 | 213 | 118 | 83.838 | 84.694 | 49.5 | 170 |
| A91 | 213 | 128 | 83.838 | 84.694 | 44.6 | 130 |
| A92 | 213 | 138 | 83.838 | 84.694 | 48.3 | 160 |
| A93 | 213 | 148 | 83.838 | 86.735 | 45.4 | 180 |
| A94 | 213 | 158 | 83.838 | 85.714 | 28.8 | 190 |
| A95 | 213 | 168 | 83.838 | 85.714 | 44.2 | 190 |
| A96 | 213 | 178 | 83.838 | 85.714 | 55.2 | 190 |
| A97 | 223 | 108 | 82.828 | 84.694 | 70.7 | 140 |
| A98 | 223 | 118 | 82.828 | 84.694 | 83.2 | 120 |
| A99 | 223 | 128 | 82.828 | 84.694 | 79.7 | 110 |
| A100 | 223 | 138 | 82.828 | 84.694 | 83.1 | 130 |
| A101 | 223 | 148 | 82.828 | 86.735 | 78.3 | 170 |
| A102 | 223 | 158 | 82.828 | 85.714 | 58.4 | 150 |
| A103 | 223 | 168 | 82.828 | 85.714 | 86.9 | 150 |
| A104 | 223 | 178 | 82.828 | 85.714 | 88.1 | 170 |
| A105 | 233 | 108 | 84 | 84.694 | 98.3 | 420 |
| A106 | 233 | 118 | 84 | 84.694 | 60.7 | 430 |
| A107 | 233 | 128 | 84 | 84.694 | 107.9 | 370 |
| A108 | 233 | 138 | 84 | 84.694 | 116.2 | 420 |
| A109 | 233 | 148 | 84 | 86.735 | 106.3 | 440 |
| A110 | 233 | 158 | 84 | 85.714 | 81.5 | 450 |
| A111 | 233 | 168 | 84 | 85.714 | 107.4 | 510 |
| A112 | 233 | 178 | 84 | 85.714 | 108.8 | 480 |
| A113 | 243 | 108 | 84 | 84.694 | 78.3 | 760 |
| A114 | 243 | 118 | 84 | 84.694 | 89.2 | 750 |
| A115 | 243 | 128 | 84 | 84.694 | 94.2 | 660 |
| A116 | 243 | 138 | 84 | 84.694 | 91.3 | 690 |
| A117 | 243 | 148 | 84 | 86.735 | 89.3 | 810 |
| A118 | 243 | 158 | 84 | 85.714 | 65.4 | 810 |
| A119 | 243 | 168 | 84 | 85.714 | 91.1 | 910 |
| A120 | 243 | 178 | 84 | 85.714 | 97 | 840 |
| A121 | 253 | 108 | 82.828 | 84.694 | 74.3 | 240 |
| A122 | 253 | 118 | 82.828 | 84.694 | 73.7 | 230 |
| A123 | 253 | 128 | 82.828 | 84.694 | 81.3 | 210 |
| A124 | 253 | 138 | 82.828 | 84.694 | 83.9 | 250 |
| A125 | 253 | 148 | 82.828 | 86.735 | 87.4 | 260 |
| A126 | 253 | 158 | 82.828 | 85.714 | 58.2 | 270 |
| A127 | 253 | 168 | 82.828 | 85.714 | 81.9 | 280 |
| A128 | 253 | 178 | 82.828 | 85.714 | 101.7 | 270 |

Antibody A97 was selected for further characterization. Unless indicated otherwise, antibodies used in the following examples are full length antibodies, e.g., full length Antibody A97 having the heavy chain of SEQ ID NO: 109 and the light chain of SEQ ID NO: 224.

### Example 3. Effects of Anti-CTGF Antibodies on Cleavage of CTGF

This example tests the ability of anti-CTGF antibodies to prevent the cleavage of CTGF by MMP7 in vitro.

### Methods

Samples are prepared in Diluent A (Quanterix) by separately mixing each anti-CTGF antibody (Antibody A97 or Antibody F1, having the light chain of SEQ ID NO: 20 and the heavy chain of SEQ ID NO: 21) and isotype human IgG control with full length human CTGF (R&D Systems 9190-CC) in microcentrifuge tubes with or without the addition of MMP7 (R&D Systems 907-MP-010 and Millipore 444270100UG, 100 to 1000:1 MMP7 to anti-CTGF molar ratios). Full length CTGF only control samples are prepared with or without MMP7 for signal normalization and assay verification. Samples are incubated for 2 hours at 37 °C to activate MMP7, which cleaves full length CTGF at the hinge region connecting N-terminal and C-terminal domains. After two hours, samples are analyzed (neat) on a sandwich immunoassay (Homebrew methods for Planar Array SP-X by Quantertix, 100-0461), utilizing 1 μg/ml N-terminal capture (rat clone ZX-9B6 anti-human CTGF monoclonal antibody, conjugated to Quanterix antibody Homebrew tag) and 1 μg/ml C-terminal antibody detection (R&D systems anti-human CTGF mAb9190 or AF660, biotinylated following Quanterix protocol) and utilizing all consumables, reagents and assay protocols according to the manufacturer (Quanterix SP-X Homebrew methods and kit 100-0461). After reading the plate on SP-X Imager at short (20s) and long (300s) exposures, raw signals are compared (IV units; dual imaging chemiluminescent signal with high resolution CCD camera) and normalized to the signal of full length (intact) CTGF without MMPs as baseline. As the assay methods are stepwise, utilizing an N-terminal specific CTGF capture (binds to non-competing epitope to both Antibody 97 and Antibody F1) with a C-terminal specific CTGF detection can only detect intact CTGF, as any cleaved fragment alone either will not bind during capture step (washed away) or will not produce signal during detection step.

### Results

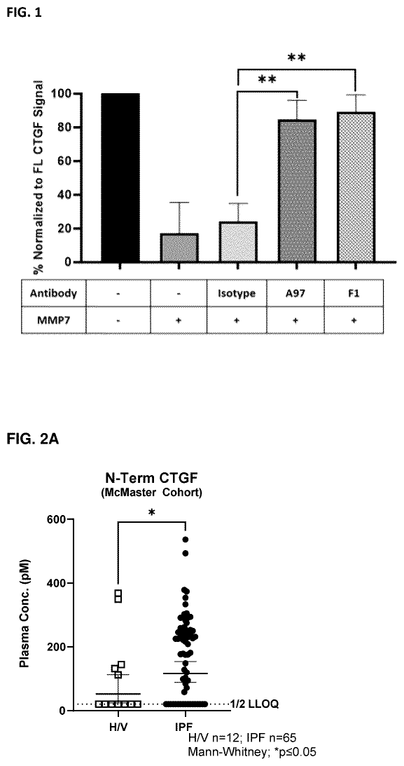

As shown in FIG. 1, the addition of Antibody 97 (p<0.005) or Antibody F1 (p<0.01) significantly protected in vitro MMP7-mediated cleavage when compared to isotype control incubated with MMP7 (averaged multiple runs and MMP7 concentrations) with normalized signal approaching full length CTGF normalized signal control without addition of MMP7.

### Example 4. N-Terminal and Full Length CTGF Measurement in Patient Samples

To assess the concentration of N-Terminal and full length CTGF in patient plasma samples, antibodies that bind to the N- and C-Terminal regions of CTGF are labeled with Homebrew and Biotin tags according to the manufacturer's protocol (Simoa Planar Array Homebrew, 100-0461, Quanterix). The N-Terminal CTGF Assay uses a rat clone ZX-9B6 anti-Human CTGF monoclonal antibody with Homebrew antibody tag for the capture and Antibody 97 labeled with Biotin for the detection. The full length CTGF assay uses Millipore pAb (ABS1628) to CTGF C-Terminus labeled with Homebrew antibody tag for capture and Antibody F1 labeled with Biotin for detection. The immunoassay procedure is followed according to the manufacturer's protocol (Simoa Planar Array Homebrew, 100-0461, Quanterix). Samples and standards are prepared in the kit-provided sample diluent A, with the full length standard (Recombinant Human CTGF, 9190-CC, R&D Systems) starting at 138 ng/ml and serially diluted 1:4. The N-terminal standard is an N-terminal fragment consisting of the first two domains of CTGF through the hinge region, starting at 200 ng/ml and serially diluted 1:4. Samples are diluted 1:4 for the N-terminal assay and used without dilution for the full length assay. Samples are read on the SP-X imager, optimizing the exposure times to the top standard concentration on the respective plates and reported as picomolar (pM) concentrations.

Total MMP7 levels are assessed with in patient plasma samples by immuno-assay (U-Plex Human MMP7, K151AHEK, Meso Scale Discovery) following the manufacturer's protocol. Samples are diluted 1:4 and results are reported as ng/ml.

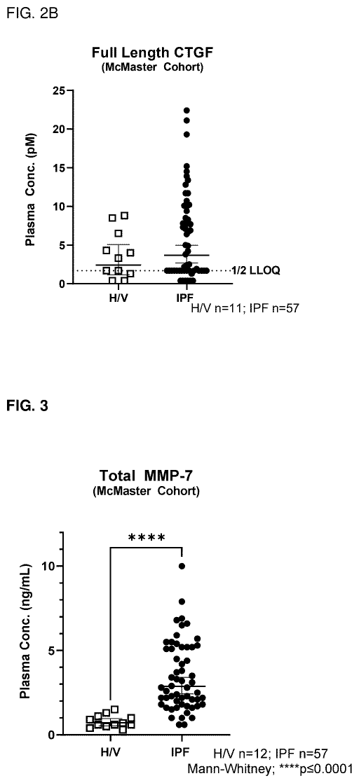

Results are shown for measurement of N-terminal CTGF (FIG. 2A), full length CTGF (FIG. 2B) and MMP7 (FIG. 3) in healthy volunteers (“H/V”) and IPF patients (“IPF”). N-terminal CTGF concentration was significantly elevated in IPF patients (p≤0.05). Full length CTGF concentration was not significantly different between IPF patients and healthy volunteers. MMP7 concentration was significantly elevated in IPF patients (p≤0.0001).

### Example 5. Alpha Smooth Muscle Actin Effects in Patient-Derived Multicellular Systems

To test the efficacy of a blocking anti-CTGF antibody in reducing fibrotic progression, primary progenitor basal cells isolated from IPF patient lungs are cultured for four weeks under air-liquid interphase (ALI) transwell condition to fully differentiated small airway epithelium. The differentiated lung epithelium is then cocultured with primary human fibroblasts isolated from IPF patients in the basolateral compartment of the transwell culture.

During a 72 hour stimulation with an IPF cytokine cocktail (IPF-RC, Schruf et al., FASEB J. 2020 June; 34(6):7825-7846), the upper epithelium layer starts to produce and secrete metalloproteases (MMPs, including MMP7) and CTGF. The activated fibroblasts underneath further produce large amounts of CTGF which then are cleaved by MMP7 into its active fragments (N-term and C-term fragments) which induce pro-fibrotic stimulation and transition of fibroblasts into myofibroblast cells (FMT). The fibrotic transitioning phase can be quantified by measuring alpha smooth muscle actin (a-SMA) in fibroblast lysates or by measuring the release of matrix modulating collagen components (e.g., COL1A1) in the coculture supernatant. A treatment of the IPF coculture with an anti-CTGF antibody after a 24 hour IPF cocktail pre-stimulation time was tested for its ability reduce fibrotic progression.

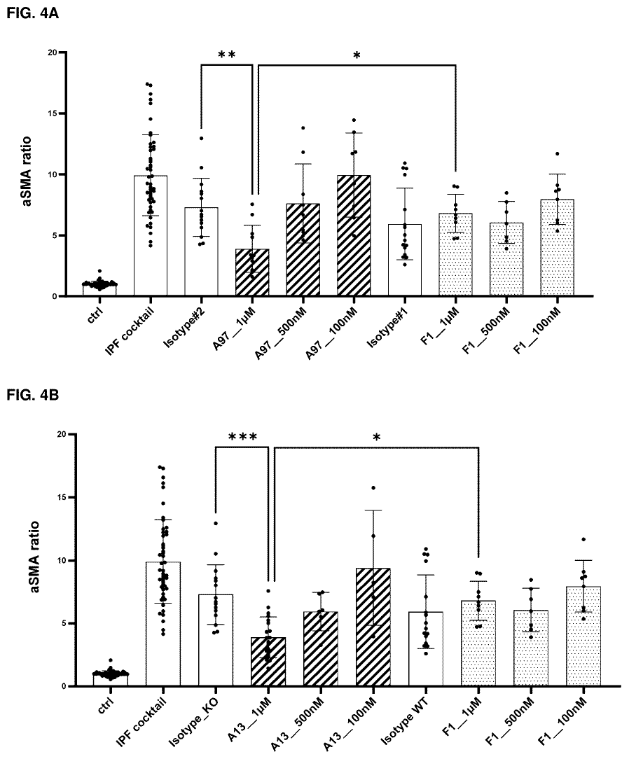

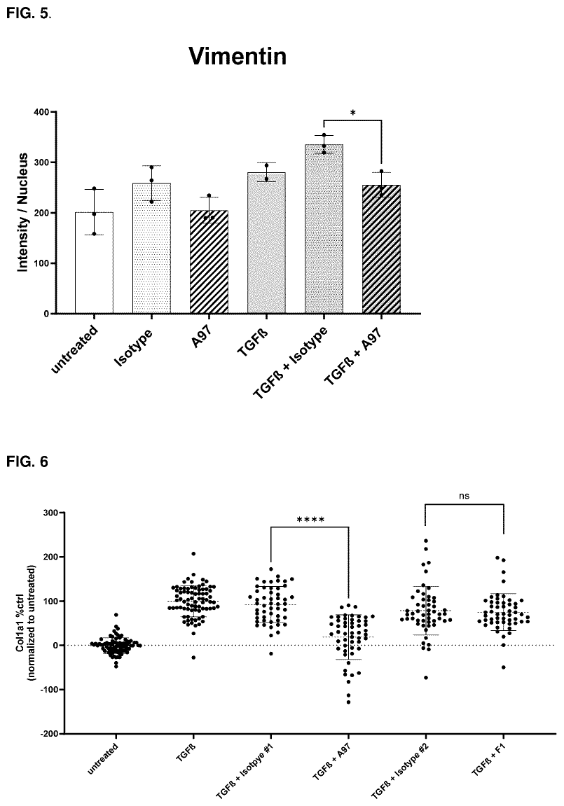

The IPF cocktail treatment induced a 10×-fold increase of a-SMA expression in fibroblasts, which represents the profibrotic FMT transition (FIG. 4A and FIG. 4B). The anti-CTGF antibody A97 (FIG. 4A) and antibody A13 (FIG. 4B) each significantly reduced the a-SMA expression in a dose dependent manner, while isotype controls and antibody F1 were not able to lower the a-SMA expression. Thus, anti-CTGF antibody A97 and A13 reduced fibrotic signaling and fibrotic progression in a patient-derived multicellular system.

### Example 6. Blockade of CTGF-Induced EMT in Primary Alveolar Cells

CTGF has been proposed to induce epithelial-to-mesenchymal transition (EMT) in alveolar stem cells, which contributes to the blockage of human alveolar type I epithelial cells (AT1) differentiation observed in disease. Alveolar cells undergo epithelial-mesenchymal transition in pulmonary fibrosis leading to a failure of the progenitor cells to repair alveoli and increased signal to the fibroblasts to produce matrix. CTGF has been involved in this process. As AT1 stem from human alveolar type II epithelial cells (AT2) and build the gas-exchange surface, this is an important hallmark of the pathogenesis in pulmonary fibrosis, leading to insufficient oxygenation.

In order to address this experimentally, we set up the isolation of the alveolar cells from explanted lungs, and assayed the ability of antibody A97 to block induction of EMT.

Primary human AT2 are seeded in 2D culture plates, then starved in minimal medium overnight before treatment for 24 hours or 72 hours with TGFβ1 to induce a pro-fibrotic epithelial to mesenchymal cell transition (EMT). Antibody A97, antibody F1, or isotype control antibody (dose range 0.1-1 pM) is added simultaneously with TGFβ1. Blocking CTGF cleavage and/or inhibiting the N-terminal CTGF fragment effects were hypothesized to inhibit/reduce the TGFβ1 induced EMT effect and help to stabilize the alveolar epithelium to maintain its biological function (mainly differentiation into AT1). The progression of alveolar to mesenchymal cell transition is measured via intracellular Vimentin staining (cytoskeletal modulation during EMT), and by collagen release quantification measured by ELISA (COL1A1 in AT2 cell lysates and supernatant) ELISA.

Treatment with Antibody A97 reduced Vimentin upregulation 72 hours after TGFβ1 stimulation (FIG. 5) and reduced the level of collagen 1A1 to unstimulated control levels 24 hours after TGFβ1 treatment (FIG. 6). Treatment with Antibody F1 did not significantly reduce the level of collagen 1A1 (FIG. 6). These results support the use of antibody A97 to promote the maintenance and functionality of the alveolar epithelial stem cells.

### Example 7. Proteomic Analysis

### Introduction

CTGF is a secreted growth factor that modulates many profibrotic processes and is strongly up-regulated in idiopathic pulmonary fibrosis (IPF). It is composed of two N-terminal domains (together NTF) and two C-terminal domains (together CTF) bound by a hinge peptide containing cleavage sequences for matrix metalloproteases (MMP), especially MMP7. The cleavage of CTGF by MMP7 activates the pathway since the fragments are biologically active. Anti-CTGF antibody A97 binds CTGF and inhibits its cleavage (see Example 3 above) and thus reduces the occurrence of fragments, and also binds the N-terminal fragment, thus potentially reducing fibrogenesis and fibrillar collagen production.

This example utilizes a patient-derived system containing the identified fibrotic niche. We isolate primary small airway epithelial cells from IPF donors as well as pulmonary fibroblasts. The small airways are then raised at the air-liquid-interface for five weeks in an organotypic culture. After priming with TGFb, a pro-fibrotic factor upstream of CTGF, the small airways and the fibroblast are put in cocultures and treated with the IPF cocktail (Schruf et al, 2020). This results in the induction of CTGF and MMP7, resulting in fibrogenesis. In this experimental setup, we test the modulation of fibrosis protein biomarkers in cells treated with the IPF cocktail by anti-CTGF antibodies A13, A97 or F1.

Co-culture supernatant proteomes are analyzed by liquid chromatography coupled to mass spectrometry (LC-MS) to quantify protein expression values that are modulated by the tested antibodies. Quantification includes the fibrosis PoCP biomarker Pro-Collagen III (ProC3), which, among other proteins, represents the production of the fibrillar collagen III, a major constituent of fibrotic extracellular matrix and tissue stiffening biomarker. We furthermore quantify additional proteins that are related to fibrosis and tested their relation to CTGF expression values.

### Methods

Sample preparation is performed in a 96-well format (twin.tec PCR Plate 96 LoBind, Eppendorf) following the in-StageTip protocol (Kulak et. A I; doi: 10.1038/nmeth.2834) (PreOmics IST-Kit catalogue number P.O.00027). 100 μl of each cell culture supernatant is dried (Concentrator plus, Eppendorf). 50 μl of PreOmics lysis buffer is added per well followed by a boiling step at 95 °C, 1200 rpm (ThermoMixer C, Eppendorf) for 20 min to solubilize proteins, reduce disulfide bridges, and alkylate free cysteines. After a 5 min cool down of the samples, the PreOmics digestion mix is added in the recommended amount followed by overnight digestion at 37 °C, 1200 rpm (ThermoMixer C, Eppendorf). Digestion is stopped adding a 1:1 volume of PreOmics stopping buffer, followed by a sample clean-up utilizing the PreOmics IST protocol. After elution, clean peptides are dried (Concentrator plus, Eppendorf) and resuspended in 25 μl of PreOmics LC-buffer with a 30 min incubation step at 30 °C, 1200 rpm (ThermoMixer C, Eppendorf). The peptide concentration per well is quantified utilizing a colorimetric quantitative peptide assay (catalogue number 23275, Pierce, Quantitative Peptide Assay, Thermo Fisher Scientific).

Proteome analysis is performed utilizing the Vanquish Neo system (Thermo Fisher Scientific) coupled to an Orbitrap Eclipse mass spectrometer (Thermo Fisher Scientific) with a nano-electrospray source. 500 ng of each sample is injected and separated with a 15 cm Easy-Spray PepMap Neo column (75 μm ID, 150 mm length, PN ES75150PN, Thermo Fisher Scientific) heated to 40 °C and run at a 450nl/min flow rate. The chromatography gradient is set to 45 min total, separating peptides by hydrophobicity for 37 minutes starting with 1% Buffer B (80% ACN™, 19.9% ddH2O, 0.1% FA), 99% Buffer A (99.9% ddH2O, 0.1% FA) and ending with 35% Buffer B and 65% Buffer A. For column wash and regeneration, Buffer B is ramped up to 95% in 4 minutes and kept for 4 minutes at 95% Buffer B until the end of the gradient. Column regeneration is performed automatically before sample pickup for the next run. Electrospray voltage is set to 1900V in positive ion mode.

Mass spectrometry analysis is performed using a data independent acquisition (DIA) scan mode. One full scan event (MS1) is followed by 33 MS2 (MSMS) scans covering a 300-1650 m/z range. MS1 resolution is set to 120,000, AGC target to 2E6 with a maximum injection time of 60 ms. MS2 resolution is set to 15,000, AGC target to 2E6 with a maximum injection time of 22 ms.

Mass spectrometry raw data are processed within the Spectronaut software suite (v17.4, Biognosys AG, Schlieren, Switzerland) utilizing the directDIA search algorithm with standard settings. Searches are performed against the human UniProt reference proteome of canonical and isoform sequences (UP000005640_9606.fa, UP000005640_9606_additional.fa).

Data analysis is performed in the Perseus environment (Tyanova et al.; doi: 10.1038/nmeth.3901) and GraphPad Prism. COL3A1 quantification events are transferred into GraphPad Prism and visualized as column bar plot highlighting mean and standard deviation. Statistical analysis is performed using a one-way ANOVA for all groups against the positive control (IPF-cocktail treatment). For global protein correlation analysis, the data matrix is filtered for at least 50% protein quantification events across the experiment. Missing values are imputed from 1.8 standard deviations downshifted normal distribution with a spread of 0.3 standard deviations. The data matrix is transposed by genes, followed by protein profile expression correlation across samples by Pearson. The resulting protein profile correlation matrix is clustered hierarchically by column and row to identify co-regulated protein clusters. Zooming into the cluster containing the CTGF (CCN2 gene), proteins are identified whose expression correlates with CTGF protein levels.

### Results

Antibody A97 prevents CTGF activation by cleavage inhibition and thus reduces the occurrence of fragments (see Example 3) and hinders the N-terminal fragment binding to fibroblasts (see Example 9), thus reducing fibrogenesis and fibrillar collagen production.

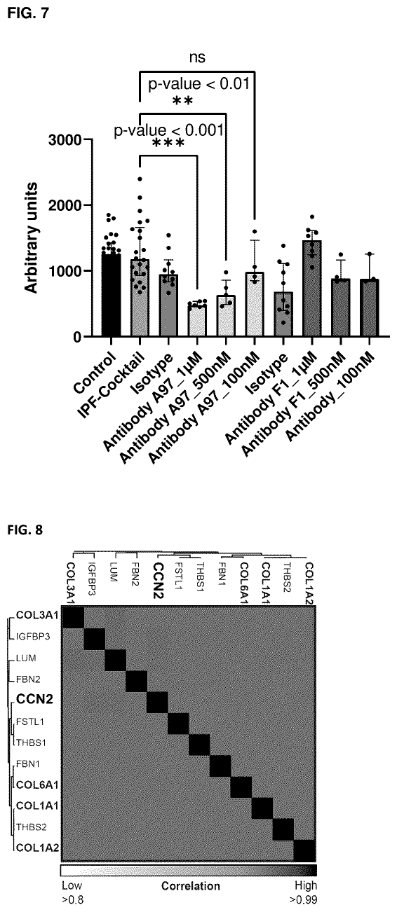

Procollagen 3 (ProC3) is a prognostic biomarker for disease progression in IPF and PPF. The results in this example demonstrate that A97 significantly modulated ProC3 levels in cells treated with the IPF cocktail (FIG. 7) (***p<0.001; **p<0.01). This effect appeared to be dose-dependent. By contrast, Antibody F1 showed no significant effect on Procollagen 3 production in cells treated with the IPF cocktail (FIG. 7).

Furthermore, the global correlation of protein expression profiles across experimental groups revealed that CTGF (Gene: CCN2) protein expression levels in the co-culture supernatant are strongly correlated to the production of other fibrillar collagens such as COL6A1, COL1A1 and COL1A2 besides the already presented COL3A1 (FIG. 8). In addition to collagens, additional potential biomarkers were modulated. All proteins highlighted in this cluster have a very high protein expression profile correlation with each other with >0.8 according to Pearson.

The results showed that CTGF expression levels were tightly correlated to fibrosis progression in this system, and antibody A97 was an effective inhibitor of collagen production, thereby modulating fibrosis progression.

Functional analysis of proteomic data revealed differences between the F1 and A97 antibodies in modulating collagen production using Hallmark protein annotations (Liberzon et al., The Molecular Signatures Database (MSigDB) hallmark gene set collection. Cell Syst. 2015 Dec. 23; 1(6):417-425). Results are shown in Table 16. The term “Odds Ratio” indicates the over-representation of the protein set. For example, an odds ratio of 2 means that the protein annotation is twice as frequent as expected by random chance. The term “FDR-adjusted p-value” represents the Bonferroni correction for multiple testing applied to the enrichment p-value of “hallmark” protein set annotations. It indicates the probability of observing the enrichment by chance after adjusting for multiple comparisons. The term “Combined Score” is the natural logarithm of the p-value multiplied by the z-score, where the z-score is the deviation from the expected rank, providing a comprehensive measure of the significance and effect size of the enrichment. The results show that proteins whose values were significantly higher in the F1-treated sample compared to the A97-treated sample were enriched in for the hallmark annotation ““Epithelial to mesenchymal transition”. Protein annotated as “Epithelial to mesenchymal” transition were not enriched for samples treated with A13 or A97, but were enriched for “IPF cocktail”. The results show that antibodies A13 and A97 were each able to prevent the increase in epithelial to mesenchymal transition gene expression induced by the IPF cocktail. By contrast, antibody F1 was not able to prevent the statistically significant increase in epithelial to mesenchymal transition gene expression.

TABLE 16
Functional enrichment analysis for the Hallmark term:
“Epithelial mesenchymal transition”.

| | Odds | | Combined |
|---|---|---|---|
| | ratio | FDR p-value | score |
| IPF cocktail | 11.53 | 2.35E−16 | 456.18 |
| F1 1 uM | 11.65 | 2.54E−09 | 270.14 |
| A13 1 uM | 4.91 | 0.10 | 20.12 |
| Ctrl | 1.80 | 1.00 | 5.06 |
| A97 1 uM | 1.12 | 0.89 | 0.48 |

### Example 8. Free CTGF Measurement

The purpose of this bioanalysis was to determine target engagement of the antibodies A13, A97 and F1 from a small airway epithelial cell (SAEC) co-culture study to correlate functional effects with target binding. For this, CTGF is quantified using a competitive ligand binding assay approach. Full target engagement by an antibody under the applied assay conditions (“conditional target engagement”) is expected to result in no quantifiable free CTGF. Lower target engagement will result in the detection of free CTGF levels.

Samples from cell co-culture experiments described in Example 5 are used for measurement of human CTGF levels. Cell co-culture samples are taken and concentrations of human CTGF are determined on the Gyrolab immunoassay platform using an assay targeting two different epitopes of the protein. As shown in Example 12, antibodies A97 and A13 do not compete with F1 for binding to CTGF. Using Antibody F1 and Antibody A97 as capturing and detection reagents, respectively, allows discrimination between free CTGF and CTGF engaged by one or the other antibody at the applied assay conditions (“conditional target engagement”). In this assay, CTGF is only detected if not bound by either A97, A13 or F1. Therefore, reduced levels in detected free CTGF are a measure for target engagement by the antibodies in these samples.

Human CTGF concentrations are determined using a semi-automated ligand binding assay (LBA) platform (Gyros Protein Technologies AB, xPand). For the measurement, the samples are measured as 1:100 dilution in Rexxip AN buffer (Gyros Protein Technologies AB, #P0004994). The analyte, human CTGF, is immobilized on streptavidin-coated Gyrolab Bioaffy 1000 CDs (Gyros Protein Technologies AB, #P0004253) using a biotin-tagged (Thermo Scientific, #21335) derivative of Antibody F1 (100 μg/ml in PBS/0.01% Tween 20) and is detected by recording the fluorescence of a fluorophore-labeled (Thermo Scientific, #A20006) derivative of Antibody A97 used at 20 nM in Rexxip F buffer (Gyros Protein Technologies AB, #P0004825). Biotinylation and fluorophore-conjugation of the unlabeled antibodies is performed for 1 hour at room temperature after mixing 1.67 UM antibody, 16.7 UM label reagent, and 0.1 M NaHCO₃, all dissolved in PBS.

For the absolute quantification of the analyte, calibration curves are generated in 1% matrix in Rexxip AN buffer. From these curves a lower limit of quantification (LLoQ) is derived for each individual CD by multiplying the concentration at three-fold blank response (limit of detection) with the applied dilution factor (100), resulting in a value of 0.32 nM. Response signals achieved from unknown samples are matched against the calibration curves to derive the corresponding analyte concentration.

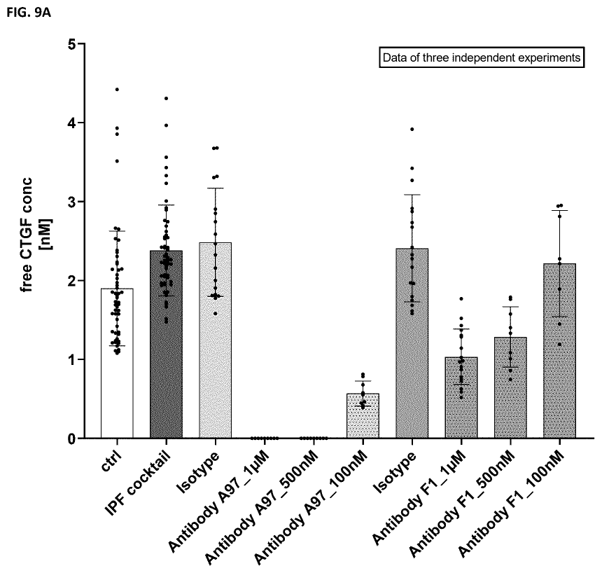

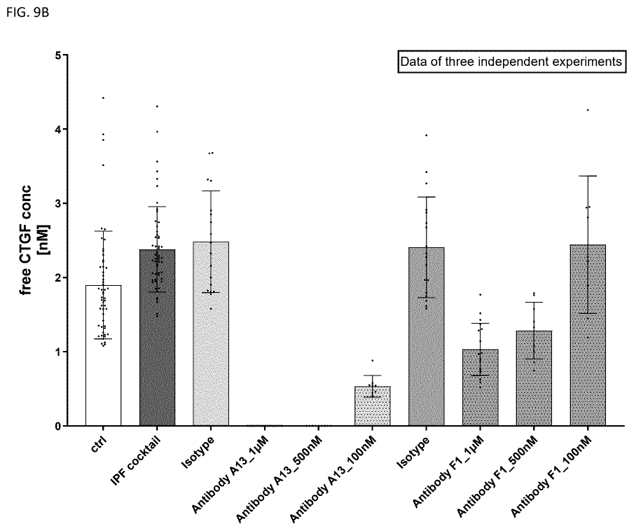

The quantification of free CTGF in the samples recovered from the IPF SAEC ALI coculture assay showed increased levels of CTGF upon treatment with IPF (“IPF cocktail”) with or without isotype control antibodies (“isotype”) compared with non-treated controls (“ctrl”) (FIG. 9A and FIG. 9B). Results are shown from three independent experiments. For the isotype controls (“isotype”), similar levels of free CTGF were determined as for the respective control sample (“IPF cocktail”).

For the anti-CTGF antibodies A13, A97 and F1, a dose-dependent decrease in free CTGF levels was observed, with no detectable levels for 1 UM and 500 nM with both A13 and A97. Comparing the determined concentrations of free CTGF between anti-CTGF antibodies A13, A97 and F1 at equal concentrations, we conclude that A13 and A97 engaged CTGF more effectively than F1, with F1 being unable to fully engage CTGF (i.e., free CTGF was detected) even at the highest antibody concentration tested.

### Example 9. Cell Binding Assay

CTGF and fragments thereof have been proposed to signal through multiple receptors, however, over 200 receptors have been proposed and little evidence on the actual interaction has been shown. We aimed to assess whether an antibody against CTGF could abrogate binding to the cell surface of multiple cell types in order to understand the ability of the antibody to reduce CTGF signaling. For that a binding assay was developed and applied to several cell types, specifically, alveolar type II cells, fibroblasts and A549 cells.

To assess the impact of the CTGF antibodies on the binding of the CTGF to the cell membrane, recombinant CTGF is labeled with R-Phycoerythrin (RPE) according to the manufacturer's protocol (LYNX Rapid RPE Antibody Conjugation Kit™ (R), LNK024RPE, Bio-Rad) and pre-incubated with either Isotype control (“Iso Ctrl”) or anti-CTGF antibodies A97, F1, H1, or H2, or compound P1 (30 nM CTGF labeled protein and 1 pM of respective antibody or compound) for 1 hour (A549 cells and primary human fibroblasts (phFb)) or 4 hours (primary human alveolar type II cells (phAT2)). Cells are seeded 24 hours prior to the experiment, in 96 well plate: 8000 phFb cells/well in Dulbecco's Modified Eagle Medium (DMEM) containing GlutaMAX Supplement™ (Thermo Fisher Scientific), with the addition of 10% fetal bovine serum (FCS), 1% Penicillin/Streptomycin (P/S) and 1% non-essential amino acids; 10000 AT2 cells/well in basal media (Stemcell #100-0848) with addition of 10× supplements (Stemcell #100-0849) and Antibiotic-Antimycotic (Gibco #15240062); 10000 A549 cells/well in DMEM supplemented with 10% FCS and 1% PS. The prepared mixture of RPE-labeled CTGF and antibodies is added to the cell cultures and incubated for the indicated time at 37 °C. Afterwards, cells are washed 3 times with PBS and the fresh media is added. Finally, cells are scanned with IncuCyte and the RPE signal, indicating CTGF binding to the cell membrane, is measured. Values are normalized to the cell density (brightfield image) and shown relatively to control (media with labeled CTGF).

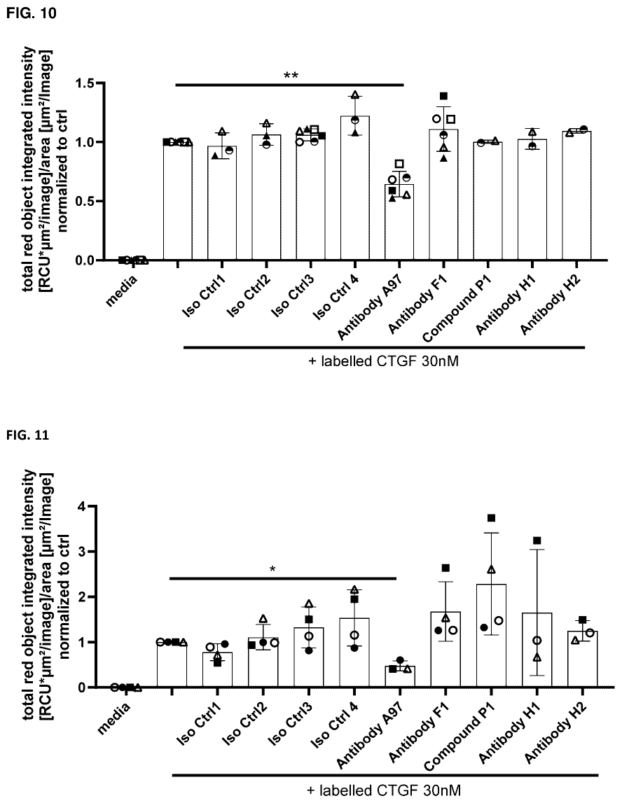

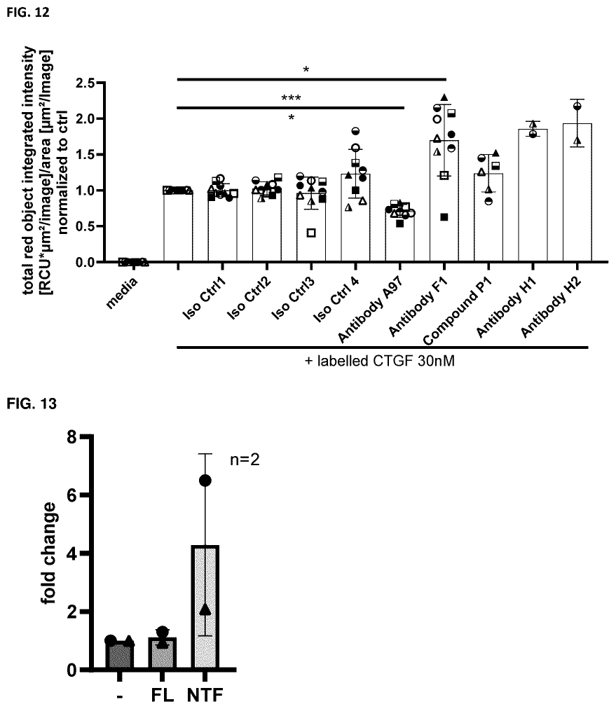

Impact of the anti-CTGF antibodies on the binding of the CTGF to the cell membrane was determined for primary human lung fibroblasts (phFb; FIG. 10; **p≤0.01), primary alveolar epithelial type II cells (ATII; FIG. 11; *p≤0.05) and A549 lung epithelial cells (FIG. 12; *p≤0.05, **p≤0.0001). These results demonstrate that the A97 antibody could prevent binding of CTGF to the cell membrane in all tested cells, while antibodies F1, H1, H2, and compound P1 could not prevent binding of CTGF to the cell membrane in any tested cell.

### Example 10. Macrophage Efferocytosis Effects of CTGF

This example shows that contacting homeostatic human macrophages (both iPSC-derived and blood-derived) with CTGF N-terminal fragment inhibited their ability to efferocytose dead cell bodies. This is accompanied by a raise in QPCT transcripts, a gene involved in efferocytosis suppression (Schloesser et al, 2022). The observed reduction in efferocytosis potential of macrophages may contribute to fibrotic disease progression, for example by allowing dead or senescent cells to accumulate and perpetuate profibrotic signaling.

### Methods

Human induced pluripotent stem cell maintenance is done according to the protocol in Bitzer et al. (SLAS Discov. 2023; 28(4):149-62). In short, line 201B7 are cultivated in a 6 well format. Cells are split once per week, and twice per week a medium exchange is performed.

Differentiation of hiPSCs into macrophages is done according to the protocol in Bitzer et al. (id.). In short, cells are plated into T175 flask and are treated as in the maintenance for 1 week. In the second week differentiation is performed by adding the specified media and cytokines. From weeks 3 to 6 progenitor floating cells are collected. Those CD34+ precursor cells are used to differentiate into macrophages.

Primary macrophage cell culture is done according to the protocol in Schloesser D, et al. (J Cell Biol. 2022; 222(2):e202207097). In short, PBMCs are isolated from whole blood using LeucoSep™ tubes and monocytes are isolated by negative selection for CD14+.

Monocytes and CD34+ precursor cells are differentiated for 7 days by adding 50 ng/ml MCSF to monocytes for the generation of monocyte-derived macrophages (MDM) and 100 ng/ml MCS to CD34+ precursor cells for the generation of iPSC-derived macrophages (IDM).

Macrophage activation is done according to the protocol in Schloesser et al. (id.). Stimulation for RNA is made in 12-well plates (500,000 cells/well) and for the efferocytosis assay in 96 well plate (15,000 cells/well). Homeostatic macrophage phenotype is induced by IL-4 (10 ng/ml) and IL-13 (10 ng/ml) for 16-18 hours. Full length CTGF or NTF (4 nM) is added at the same time.

Apoptotic corpse generation and labelling is done according to the protocol in Schloesser et al. (id.). In brief, apoptotic Raji cells are generated by UV exposure. Afterwards cells are labeled using the pHrodo@ dye from Sartorius and are added to macrophages in 96 well plate with a ratio of 1:3.3.

Efferocytosis Assay by IncuCtye S3 is done according to the protocol in Schloesser et al. (id.). In brief, 15,000 macrophages are plated per well and are stimulated overnight. After 16-18 hours, apoptotic Raji cells are added and the uptake is monitored for 24 hours.

RNA isolation and analysis is done according to the protocol in Schloesser et al. (id.). In brief, samples are lysed and RNA is isolated by using MagMax automated system. RNA is transcribed into cDNA and qPCR analysis is performed. Ct values or the fold change of untreated to stimulated are calculated (2\hat{ }(−ddCT)) (dCt normalized to a housekeeping gene, ddCt unstimulated control).

### Results

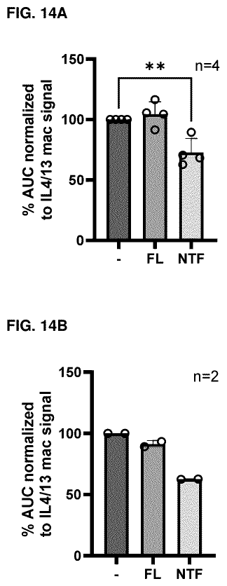

Full length (FL) CTGF didn't modify efferocytosis activity while CTGF N-terminal fragment leads to a significant reduction of efferocytotic capacity of homoestatic IL4/IL-13 activated macrophages, suggesting that abrogating cleavage may restore efferocytosis potential (FIG. 14A and FIG. 14B). CTGF N-terminal fragment (NTF) upregulated QPCT gene expression (FIG. 13). The findings were consistent in blood-derived macrophages and iPSC derived macrophages.

### Example 11. Binding Affinity of Anti-CTGF Antibodies to CTGF

Binding affinity of anti-CTGF antibodies H1 (having the light chain of SEQ ID NO: 22 and the heavy chain of SEQ ID NO: 23), H2 (having the light chain of SEQ ID NO: 24 and the heavy chain of SEQ ID NO: 25), F1, and A97 for full length human CTGF or human CTGF N-terminal fragment are determined by surface plasmon resonance (SPR).

Surface plasmon resonance is conducted using a Biacore 8K+ (Cytiva). Protein A/G (Thermo Scientific) is amine coupled to CM5 Sensor S Chip (Cytiva). For binding to hCTGF N-terminal fragment (NTF), anti-CTGF antibody (1 μg/ml) is captured on the Protein A/G surface, followed by various concentrations of antigen (100 nM to 0.78 nM; 2-fold dilutions). For binding to hCTGF full length, anti-CTGF antibody (0.2 μg/ml) is captured followed by various concentrations of antigen (10 nM to 0.156 nM; 2-fold dilutions).

Results are shown in Tables 17 and 18 below.

TABLE 17
Binding affinity of anti-CTGF antibodies H1, H2, F1,
and A97 for the human CTGF N-terminal fragment.

| mAb | kₐ (1/Ms) | k_{d} (1/s) | K_{D} (M) | K_{D} (nM) |
|---|---|---|---|---|
| H1 | 1.22E+06 | 1.07E−02 | 8.82E−09 | 8.8 |
| H2 | 3.83E+05 | 1.75E−02 | 4.57E−08 | 46 |
| A97 | 2.90E+05 | 4.00E−05 | 1.40E−10 | 0.14 |
| F1 | 3.45E+05 | 1.05E−02 | 3.03E−08 | 30 |

TABLE 18
Binding affinity of anti-CTGF antibodies H1,
H2, F1, and A97 for human full length CTGF.

| mAb | Kₐ (1/Ms) | K_{d} (1/s) | K_{D} (M) | K_{D} (PM) |
|---|---|---|---|---|
| H1 | 6.83E+06 | 6.71E−04 | 9.83E−11 | 98 |
| H2 | 2.70E+06 | 1.49E−03 | 5.54E−10 | 550 |
| A97 | 2.70E+06 | 4.80E−06 | 1.8E−12 | 1.8 |
| F1 | 1.37E+07 | 3.37E−03 | 2.47E−10 | 250 |

### Example 12. Antibody Competition for CTGF Binding

This example shows the results of testing whether antibodies A97, H1, H2, and F1 compete with one another for binding to hCTGF.

### Methods:

Surface plasmon resonance is conducted using a Biacore 8K+ (Cytiva). Protein A/G (Thermo Scientific) is amine coupled to CM5 Sensor S Chip (Cytiva). For each experiment, a first anti-CTGF antibody (100 nM) is captured on the Protein A/G, followed by blocking unused Protein A/G using an irrelevant IgG antibody (400 nM). Antigen (hCTGF N-terminal fragment (NTF) (100 nM) or hCTGF full length (20 nM)) and the second anti-CTGF antibody (100 nM) are injected in direct sequence using the dual command. Use of the “dual command” results in minimal to no intervening time between the end of antigen injection and start of mAb injection.

### Results and Discussion

Competitive binding results were obtained as shown in Table 18 and Table 19. Results were the same for full length hCTGF and hCTGF NTF. As expected, each anti-CTGF antibody competed with itself for binding to hCTGF NTF or full length hCTGF. Antibodies H1, H2, and F1 each competed with each other for binding to hCTGF NTF or to full length hCTGF. Antibody A97 did not compete with H1, H2 or F1 for binding to hCTGF NTF or to full length hCTGF.

The results indicate that the A97 does not compete with H1, H2, and F1 for binding to either hCTGF NTF or full length hCTGF.

TABLE 18
Anti-CTGF antibody competition for binding to hCTGF NTF. Each row
is labeled with the first antibody, and each column is labeled
with the second antibody. An “X” indicates that binding
competition was observed between the first antibody and the second
antibody, while an empty box indicates no competition was observed.

| | H1 | H2 | A97 | F1 |
|---|---|---|---|---|
| H1 | X | X | | X |
| H2 | X | X | | X |
| A97 | | | X | |
| F1 | X | X | | X |

TABLE 19
Anti-CTGF antibody competition for binding to full length
hCTGF. Each row is labeled with the first antibody, and
each column is labeled with the second antibody. An “X”
indicates that binding competition was observed between
the first antibody and the second antibody, while an empty
box indicates no competition was observed.

| | H1 | H2 | A97 | F1 |
|---|---|---|---|---|
| H1 | X | X | | X |
| H2 | X | X | | X |
| A97 | | | X | |
| F1 | X | X | | X |

1. A method of treating a CTGF-associated disorder, a fibrotic disease, hypertension, diabetes, myocardial infarction, arthritis, CTGF-related cell proliferative disease, atherosclerosis, glaucoma, or a cancer, comprising administering an effective amount of an anti-CTGF antibody or an antigen-binding fragment thereof to a patient in need thereof, wherein the anti-CTGF antibody or an antigen-binding fragment thereof comprises:
a light chain variable region comprising a light chain complementarity determining region 1 (L-CDR1) having the amino acid sequence of Y1-SSQSIVHYN-Y2-Y3-TYLE where Y1 is Kor R, Y2 is A, E, G, or T and Y3 is Kor N (SEQ ID NO: 10), a light chain complementarity determining region 2 (L-CDR2) having the amino acid sequence of KVS-Z1-R-Z2-S where Z1 is N or S and Z2 is A or F (SEQ ID NO: 11), and a light chain complementarity determining region 3 (L-CDR3) having the amino acid sequence of FQGSHFPLT (SEQ ID NO: 12); and
a heavy chain variable region comprising a heavy chain complementarity determining region 1 (H-CDR1) having the amino acid sequence of DYYMA (SEQ ID NO: 15), a heavy chain complementarity determining region 2 (H-CDR2) having the amino acid sequence of NINY-X1-GSRTDLL-X2-SLKS where X1 is D, E, or Y and X2 is A, D, or E (SEQ ID NO: 16), and a heavy chain complementarity determining region 3 (H-CDR3) having the amino acid sequence of DTSRGSYFDV (SEQ ID NO: 17).

2. The method according to claim 1, wherein the anti-CTGF antibody or an antigen-binding fragment thereof comprises:
i. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
ii. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
iii. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
iv. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
v. a light chain variable region comprising an L-CDR1 having the amino acid sequence of SEQ ID NO: 130, an L-CDR2 having the amino acid sequence of SEQ ID NO: 131, and an L-CDR3 having the amino acid sequence of SEQ ID NO: 132; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
vi. a light chain variable region comprising an L-CDR1 having the amino acid sequence of SEQ ID NO: 150, an L-CDR2 having the amino acid sequence of SEQ ID NO: 151, and an L-CDR3 having the amino acid sequence of SEQ ID NO: 152; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
vii. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
viii. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
ix. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
x. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
xi. a light chain variable region comprising an L-CDR1 having the amino acid sequence of SEQ ID NO: 200, an L-CDR2 having the amino acid sequence of SEQ ID NO: 201, and an L-CDR3 having the amino acid sequence of SEQ ID NO: 202; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
xii. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
xiii. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
xiv. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
xv. a light chain variable region comprising an L-CDR1 having the amino acid sequence of SEQ ID NO: 250, an L-CDR2 having the amino acid sequence of SEQ ID NO: 251, and an L-CDR3 having the amino acid sequence of SEQ ID NO: 252; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
xvi. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xvii. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xviii. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xix. a light chain variable region comprising an L-CDR1 having the amino acid sequence of SEQ ID NO: 150, an L-CDR2 having the amino acid sequence of SEQ ID NO: 151, and an L-CDR3 having the amino acid sequence of SEQ ID NO: 152; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xx. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxi. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxii. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxiii. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxiv. a light chain variable region comprising an L-CDR1 having the amino acid sequence of SEQ ID NO: 200, an L-CDR2 having the amino acid sequence of SEQ ID NO: 201, and an L-CDR3 having the amino acid sequence of SEQ ID NO: 202; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxv. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxvi. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxvii. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxviii. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxix. a light chain variable region comprising an L-CDR1 having the amino acid sequence of SEQ ID NO: 250, an L-CDR2 having the amino acid sequence of SEQ ID NO: 251, and an L-CDR3 having the amino acid sequence of SEQ ID NO: 252; and a heavy chain variable region comprising an H-CDR1 having the amino acid sequence of SEQ ID NO: 115, an H-CDR2 having the amino acid sequence of SEQ ID NO: 116, and an H-CDR3 having the amino acid sequence of SEQ ID NO: 117; or
xxx. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xxxi. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xxxii. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xxxiii. a light chain variable region comprising an L-CDR1 having the amino acid sequence of SEQ ID NO: 150, an L-CDR2 having the amino acid sequence of SEQ ID NO: 151, and an L-CDR3 having the amino acid sequence of SEQ ID NO: 152; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xxxiv. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xxxv. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xxxvi. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xxxvii. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xxxviii. a light chain variable region comprising an L-CDR1 having the amino acid sequence of SEQ ID NO: 200, an L-CDR2 having the amino acid sequence of SEQ ID NO: 201, and an L-CDR3 having the amino acid sequence of SEQ ID NO: 202; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xxxix. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xl. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xli. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xlii. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xliii. a light chain variable region comprising an L-CDR1 having the amino acid sequence of SEQ ID NO: 250, an L-CDR2 having the amino acid sequence of SEQ ID NO: 251, and an L-CDR3 having the amino acid sequence of SEQ ID NO: 252; and a heavy chain variable region comprising an H-CDR1 having the amino acid sequence of SEQ ID NO: 125, an H-CDR2 having the amino acid sequence of SEQ ID NO: 126, and an H-CDR3 having the amino acid sequence of SEQ ID NO: 127; or
xliv. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
xlv. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
xlvi. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
xlvii. a light chain variable region comprising an L-CDR1 having the amino acid sequence of SEQ ID NO: 150, an L-CDR2 having the amino acid sequence of SEQ ID NO: 151, and an L-CDR3 having the amino acid sequence of SEQ ID NO: 152; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
xlviii. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
xlix. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
l. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
li. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
lii. a light chain variable region comprising an L-CDR1 having the amino acid sequence of SEQ ID NO: 200, an L-CDR2 having the amino acid sequence of SEQ ID NO: 201, and an L-CDR3 having the amino acid sequence of SEQ ID NO: 202; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
liii. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
liv. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
lv. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
lvi. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
lvii. a light chain variable region comprising an L-CDR1 having the amino acid sequence of SEQ ID NO: 250, an L-CDR2 having the amino acid sequence of SEQ ID NO: 251, and an L-CDR3 having the amino acid sequence of SEQ ID NO: 252; and a heavy chain variable region comprising an H-CDR1 having the amino acid sequence of SEQ ID NO: 135, an H-CDR2 having the amino acid sequence of SEQ ID NO: 136, and an H-CDR3 having the amino acid sequence of SEQ ID NO: 137; or
lviii. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lix. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lx. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxi. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxii. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxiii. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxiv. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxv. a light chain variable region comprising an L-CDR1 having the amino acid sequence of SEQ ID NO: 200, an L-CDR2 having the amino acid sequence of SEQ ID NO: 201, and an L-CDR3 having the amino acid sequence of SEQ ID NO: 202; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxvi. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxvii. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxviii. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxix. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxx. a light chain variable region comprising an L-CDR1 having the amino acid sequence of SEQ ID NO: 250, an L-CDR2 having the amino acid sequence of SEQ ID NO: 251, and an L-CDR3 having the amino acid sequence of SEQ ID NO: 252; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
lxxi. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxii. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxiii. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxiv. a light chain variable region comprising an L-CDR1 having the amino acid sequence of SEQ ID NO: 150, an L-CDR2 having the amino acid sequence of SEQ ID NO: 151, and an L-CDR3 having the amino acid sequence of SEQ ID NO: 152; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxv. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxvi. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxvii. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxviii. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxix. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxx. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxxi. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxxii. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxxiii. a light chain variable region comprising an L-CDR1 having the amino acid sequence of SEQ ID NO: 250, an L-CDR2 having the amino acid sequence of SEQ ID NO: 251, and an L-CDR3 having the amino acid sequence of SEQ ID NO: 252; and a heavy chain variable region comprising an H-CDR1 having the amino acid sequence of SEQ ID NO: 155, an H-CDR2 having the amino acid sequence of SEQ ID NO: 156, and an H-CDR3 having the amino acid sequence of SEQ ID NO: 157; or
lxxxiv. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
lxxxv. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
lxxxvi. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
lxxxvii. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
lxxxviii. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
lxxxix. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
xc. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
xci. a light chain variable region comprising an L-CDR1 having the amino acid sequence of SEQ ID NO: 200, an L-CDR2 having the amino acid sequence of SEQ ID NO: 201, and an L-CDR3 having the amino acid sequence of SEQ ID NO: 202; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
xcii. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
xciii. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
xciv. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
xcv. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
xcvi. a light chain variable region comprising an L-CDR1 having the amino acid sequence of SEQ ID NO: 250, an L-CDR2 having the amino acid sequence of SEQ ID NO: 251, and an L-CDR3 having the amino acid sequence of SEQ ID NO: 252; and a heavy chain variable region comprising an H-CDR1 having the amino acid sequence of SEQ ID NO: 165, an H-CDR2 having the amino acid sequence of SEQ ID NO: 166, and an H-CDR3 having the amino acid sequence of SEQ ID NO: 167; or
xcvii. a light chain variable region comprising an L-CDR1 having the amino acid sequence of SEQ ID NO: 100, an L-CDR2 having the amino acid sequence of SEQ ID NO: 101, and an L-CDR3 having the amino acid sequence of SEQ ID NO: 102; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
xcviii. a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
xcix. a light chain variable region comprising an L-CDR1 having the amino acid sequence of SEQ ID NO: 120, an L-CDR2 having the amino acid sequence of SEQ ID NO: 121, and an L-CDR3 having the amino acid sequence of SEQ ID NO: 122; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
c. a light chain variable region comprising an L-CDR1 having the amino acid sequence of SEQ ID NO: 150, an L-CDR2 having the amino acid sequence of SEQ ID NO: 151, and an L-CDR3 having the amino acid sequence of SEQ ID NO: 152; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
ci. a light chain variable region comprising an L-CDR1 having the amino acid sequence of SEQ ID NO: 160, an L-CDR2 having the amino acid sequence of SEQ ID NO: 161, and an L-CDR3 having the amino acid sequence of SEQ ID NO: 162; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cii. a light chain variable region comprising an L-CDR1 having the amino acid sequence of SEQ ID NO: 170, an L-CDR2 having the amino acid sequence of SEQ ID NO: 171, and an L-CDR3 having the amino acid sequence of SEQ ID NO: 172; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
ciii. a light chain variable region comprising an L-CDR1 having the amino acid sequence of SEQ ID NO: 180, an L-CDR2 having the amino acid sequence of SEQ ID NO: 181, and an L-CDR3 having the amino acid sequence of SEQ ID NO: 182; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
civ. a light chain variable region comprising an L-CDR1 having the amino acid sequence of SEQ ID NO: 190, an L-CDR2 having the amino acid sequence of SEQ ID NO: 191, and an L-CDR3 having the amino acid sequence of SEQ ID NO: 192; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cv. a light chain variable region comprising an L-CDR1 having the amino acid sequence of SEQ ID NO: 200, an L-CDR2 having the amino acid sequence of SEQ ID NO: 201, and an L-CDR3 having the amino acid sequence of SEQ ID NO: 202; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cvi. a light chain variable region comprising an L-CDR1 having the amino acid sequence of SEQ ID NO: 210, an L-CDR2 having the amino acid sequence of SEQ ID NO: 211, and an L-CDR3 having the amino acid sequence of SEQ ID NO: 212; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cvii. a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cviii. a light chain variable region comprising an L-CDR1 having the amino acid sequence of SEQ ID NO: 230, an L-CDR2 having the amino acid sequence of SEQ ID NO: 231, and an L-CDR3 having the amino acid sequence of SEQ ID NO: 232; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177; or
cix. a light chain variable region comprising an L-CDR1 having the amino acid sequence of SEQ ID NO: 240, an L-CDR2 having the amino acid sequence of SEQ ID NO: 241, and an L-CDR3 having the amino acid sequence of SEQ ID NO: 242; and a heavy chain variable region comprising an H-CDR1 having the amino acid sequence of SEQ ID NO: 175, an H-CDR2 having the amino acid sequence of SEQ ID NO: 176, and an H-CDR3 having the amino acid sequence of SEQ ID NO: 177.

3. The method according to claim 1, wherein the anti-CTGF antibody or an antigen-binding fragment thereof comprises:
i. a light chain variable region comprising an amino acid sequence at least 80% identical to the amino acid sequence of SEQ ID NO: 223, and a heavy chain variable region comprising an amino acid sequence at least 80% identical to the amino acid sequence of SEQ ID NO: 108; or
ii. a light chain variable region comprising an amino acid sequence at least 80% identical to the amino acid sequence of SEQ ID NO: 103, and a heavy chain variable region comprising an amino acid sequence at least 80% identical to the amino acid sequence of SEQ ID NO: 108; or
iii. a light chain variable region comprising an amino acid sequence at least 80% identical to the amino acid sequence of SEQ ID NO: 113, and a heavy chain variable region comprising an amino acid sequence at least 80% identical to the amino acid sequence of SEQ ID NO: 108; or
iv. a light chain variable region comprising an amino acid sequence at least 80% identical to the amino acid sequence of SEQ ID NO: 123, and a heavy chain variable region comprising an amino acid sequence at least 80% identical to the amino acid sequence of SEQ ID NO: 108; or
v. a light chain variable region comprising an amino acid sequence at least 80% identical to the amino acid sequence of SEQ ID NO: 133, and a heavy chain variable region comprising an amino acid sequence at least 80% identical to the amino acid sequence of SEQ ID NO: 108; or
vi. a light chain variable region comprising an amino acid sequence at least 80% identical to the amino acid sequence of SEQ ID NO: 153, and a heavy chain variable region comprising an amino acid sequence at least 80% identical to the amino acid sequence of SEQ ID NO: 108; or
vii. a light chain variable region comprising an amino acid sequence at least 80% identical to the amino acid sequence of SEQ ID NO: 163, and a heavy chain variable region comprising an amino acid sequence at least 80% identical to the amino acid sequence of SEQ ID NO: 108; or
viii. a light chain variable region comprising an amino acid sequence at least 80% identical to the amino acid sequence of SEQ ID NO: 173, and a heavy chain variable region comprising an amino acid sequence at least 80% identical to the amino acid sequence of SEQ ID NO: 108; or
ix. a light chain variable region comprising an amino acid sequence at least 80% identical to the amino acid sequence of SEQ ID NO: 183, and a heavy chain variable region comprising an amino acid sequence at least 80% identical to the amino acid sequence of SEQ ID NO: 108; or
x. a light chain variable region comprising an amino acid sequence at least 80% identical to the amino acid sequence of SEQ ID NO: 193, and a heavy chain variable region comprising an amino acid sequence at least 80% identical to the amino acid sequence of SEQ ID NO: 108; or
xi. a light chain variable region comprising an amino acid sequence at least 80% identical to the amino acid sequence of SEQ ID NO: 203, and a heavy chain variable region comprising an amino acid sequence at least 80% identical to the amino acid sequence of SEQ ID NO: 108; or
xii. a light chain variable region comprising an amino acid sequence at least 80% identical to the amino acid sequence of SEQ ID NO: 213, and a heavy chain variable region comprising an amino acid sequence at least 80% identical to the amino acid sequence of SEQ ID NO: 108; or
xiii. a light chain variable region comprising an amino acid sequence at least 80% identical to the amino acid sequence of SEQ ID NO: 233, and a heavy chain variable region comprising an amino acid sequence at least 80% identical to the amino acid sequence of SEQ ID NO: 108; or
xiv. a light chain variable region comprising an amino acid sequence at least 80% identical to the amino acid sequence of SEQ ID NO: 243, and a heavy chain variable region comprising an amino acid sequence at least 80% identical to the amino acid sequence of SEQ ID NO: 108; or
xv. a light chain variable region comprising an amino acid sequence at least 80% identical to the amino acid sequence of SEQ ID NO: 253, and a heavy chain variable region comprising an amino acid sequence at least 80% identical to the amino acid sequence of SEQ ID NO: 108; or
xvi. a light chain variable region comprising an amino acid sequence at least 80% identical to the amino acid sequence of SEQ ID NO: 103, and a heavy chain variable region comprising an amino acid sequence at least 80% identical to the amino acid sequence of SEQ ID NO: 118; or
xvii. a light chain variable region comprising an amino acid sequence at least 80% identical to the amino acid sequence of SEQ ID NO: 113, and a heavy chain variable region comprising an amino acid sequence at least 80% identical to the amino acid sequence of SEQ ID NO: 118; or
xviii. a light chain variable region comprising an amino acid sequence at least 80% identical to the amino acid sequence of SEQ ID NO: 123, and a heavy chain variable region comprising an amino acid sequence at least 80% identical to the amino acid sequence of SEQ ID NO: 118; or
xix. a light chain variable region comprising an amino acid sequence at least 80% identical to the amino acid sequence of SEQ ID NO: 153, and a heavy chain variable region comprising an amino acid sequence at least 80% identical to the amino acid sequence of SEQ ID NO: 118; or
xx. a light chain variable region comprising an amino acid sequence at least 80% identical to the amino acid sequence of SEQ ID NO: 163, and a heavy chain variable region comprising an amino acid sequence at least 80% identical to the amino acid sequence of SEQ ID NO: 118; or
xxi. a light chain variable region comprising an amino acid sequence at least 80% identical to the amino acid sequence of SEQ ID NO: 173, and a heavy chain variable region comprising an amino acid sequence at least 80% identical to the amino acid sequence of SEQ ID NO: 118; or
xxii. a light chain variable region comprising an amino acid sequence at least 80% identical to the amino acid sequence of SEQ ID NO: 183, and a heavy chain variable region comprising an amino acid sequence at least 80% identical to the amino acid sequence of SEQ ID NO: 118; or
xxiii. a light chain variable region comprising an amino acid sequence at least 80% identical to the amino acid sequence of SEQ ID NO: 193, and a heavy chain variable region comprising an amino acid sequence at least 80% identical to the amino acid sequence of SEQ ID NO: 118; or
xxiv. a light chain variable region comprising an amino acid sequence at least 80% identical to the amino acid sequence of SEQ ID NO: 203, and a heavy chain variable region comprising an amino acid sequence at least 80% identical to the amino acid sequence of SEQ ID NO: 118; or
xxv. a light chain variable region comprising an amino acid sequence at least 80% identical to the amino acid sequence of SEQ ID NO: 213, and a heavy chain variable region comprising an amino acid sequence at least 80% identical to the amino acid sequence of SEQ ID NO: 118; or
xxvi. a light chain variable region comprising an amino acid sequence at least 80% identical to the amino acid sequence of SEQ ID NO: 223, and a heavy chain variable region comprising an amino acid sequence at least 80% identical to the amino acid sequence of SEQ ID NO: 118; or
xxvii. a light chain variable region comprising an amino acid sequence at least 80% identical to the amino acid sequence of SEQ ID NO: 233, and a heavy chain variable region comprising an amino acid sequence at least 80% identical to the amino acid sequence of SEQ ID NO: 118; or
xxviii. a light chain variable region comprising an amino acid sequence at least 80% identical to the amino acid sequence of SEQ ID NO: 243, and a heavy chain variable region comprising an amino acid sequence at least 80% identical to the amino acid sequence of SEQ ID NO: 118; or
xxix. a light chain variable region comprising an amino acid sequence at least 80% identical to the amino acid sequence of SEQ ID NO: 253, and a heavy chain variable region comprising an amino acid sequence at least 80% identical to the amino acid sequence of SEQ ID NO: 118; or
xxx. a light chain variable region comprising an amino acid sequence at least 80% identical to the amino acid sequence of SEQ ID NO: 103, and a heavy chain variable region comprising an amino acid sequence at least 80% identical to the amino acid sequence of SEQ ID NO: 128; or
xxxi. a light chain variable region comprising an amino acid sequence at least 80% identical to the amino acid sequence of SEQ ID NO: 113, and a heavy chain variable region comprising an amino acid sequence at least 80% identical to the amino acid sequence of SEQ ID NO: 128; or
xxxii. a light chain variable region comprising an amino acid sequence at least 80% identical to the amino acid sequence of SEQ ID NO: 123, and a heavy chain variable region comprising an amino acid sequence at least 80% identical to the amino acid sequence of SEQ ID NO: 128; or
xxxiii. a light chain variable region comprising an amino acid sequence at least 80% identical to the amino acid sequence of SEQ ID NO: 153, and a heavy chain variable region comprising an amino acid sequence at least 80% identical to the amino acid sequence of SEQ ID NO: 128; or
xxxiv. a light chain variable region comprising an amino acid sequence at least 80% identical to the amino acid sequence of SEQ ID NO: 163, and a heavy chain variable region comprising an amino acid sequence at least 80% identical to the amino acid sequence of SEQ ID NO: 128; or
xxxv. a light chain variable region comprising an amino acid sequence at least 80% identical to the amino acid sequence of SEQ ID NO: 173, and a heavy chain variable region comprising an amino acid sequence at least 80% identical to the amino acid sequence of SEQ ID NO: 128; or
xxxvi. a light chain variable region comprising an amino acid sequence at least 80% identical to the amino acid sequence of SEQ ID NO: 183, and a heavy chain variable region comprising an amino acid sequence at least 80% identical to the amino acid sequence of SEQ ID NO: 128; or
xxxvii. a light chain variable region comprising an amino acid sequence at least 80% identical to the amino acid sequence of SEQ ID NO: 193, and a heavy chain variable region comprising an amino acid sequence at least 80% identical to the amino acid sequence of SEQ ID NO: 128; or
xxxviii. a light chain variable region comprising an amino acid sequence at least 80% identical to the amino acid sequence of SEQ ID NO: 203, and a heavy chain variable region comprising an amino acid sequence at least 80% identical to the amino acid sequence of SEQ ID NO: 128; or
xxxix. a light chain variable region comprising an amino acid sequence at least 80% identical to the amino acid sequence of SEQ ID NO: 213, and a heavy chain variable region comprising an amino acid sequence at least 80% identical to the amino acid sequence of SEQ ID NO: 128; or
xl. a light chain variable region comprising an amino acid sequence at least 80% identical to the amino acid sequence of SEQ ID NO: 223, and a heavy chain variable region comprising an amino acid sequence at least 80% identical to the amino acid sequence of SEQ ID NO: 128; or
xli. a light chain variable region comprising an amino acid sequence at least 80% identical to the amino acid sequence of SEQ ID NO: 233, and a heavy chain variable region comprising an amino acid sequence at least 80% identical to the amino acid sequence of SEQ ID NO: 128; or
xlii. a light chain variable region comprising an amino acid sequence at least 80% identical to the amino acid sequence of SEQ ID NO: 243, and a heavy chain variable region comprising an amino acid sequence at least 80% identical to the amino acid sequence of SEQ ID NO: 128; or
xliii. a light chain variable region comprising an amino acid sequence at least 80% identical to the amino acid sequence of SEQ ID NO: 253, and a heavy chain variable region comprising an amino acid sequence at least 80% identical to the amino acid sequence of SEQ ID NO: 128; or
xliv. a light chain variable region comprising an amino acid sequence at least 80% identical to the amino acid sequence of SEQ ID NO: 103, and a heavy chain variable region comprising an amino acid sequence at least 80% identical to the amino acid sequence of SEQ ID NO: 138; or
xlv. a light chain variable region comprising an amino acid sequence at least 80% identical to the amino acid sequence of SEQ ID NO: 113, and a heavy chain variable region comprising an amino acid sequence at least 80% identical to the amino acid sequence of SEQ ID NO: 138; or
xlvi. a light chain variable region comprising an amino acid sequence at least 80% identical to the amino acid sequence of SEQ ID NO: 123, and a heavy chain variable region comprising an amino acid sequence at least 80% identical to the amino acid sequence of SEQ ID NO: 138; or
xlvii. a light chain variable region comprising an amino acid sequence at least 80% identical to the amino acid sequence of SEQ ID NO: 153, and a heavy chain variable region comprising an amino acid sequence at least 80% identical to the amino acid sequence of SEQ ID NO: 138; or
xlviii. a light chain variable region comprising an amino acid sequence at least 80% identical to the amino acid sequence of SEQ ID NO: 163, and a heavy chain variable region comprising an amino acid sequence at least 80% identical to the amino acid sequence of SEQ ID NO: 138; or
xlix. a light chain variable region comprising an amino acid sequence at least 80% identical to the amino acid sequence of SEQ ID NO: 173, and a heavy chain variable region comprising an amino acid sequence at least 80% identical to the amino acid sequence of SEQ ID NO: 138; or
l. a light chain variable region comprising an amino acid sequence at least 80% identical to the amino acid sequence of SEQ ID NO: 183, and a heavy chain variable region comprising an amino acid sequence at least 80% identical to the amino acid sequence of SEQ ID NO: 138; or
li. a light chain variable region comprising an amino acid sequence at least 80% identical to the amino acid sequence of SEQ ID NO: 193, and a heavy chain variable region comprising an amino acid sequence at least 80% identical to the amino acid sequence of SEQ ID NO: 138; or
lii. a light chain variable region comprising an amino acid sequence at least 80% identical to the amino acid sequence of SEQ ID NO: 203, and a heavy chain variable region comprising an amino acid sequence at least 80% identical to the amino acid sequence of SEQ ID NO: 138; or
liii. a light chain variable region comprising an amino acid sequence at least 80% identical to the amino acid sequence of SEQ ID NO: 213, and a heavy chain variable region comprising an amino acid sequence at least 80% identical to the amino acid sequence of SEQ ID NO: 138; or
liv. a light chain variable region comprising an amino acid sequence at least 80% identical to the amino acid sequence of SEQ ID NO: 223, and a heavy chain variable region comprising an amino acid sequence at least 80% identical to the amino acid sequence of SEQ ID NO: 138; or
lv. a light chain variable region comprising an amino acid sequence at least 80% identical to the amino acid sequence of SEQ ID NO: 233, and a heavy chain variable region comprising an amino acid sequence at least 80% identical to the amino acid sequence of SEQ ID NO: 138; or
lvi. a light chain variable region comprising an amino acid sequence at least 80% identical to the amino acid sequence of SEQ ID NO: 243, and a heavy chain variable region comprising an amino acid sequence at least 80% identical to the amino acid sequence of SEQ ID NO: 138; or
lvii. a light chain variable region comprising an amino acid sequence at least 80% identical to the amino acid sequence of SEQ ID NO: 253, and a heavy chain variable region comprising an amino acid sequence at least 80% identical to the amino acid sequence of SEQ ID NO: 138; or
lviii. a light chain variable region comprising an amino acid sequence at least 80% identical to the amino acid sequence of SEQ ID NO: 103, and a heavy chain variable region comprising an amino acid sequence at least 80% identical to the amino acid sequence of SEQ ID NO: 148; or
lix. a light chain variable region comprising an amino acid sequence at least 80% identical to the amino acid sequence of SEQ ID NO: 113, and a heavy chain variable region comprising an amino acid sequence at least 80% identical to the amino acid sequence of SEQ ID NO: 148; or
lx. a light chain variable region comprising an amino acid sequence at least 80% identical to the amino acid sequence of SEQ ID NO: 123, and a heavy chain variable region comprising an amino acid sequence at least 80% identical to the amino acid sequence of SEQ ID NO: 148; or
lxi. a light chain variable region comprising an amino acid sequence at least 80% identical to the amino acid sequence of SEQ ID NO: 163, and a heavy chain variable region comprising an amino acid sequence at least 80% identical to the amino acid sequence of SEQ ID NO: 148; or
lxii. a light chain variable region comprising an amino acid sequence at least 80% identical to the amino acid sequence of SEQ ID NO: 173, and a heavy chain variable region comprising an amino acid sequence at least 80% identical to the amino acid sequence of SEQ ID NO: 148; or
lxiii. a light chain variable region comprising an amino acid sequence at least 80% identical to the amino acid sequence of SEQ ID NO: 183, and a heavy chain variable region comprising an amino acid sequence at least 80% identical to the amino acid sequence of SEQ ID NO: 148; or
lxiv. a light chain variable region comprising an amino acid sequence at least 80% identical to the amino acid sequence of SEQ ID NO: 193, and a heavy chain variable region comprising an amino acid sequence at least 80% identical to the amino acid sequence of SEQ ID NO: 148; or
lxv. a light chain variable region comprising an amino acid sequence at least 80% identical to the amino acid sequence of SEQ ID NO: 203, and a heavy chain variable region comprising an amino acid sequence at least 80% identical to the amino acid sequence of SEQ ID NO: 148; or
lxvi. a light chain variable region comprising an amino acid sequence at least 80% identical to the amino acid sequence of SEQ ID NO: 213, and a heavy chain variable region comprising an amino acid sequence at least 80% identical to the amino acid sequence of SEQ ID NO: 148; or
lxvii. a light chain variable region comprising an amino acid sequence at least 80% identical to the amino acid sequence of SEQ ID NO: 223, and a heavy chain variable region comprising an amino acid sequence at least 80% identical to the amino acid sequence of SEQ ID NO: 148; or
lxviii. a light chain variable region comprising an amino acid sequence at least 80% identical to the amino acid sequence of SEQ ID NO: 233, and a heavy chain variable region comprising an amino acid sequence at least 80% identical to the amino acid sequence of SEQ ID NO: 148; or
lxix. a light chain variable region comprising an amino acid sequence at least 80% identical to the amino acid sequence of SEQ ID NO: 243, and a heavy chain variable region comprising an amino acid sequence at least 80% identical to the amino acid sequence of SEQ ID NO: 148; or
lxx. a light chain variable region comprising an amino acid sequence at least 80% identical to the amino acid sequence of SEQ ID NO: 253, and a heavy chain variable region comprising an amino acid sequence at least 80% identical to the amino acid sequence of SEQ ID NO: 148; or
lxxi. a light chain variable region comprising an amino acid sequence at least 80% identical to the amino acid sequence of SEQ ID NO: 103, and a heavy chain variable region comprising an amino acid sequence at least 80% identical to the amino acid sequence of SEQ ID NO: 158; or
lxxii. a light chain variable region comprising an amino acid sequence at least 80% identical to the amino acid sequence of SEQ ID NO: 113, and a heavy chain variable region comprising an amino acid sequence at least 80% identical to the amino acid sequence of SEQ ID NO: 158; or
lxxiii. a light chain variable region comprising an amino acid sequence at least 80% identical to the amino acid sequence of SEQ ID NO: 123, and a heavy chain variable region comprising an amino acid sequence at least 80% identical to the amino acid sequence of SEQ ID NO: 158; or
lxxiv. a light chain variable region comprising an amino acid sequence at least 80% identical to the amino acid sequence of SEQ ID NO: 153, and a heavy chain variable region comprising an amino acid sequence at least 80% identical to the amino acid sequence of SEQ ID NO: 158; or
lxxv. a light chain variable region comprising an amino acid sequence at least 80% identical to the amino acid sequence of SEQ ID NO: 163, and a heavy chain variable region comprising an amino acid sequence at least 80% identical to the amino acid sequence of SEQ ID NO: 158; or
lxxvi. a light chain variable region comprising an amino acid sequence at least 80% identical to the amino acid sequence of SEQ ID NO: 173, and a heavy chain variable region comprising an amino acid sequence at least 80% identical to the amino acid sequence of SEQ ID NO: 158; or
lxxvii. a light chain variable region comprising an amino acid sequence at least 80% identical to the amino acid sequence of SEQ ID NO: 183, and a heavy chain variable region comprising an amino acid sequence at least 80% identical to the amino acid sequence of SEQ ID NO: 158; or
lxxviii. a light chain variable region comprising an amino acid sequence at least 80% identical to the amino acid sequence of SEQ ID NO: 193, and a heavy chain variable region comprising an amino acid sequence at least 80% identical to the amino acid sequence of SEQ ID NO: 158; or
lxxix. a light chain variable region comprising an amino acid sequence at least 80% identical to the amino acid sequence of SEQ ID NO: 213, and a heavy chain variable region comprising an amino acid sequence at least 80% identical to the amino acid sequence of SEQ ID NO: 158; or
lxxx. a light chain variable region comprising an amino acid sequence at least 80% identical to the amino acid sequence of SEQ ID NO: 223, and a heavy chain variable region comprising an amino acid sequence at least 80% identical to the amino acid sequence of SEQ ID NO: 158; or
lxxxi. a light chain variable region comprising an amino acid sequence at least 80% identical to the amino acid sequence of SEQ ID NO: 233, and a heavy chain variable region comprising an amino acid sequence at least 80% identical to the amino acid sequence of SEQ ID NO: 158; or
lxxxii. a light chain variable region comprising an amino acid sequence at least 80% identical to the amino acid sequence of SEQ ID NO: 243, and a heavy chain variable region comprising an amino acid sequence at least 80% identical to the amino acid sequence of SEQ ID NO: 158; or
lxxxiii. a light chain variable region comprising an amino acid sequence at least 80% identical to the amino acid sequence of SEQ ID NO: 253, and a heavy chain variable region comprising an amino acid sequence at least 80% identical to the amino acid sequence of SEQ ID NO: 158; or
lxxxiv. a light chain variable region comprising an amino acid sequence at least 80% identical to the amino acid sequence of SEQ ID NO: 103, and a heavy chain variable region comprising an amino acid sequence at least 80% identical to the amino acid sequence of SEQ ID NO: 168; or
lxxxv. a light chain variable region comprising an amino acid sequence at least 80% identical to the amino acid sequence of SEQ ID NO: 113, and a heavy chain variable region comprising an amino acid sequence at least 80% identical to the amino acid sequence of SEQ ID NO: 168; or
lxxxvi. a light chain variable region comprising an amino acid sequence at least 80% identical to the amino acid sequence of SEQ ID NO: 123, and a heavy chain variable region comprising an amino acid sequence at least 80% identical to the amino acid sequence of SEQ ID NO: 168; or
lxxxvii. a light chain variable region comprising an amino acid sequence at least 80% identical to the amino acid sequence of SEQ ID NO: 163, and a heavy chain variable region comprising an amino acid sequence at least 80% identical to the amino acid sequence of SEQ ID NO: 168; or
lxxxviii. a light chain variable region comprising an amino acid sequence at least 80% identical to the amino acid sequence of SEQ ID NO: 173, and a heavy chain variable region comprising an amino acid sequence at least 80% identical to the amino acid sequence of SEQ ID NO: 168; or
lxxxix. a light chain variable region comprising an amino acid sequence at least 80% identical to the amino acid sequence of SEQ ID NO: 183, and a heavy chain variable region comprising an amino acid sequence at least 80% identical to the amino acid sequence of SEQ ID NO: 168; or
xc. a light chain variable region comprising an amino acid sequence at least 80% identical to the amino acid sequence of SEQ ID NO: 193, and a heavy chain variable region comprising an amino acid sequence at least 80% identical to the amino acid sequence of SEQ ID NO: 168; or
xci. a light chain variable region comprising an amino acid sequence at least 80% identical to the amino acid sequence of SEQ ID NO: 203, and a heavy chain variable region comprising an amino acid sequence at least 80% identical to the amino acid sequence of SEQ ID NO: 168; or
xcii. a light chain variable region comprising an amino acid sequence at least 80% identical to the amino acid sequence of SEQ ID NO: 213, and a heavy chain variable region comprising an amino acid sequence at least 80% identical to the amino acid sequence of SEQ ID NO: 168; or
xciii. a light chain variable region comprising an amino acid sequence at least 80% identical to the amino acid sequence of SEQ ID NO: 223, and a heavy chain variable region comprising an amino acid sequence at least 80% identical to the amino acid sequence of SEQ ID NO: 168; or
xciv. a light chain variable region comprising an amino acid sequence at least 80% identical to the amino acid sequence of SEQ ID NO: 233, and a heavy chain variable region comprising an amino acid sequence at least 80% identical to the amino acid sequence of SEQ ID NO: 168; or
xcv. a light chain variable region comprising an amino acid sequence at least 80% identical to the amino acid sequence of SEQ ID NO: 243, and a heavy chain variable region comprising an amino acid sequence at least 80% identical to the amino acid sequence of SEQ ID NO: 168; or
xcvi. a light chain variable region comprising an amino acid sequence at least 80% identical to the amino acid sequence of SEQ ID NO: 253, and a heavy chain variable region comprising an amino acid sequence at least 80% identical to the amino acid sequence of SEQ ID NO: 168; or
xcvii. a light chain variable region comprising an amino acid sequence at least 80% identical to the amino acid sequence of SEQ ID NO: 103, and a heavy chain variable region comprising an amino acid sequence at least 80% identical to the amino acid sequence of SEQ ID NO: 178; or
xcviii. a light chain variable region comprising an amino acid sequence at least 80% identical to the amino acid sequence of SEQ ID NO: 113, and a heavy chain variable region comprising an amino acid sequence at least 80% identical to the amino acid sequence of SEQ ID NO: 178; or
xcix. a light chain variable region comprising an amino acid sequence at least 80% identical to the amino acid sequence of SEQ ID NO: 123, and a heavy chain variable region comprising an amino acid sequence at least 80% identical to the amino acid sequence of SEQ ID NO: 178; or
c. a light chain variable region comprising an amino acid sequence at least 80% identical to the amino acid sequence of SEQ ID NO: 153, and a heavy chain variable region comprising an amino acid sequence at least 80% identical to the amino acid sequence of SEQ ID NO: 178; or
ci. a light chain variable region comprising an amino acid sequence at least 80% identical to the amino acid sequence of SEQ ID NO: 163, and a heavy chain variable region comprising an amino acid sequence at least 80% identical to the amino acid sequence of SEQ ID NO: 178; or
cii. a light chain variable region comprising an amino acid sequence at least 80% identical to the amino acid sequence of SEQ ID NO: 173, and a heavy chain variable region comprising an amino acid sequence at least 80% identical to the amino acid sequence of SEQ ID NO: 178; or
ciii. a light chain variable region comprising an amino acid sequence at least 80% identical to the amino acid sequence of SEQ ID NO: 183, and a heavy chain variable region comprising an amino acid sequence at least 80% identical to the amino acid sequence of SEQ ID NO: 178; or
civ. a light chain variable region comprising an amino acid sequence at least 80% identical to the amino acid sequence of SEQ ID NO: 193, and a heavy chain variable region comprising an amino acid sequence at least 80% identical to the amino acid sequence of SEQ ID NO: 178; or
cv. a light chain variable region comprising an amino acid sequence at least 80% identical to the amino acid sequence of SEQ ID NO: 203, and a heavy chain variable region comprising an amino acid sequence at least 80% identical to the amino acid sequence of SEQ ID NO: 178; or
cvi. a light chain variable region comprising an amino acid sequence at least 80% identical to the amino acid sequence of SEQ ID NO: 213, and a heavy chain variable region comprising an amino acid sequence at least 80% identical to the amino acid sequence of SEQ ID NO: 178; or
cvii. a light chain variable region comprising an amino acid sequence at least 80% identical to the amino acid sequence of SEQ ID NO: 223, and a heavy chain variable region comprising an amino acid sequence at least 80% identical to the amino acid sequence of SEQ ID NO: 178; or
cviii. a light chain variable region comprising an amino acid sequence at least 80% identical to the amino acid sequence of SEQ ID NO: 233, and a heavy chain variable region comprising an amino acid sequence at least 80% identical to the amino acid sequence of SEQ ID NO: 178; or
cix. a light chain variable region comprising an amino acid sequence at least 80% identical to the amino acid sequence of SEQ ID NO: 243, and a heavy chain variable region comprising an amino acid sequence at least 80% identical to the amino acid sequence of SEQ ID NO: 178.

4. The method according to claim 1, wherein the anti-CTGF antibody or an antigen-binding fragment thereof comprises:
i. a light chain variable region comprising an amino acid sequence at least 85% identical to the amino acid sequence of SEQ ID NO: 223, and a heavy chain variable region comprising an amino acid sequence at least 85% identical to the amino acid sequence of SEQ ID NO: 108; or
ii. a light chain variable region comprising an amino acid sequence at least 85% identical to the amino acid sequence of SEQ ID NO: 103, and a heavy chain variable region comprising an amino acid sequence at least 85% identical to the amino acid sequence of SEQ ID NO: 108; or
iii. a light chain variable region comprising an amino acid sequence at least 85% identical to the amino acid sequence of SEQ ID NO: 113, and a heavy chain variable region comprising an amino acid sequence at least 85% identical to the amino acid sequence of SEQ ID NO: 108; or
iv. a light chain variable region comprising an amino acid sequence at least 85% identical to the amino acid sequence of SEQ ID NO: 123, and a heavy chain variable region comprising an amino acid sequence at least 85% identical to the amino acid sequence of SEQ ID NO: 108; or
v. a light chain variable region comprising an amino acid sequence at least 85% identical to the amino acid sequence of SEQ ID NO: 133, and a heavy chain variable region comprising an amino acid sequence at least 85% identical to the amino acid sequence of SEQ ID NO: 108; or
vi. a light chain variable region comprising an amino acid sequence at least 85% identical to the amino acid sequence of SEQ ID NO: 153, and a heavy chain variable region comprising an amino acid sequence at least 85% identical to the amino acid sequence of SEQ ID NO: 108; or
vii. a light chain variable region comprising an amino acid sequence at least 85% identical to the amino acid sequence of SEQ ID NO: 163, and a heavy chain variable region comprising an amino acid sequence at least 85% identical to the amino acid sequence of SEQ ID NO: 108; or
viii. a light chain variable region comprising an amino acid sequence at least 85% identical to the amino acid sequence of SEQ ID NO: 173, and a heavy chain variable region comprising an amino acid sequence at least 85% identical to the amino acid sequence of SEQ ID NO: 108; or
ix. a light chain variable region comprising an amino acid sequence at least 85% identical to the amino acid sequence of SEQ ID NO: 183, and a heavy chain variable region comprising an amino acid sequence at least 85% identical to the amino acid sequence of SEQ ID NO: 108; or
x. a light chain variable region comprising an amino acid sequence at least 85% identical to the amino acid sequence of SEQ ID NO: 193, and a heavy chain variable region comprising an amino acid sequence at least 85% identical to the amino acid sequence of SEQ ID NO: 108; or
xi. a light chain variable region comprising an amino acid sequence at least 85% identical to the amino acid sequence of SEQ ID NO: 203, and a heavy chain variable region comprising an amino acid sequence at least 85% identical to the amino acid sequence of SEQ ID NO: 108; or
xii. a light chain variable region comprising an amino acid sequence at least 85% identical to the amino acid sequence of SEQ ID NO: 213, and a heavy chain variable region comprising an amino acid sequence at least 85% identical to the amino acid sequence of SEQ ID NO: 108; or
xiii. a light chain variable region comprising an amino acid sequence at least 85% identical to the amino acid sequence of SEQ ID NO: 233, and a heavy chain variable region comprising an amino acid sequence at least 85% identical to the amino acid sequence of SEQ ID NO: 108; or
xiv. a light chain variable region comprising an amino acid sequence at least 85% identical to the amino acid sequence of SEQ ID NO: 243, and a heavy chain variable region comprising an amino acid sequence at least 85% identical to the amino acid sequence of SEQ ID NO: 108; or
xv. a light chain variable region comprising an amino acid sequence at least 85% identical to the amino acid sequence of SEQ ID NO: 253, and a heavy chain variable region comprising an amino acid sequence at least 85% identical to the amino acid sequence of SEQ ID NO: 108; or
xvi. a light chain variable region comprising an amino acid sequence at least 85% identical to the amino acid sequence of SEQ ID NO: 103, and a heavy chain variable region comprising an amino acid sequence at least 85% identical to the amino acid sequence of SEQ ID NO: 118; or
xvii. a light chain variable region comprising an amino acid sequence at least 85% identical to the amino acid sequence of SEQ ID NO: 113, and a heavy chain variable region comprising an amino acid sequence at least 85% identical to the amino acid sequence of SEQ ID NO: 118; or
xviii. a light chain variable region comprising an amino acid sequence at least 85% identical to the amino acid sequence of SEQ ID NO: 123, and a heavy chain variable region comprising an amino acid sequence at least 85% identical to the amino acid sequence of SEQ ID NO: 118; or
xix. a light chain variable region comprising an amino acid sequence at least 85% identical to the amino acid sequence of SEQ ID NO: 153, and a heavy chain variable region comprising an amino acid sequence at least 85% identical to the amino acid sequence of SEQ ID NO: 118; or
xx. a light chain variable region comprising an amino acid sequence at least 85% identical to the amino acid sequence of SEQ ID NO: 163, and a heavy chain variable region comprising an amino acid sequence at least 85% identical to the amino acid sequence of SEQ ID NO: 118; or
xxi. a light chain variable region comprising an amino acid sequence at least 85% identical to the amino acid sequence of SEQ ID NO: 173, and a heavy chain variable region comprising an amino acid sequence at least 85% identical to the amino acid sequence of SEQ ID NO: 118; or
xxii. a light chain variable region comprising an amino acid sequence at least 85% identical to the amino acid sequence of SEQ ID NO: 183, and a heavy chain variable region comprising an amino acid sequence at least 85% identical to the amino acid sequence of SEQ ID NO: 118; or
xxiii. a light chain variable region comprising an amino acid sequence at least 85% identical to the amino acid sequence of SEQ ID NO: 193, and a heavy chain variable region comprising an amino acid sequence at least 85% identical to the amino acid sequence of SEQ ID NO: 118; or
xxiv. a light chain variable region comprising an amino acid sequence at least 85% identical to the amino acid sequence of SEQ ID NO: 203, and a heavy chain variable region comprising an amino acid sequence at least 85% identical to the amino acid sequence of SEQ ID NO: 118; or
xxv. a light chain variable region comprising an amino acid sequence at least 85% identical to the amino acid sequence of SEQ ID NO: 213, and a heavy chain variable region comprising an amino acid sequence at least 85% identical to the amino acid sequence of SEQ ID NO: 118; or
xxvi. a light chain variable region comprising an amino acid sequence at least 85% identical to the amino acid sequence of SEQ ID NO: 223, and a heavy chain variable region comprising an amino acid sequence at least 85% identical to the amino acid sequence of SEQ ID NO: 118; or
xxvii. a light chain variable region comprising an amino acid sequence at least 85% identical to the amino acid sequence of SEQ ID NO: 233, and a heavy chain variable region comprising an amino acid sequence at least 85% identical to the amino acid sequence of SEQ ID NO: 118; or
xxviii. a light chain variable region comprising an amino acid sequence at least 85% identical to the amino acid sequence of SEQ ID NO: 243, and a heavy chain variable region comprising an amino acid sequence at least 85% identical to the amino acid sequence of SEQ ID NO: 118; or
xxix. a light chain variable region comprising an amino acid sequence at least 85% identical to the amino acid sequence of SEQ ID NO: 253, and a heavy chain variable region comprising an amino acid sequence at least 85% identical to the amino acid sequence of SEQ ID NO: 118; or
xxx. a light chain variable region comprising an amino acid sequence at least 85% identical to the amino acid sequence of SEQ ID NO: 103, and a heavy chain variable region comprising an amino acid sequence at least 85% identical to the amino acid sequence of SEQ ID NO: 128; or
xxxi. a light chain variable region comprising an amino acid sequence at least 85% identical to the amino acid sequence of SEQ ID NO: 113, and a heavy chain variable region comprising an amino acid sequence at least 85% identical to the amino acid sequence of SEQ ID NO: 128; or
xxxii. a light chain variable region comprising an amino acid sequence at least 85% identical to the amino acid sequence of SEQ ID NO: 123, and a heavy chain variable region comprising an amino acid sequence at least 85% identical to the amino acid sequence of SEQ ID NO: 128; or
xxxiii. a light chain variable region comprising an amino acid sequence at least 85% identical to the amino acid sequence of SEQ ID NO: 153, and a heavy chain variable region comprising an amino acid sequence at least 85% identical to the amino acid sequence of SEQ ID NO: 128; or
xxxiv. a light chain variable region comprising an amino acid sequence at least 85% identical to the amino acid sequence of SEQ ID NO: 163, and a heavy chain variable region comprising an amino acid sequence at least 85% identical to the amino acid sequence of SEQ ID NO: 128; or
xxxv. a light chain variable region comprising an amino acid sequence at least 85% identical to the amino acid sequence of SEQ ID NO: 173, and a heavy chain variable region comprising an amino acid sequence at least 85% identical to the amino acid sequence of SEQ ID NO: 128; or
xxxvi. a light chain variable region comprising an amino acid sequence at least 85% identical to the amino acid sequence of SEQ ID NO: 183, and a heavy chain variable region comprising an amino acid sequence at least 85% identical to the amino acid sequence of SEQ ID NO: 128; or
xxxvii. a light chain variable region comprising an amino acid sequence at least 85% identical to the amino acid sequence of SEQ ID NO: 193, and a heavy chain variable region comprising an amino acid sequence at least 85% identical to the amino acid sequence of SEQ ID NO: 128; or
xxxviii. a light chain variable region comprising an amino acid sequence at least 85% identical to the amino acid sequence of SEQ ID NO: 203, and a heavy chain variable region comprising an amino acid sequence at least 85% identical to the amino acid sequence of SEQ ID NO: 128; or
xxxix. a light chain variable region comprising an amino acid sequence at least 85% identical to the amino acid sequence of SEQ ID NO: 213, and a heavy chain variable region comprising an amino acid sequence at least 85% identical to the amino acid sequence of SEQ ID NO: 128; or
xl. a light chain variable region comprising an amino acid sequence at least 85% identical to the amino acid sequence of SEQ ID NO: 223, and a heavy chain variable region comprising an amino acid sequence at least 85% identical to the amino acid sequence of SEQ ID NO: 128; or
xli. a light chain variable region comprising an amino acid sequence at least 85% identical to the amino acid sequence of SEQ ID NO: 233, and a heavy chain variable region comprising an amino acid sequence at least 85% identical to the amino acid sequence of SEQ ID NO: 128; or
xlii. a light chain variable region comprising an amino acid sequence at least 85% identical to the amino acid sequence of SEQ ID NO: 243, and a heavy chain variable region comprising an amino acid sequence at least 85% identical to the amino acid sequence of SEQ ID NO: 128; or
xliii. a light chain variable region comprising an amino acid sequence at least 85% identical to the amino acid sequence of SEQ ID NO: 253, and a heavy chain variable region comprising an amino acid sequence at least 85% identical to the amino acid sequence of SEQ ID NO: 128; or
xliv. a light chain variable region comprising an amino acid sequence at least 85% identical to the amino acid sequence of SEQ ID NO: 103, and a heavy chain variable region comprising an amino acid sequence at least 85% identical to the amino acid sequence of SEQ ID NO: 138; or
xlv. a light chain variable region comprising an amino acid sequence at least 85% identical to the amino acid sequence of SEQ ID NO: 113, and a heavy chain variable region comprising an amino acid sequence at least 85% identical to the amino acid sequence of SEQ ID NO: 138; or
xlvi. a light chain variable region comprising an amino acid sequence at least 85% identical to the amino acid sequence of SEQ ID NO: 123, and a heavy chain variable region comprising an amino acid sequence at least 85% identical to the amino acid sequence of SEQ ID NO: 138; or
xlvii. a light chain variable region comprising an amino acid sequence at least 85% identical to the amino acid sequence of SEQ ID NO: 153, and a heavy chain variable region comprising an amino acid sequence at least 85% identical to the amino acid sequence of SEQ ID NO: 138; or
xlviii. a light chain variable region comprising an amino acid sequence at least 85% identical to the amino acid sequence of SEQ ID NO: 163, and a heavy chain variable region comprising an amino acid sequence at least 85% identical to the amino acid sequence of SEQ ID NO: 138; or
xlix. a light chain variable region comprising an amino acid sequence at least 85% identical to the amino acid sequence of SEQ ID NO: 173, and a heavy chain variable region comprising an amino acid sequence at least 85% identical to the amino acid sequence of SEQ ID NO: 138; or
l. a light chain variable region comprising an amino acid sequence at least 85% identical to the amino acid sequence of SEQ ID NO: 183, and a heavy chain variable region comprising an amino acid sequence at least 85% identical to the amino acid sequence of SEQ ID NO: 138; or
li. a light chain variable region comprising an amino acid sequence at least 85% identical to the amino acid sequence of SEQ ID NO: 193, and a heavy chain variable region comprising an amino acid sequence at least 85% identical to the amino acid sequence of SEQ ID NO: 138; or
lii. a light chain variable region comprising an amino acid sequence at least 85% identical to the amino acid sequence of SEQ ID NO: 203, and a heavy chain variable region comprising an amino acid sequence at least 85% identical to the amino acid sequence of SEQ ID NO: 138; or
liii. a light chain variable region comprising an amino acid sequence at least 85% identical to the amino acid sequence of SEQ ID NO: 213, and a heavy chain variable region comprising an amino acid sequence at least 85% identical to the amino acid sequence of SEQ ID NO: 138; or
liv. a light chain variable region comprising an amino acid sequence at least 85% identical to the amino acid sequence of SEQ ID NO: 223, and a heavy chain variable region comprising an amino acid sequence at least 85% identical to the amino acid sequence of SEQ ID NO: 138; or
lv. a light chain variable region comprising an amino acid sequence at least 85% identical to the amino acid sequence of SEQ ID NO: 233, and a heavy chain variable region comprising an amino acid sequence at least 85% identical to the amino acid sequence of SEQ ID NO: 138; or
lvi. a light chain variable region comprising an amino acid sequence at least 85% identical to the amino acid sequence of SEQ ID NO: 243, and a heavy chain variable region comprising an amino acid sequence at least 85% identical to the amino acid sequence of SEQ ID NO: 138; or
lvii. a light chain variable region comprising an amino acid sequence at least 85% identical to the amino acid sequence of SEQ ID NO: 253, and a heavy chain variable region comprising an amino acid sequence at least 85% identical to the amino acid sequence of SEQ ID NO: 138; or
lviii. a light chain variable region comprising an amino acid sequence at least 85% identical to the amino acid sequence of SEQ ID NO: 103, and a heavy chain variable region comprising an amino acid sequence at least 85% identical to the amino acid sequence of SEQ ID NO: 148; or
lix. a light chain variable region comprising an amino acid sequence at least 85% identical to the amino acid sequence of SEQ ID NO: 113, and a heavy chain variable region comprising an amino acid sequence at least 85% identical to the amino acid sequence of SEQ ID NO: 148; or
lx. a light chain variable region comprising an amino acid sequence at least 85% identical to the amino acid sequence of SEQ ID NO: 123, and a heavy chain variable region comprising an amino acid sequence at least 85% identical to the amino acid sequence of SEQ ID NO: 148; or
lxi. a light chain variable region comprising an amino acid sequence at least 85% identical to the amino acid sequence of SEQ ID NO: 163, and a heavy chain variable region comprising an amino acid sequence at least 85% identical to the amino acid sequence of SEQ ID NO: 148; or
lxii. a light chain variable region comprising an amino acid sequence at least 85% identical to the amino acid sequence of SEQ ID NO: 173, and a heavy chain variable region comprising an amino acid sequence at least 85% identical to the amino acid sequence of SEQ ID NO: 148; or
lxiii. a light chain variable region comprising an amino acid sequence at least 85% identical to the amino acid sequence of SEQ ID NO: 183, and a heavy chain variable region comprising an amino acid sequence at least 85% identical to the amino acid sequence of SEQ ID NO: 148; or
lxiv. a light chain variable region comprising an amino acid sequence at least 85% identical to the amino acid sequence of SEQ ID NO: 193, and a heavy chain variable region comprising an amino acid sequence at least 85% identical to the amino acid sequence of SEQ ID NO: 148; or
lxv. a light chain variable region comprising an amino acid sequence at least 85% identical to the amino acid sequence of SEQ ID NO: 203, and a heavy chain variable region comprising an amino acid sequence at least 85% identical to the amino acid sequence of SEQ ID NO: 148; or
lxvi. a light chain variable region comprising an amino acid sequence at least 85% identical to the amino acid sequence of SEQ ID NO: 213, and a heavy chain variable region comprising an amino acid sequence at least 85% identical to the amino acid sequence of SEQ ID NO: 148; or
lxvii. a light chain variable region comprising an amino acid sequence at least 85% identical to the amino acid sequence of SEQ ID NO: 223, and a heavy chain variable region comprising an amino acid sequence at least 85% identical to the amino acid sequence of SEQ ID NO: 148; or
lxviii. a light chain variable region comprising an amino acid sequence at least 85% identical to the amino acid sequence of SEQ ID NO: 233, and a heavy chain variable region comprising an amino acid sequence at least 85% identical to the amino acid sequence of SEQ ID NO: 148; or
lxix. a light chain variable region comprising an amino acid sequence at least 85% identical to the amino acid sequence of SEQ ID NO: 243, and a heavy chain variable region comprising an amino acid sequence at least 85% identical to the amino acid sequence of SEQ ID NO: 148; or
lxx. a light chain variable region comprising an amino acid sequence at least 85% identical to the amino acid sequence of SEQ ID NO: 253, and a heavy chain variable region comprising an amino acid sequence at least 85% identical to the amino acid sequence of SEQ ID NO: 148; or
lxxi. a light chain variable region comprising an amino acid sequence at least 85% identical to the amino acid sequence of SEQ ID NO: 103, and a heavy chain variable region comprising an amino acid sequence at least 85% identical to the amino acid sequence of SEQ ID NO: 158; or
lxxii. a light chain variable region comprising an amino acid sequence at least 85% identical to the amino acid sequence of SEQ ID NO: 113, and a heavy chain variable region comprising an amino acid sequence at least 85% identical to the amino acid sequence of SEQ ID NO: 158; or
lxxiii. a light chain variable region comprising an amino acid sequence at least 85% identical to the amino acid sequence of SEQ ID NO: 123, and a heavy chain variable region comprising an amino acid sequence at least 85% identical to the amino acid sequence of SEQ ID NO: 158; or
lxxiv. a light chain variable region comprising an amino acid sequence at least 85% identical to the amino acid sequence of SEQ ID NO: 153, and a heavy chain variable region comprising an amino acid sequence at least 85% identical to the amino acid sequence of SEQ ID NO: 158; or
lxxv. a light chain variable region comprising an amino acid sequence at least 85% identical to the amino acid sequence of SEQ ID NO: 163, and a heavy chain variable region comprising an amino acid sequence at least 85% identical to the amino acid sequence of SEQ ID NO: 158; or
lxxvi. a light chain variable region comprising an amino acid sequence at least 85% identical to the amino acid sequence of SEQ ID NO: 173, and a heavy chain variable region comprising an amino acid sequence at least 85% identical to the amino acid sequence of SEQ ID NO: 158; or
lxxvii. a light chain variable region comprising an amino acid sequence at least 85% identical to the amino acid sequence of SEQ ID NO: 183, and a heavy chain variable region comprising an amino acid sequence at least 85% identical to the amino acid sequence of SEQ ID NO: 158; or
lxxviii. a light chain variable region comprising an amino acid sequence at least 85% identical to the amino acid sequence of SEQ ID NO: 193, and a heavy chain variable region comprising an amino acid sequence at least 85% identical to the amino acid sequence of SEQ ID NO: 158; or
lxxix. a light chain variable region comprising an amino acid sequence at least 85% identical to the amino acid sequence of SEQ ID NO: 213, and a heavy chain variable region comprising an amino acid sequence at least 85% identical to the amino acid sequence of SEQ ID NO: 158; or
lxxx. a light chain variable region comprising an amino acid sequence at least 85% identical to the amino acid sequence of SEQ ID NO: 223, and a heavy chain variable region comprising an amino acid sequence at least 85% identical to the amino acid sequence of SEQ ID NO: 158; or
lxxxi. a light chain variable region comprising an amino acid sequence at least 85% identical to the amino acid sequence of SEQ ID NO: 233, and a heavy chain variable region comprising an amino acid sequence at least 85% identical to the amino acid sequence of SEQ ID NO: 158; or
lxxxii. a light chain variable region comprising an amino acid sequence at least 85% identical to the amino acid sequence of SEQ ID NO: 243, and a heavy chain variable region comprising an amino acid sequence at least 85% identical to the amino acid sequence of SEQ ID NO: 158; or
lxxxiii. a light chain variable region comprising an amino acid sequence at least 85% identical to the amino acid sequence of SEQ ID NO: 253, and a heavy chain variable region comprising an amino acid sequence at least 85% identical to the amino acid sequence of SEQ ID NO: 158; or
lxxxiv. a light chain variable region comprising an amino acid sequence at least 85% identical to the amino acid sequence of SEQ ID NO: 103, and a heavy chain variable region comprising an amino acid sequence at least 85% identical to the amino acid sequence of SEQ ID NO: 168; or
lxxxv. a light chain variable region comprising an amino acid sequence at least 85% identical to the amino acid sequence of SEQ ID NO: 113, and a heavy chain variable region comprising an amino acid sequence at least 85% identical to the amino acid sequence of SEQ ID NO: 168; or
lxxxvi. a light chain variable region comprising an amino acid sequence at least 85% identical to the amino acid sequence of SEQ ID NO: 123, and a heavy chain variable region comprising an amino acid sequence at least 85% identical to the amino acid sequence of SEQ ID NO: 168; or
lxxxvii. a light chain variable region comprising an amino acid sequence at least 85% identical to the amino acid sequence of SEQ ID NO: 163, and a heavy chain variable region comprising an amino acid sequence at least 85% identical to the amino acid sequence of SEQ ID NO: 168; or
lxxxviii. a light chain variable region comprising an amino acid sequence at least 85% identical to the amino acid sequence of SEQ ID NO: 173, and a heavy chain variable region comprising an amino acid sequence at least 85% identical to the amino acid sequence of SEQ ID NO: 168; or
lxxxix. a light chain variable region comprising an amino acid sequence at least 85% identical to the amino acid sequence of SEQ ID NO: 183, and a heavy chain variable region comprising an amino acid sequence at least 85% identical to the amino acid sequence of SEQ ID NO: 168; or
xc. a light chain variable region comprising an amino acid sequence at least 85% identical to the amino acid sequence of SEQ ID NO: 193, and a heavy chain variable region comprising an amino acid sequence at least 85% identical to the amino acid sequence of SEQ ID NO: 168; or
xci. a light chain variable region comprising an amino acid sequence at least 85% identical to the amino acid sequence of SEQ ID NO: 203, and a heavy chain variable region comprising an amino acid sequence at least 85% identical to the amino acid sequence of SEQ ID NO: 168; or
xcii. a light chain variable region comprising an amino acid sequence at least 85% identical to the amino acid sequence of SEQ ID NO: 213, and a heavy chain variable region comprising an amino acid sequence at least 85% identical to the amino acid sequence of SEQ ID NO: 168; or
xciii. a light chain variable region comprising an amino acid sequence at least 85% identical to the amino acid sequence of SEQ ID NO: 223, and a heavy chain variable region comprising an amino acid sequence at least 85% identical to the amino acid sequence of SEQ ID NO: 168; or
xciv. a light chain variable region comprising an amino acid sequence at least 85% identical to the amino acid sequence of SEQ ID NO: 233, and a heavy chain variable region comprising an amino acid sequence at least 85% identical to the amino acid sequence of SEQ ID NO: 168; or
xcv. a light chain variable region comprising an amino acid sequence at least 85% identical to the amino acid sequence of SEQ ID NO: 243, and a heavy chain variable region comprising an amino acid sequence at least 85% identical to the amino acid sequence of SEQ ID NO: 168; or
xcvi. a light chain variable region comprising an amino acid sequence at least 85% identical to the amino acid sequence of SEQ ID NO: 253, and a heavy chain variable region comprising an amino acid sequence at least 85% identical to the amino acid sequence of SEQ ID NO: 168; or
xcvii. a light chain variable region comprising an amino acid sequence at least 85% identical to the amino acid sequence of SEQ ID NO: 103, and a heavy chain variable region comprising an amino acid sequence at least 85% identical to the amino acid sequence of SEQ ID NO: 178; or
xcviii. a light chain variable region comprising an amino acid sequence at least 85% identical to the amino acid sequence of SEQ ID NO: 113, and a heavy chain variable region comprising an amino acid sequence at least 85% identical to the amino acid sequence of SEQ ID NO: 178; or
xcix. a light chain variable region comprising an amino acid sequence at least 85% identical to the amino acid sequence of SEQ ID NO: 123, and a heavy chain variable region comprising an amino acid sequence at least 85% identical to the amino acid sequence of SEQ ID NO: 178; or
c. a light chain variable region comprising an amino acid sequence at least 85% identical to the amino acid sequence of SEQ ID NO: 153, and a heavy chain variable region comprising an amino acid sequence at least 85% identical to the amino acid sequence of SEQ ID NO: 178; or
ci. a light chain variable region comprising an amino acid sequence at least 85% identical to the amino acid sequence of SEQ ID NO: 163, and a heavy chain variable region comprising an amino acid sequence at least 85% identical to the amino acid sequence of SEQ ID NO: 178; or
cii. a light chain variable region comprising an amino acid sequence at least 85% identical to the amino acid sequence of SEQ ID NO: 173, and a heavy chain variable region comprising an amino acid sequence at least 85% identical to the amino acid sequence of SEQ ID NO: 178; or
ciii. a light chain variable region comprising an amino acid sequence at least 85% identical to the amino acid sequence of SEQ ID NO: 183, and a heavy chain variable region comprising an amino acid sequence at least 85% identical to the amino acid sequence of SEQ ID NO: 178; or
civ. a light chain variable region comprising an amino acid sequence at least 85% identical to the amino acid sequence of SEQ ID NO: 193, and a heavy chain variable region comprising an amino acid sequence at least 85% identical to the amino acid sequence of SEQ ID NO: 178; or
cv. a light chain variable region comprising an amino acid sequence at least 85% identical to the amino acid sequence of SEQ ID NO: 203, and a heavy chain variable region comprising an amino acid sequence at least 85% identical to the amino acid sequence of SEQ ID NO: 178; or
cvi. a light chain variable region comprising an amino acid sequence at least 85% identical to the amino acid sequence of SEQ ID NO: 213, and a heavy chain variable region comprising an amino acid sequence at least 85% identical to the amino acid sequence of SEQ ID NO: 178; or
cvii. a light chain variable region comprising an amino acid sequence at least 85% identical to the amino acid sequence of SEQ ID NO: 223, and a heavy chain variable region comprising an amino acid sequence at least 85% identical to the amino acid sequence of SEQ ID NO: 178; or
cviii. a light chain variable region comprising an amino acid sequence at least 85% identical to the amino acid sequence of SEQ ID NO: 233, and a heavy chain variable region comprising an amino acid sequence at least 85% identical to the amino acid sequence of SEQ ID NO: 178; or
cix. a light chain variable region comprising an amino acid sequence at least 85% identical to the amino acid sequence of SEQ ID NO: 243, and a heavy chain variable region comprising an amino acid sequence at least 85% identical to the amino acid sequence of SEQ ID NO: 178.

5. The method according to claim 1, wherein the anti-CTGF antibody or an antigen-binding fragment thereof comprises:
i. a light chain variable region comprising an amino acid sequence at least 90% identical to the amino acid sequence of SEQ ID NO: 223, and a heavy chain variable region comprising an amino acid sequence at least 90% identical to the amino acid sequence of SEQ ID NO: 108; or
ii. a light chain variable region comprising an amino acid sequence at least 90% identical to the amino acid sequence of SEQ ID NO: 103, and a heavy chain variable region comprising an amino acid sequence at least 90% identical to the amino acid sequence of SEQ ID NO: 108; or
iii. a light chain variable region comprising an amino acid sequence at least 90% identical to the amino acid sequence of SEQ ID NO: 113, and a heavy chain variable region comprising an amino acid sequence at least 90% identical to the amino acid sequence of SEQ ID NO: 108; or
iv. a light chain variable region comprising an amino acid sequence at least 90% identical to the amino acid sequence of SEQ ID NO: 123, and a heavy chain variable region comprising an amino acid sequence at least 90% identical to the amino acid sequence of SEQ ID NO: 108; or
v. a light chain variable region comprising an amino acid sequence at least 90% identical to the amino acid sequence of SEQ ID NO: 133, and a heavy chain variable region comprising an amino acid sequence at least 90% identical to the amino acid sequence of SEQ ID NO: 108; or
vi. a light chain variable region comprising an amino acid sequence at least 90% identical to the amino acid sequence of SEQ ID NO: 153, and a heavy chain variable region comprising an amino acid sequence at least 90% identical to the amino acid sequence of SEQ ID NO: 108; or
vii. a light chain variable region comprising an amino acid sequence at least 90% identical to the amino acid sequence of SEQ ID NO: 163, and a heavy chain variable region comprising an amino acid sequence at least 90% identical to the amino acid sequence of SEQ ID NO: 108; or
viii a light chain variable region comprising an amino acid sequence at least 90% identical to the amino acid sequence of SEQ ID NO: 173, and a heavy chain variable region comprising an amino acid sequence at least 90% identical to the amino acid sequence of SEQ ID NO: 108; or
ix. a light chain variable region comprising an amino acid sequence at least 90% identical to the amino acid sequence of SEQ ID NO: 183, and a heavy chain variable region comprising an amino acid sequence at least 90% identical to the amino acid sequence of SEQ ID NO: 108; or
x. a light chain variable region comprising an amino acid sequence at least 90% identical to the amino acid sequence of SEQ ID NO: 193, and a heavy chain variable region comprising an amino acid sequence at least 90% identical to the amino acid sequence of SEQ ID NO: 108; or
xi. a light chain variable region comprising an amino acid sequence at least 90% identical to the amino acid sequence of SEQ ID NO: 203, and a heavy chain variable region comprising an amino acid sequence at least 90% identical to the amino acid sequence of SEQ ID NO: 108; or
xii. a light chain variable region comprising an amino acid sequence at least 90% identical to the amino acid sequence of SEQ ID NO: 213, and a heavy chain variable region comprising an amino acid sequence at least 90% identical to the amino acid sequence of SEQ ID NO: 108; or
xiii. a light chain variable region comprising an amino acid sequence at least 90% identical to the amino acid sequence of SEQ ID NO: 233, and a heavy chain variable region comprising an amino acid sequence at least 90% identical to the amino acid sequence of SEQ ID NO: 108; or
xiv. a light chain variable region comprising an amino acid sequence at least 90% identical to the amino acid sequence of SEQ ID NO: 243, and a heavy chain variable region comprising an amino acid sequence at least 90% identical to the amino acid sequence of SEQ ID NO: 108; or
xv. a light chain variable region comprising an amino acid sequence at least 90% identical to the amino acid sequence of SEQ ID NO: 253, and a heavy chain variable region comprising an amino acid sequence at least 90% identical to the amino acid sequence of SEQ ID NO: 108; or
xvi. a light chain variable region comprising an amino acid sequence at least 90% identical to the amino acid sequence of SEQ ID NO: 103, and a heavy chain variable region comprising an amino acid sequence at least 90% identical to the amino acid sequence of SEQ ID NO: 118; or
xvii. a light chain variable region comprising an amino acid sequence at least 90% identical to the amino acid sequence of SEQ ID NO: 113, and a heavy chain variable region comprising an amino acid sequence at least 90% identical to the amino acid sequence of SEQ ID NO: 118; or
xviii. a light chain variable region comprising an amino acid sequence at least 90% identical to the amino acid sequence of SEQ ID NO: 123, and a heavy chain variable region comprising an amino acid sequence at least 90% identical to the amino acid sequence of SEQ ID NO: 118; or
xix. a light chain variable region comprising an amino acid sequence at least 90% identical to the amino acid sequence of SEQ ID NO: 153, and a heavy chain variable region comprising an amino acid sequence at least 90% identical to the amino acid sequence of SEQ ID NO: 118; or
xx. a light chain variable region comprising an amino acid sequence at least 90% identical to the amino acid sequence of SEQ ID NO: 163, and a heavy chain variable region comprising an amino acid sequence at least 90% identical to the amino acid sequence of SEQ ID NO: 118; or
xxi. a light chain variable region comprising an amino acid sequence at least 90% identical to the amino acid sequence of SEQ ID NO: 173, and a heavy chain variable region comprising an amino acid sequence at least 90% identical to the amino acid sequence of SEQ ID NO: 118; or
xxii. a light chain variable region comprising an amino acid sequence at least 90% identical to the amino acid sequence of SEQ ID NO: 183, and a heavy chain variable region comprising an amino acid sequence at least 90% identical to the amino acid sequence of SEQ ID NO: 118; or
xxiii. a light chain variable region comprising an amino acid sequence at least 90% identical to the amino acid sequence of SEQ ID NO: 193, and a heavy chain variable region comprising an amino acid sequence at least 90% identical to the amino acid sequence of SEQ ID NO: 118; or
xxiv. a light chain variable region comprising an amino acid sequence at least 90% identical to the amino acid sequence of SEQ ID NO: 203, and a heavy chain variable region comprising an amino acid sequence at least 90% identical to the amino acid sequence of SEQ ID NO: 118; or
xxv. a light chain variable region comprising an amino acid sequence at least 90% identical to the amino acid sequence of SEQ ID NO: 213, and a heavy chain variable region comprising an amino acid sequence at least 90% identical to the amino acid sequence of SEQ ID NO: 118; or
xxvi. a light chain variable region comprising an amino acid sequence at least 90% identical to the amino acid sequence of SEQ ID NO: 223, and a heavy chain variable region comprising an amino acid sequence at least 90% identical to the amino acid sequence of SEQ ID NO: 118; or
xxvii. a light chain variable region comprising an amino acid sequence at least 90% identical to the amino acid sequence of SEQ ID NO: 233, and a heavy chain variable region comprising an amino acid sequence at least 90% identical to the amino acid sequence of SEQ ID NO: 118; or
xxviii. a light chain variable region comprising an amino acid sequence at least 90% identical to the amino acid sequence of SEQ ID NO: 243, and a heavy chain variable region comprising an amino acid sequence at least 90% identical to the amino acid sequence of SEQ ID NO: 118; or
xxix. a light chain variable region comprising an amino acid sequence at least 90% identical to the amino acid sequence of SEQ ID NO: 253, and a heavy chain variable region comprising an amino acid sequence at least 90% identical to the amino acid sequence of SEQ ID NO: 118; or
xxx. a light chain variable region comprising an amino acid sequence at least 90% identical to the amino acid sequence of SEQ ID NO: 103, and a heavy chain variable region comprising an amino acid sequence at least 90% identical to the amino acid sequence of SEQ ID NO: 128; or
xxxi. a light chain variable region comprising an amino acid sequence at least 90% identical to the amino acid sequence of SEQ ID NO: 113, and a heavy chain variable region comprising an amino acid sequence at least 90% identical to the amino acid sequence of SEQ ID NO: 128; or
xxxii. a light chain variable region comprising an amino acid sequence at least 90% identical to the amino acid sequence of SEQ ID NO: 123, and a heavy chain variable region comprising an amino acid sequence at least 90% identical to the amino acid sequence of SEQ ID NO: 128; or
xxxiii. a light chain variable region comprising an amino acid sequence at least 90% identical to the amino acid sequence of SEQ ID NO: 153, and a heavy chain variable region comprising an amino acid sequence at least 90% identical to the amino acid sequence of SEQ ID NO: 128; or
xxxiv. a light chain variable region comprising an amino acid sequence at least 90% identical to the amino acid sequence of SEQ ID NO: 163, and a heavy chain variable region comprising an amino acid sequence at least 90% identical to the amino acid sequence of SEQ ID NO: 128; or
xxxv. a light chain variable region comprising an amino acid sequence at least 90% identical to the amino acid sequence of SEQ ID NO: 173, and a heavy chain variable region comprising an amino acid sequence at least 90% identical to the amino acid sequence of SEQ ID NO: 128; or
xxxvi. a light chain variable region comprising an amino acid sequence at least 90% identical to the amino acid sequence of SEQ ID NO: 183, and a heavy chain variable region comprising an amino acid sequence at least 90% identical to the amino acid sequence of SEQ ID NO: 128; or
xxxvii. a light chain variable region comprising an amino acid sequence at least 90% identical to the amino acid sequence of SEQ ID NO: 193, and a heavy chain variable region comprising an amino acid sequence at least 90% identical to the amino acid sequence of SEQ ID NO: 128; or
xxxviii. a light chain variable region comprising an amino acid sequence at least 90% identical to the amino acid sequence of SEQ ID NO: 203, and a heavy chain variable region comprising an amino acid sequence at least 90% identical to the amino acid sequence of SEQ ID NO: 128; or
xxxix. a light chain variable region comprising an amino acid sequence at least 90% identical to the amino acid sequence of SEQ ID NO: 213, and a heavy chain variable region comprising an amino acid sequence at least 90% identical to the amino acid sequence of SEQ ID NO: 128; or
xl. a light chain variable region comprising an amino acid sequence at least 90% identical to the amino acid sequence of SEQ ID NO: 223, and a heavy chain variable region comprising an amino acid sequence at least 90% identical to the amino acid sequence of SEQ ID NO: 128; or
xli. a light chain variable region comprising an amino acid sequence at least 90% identical to the amino acid sequence of SEQ ID NO: 233, and a heavy chain variable region comprising an amino acid sequence at least 90% identical to the amino acid sequence of SEQ ID NO: 128; or
xlii. a light chain variable region comprising an amino acid sequence at least 90% identical to the amino acid sequence of SEQ ID NO: 243, and a heavy chain variable region comprising an amino acid sequence at least 90% identical to the amino acid sequence of SEQ ID NO: 128; or
xliii. a light chain variable region comprising an amino acid sequence at least 90% identical to the amino acid sequence of SEQ ID NO: 253, and a heavy chain variable region comprising an amino acid sequence at least 90% identical to the amino acid sequence of SEQ ID NO: 128; or
xliv. a light chain variable region comprising an amino acid sequence at least 90% identical to the amino acid sequence of SEQ ID NO: 103, and a heavy chain variable region comprising an amino acid sequence at least 90% identical to the amino acid sequence of SEQ ID NO: 138; or
xlv. a light chain variable region comprising an amino acid sequence at least 90% identical to the amino acid sequence of SEQ ID NO: 113, and a heavy chain variable region comprising an amino acid sequence at least 90% identical to the amino acid sequence of SEQ ID NO: 138; or
xlvi. a light chain variable region comprising an amino acid sequence at least 90% identical to the amino acid sequence of SEQ ID NO: 123, and a heavy chain variable region comprising an amino acid sequence at least 90% identical to the amino acid sequence of SEQ ID NO: 138; or
xlvii. a light chain variable region comprising an amino acid sequence at least 90% identical to the amino acid sequence of SEQ ID NO: 153, and a heavy chain variable region comprising an amino acid sequence at least 90% identical to the amino acid sequence of SEQ ID NO: 138; or
xlviii. a light chain variable region comprising an amino acid sequence at least 90% identical to the amino acid sequence of SEQ ID NO: 163, and a heavy chain variable region comprising an amino acid sequence at least 90% identical to the amino acid sequence of SEQ ID NO: 138; or
xlix. a light chain variable region comprising an amino acid sequence at least 90% identical to the amino acid sequence of SEQ ID NO: 173, and a heavy chain variable region comprising an amino acid sequence at least 90% identical to the amino acid sequence of SEQ ID NO: 138; or
l. a light chain variable region comprising an amino acid sequence at least 90% identical to the amino acid sequence of SEQ ID NO: 183, and a heavy chain variable region comprising an amino acid sequence at least 90% identical to the amino acid sequence of SEQ ID NO: 138; or
li. a light chain variable region comprising an amino acid sequence at least 90% identical to the amino acid sequence of SEQ ID NO: 193, and a heavy chain variable region comprising an amino acid sequence at least 90% identical to the amino acid sequence of SEQ ID NO: 138; or
lii. a light chain variable region comprising an amino acid sequence at least 90% identical to the amino acid sequence of SEQ ID NO: 203, and a heavy chain variable region comprising an amino acid sequence at least 90% identical to the amino acid sequence of SEQ ID NO: 138; or
liii. a light chain variable region comprising an amino acid sequence at least 90% identical to the amino acid sequence of SEQ ID NO: 213, and a heavy chain variable region comprising an amino acid sequence at least 90% identical to the amino acid sequence of SEQ ID NO: 138; or
liv. a light chain variable region comprising an amino acid sequence at least 90% identical to the amino acid sequence of SEQ ID NO: 223, and a heavy chain variable region comprising an amino acid sequence at least 90% identical to the amino acid sequence of SEQ ID NO: 138; or
lv. a light chain variable region comprising an amino acid sequence at least 90% identical to the amino acid sequence of SEQ ID NO: 233, and a heavy chain variable region comprising an amino acid sequence at least 90% identical to the amino acid sequence of SEQ ID NO: 138; or
lvi. a light chain variable region comprising an amino acid sequence at least 90% identical to the amino acid sequence of SEQ ID NO: 243, and a heavy chain variable region comprising an amino acid sequence at least 90% identical to the amino acid sequence of SEQ ID NO: 138; or
lvii. a light chain variable region comprising an amino acid sequence at least 90% identical to the amino acid sequence of SEQ ID NO: 253, and a heavy chain variable region comprising an amino acid sequence at least 90% identical to the amino acid sequence of SEQ ID NO: 138; or
lviii. a light chain variable region comprising an amino acid sequence at least 90% identical to the amino acid sequence of SEQ ID NO: 103, and a heavy chain variable region comprising an amino acid sequence at least 90% identical to the amino acid sequence of SEQ ID NO: 148; or
lix. a light chain variable region comprising an amino acid sequence at least 90% identical to the amino acid sequence of SEQ ID NO: 113, and a heavy chain variable region comprising an amino acid sequence at least 90% identical to the amino acid sequence of SEQ ID NO: 148; or
lx. a light chain variable region comprising an amino acid sequence at least 90% identical to the amino acid sequence of SEQ ID NO: 123, and a heavy chain variable region comprising an amino acid sequence at least 90% identical to the amino acid sequence of SEQ ID NO: 148; or
lxi. a light chain variable region comprising an amino acid sequence at least 90% identical to the amino acid sequence of SEQ ID NO: 163, and a heavy chain variable region comprising an amino acid sequence at least 90% identical to the amino acid sequence of SEQ ID NO: 148; or
lxii. a light chain variable region comprising an amino acid sequence at least 90% identical to the amino acid sequence of SEQ ID NO: 173, and a heavy chain variable region comprising an amino acid sequence at least 90% identical to the amino acid sequence of SEQ ID NO: 148; or
lxiii. a light chain variable region comprising an amino acid sequence at least 90% identical to the amino acid sequence of SEQ ID NO: 183, and a heavy chain variable region comprising an amino acid sequence at least 90% identical to the amino acid sequence of SEQ ID NO: 148; or
lxiv. a light chain variable region comprising an amino acid sequence at least 90% identical to the amino acid sequence of SEQ ID NO: 193, and a heavy chain variable region comprising an amino acid sequence at least 90% identical to the amino acid sequence of SEQ ID NO: 148; or
lxv. a light chain variable region comprising an amino acid sequence at least 90% identical to the amino acid sequence of SEQ ID NO: 203, and a heavy chain variable region comprising an amino acid sequence at least 90% identical to the amino acid sequence of SEQ ID NO: 148; or
lxvi. a light chain variable region comprising an amino acid sequence at least 90% identical to the amino acid sequence of SEQ ID NO: 213, and a heavy chain variable region comprising an amino acid sequence at least 90% identical to the amino acid sequence of SEQ ID NO: 148; or
lxvii. a light chain variable region comprising an amino acid sequence at least 90% identical to the amino acid sequence of SEQ ID NO: 223, and a heavy chain variable region comprising an amino acid sequence at least 90% identical to the amino acid sequence of SEQ ID NO: 148; or
lxviii. a light chain variable region comprising an amino acid sequence at least 90% identical to the amino acid sequence of SEQ ID NO: 233, and a heavy chain variable region comprising an amino acid sequence at least 90% identical to the amino acid sequence of SEQ ID NO: 148; or
lxix. a light chain variable region comprising an amino acid sequence at least 90% identical to the amino acid sequence of SEQ ID NO: 243, and a heavy chain variable region comprising an amino acid sequence at least 90% identical to the amino acid sequence of SEQ ID NO: 148; or
lxx. a light chain variable region comprising an amino acid sequence at least 90% identical to the amino acid sequence of SEQ ID NO: 253, and a heavy chain variable region comprising an amino acid sequence at least 90% identical to the amino acid sequence of SEQ ID NO: 148; or
lxxi. a light chain variable region comprising an amino acid sequence at least 90% identical to the amino acid sequence of SEQ ID NO: 103, and a heavy chain variable region comprising an amino acid sequence at least 90% identical to the amino acid sequence of SEQ ID NO: 158; or
lxxii. a light chain variable region comprising an amino acid sequence at least 90% identical to the amino acid sequence of SEQ ID NO: 113, and a heavy chain variable region comprising an amino acid sequence at least 90% identical to the amino acid sequence of SEQ ID NO: 158; or
lxxiii. a light chain variable region comprising an amino acid sequence at least 90% identical to the amino acid sequence of SEQ ID NO: 123, and a heavy chain variable region comprising an amino acid sequence at least 90% identical to the amino acid sequence of SEQ ID NO: 158; or
lxxiv. a light chain variable region comprising an amino acid sequence at least 90% identical to the amino acid sequence of SEQ ID NO: 153, and a heavy chain variable region comprising an amino acid sequence at least 90% identical to the amino acid sequence of SEQ ID NO: 158; or
lxxv. a light chain variable region comprising an amino acid sequence at least 90% identical to the amino acid sequence of SEQ ID NO: 163, and a heavy chain variable region comprising an amino acid sequence at least 90% identical to the amino acid sequence of SEQ ID NO: 158; or
lxxvi. a light chain variable region comprising an amino acid sequence at least 90% identical to the amino acid sequence of SEQ ID NO: 173, and a heavy chain variable region comprising an amino acid sequence at least 90% identical to the amino acid sequence of SEQ ID NO: 158; or
lxxvii. a light chain variable region comprising an amino acid sequence at least 90% identical to the amino acid sequence of SEQ ID NO: 183, and a heavy chain variable region comprising an amino acid sequence at least 90% identical to the amino acid sequence of SEQ ID NO: 158; or
lxxviii. a light chain variable region comprising an amino acid sequence at least 90% identical to the amino acid sequence of SEQ ID NO: 193, and a heavy chain variable region comprising an amino acid sequence at least 90% identical to the amino acid sequence of SEQ ID NO: 158; or
lxxix. a light chain variable region comprising an amino acid sequence at least 90% identical to the amino acid sequence of SEQ ID NO: 213, and a heavy chain variable region comprising an amino acid sequence at least 90% identical to the amino acid sequence of SEQ ID NO: 158; or
lxxx. a light chain variable region comprising an amino acid sequence at least 90% identical to the amino acid sequence of SEQ ID NO: 223, and a heavy chain variable region comprising an amino acid sequence at least 90% identical to the amino acid sequence of SEQ ID NO: 158; or
lxxxi. a light chain variable region comprising an amino acid sequence at least 90% identical to the amino acid sequence of SEQ ID NO: 233, and a heavy chain variable region comprising an amino acid sequence at least 90% identical to the amino acid sequence of SEQ ID NO: 158; or
xxxii. a light chain variable region comprising an amino acid sequence at least 90% identical to the amino acid sequence of SEQ ID NO: 243, and a heavy chain variable region comprising an amino acid sequence at least 90% identical to the amino acid sequence of SEQ ID NO: 158; or
lxxxiii. a light chain variable region comprising an amino acid sequence at least 90% identical to the amino acid sequence of SEQ ID NO: 253, and a heavy chain variable region comprising an amino acid sequence at least 90% identical to the amino acid sequence of SEQ ID NO: 158; or
lxxxiv. a light chain variable region comprising an amino acid sequence at least 90% identical to the amino acid sequence of SEQ ID NO: 103, and a heavy chain variable region comprising an amino acid sequence at least 90% identical to the amino acid sequence of SEQ ID NO: 168; or
lxxxv. a light chain variable region comprising an amino acid sequence at least 90% identical to the amino acid sequence of SEQ ID NO: 113, and a heavy chain variable region comprising an amino acid sequence at least 90% identical to the amino acid sequence of SEQ ID NO: 168; or
lxxxvi. a light chain variable region comprising an amino acid sequence at least 90% identical to the amino acid sequence of SEQ ID NO: 123, and a heavy chain variable region comprising an amino acid sequence at least 90% identical to the amino acid sequence of SEQ ID NO: 168; or
lxxxvii. a light chain variable region comprising an amino acid sequence at least 90% identical to the amino acid sequence of SEQ ID NO: 163, and a heavy chain variable region comprising an amino acid sequence at least 90% identical to the amino acid sequence of SEQ ID NO: 168; or
lxxxviii. a light chain variable region comprising an amino acid sequence at least 90% identical to the amino acid sequence of SEQ ID NO: 173, and a heavy chain variable region comprising an amino acid sequence at least 90% identical to the amino acid sequence of SEQ ID NO: 168; or
lxxxix. a light chain variable region comprising an amino acid sequence at least 90% identical to the amino acid sequence of SEQ ID NO: 183, and a heavy chain variable region comprising an amino acid sequence at least 90% identical to the amino acid sequence of SEQ ID NO: 168; or
xc. a light chain variable region comprising an amino acid sequence at least 90% identical to the amino acid sequence of SEQ ID NO: 193, and a heavy chain variable region comprising an amino acid sequence at least 90% identical to the amino acid sequence of SEQ ID NO: 168; or
xci. a light chain variable region comprising an amino acid sequence at least 90% identical to the amino acid sequence of SEQ ID NO: 203, and a heavy chain variable region comprising an amino acid sequence at least 90% identical to the amino acid sequence of SEQ ID NO: 168; or
xcii. a light chain variable region comprising an amino acid sequence at least 90% identical to the amino acid sequence of SEQ ID NO: 213, and a heavy chain variable region comprising an amino acid sequence at least 90% identical to the amino acid sequence of SEQ ID NO: 168; or
xciii. a light chain variable region comprising an amino acid sequence at least 90% identical to the amino acid sequence of SEQ ID NO: 223, and a heavy chain variable region comprising an amino acid sequence at least 90% identical to the amino acid sequence of SEQ ID NO: 168; or
xciv. a light chain variable region comprising an amino acid sequence at least 90% identical to the amino acid sequence of SEQ ID NO: 233, and a heavy chain variable region comprising an amino acid sequence at least 90% identical to the amino acid sequence of SEQ ID NO: 168; or
xcv. a light chain variable region comprising an amino acid sequence at least 90% identical to the amino acid sequence of SEQ ID NO: 243, and a heavy chain variable region comprising an amino acid sequence at least 90% identical to the amino acid sequence of SEQ ID NO: 168; or
xcvi. a light chain variable region comprising an amino acid sequence at least 90% identical to the amino acid sequence of SEQ ID NO: 253, and a heavy chain variable region comprising an amino acid sequence at least 90% identical to the amino acid sequence of SEQ ID NO: 168; or
xcvii. a light chain variable region comprising an amino acid sequence at least 90% identical to the amino acid sequence of SEQ ID NO: 103, and a heavy chain variable region comprising an amino acid sequence at least 90% identical to the amino acid sequence of SEQ ID NO: 178; or
xcviii. a light chain variable region comprising an amino acid sequence at least 90% identical to the amino acid sequence of SEQ ID NO: 113, and a heavy chain variable region comprising an amino acid sequence at least 90% identical to the amino acid sequence of SEQ ID NO: 178; or
xcix. a light chain variable region comprising an amino acid sequence at least 90% identical to the amino acid sequence of SEQ ID NO: 123, and a heavy chain variable region comprising an amino acid sequence at least 90% identical to the amino acid sequence of SEQ ID NO: 178; or
c. a light chain variable region comprising an amino acid sequence at least 90% identical to the amino acid sequence of SEQ ID NO: 153, and a heavy chain variable region comprising an amino acid sequence at least 90% identical to the amino acid sequence of SEQ ID NO: 178; or
ci. a light chain variable region comprising an amino acid sequence at least 90% identical to the amino acid sequence of SEQ ID NO: 163, and a heavy chain variable region comprising an amino acid sequence at least 90% identical to the amino acid sequence of SEQ ID NO: 178; or
cii. a light chain variable region comprising an amino acid sequence at least 90% identical to the amino acid sequence of SEQ ID NO: 173, and a heavy chain variable region comprising an amino acid sequence at least 90% identical to the amino acid sequence of SEQ ID NO: 178; or
ciii. a light chain variable region comprising an amino acid sequence at least 90% identical to the amino acid sequence of SEQ ID NO: 183, and a heavy chain variable region comprising an amino acid sequence at least 90% identical to the amino acid sequence of SEQ ID NO: 178; or
civ. a light chain variable region comprising an amino acid sequence at least 90% identical to the amino acid sequence of SEQ ID NO: 193, and a heavy chain variable region comprising an amino acid sequence at least 90% identical to the amino acid sequence of SEQ ID NO: 178; or
cv. a light chain variable region comprising an amino acid sequence at least 90% identical to the amino acid sequence of SEQ ID NO: 203, and a heavy chain variable region comprising an amino acid sequence at least 90% identical to the amino acid sequence of SEQ ID NO: 178; or
cvi. a light chain variable region comprising an amino acid sequence at least 90% identical to the amino acid sequence of SEQ ID NO: 213, and a heavy chain variable region comprising an amino acid sequence at least 90% identical to the amino acid sequence of SEQ ID NO: 178; or
cvii. a light chain variable region comprising an amino acid sequence at least 90% identical to the amino acid sequence of SEQ ID NO: 223, and a heavy chain variable region comprising an amino acid sequence at least 90% identical to the amino acid sequence of SEQ ID NO: 178; or
cviii. a light chain variable region comprising an amino acid sequence at least 90% identical to the amino acid sequence of SEQ ID NO: 233, and a heavy chain variable region comprising an amino acid sequence at least 90% identical to the amino acid sequence of SEQ ID NO: 178; or
cix. a light chain variable region comprising an amino acid sequence at least 90% identical to the amino acid sequence of SEQ ID NO: 243, and a heavy chain variable region comprising an amino acid sequence at least 90% identical to the amino acid sequence of SEQ ID NO: 178.

6. The method according to claim 1, wherein the anti-CTGF antibody or an antigen-binding fragment thereof comprises:
i. a light chain variable region comprising an amino acid sequence at least 95% identical to the amino acid sequence of SEQ ID NO: 223, and a heavy chain variable region comprising an amino acid sequence at least 95% identical to the amino acid sequence of SEQ ID NO: 108; or
ii. a light chain variable region comprising an amino acid sequence at least 95% identical to the amino acid sequence of SEQ ID NO: 103, and a heavy chain variable region comprising an amino acid sequence at least 95% identical to the amino acid sequence of SEQ ID NO: 108; or
iii. a light chain variable region comprising an amino acid sequence at least 95% identical to the amino acid sequence of SEQ ID NO: 113, and a heavy chain variable region comprising an amino acid sequence at least 95% identical to the amino acid sequence of SEQ ID NO: 108; or
iv. a light chain variable region comprising an amino acid sequence at least 95% identical to the amino acid sequence of SEQ ID NO: 123, and a heavy chain variable region comprising an amino acid sequence at least 95% identical to the amino acid sequence of SEQ ID NO: 108; or
v. a light chain variable region comprising an amino acid sequence at least 95% identical to the amino acid sequence of SEQ ID NO: 133, and a heavy chain variable region comprising an amino acid sequence at least 95% identical to the amino acid sequence of SEQ ID NO: 108; or
vi. a light chain variable region comprising an amino acid sequence at least 95% identical to the amino acid sequence of SEQ ID NO: 153, and a heavy chain variable region comprising an amino acid sequence at least 95% identical to the amino acid sequence of SEQ ID NO: 108; or
vii. a light chain variable region comprising an amino acid sequence at least 95% identical to the amino acid sequence of SEQ ID NO: 163, and a heavy chain variable region comprising an amino acid sequence at least 95% identical to the amino acid sequence of SEQ ID NO: 108; or
viii. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 108; or
ix. a light chain variable region comprising an amino acid sequence at least 95% identical to the amino acid sequence of SEQ ID NO: 183, and a heavy chain variable region comprising an amino acid sequence at least 95% identical to the amino acid sequence of SEQ ID NO: 108; or
x. a light chain variable region comprising an amino acid sequence at least 95% identical to the amino acid sequence of SEQ ID NO: 193, and a heavy chain variable region comprising an amino acid sequence at least 95% identical to the amino acid sequence of SEQ ID NO: 108; or
xi. a light chain variable region comprising an amino acid sequence at least 95% identical to the amino acid sequence of SEQ ID NO: 203, and a heavy chain variable region comprising an amino acid sequence at least 95% identical to the amino acid sequence of SEQ ID NO: 108; or
xii. a light chain variable region comprising an amino acid sequence at least 95% identical to the amino acid sequence of SEQ ID NO: 213, and a heavy chain variable region comprising an amino acid sequence at least 95% identical to the amino acid sequence of SEQ ID NO: 108; or
xiii. a light chain variable region comprising an amino acid sequence at least 95% identical to the amino acid sequence of SEQ ID NO: 233, and a heavy chain variable region comprising an amino acid sequence at least 95% identical to the amino acid sequence of SEQ ID NO: 108; or
xiv. a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 108; or
xv. a light chain variable region comprising an amino acid sequence at least 95% identical to the amino acid sequence of SEQ ID NO: 253, and a heavy chain variable region comprising an amino acid sequence at least 95% identical to the amino acid sequence of SEQ ID NO: 108; or
xvi. a light chain variable region comprising an amino acid sequence at least 95% identical to the amino acid sequence of SEQ ID NO: 103, and a heavy chain variable region comprising an amino acid sequence at least 95% identical to the amino acid sequence of SEQ ID NO: 118; or
xvii. a light chain variable region comprising an amino acid sequence at least 95% identical to the amino acid sequence of SEQ ID NO: 113, and a heavy chain variable region comprising an amino acid sequence at least 95% identical to the amino acid sequence of SEQ ID NO: 118; or
xviii. a light chain variable region comprising an amino acid sequence at least 95% identical to the amino acid sequence of SEQ ID NO: 123, and a heavy chain variable region comprising an amino acid sequence at least 95% identical to the amino acid sequence of SEQ ID NO: 118; or
xix. a light chain variable region comprising an amino acid sequence at least 95% identical to the amino acid sequence of SEQ ID NO: 153, and a heavy chain variable region comprising an amino acid sequence at least 95% identical to the amino acid sequence of SEQ ID NO: 118; or
xx. a light chain variable region comprising an amino acid sequence at least 95% identical to the amino acid sequence of SEQ ID NO: 163, and a heavy chain variable region comprising an amino acid sequence at least 95% identical to the amino acid sequence of SEQ ID NO: 118; or
xxi. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 118; or
xxii. a light chain variable region comprising an amino acid sequence at least 95% identical to the amino acid sequence of SEQ ID NO: 183, and a heavy chain variable region comprising an amino acid sequence at least 95% identical to the amino acid sequence of SEQ ID NO: 118; or
xxiii. a light chain variable region comprising an amino acid sequence at least 95% identical to the amino acid sequence of SEQ ID NO: 193, and a heavy chain variable region comprising an amino acid sequence at least 95% identical to the amino acid sequence of SEQ ID NO: 118; or
xxiv. a light chain variable region comprising an amino acid sequence at least 95% identical to the amino acid sequence of SEQ ID NO: 203, and a heavy chain variable region comprising an amino acid sequence at least 95% identical to the amino acid sequence of SEQ ID NO: 118; or
xxv. a light chain variable region comprising an amino acid sequence at least 95% identical to the amino acid sequence of SEQ ID NO: 213, and a heavy chain variable region comprising an amino acid sequence at least 95% identical to the amino acid sequence of SEQ ID NO: 118; or
xxvi. a light chain variable region comprising an amino acid sequence at least 95% identical to the amino acid sequence of SEQ ID NO: 223, and a heavy chain variable region comprising an amino acid sequence at least 95% identical to the amino acid sequence of SEQ ID NO: 118; or
xxvii. a light chain variable region comprising an amino acid sequence at least 95% identical to the amino acid sequence of SEQ ID NO: 233, and a heavy chain variable region comprising an amino acid sequence at least 95% identical to the amino acid sequence of SEQ ID NO: 118; or
xxviii. a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 118; or
xxix. a light chain variable region comprising an amino acid sequence at least 95% identical to the amino acid sequence of SEQ ID NO: 253, and a heavy chain variable region comprising an amino acid sequence at least 95% identical to the amino acid sequence of SEQ ID NO: 118; or
xxx. a light chain variable region comprising an amino acid sequence at least 95% identical to the amino acid sequence of SEQ ID NO: 103, and a heavy chain variable region comprising an amino acid sequence at least 95% identical to the amino acid sequence of SEQ ID NO: 128; or
xxxi. a light chain variable region comprising an amino acid sequence at least 95% identical to the amino acid sequence of SEQ ID NO: 113, and a heavy chain variable region comprising an amino acid sequence at least 95% identical to the amino acid sequence of SEQ ID NO: 128; or
xxxii. a light chain variable region comprising an amino acid sequence at least 95% identical to the amino acid sequence of SEQ ID NO: 123, and a heavy chain variable region comprising an amino acid sequence at least 95% identical to the amino acid sequence of SEQ ID NO: 128; or
xxxiii. a light chain variable region comprising an amino acid sequence at least 95% identical to the amino acid sequence of SEQ ID NO: 153, and a heavy chain variable region comprising an amino acid sequence at least 95% identical to the amino acid sequence of SEQ ID NO: 128; or
xxxiv. a light chain variable region comprising an amino acid sequence at least 95% identical to the amino acid sequence of SEQ ID NO: 163, and a heavy chain variable region comprising an amino acid sequence at least 95% identical to the amino acid sequence of SEQ ID NO: 128; or
xxxv. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 128; or
xxxvi. a light chain variable region comprising an amino acid sequence at least 95% identical to the amino acid sequence of SEQ ID NO: 183, and a heavy chain variable region comprising an amino acid sequence at least 95% identical to the amino acid sequence of SEQ ID NO: 128; or
xxxvii. a light chain variable region comprising an amino acid sequence at least 95% identical to the amino acid sequence of SEQ ID NO: 193, and a heavy chain variable region comprising an amino acid sequence at least 95% identical to the amino acid sequence of SEQ ID NO: 128; or
xxxviii. a light chain variable region comprising an amino acid sequence at least 95% identical to the amino acid sequence of SEQ ID NO: 203, and a heavy chain variable region comprising an amino acid sequence at least 95% identical to the amino acid sequence of SEQ ID NO: 128; or
xxxix. a light chain variable region comprising an amino acid sequence at least 95% identical to the amino acid sequence of SEQ ID NO: 213, and a heavy chain variable region comprising an amino acid sequence at least 95% identical to the amino acid sequence of SEQ ID NO: 128; or
xl. a light chain variable region comprising an amino acid sequence at least 95% identical to the amino acid sequence of SEQ ID NO: 223, and a heavy chain variable region comprising an amino acid sequence at least 95% identical to the amino acid sequence of SEQ ID NO: 128; or
xli. a light chain variable region comprising an amino acid sequence at least 95% identical to the amino acid sequence of SEQ ID NO: 233, and a heavy chain variable region comprising an amino acid sequence at least 95% identical to the amino acid sequence of SEQ ID NO: 128; or
xlii a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 128; or
xliii. a light chain variable region comprising an amino acid sequence at least 95% identical to the amino acid sequence of SEQ ID NO: 253, and a heavy chain variable region comprising an amino acid sequence at least 95% identical to the amino acid sequence of SEQ ID NO: 128; or
xliv. a light chain variable region comprising an amino acid sequence at least 95% identical to the amino acid sequence of SEQ ID NO: 103, and a heavy chain variable region comprising an amino acid sequence at least 95% identical to the amino acid sequence of SEQ ID NO: 138; or
xlv. a light chain variable region comprising an amino acid sequence at least 95% identical to the amino acid sequence of SEQ ID NO: 113, and a heavy chain variable region comprising an amino acid sequence at least 95% identical to the amino acid sequence of SEQ ID NO: 138; or
xlvi. a light chain variable region comprising an amino acid sequence at least 95% identical to the amino acid sequence of SEQ ID NO: 123, and a heavy chain variable region comprising an amino acid sequence at least 95% identical to the amino acid sequence of SEQ ID NO: 138; or
xlvii. a light chain variable region comprising an amino acid sequence at least 95% identical to the amino acid sequence of SEQ ID NO: 153, and a heavy chain variable region comprising an amino acid sequence at least 95% identical to the amino acid sequence of SEQ ID NO: 138; or
xlviii. a light chain variable region comprising an amino acid sequence at least 95% identical to the amino acid sequence of SEQ ID NO: 163, and a heavy chain variable region comprising an amino acid sequence at least 95% identical to the amino acid sequence of SEQ ID NO: 138; or
xlix. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 138; or
l. a light chain variable region comprising an amino acid sequence at least 95% identical to the amino acid sequence of SEQ ID NO: 183, and a heavy chain variable region comprising an amino acid sequence at least 95% identical to the amino acid sequence of SEQ ID NO: 138; or
li. a light chain variable region comprising an amino acid sequence at least 95% identical to the amino acid sequence of SEQ ID NO: 193, and a heavy chain variable region comprising an amino acid sequence at least 95% identical to the amino acid sequence of SEQ ID NO: 138; or
lii. a light chain variable region comprising an amino acid sequence at least 95% identical to the amino acid sequence of SEQ ID NO: 203, and a heavy chain variable region comprising an amino acid sequence at least 95% identical to the amino acid sequence of SEQ ID NO: 138; or
liii. a light chain variable region comprising an amino acid sequence at least 95% identical to the amino acid sequence of SEQ ID NO: 213, and a heavy chain variable region comprising an amino acid sequence at least 95% identical to the amino acid sequence of SEQ ID NO: 138; or
liv. a light chain variable region comprising an amino acid sequence at least 95% identical to the amino acid sequence of SEQ ID NO: 223, and a heavy chain variable region comprising an amino acid sequence at least 95% identical to the amino acid sequence of SEQ ID NO: 138; or
lv. a light chain variable region comprising an amino acid sequence at least 95% identical to the amino acid sequence of SEQ ID NO: 233, and a heavy chain variable region comprising an amino acid sequence at least 95% identical to the amino acid sequence of SEQ ID NO: 138; or
lvi. a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 138; or
lvii. a light chain variable region comprising an amino acid sequence at least 95% identical to the amino acid sequence of SEQ ID NO: 253, and a heavy chain variable region comprising an amino acid sequence at least 95% identical to the amino acid sequence of SEQ ID NO: 138; or
lviii. a light chain variable region comprising an amino acid sequence at least 95% identical to the amino acid sequence of SEQ ID NO: 103, and a heavy chain variable region comprising an amino acid sequence at least 95% identical to the amino acid sequence of SEQ ID NO: 148; or
lix. a light chain variable region comprising an amino acid sequence at least 95% identical to the amino acid sequence of SEQ ID NO: 113, and a heavy chain variable region comprising an amino acid sequence at least 95% identical to the amino acid sequence of SEQ ID NO: 148; or
lx. a light chain variable region comprising an amino acid sequence at least 95% identical to the amino acid sequence of SEQ ID NO: 123, and a heavy chain variable region comprising an amino acid sequence at least 95% identical to the amino acid sequence of SEQ ID NO: 148; or
lxi. a light chain variable region comprising an amino acid sequence at least 95% identical to the amino acid sequence of SEQ ID NO: 163, and a heavy chain variable region comprising an amino acid sequence at least 95% identical to the amino acid sequence of SEQ ID NO: 148; or
lxii. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 148; or
lxiii. a light chain variable region comprising an amino acid sequence at least 95% identical to the amino acid sequence of SEQ ID NO: 183, and a heavy chain variable region comprising an amino acid sequence at least 95% identical to the amino acid sequence of SEQ ID NO: 148; or
lxiv. a light chain variable region comprising an amino acid sequence at least 95% identical to the amino acid sequence of SEQ ID NO: 193, and a heavy chain variable region comprising an amino acid sequence at least 95% identical to the amino acid sequence of SEQ ID NO: 148; or
lxv. a light chain variable region comprising an amino acid sequence at least 95% identical to the amino acid sequence of SEQ ID NO: 203, and a heavy chain variable region comprising an amino acid sequence at least 95% identical to the amino acid sequence of SEQ ID NO: 148; or
lxvi. a light chain variable region comprising an amino acid sequence at least 95% identical to the amino acid sequence of SEQ ID NO: 213, and a heavy chain variable region comprising an amino acid sequence at least 95% identical to the amino acid sequence of SEQ ID NO: 148; or
lxvii. a light chain variable region comprising an amino acid sequence at least 95% identical to the amino acid sequence of SEQ ID NO: 223, and a heavy chain variable region comprising an amino acid sequence at least 95% identical to the amino acid sequence of SEQ ID NO: 148; or
lxviii. a light chain variable region comprising an amino acid sequence at least 95% identical to the amino acid sequence of SEQ ID NO: 233, and a heavy chain variable region comprising an amino acid sequence at least 95% identical to the amino acid sequence of SEQ ID NO: 148; or
lxix. a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 148; or
lxx. a light chain variable region comprising an amino acid sequence at least 95% identical to the amino acid sequence of SEQ ID NO: 253, and a heavy chain variable region comprising an amino acid sequence at least 95% identical to the amino acid sequence of SEQ ID NO: 148; or
lxxi. a light chain variable region comprising an amino acid sequence at least 95% identical to the amino acid sequence of SEQ ID NO: 103, and a heavy chain variable region comprising an amino acid sequence at least 95% identical to the amino acid sequence of SEQ ID NO: 158; or
lxxii. a light chain variable region comprising an amino acid sequence at least 95% identical to the amino acid sequence of SEQ ID NO: 113, and a heavy chain variable region comprising an amino acid sequence at least 95% identical to the amino acid sequence of SEQ ID NO: 158; or
lxxiii. a light chain variable region comprising an amino acid sequence at least 95% identical to the amino acid sequence of SEQ ID NO: 123, and a heavy chain variable region comprising an amino acid sequence at least 95% identical to the amino acid sequence of SEQ ID NO: 158; or
lxxiv. a light chain variable region comprising an amino acid sequence at least 95% identical to the amino acid sequence of SEQ ID NO: 153, and a heavy chain variable region comprising an amino acid sequence at least 95% identical to the amino acid sequence of SEQ ID NO: 158; or
lxxv. a light chain variable region comprising an amino acid sequence at least 95% identical to the amino acid sequence of SEQ ID NO: 163, and a heavy chain variable region comprising an amino acid sequence at least 95% identical to the amino acid sequence of SEQ ID NO: 158; or
lxxvi. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 158; or
lxxvii. a light chain variable region comprising an amino acid sequence at least 95% identical to the amino acid sequence of SEQ ID NO: 183, and a heavy chain variable region comprising an amino acid sequence at least 95% identical to the amino acid sequence of SEQ ID NO: 158; or
lxxviii. a light chain variable region comprising an amino acid sequence at least 95% identical to the amino acid sequence of SEQ ID NO: 193, and a heavy chain variable region comprising an amino acid sequence at least 95% identical to the amino acid sequence of SEQ ID NO: 158; or
lxxix. a light chain variable region comprising an amino acid sequence at least 95% identical to the amino acid sequence of SEQ ID NO: 213, and a heavy chain variable region comprising an amino acid sequence at least 95% identical to the amino acid sequence of SEQ ID NO: 158; or
lxxx. a light chain variable region comprising an amino acid sequence at least 95% identical to the amino acid sequence of SEQ ID NO: 223, and a heavy chain variable region comprising an amino acid sequence at least 95% identical to the amino acid sequence of SEQ ID NO: 158; or
lxxxi. a light chain variable region comprising an amino acid sequence at least 95% identical to the amino acid sequence of SEQ ID NO: 233, and a heavy chain variable region comprising an amino acid sequence at least 95% identical to the amino acid sequence of SEQ ID NO: 158; or
lxxxii. a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 158; or
lxxxiii. a light chain variable region comprising an amino acid sequence at least 95% identical to the amino acid sequence of SEQ ID NO: 253, and a heavy chain variable region comprising an amino acid sequence at least 95% identical to the amino acid sequence of SEQ ID NO: 158; or
lxxxiv. a light chain variable region comprising an amino acid sequence at least 95% identical to the amino acid sequence of SEQ ID NO: 103, and a heavy chain variable region comprising an amino acid sequence at least 95% identical to the amino acid sequence of SEQ ID NO: 168; or
lxxxv. a light chain variable region comprising an amino acid sequence at least 95% identical to the amino acid sequence of SEQ ID NO: 113, and a heavy chain variable region comprising an amino acid sequence at least 95% identical to the amino acid sequence of SEQ ID NO: 168; or
lxxxvi. a light chain variable region comprising an amino acid sequence at least 95% identical to the amino acid sequence of SEQ ID NO: 123, and a heavy chain variable region comprising an amino acid sequence at least 95% identical to the amino acid sequence of SEQ ID NO: 168; or
lxxxvii. a light chain variable region comprising an amino acid sequence at least 95% identical to the amino acid sequence of SEQ ID NO: 163, and a heavy chain variable region comprising an amino acid sequence at least 95% identical to the amino acid sequence of SEQ ID NO: 168; or
lxxxviii. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 168; or
lxxxix. a light chain variable region comprising an amino acid sequence at least 95% identical to the amino acid sequence of SEQ ID NO: 183, and a heavy chain variable region comprising an amino acid sequence at least 95% identical to the amino acid sequence of SEQ ID NO: 168; or
xc. a light chain variable region comprising an amino acid sequence at least 95% identical to the amino acid sequence of SEQ ID NO: 193, and a heavy chain variable region comprising an amino acid sequence at least 95% identical to the amino acid sequence of SEQ ID NO: 168; or
xci. a light chain variable region comprising an amino acid sequence at least 95% identical to the amino acid sequence of SEQ ID NO: 203, and a heavy chain variable region comprising an amino acid sequence at least 95% identical to the amino acid sequence of SEQ ID NO: 168; or
xcii. a light chain variable region comprising an amino acid sequence at least 95% identical to the amino acid sequence of SEQ ID NO: 213, and a heavy chain variable region comprising an amino acid sequence at least 95% identical to the amino acid sequence of SEQ ID NO: 168; or
xciii. a light chain variable region comprising an amino acid sequence at least 95% identical to the amino acid sequence of SEQ ID NO: 223, and a heavy chain variable region comprising an amino acid sequence at least 95% identical to the amino acid sequence of SEQ ID NO: 168; or
xciv. a light chain variable region comprising an amino acid sequence at least 95% identical to the amino acid sequence of SEQ ID NO: 233, and a heavy chain variable region comprising an amino acid sequence at least 95% identical to the amino acid sequence of SEQ ID NO: 168; or
xcv. a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 168; or
xcvi. a light chain variable region comprising an amino acid sequence at least 95% identical to the amino acid sequence of SEQ ID NO: 253, and a heavy chain variable region comprising an amino acid sequence at least 95% identical to the amino acid sequence of SEQ ID NO: 168; or
xcvii. a light chain variable region comprising an amino acid sequence at least 95% identical to the amino acid sequence of SEQ ID NO: 103, and a heavy chain variable region comprising an amino acid sequence at least 95% identical to the amino acid sequence of SEQ ID NO: 178; or
xcviii. a light chain variable region comprising an amino acid sequence at least 95% identical to the amino acid sequence of SEQ ID NO: 113, and a heavy chain variable region comprising an amino acid sequence at least 95% identical to the amino acid sequence of SEQ ID NO: 178; or
xcix. a light chain variable region comprising an amino acid sequence at least 95% identical to the amino acid sequence of SEQ ID NO: 123, and a heavy chain variable region comprising an amino acid sequence at least 95% identical to the amino acid sequence of SEQ ID NO: 178; or
c. a light chain variable region comprising an amino acid sequence at least 95% identical to the amino acid sequence of SEQ ID NO: 153, and a heavy chain variable region comprising an amino acid sequence at least 95% identical to the amino acid sequence of SEQ ID NO: 178; or
ci. a light chain variable region comprising an amino acid sequence at least 95% identical to the amino acid sequence of SEQ ID NO: 163, and a heavy chain variable region comprising an amino acid sequence at least 95% identical to the amino acid sequence of SEQ ID NO: 178; or
cii. a light chain variable region comprising an amino acid sequence at least 95% identical to the amino acid sequence of SEQ ID NO: 173, and a heavy chain variable region comprising an amino acid sequence at least 95% identical to the amino acid sequence of SEQ ID NO: 178; or
ciii. a light chain variable region comprising an amino acid sequence at least 95% identical to the amino acid sequence of SEQ ID NO: 183, and a heavy chain variable region comprising an amino acid sequence at least 95% identical to the amino acid sequence of SEQ ID NO: 178; or
civ. a light chain variable region comprising an amino acid sequence at least 95% identical to the amino acid sequence of SEQ ID NO: 193, and a heavy chain variable region comprising an amino acid sequence at least 95% identical to the amino acid sequence of SEQ ID NO: 178; or
cv. a light chain variable region comprising an amino acid sequence at least 95% identical to the amino acid sequence of SEQ ID NO: 203, and a heavy chain variable region comprising an amino acid sequence at least 95% identical to the amino acid sequence of SEQ ID NO: 178; or
cvi. a light chain variable region comprising an amino acid sequence at least 95% identical to the amino acid sequence of SEQ ID NO: 213, and a heavy chain variable region comprising an amino acid sequence at least 95% identical to the amino acid sequence of SEQ ID NO: 178; or
cvii. a light chain variable region comprising an amino acid sequence at least 95% identical to the amino acid sequence of SEQ ID NO: 223, and a heavy chain variable region comprising an amino acid sequence at least 95% identical to the amino acid sequence of SEQ ID NO: 178; or
cviii. a light chain variable region comprising an amino acid sequence at least 95% identical to the amino acid sequence of SEQ ID NO: 233, and a heavy chain variable region comprising an amino acid sequence at least 95% identical to the amino acid sequence of SEQ ID NO: 178; or
cix. a light chain variable region comprising an amino acid sequence at least 95% identical to the amino acid sequence of SEQ ID NO: 243, and a heavy chain variable region comprising an amino acid sequence at least 95% identical to the amino acid sequence of SEQ ID NO: 178.

7. The method according to claim 1, anti-CTGF antibody or an antigen binding fragment thereof according to any one of the foregoing claims, wherein the anti-CTGF antibody or an antigen-binding fragment thereof comprises:
i. a light chain variable region comprising the amino acid sequence of SEQ ID NO: 223, and a heavy chain variable region comprising the amino acid sequence of SEQ ID NO: 108; or
ii. a light chain variable region comprising the amino acid sequence of SEQ ID NO: 103, and a heavy chain variable region comprising the amino acid sequence of SEQ ID NO: 108; or
iii. a light chain variable region comprising the amino acid sequence of SEQ ID NO: 113, and a heavy chain variable region comprising the amino acid sequence of SEQ ID NO: 108; or
iv. a light chain variable region comprising the amino acid sequence of SEQ ID NO: 123, and a heavy chain variable region comprising the amino acid sequence of SEQ ID NO: 108; or
v. a light chain variable region comprising the amino acid sequence of SEQ ID NO: 133, and a heavy chain variable region comprising the amino acid sequence of SEQ ID NO: 108; or
vi. a light chain variable region comprising the amino acid sequence of SEQ ID NO: 153, and a heavy chain variable region comprising the amino acid sequence of SEQ ID NO: 108; or
vii. a light chain variable region comprising the amino acid sequence of SEQ ID NO: 163, and a heavy chain variable region comprising the amino acid sequence of SEQ ID NO: 108; or
viii. a light chain variable region comprising the amino acid sequence of SEQ ID NO: 173, and a heavy chain variable region comprising the amino acid sequence of SEQ ID NO: 108; or
ix. a light chain variable region comprising the amino acid sequence of SEQ ID NO: 183, and a heavy chain variable region comprising the amino acid sequence of SEQ ID NO: 108; or
x. a light chain variable region comprising the amino acid sequence of SEQ ID NO: 193, and a heavy chain variable region comprising the amino acid sequence of SEQ ID NO: 108; or
xi. a light chain variable region comprising the amino acid sequence of SEQ ID NO: 203, and a heavy chain variable region comprising the amino acid sequence of SEQ ID NO: 108; or
xii. a light chain variable region comprising the amino acid sequence of SEQ ID NO: 213, and a heavy chain variable region comprising the amino acid sequence of SEQ ID NO: 108; or
xiii. a light chain variable region comprising the amino acid sequence of SEQ ID NO: 233, and a heavy chain variable region comprising the amino acid sequence of SEQ ID NO: 108; or
xiv. a light chain variable region comprising the amino acid sequence of SEQ ID NO: 243, and a heavy chain variable region comprising the amino acid sequence of SEQ ID NO: 108; or
xv. a light chain variable region comprising the amino acid sequence of SEQ ID NO: 253, and a heavy chain variable region comprising the amino acid sequence of SEQ ID NO: 108; or
xvi. a light chain variable region comprising the amino acid sequence of SEQ ID NO: 103, and a heavy chain variable region comprising the amino acid sequence of SEQ ID NO: 118; or
xvii. a light chain variable region comprising the amino acid sequence of SEQ ID NO: 113, and a heavy chain variable region comprising the amino acid sequence of SEQ ID NO: 118; or
xviii. a light chain variable region comprising the amino acid sequence of SEQ ID NO: 123, and a heavy chain variable region comprising the amino acid sequence of SEQ ID NO: 118; or
xix. a light chain variable region comprising the amino acid sequence of SEQ ID NO: 153, and a heavy chain variable region comprising the amino acid sequence of SEQ ID NO: 118; or
xx. a light chain variable region comprising the amino acid sequence of SEQ ID NO: 163, and a heavy chain variable region comprising the amino acid sequence of SEQ ID NO: 118; or
xxi. a light chain variable region comprising the amino acid sequence of SEQ ID NO: 173, and a heavy chain variable region comprising the amino acid sequence of SEQ ID NO: 118; or
xxii. a light chain variable region comprising the amino acid sequence of SEQ ID NO: 183, and a heavy chain variable region comprising the amino acid sequence of SEQ ID NO: 118; or
xxiii. a light chain variable region comprising the amino acid sequence of SEQ ID NO: 193, and a heavy chain variable region comprising the amino acid sequence of SEQ ID NO: 118; or
xxiv. a light chain variable region comprising the amino acid sequence of SEQ ID NO: 203, and a heavy chain variable region comprising the amino acid sequence of SEQ ID NO: 118; or
xxv. a light chain variable region comprising the amino acid sequence of SEQ ID NO: 213, and a heavy chain variable region comprising the amino acid sequence of SEQ ID NO: 118; or
xxvi. a light chain variable region comprising the amino acid sequence of SEQ ID NO: 223, and a heavy chain variable region comprising the amino acid sequence of SEQ ID NO: 118; or
xxvii. a light chain variable region comprising the amino acid sequence of SEQ ID NO: 233, and a heavy chain variable region comprising the amino acid sequence of SEQ ID NO: 118; or
xxviii. a light chain variable region comprising the amino acid sequence of SEQ ID NO: 243, and a heavy chain variable region comprising the amino acid sequence of SEQ ID NO: 118; or
xxix. a light chain variable region comprising the amino acid sequence of SEQ ID NO: 253, and a heavy chain variable region comprising the amino acid sequence of SEQ ID NO: 118; or
xxx. a light chain variable region comprising the amino acid sequence of SEQ ID NO: 103, and a heavy chain variable region comprising the amino acid sequence of SEQ ID NO: 128; or
xxxi. a light chain variable region comprising the amino acid sequence of SEQ ID NO: 113, and a heavy chain variable region comprising the amino acid sequence of SEQ ID NO: 128; or
xxxii. a light chain variable region comprising the amino acid sequence of SEQ ID NO: 123, and a heavy chain variable region comprising the amino acid sequence of SEQ ID NO: 128; or
xxxiii. a light chain variable region comprising the amino acid sequence of SEQ ID NO: 153, and a heavy chain variable region comprising the amino acid sequence of SEQ ID NO: 128; or
xxxiv. a light chain variable region comprising the amino acid sequence of SEQ ID NO: 163, and a heavy chain variable region comprising the amino acid sequence of SEQ ID NO: 128; or
xxxv. a light chain variable region comprising the amino acid sequence of SEQ ID NO: 173, and a heavy chain variable region comprising the amino acid sequence of SEQ ID NO: 128; or
xxxvi. a light chain variable region comprising the amino acid sequence of SEQ ID NO: 183, and a heavy chain variable region comprising the amino acid sequence of SEQ ID NO: 128; or
xxxvii. a light chain variable region comprising the amino acid sequence of SEQ ID NO: 193, and a heavy chain variable region comprising the amino acid sequence of SEQ ID NO: 128; or
xxxviii. a light chain variable region comprising the amino acid sequence of SEQ ID NO: 203, and a heavy chain variable region comprising the amino acid sequence of SEQ ID NO: 128; or
xxxix. a light chain variable region comprising the amino acid sequence of SEQ ID NO: 213, and a heavy chain variable region comprising the amino acid sequence of SEQ ID NO: 128; or
xl. a light chain variable region comprising the amino acid sequence of SEQ ID NO: 223, and a heavy chain variable region comprising the amino acid sequence of SEQ ID NO: 128; or
xli. a light chain variable region comprising the amino acid sequence of SEQ ID NO: 233, and a heavy chain variable region comprising the amino acid sequence of SEQ ID NO: 128; or
xlii. a light chain variable region comprising the amino acid sequence of SEQ ID NO: 243, and a heavy chain variable region comprising the amino acid sequence of SEQ ID NO: 128; or
xliii. a light chain variable region comprising the amino acid sequence of SEQ ID NO: 253, and a heavy chain variable region comprising the amino acid sequence of SEQ ID NO: 128; or
xliv. a light chain variable region comprising the amino acid sequence of SEQ ID NO: 103, and a heavy chain variable region comprising the amino acid sequence of SEQ ID NO: 138; or
xlv. a light chain variable region comprising the amino acid sequence of SEQ ID NO: 113, and a heavy chain variable region comprising the amino acid sequence of SEQ ID NO: 138; or
xlvi. a light chain variable region comprising the amino acid sequence of SEQ ID NO: 123, and a heavy chain variable region comprising the amino acid sequence of SEQ ID NO: 138; or
xlvii. a light chain variable region comprising the amino acid sequence of SEQ ID NO: 153, and a heavy chain variable region comprising the amino acid sequence of SEQ ID NO: 138; or
xlviii. a light chain variable region comprising the amino acid sequence of SEQ ID NO: 163, and a heavy chain variable region comprising the amino acid sequence of SEQ ID NO: 138; or
xlix. a light chain variable region comprising the amino acid sequence of SEQ ID NO: 173, and a heavy chain variable region comprising the amino acid sequence of SEQ ID NO: 138; or
l. a light chain variable region comprising the amino acid sequence of SEQ ID NO: 183, and a heavy chain variable region comprising the amino acid sequence of SEQ ID NO: 138; or
li. a light chain variable region comprising the amino acid sequence of SEQ ID NO: 193, and a heavy chain variable region comprising the amino acid sequence of SEQ ID NO: 138; or
lii. a light chain variable region comprising the amino acid sequence of SEQ ID NO: 203, and a heavy chain variable region comprising the amino acid sequence of SEQ ID NO: 138; or
liii. a light chain variable region comprising the amino acid sequence of SEQ ID NO: 213, and a heavy chain variable region comprising the amino acid sequence of SEQ ID NO: 138; or
liv. a light chain variable region comprising the amino acid sequence of SEQ ID NO: 223, and a heavy chain variable region comprising the amino acid sequence of SEQ ID NO: 138; or
lv. a light chain variable region comprising the amino acid sequence of SEQ ID NO: 233, and a heavy chain variable region comprising the amino acid sequence of SEQ ID NO: 138; or
lvi. a light chain variable region comprising the amino acid sequence of SEQ ID NO: 243, and a heavy chain variable region comprising the amino acid sequence of SEQ ID NO: 138; or
lvii. a light chain variable region comprising the amino acid sequence of SEQ ID NO: 253, and a heavy chain variable region comprising the amino acid sequence of SEQ ID NO: 138; or
lviii. a light chain variable region comprising the amino acid sequence of SEQ ID NO: 103, and a heavy chain variable region comprising the amino acid sequence of SEQ ID NO: 148; or
lix. a light chain variable region comprising the amino acid sequence of SEQ ID NO: 113, and a heavy chain variable region comprising the amino acid sequence of SEQ ID NO: 148; or
lx. a light chain variable region comprising the amino acid sequence of SEQ ID NO: 123, and a heavy chain variable region comprising the amino acid sequence of SEQ ID NO: 148; or
lxi. a light chain variable region comprising the amino acid sequence of SEQ ID NO: 163, and a heavy chain variable region comprising the amino acid sequence of SEQ ID NO: 148; or
lxii. a light chain variable region comprising the amino acid sequence of SEQ ID NO: 173, and a heavy chain variable region comprising the amino acid sequence of SEQ ID NO: 148; or
lxiii. a light chain variable region comprising the amino acid sequence of SEQ ID NO: 183, and a heavy chain variable region comprising the amino acid sequence of SEQ ID NO: 148; or
lxiv. a light chain variable region comprising the amino acid sequence of SEQ ID NO: 193, and a heavy chain variable region comprising the amino acid sequence of SEQ ID NO: 148; or
lxv. a light chain variable region comprising the amino acid sequence of SEQ ID NO: 203, and a heavy chain variable region comprising the amino acid sequence of SEQ ID NO: 148; or
lxvi. a light chain variable region comprising the amino acid sequence of SEQ ID NO: 213, and a heavy chain variable region comprising the amino acid sequence of SEQ ID NO: 148; or
lxvii. a light chain variable region comprising the amino acid sequence of SEQ ID NO: 223, and a heavy chain variable region comprising the amino acid sequence of SEQ ID NO: 148; or
lxviii. a light chain variable region comprising the amino acid sequence of SEQ ID NO: 233, and a heavy chain variable region comprising the amino acid sequence of SEQ ID NO: 148; or
lxix. a light chain variable region comprising the amino acid sequence of SEQ ID NO: 243, and a heavy chain variable region comprising the amino acid sequence of SEQ ID NO: 148; or
lxx. a light chain variable region comprising the amino acid sequence of SEQ ID NO: 253, and a heavy chain variable region comprising the amino acid sequence of SEQ ID NO: 148; or
lxxi. a light chain variable region comprising the amino acid sequence of SEQ ID NO: 103, and a heavy chain variable region comprising the amino acid sequence of SEQ ID NO: 158; or
lxxii. a light chain variable region comprising the amino acid sequence of SEQ ID NO: 113, and a heavy chain variable region comprising the amino acid sequence of SEQ ID NO: 158; or
lxxiii. a light chain variable region comprising the amino acid sequence of SEQ ID NO: 123, and a heavy chain variable region comprising the amino acid sequence of SEQ ID NO: 158; or
lxxiv. a light chain variable region comprising the amino acid sequence of SEQ ID NO: 153, and a heavy chain variable region comprising the amino acid sequence of SEQ ID NO: 158; or
lxxv. a light chain variable region comprising the amino acid sequence of SEQ ID NO: 163, and a heavy chain variable region comprising the amino acid sequence of SEQ ID NO: 158; or
lxxvi. a light chain variable region comprising the amino acid sequence of SEQ ID NO: 173, and a heavy chain variable region comprising the amino acid sequence of SEQ ID NO: 158; or
lxxvii. a light chain variable region comprising the amino acid sequence of SEQ ID NO: 183, and a heavy chain variable region comprising the amino acid sequence of SEQ ID NO: 158; or
lxxviii. a light chain variable region comprising the amino acid sequence of SEQ ID NO: 193, and a heavy chain variable region comprising the amino acid sequence of SEQ ID NO: 158; or
lxxix. a light chain variable region comprising the amino acid sequence of SEQ ID NO: 213, and a heavy chain variable region comprising the amino acid sequence of SEQ ID NO: 158; or
lxxx. a light chain variable region comprising the amino acid sequence of SEQ ID NO: 223, and a heavy chain variable region comprising the amino acid sequence of SEQ ID NO: 158; or
lxxxi. a light chain variable region comprising the amino acid sequence of SEQ ID NO: 233, and a heavy chain variable region comprising the amino acid sequence of SEQ ID NO: 158; or
lxxxii. a light chain variable region comprising the amino acid sequence of SEQ ID NO: 243, and a heavy chain variable region comprising the amino acid sequence of SEQ ID NO: 158; or
lxxxiii. a light chain variable region comprising the amino acid sequence of SEQ ID NO: 253, and a heavy chain variable region comprising the amino acid sequence of SEQ ID NO: 158; or
lxxxiv. a light chain variable region comprising the amino acid sequence of SEQ ID NO: 103, and a heavy chain variable region comprising the amino acid sequence of SEQ ID NO: 168; or
lxxxv. a light chain variable region comprising the amino acid sequence of SEQ ID NO: 113, and a heavy chain variable region comprising the amino acid sequence of SEQ ID NO: 168; or
lxxxvi. a light chain variable region comprising the amino acid sequence of SEQ ID NO: 123, and a heavy chain variable region comprising the amino acid sequence of SEQ ID NO: 168; or
lxxxvii. a light chain variable region comprising the amino acid sequence of SEQ ID NO: 163, and a heavy chain variable region comprising the amino acid sequence of SEQ ID NO: 168; or
lxxxviii. a light chain variable region comprising the amino acid sequence of SEQ ID NO: 173, and a heavy chain variable region comprising the amino acid sequence of SEQ ID NO: 168; or
lxxxix. a light chain variable region comprising the amino acid sequence of SEQ ID NO: 183, and a heavy chain variable region comprising the amino acid sequence of SEQ ID NO: 168; or
xc. a light chain variable region comprising the amino acid sequence of SEQ ID NO: 193, and a heavy chain variable region comprising the amino acid sequence of SEQ ID NO: 168; or
xci. a light chain variable region comprising the amino acid sequence of SEQ ID NO: 203, and a heavy chain variable region comprising the amino acid sequence of SEQ ID NO: 168; or
xcii. a light chain variable region comprising the amino acid sequence of SEQ ID NO: 213, and a heavy chain variable region comprising the amino acid sequence of SEQ ID NO: 168; or
xciii. a light chain variable region comprising the amino acid sequence of SEQ ID NO: 223, and a heavy chain variable region comprising the amino acid sequence of SEQ ID NO: 168; or
xciv. a light chain variable region comprising the amino acid sequence of SEQ ID NO: 233, and a heavy chain variable region comprising the amino acid sequence of SEQ ID NO: 168; or
xcv. a light chain variable region comprising the amino acid sequence of SEQ ID NO: 243, and a heavy chain variable region comprising the amino acid sequence of SEQ ID NO: 168; or
xcvi. a light chain variable region comprising the amino acid sequence of SEQ ID NO: 253, and a heavy chain variable region comprising the amino acid sequence of SEQ ID NO: 168; or
xcvii. a light chain variable region comprising the amino acid sequence of SEQ ID NO: 103, and a heavy chain variable region comprising the amino acid sequence of SEQ ID NO: 178; or
xcviii. a light chain variable region comprising the amino acid sequence of SEQ ID NO: 113, and a heavy chain variable region comprising the amino acid sequence of SEQ ID NO: 178; or
xcix. a light chain variable region comprising the amino acid sequence of SEQ ID NO: 123, and a heavy chain variable region comprising the amino acid sequence of SEQ ID NO: 178; or
c. a light chain variable region comprising the amino acid sequence of SEQ ID NO: 153, and a heavy chain variable region comprising the amino acid sequence of SEQ ID NO: 178; or
ci. a light chain variable region comprising the amino acid sequence of SEQ ID NO: 163, and a heavy chain variable region comprising the amino acid sequence of SEQ ID NO: 178; or
cii. a light chain variable region comprising the amino acid sequence of SEQ ID NO: 173, and a heavy chain variable region comprising the amino acid sequence of SEQ ID NO: 178; or
ciii. a light chain variable region comprising the amino acid sequence of SEQ ID NO: 183, and a heavy chain variable region comprising the amino acid sequence of SEQ ID NO: 178; or
civ. a light chain variable region comprising the amino acid sequence of SEQ ID NO: 193, and a heavy chain variable region comprising the amino acid sequence of SEQ ID NO: 178; or
cv. a light chain variable region comprising the amino acid sequence of SEQ ID NO: 203, and a heavy chain variable region comprising the amino acid sequence of SEQ ID NO: 178; or
cvi. a light chain variable region comprising the amino acid sequence of SEQ ID NO: 213, and a heavy chain variable region comprising the amino acid sequence of SEQ ID NO: 178; or
cvii. a light chain variable region comprising the amino acid sequence of SEQ ID NO: 223, and a heavy chain variable region comprising the amino acid sequence of SEQ ID NO: 178; or
cviii. a light chain variable region comprising the amino acid sequence of SEQ ID NO: 233, and a heavy chain variable region comprising the amino acid sequence of SEQ ID NO: 178; or
cix. a light chain variable region comprising the amino acid sequence of SEQ ID NO: 243, and a heavy chain variable region comprising the amino acid sequence of SEQ ID NO: 178.

8. The method according to claim 1, wherein the anti-CTGE antibody or antigen-binding fragment thereof comprises:
i. a light chain comprising an amino acid sequence at least 80% identical to the amino acid sequence of SEQ ID NO: 224, and a heavy chain comprising an amino acid sequence at least 80% identical to the amino acid sequence of SEQ ID NO: 109; or
ii. a light chain comprising an amino acid sequence at least 80% identical to the amino acid sequence of SEQ ID NO: 104, and a heavy chain comprising an amino acid sequence at least 80% identical to the amino acid sequence of SEQ ID NO: 109; or
iii. a light chain comprising an amino acid sequence at least 80% identical to the amino acid sequence of SEQ ID NO: 114, and a heavy chain comprising an amino acid sequence at least 80% identical to the amino acid sequence of SEQ ID NO: 109; or
iv. a light chain comprising an amino acid sequence at least 80% identical to the amino acid sequence of SEQ ID NO: 124, and a heavy chain comprising an amino acid sequence at least 80% identical to the amino acid sequence of SEQ ID NO: 109; or
v. a light chain comprising an amino acid sequence at least 80% identical to the amino acid sequence of SEQ ID NO: 134, and a heavy chain comprising an amino acid sequence at least 80% identical to the amino acid sequence of SEQ ID NO: 109; or
vi. a light chain comprising an amino acid sequence at least 80% identical to the amino acid sequence of SEQ ID NO: 154, and a heavy chain comprising an amino acid sequence at least 80% identical to the amino acid sequence of SEQ ID NO: 109; or
vii. a light chain comprising an amino acid sequence at least 80% identical to the amino acid sequence of SEQ ID NO: 164, and a heavy chain comprising an amino acid sequence at least 80% identical to the amino acid sequence of SEQ ID NO: 109; or
viii. a light chain comprising an amino acid sequence at least 80% identical to the amino acid sequence of SEQ ID NO: 174, and a heavy chain comprising an amino acid sequence at least 80% identical to the amino acid sequence of SEQ ID NO: 109; or
ix. a light chain comprising an amino acid sequence at least 80% identical to the amino acid sequence of SEQ ID NO: 184, and a heavy chain comprising an amino acid sequence at least 80% identical to the amino acid sequence of SEQ ID NO: 109; or
x. a light chain comprising an amino acid sequence at least 80% identical to the amino acid sequence of SEQ ID NO: 194, and a heavy chain comprising an amino acid sequence at least 80% identical to the amino acid sequence of SEQ ID NO: 109; or
xi. a light chain comprising an amino acid sequence at least 80% identical to the amino acid sequence of SEQ ID NO: 204, and a heavy chain comprising an amino acid sequence at least 80% identical to the amino acid sequence of SEQ ID NO: 109; or
xii. a light chain comprising an amino acid sequence at least 80% identical to the amino acid sequence of SEQ ID NO: 214, and a heavy chain comprising an amino acid sequence at least 80% identical to the amino acid sequence of SEQ ID NO: 109; or
xiii. a light chain comprising an amino acid sequence at least 80% identical to the amino acid sequence of SEQ ID NO: 234, and a heavy chain comprising an amino acid sequence at least 80% identical to the amino acid sequence of SEQ ID NO: 109; or
xiv. a light chain comprising an amino acid sequence at least 80% identical to the amino acid sequence of SEQ ID NO: 244, and a heavy chain comprising an amino acid sequence at least 80% identical to the amino acid sequence of SEQ ID NO: 109; or
xv. a light chain comprising an amino acid sequence at least 80% identical to the amino acid sequence of SEQ ID NO: 254, and a heavy chain comprising an amino acid sequence at least 80% identical to the amino acid sequence of SEQ ID NO: 109; or
xvi. a light chain comprising an amino acid sequence at least 80% identical to the amino acid sequence of SEQ ID NO: 104, and a heavy chain comprising an amino acid sequence at least 80% identical to the amino acid sequence of SEQ ID NO: 119; or
xvii. a light chain comprising an amino acid sequence at least 80% identical to the amino acid sequence of SEQ ID NO: 114, and a heavy chain comprising an amino acid sequence at least 80% identical to the amino acid sequence of SEQ ID NO: 119; or
xviii. a light chain comprising an amino acid sequence at least 80% identical to the amino acid sequence of SEQ ID NO: 124, and a heavy chain comprising an amino acid sequence at least 80% identical to the amino acid sequence of SEQ ID NO: 119; or
xix. a light chain comprising an amino acid sequence at least 80% identical to the amino acid sequence of SEQ ID NO: 154, and a heavy chain comprising an amino acid sequence at least 80% identical to the amino acid sequence of SEQ ID NO: 119; or
xx. a light chain comprising an amino acid sequence at least 80% identical to the amino acid sequence of SEQ ID NO: 164, and a heavy chain comprising an amino acid sequence at least 80% identical to the amino acid sequence of SEQ ID NO: 119; or
xxi. a light chain comprising an amino acid sequence at least 80% identical to the amino acid sequence of SEQ ID NO: 174, and a heavy chain comprising an amino acid sequence at least 80% identical to the amino acid sequence of SEQ ID NO: 119; or
xxii. a light chain comprising an amino acid sequence at least 80% identical to the amino acid sequence of SEQ ID NO: 184, and a heavy chain comprising an amino acid sequence at least 80% identical to the amino acid sequence of SEQ ID NO: 119; or
xxiii. a light chain comprising an amino acid sequence at least 80% identical to the amino acid sequence of SEQ ID NO: 194, and a heavy chain comprising an amino acid sequence at least 80% identical to the amino acid sequence of SEQ ID NO: 119; or
xxiv. a light chain comprising an amino acid sequence at least 80% identical to the amino acid sequence of SEQ ID NO: 204, and a heavy chain comprising an amino acid sequence at least 80% identical to the amino acid sequence of SEQ ID NO: 119; or
xxv. a light chain comprising an amino acid sequence at least 80% identical to the amino acid sequence of SEQ ID NO: 214, and a heavy chain comprising an amino acid sequence at least 80% identical to the amino acid sequence of SEQ ID NO: 119; or
xxvi. a light chain comprising an amino acid sequence at least 80% identical to the amino acid sequence of SEQ ID NO: 224, and a heavy chain comprising an amino acid sequence at least 80% identical to the amino acid sequence of SEQ ID NO: 119; or
xxvii. a light chain comprising an amino acid sequence at least 80% identical to the amino acid sequence of SEQ ID NO: 234, and a heavy chain comprising an amino acid sequence at least 80% identical to the amino acid sequence of SEQ ID NO: 119; or
xxviii. a light chain comprising an amino acid sequence at least 80% identical to the amino acid sequence of SEQ ID NO: 244, and a heavy chain comprising an amino acid sequence at least 80% identical to the amino acid sequence of SEQ ID NO: 119; or
xxix. a light chain comprising an amino acid sequence at least 80% identical to the amino acid sequence of SEQ ID NO: 254, and a heavy chain comprising an amino acid sequence at least 80% identical to the amino acid sequence of SEQ ID NO: 119; or
xxx. a light chain comprising an amino acid sequence at least 80% identical to the amino acid sequence at least 80% identical to the amino acid sequence of SEQ ID NO: 129; or
xxxi. a light chain comprising an amino acid sequence at least 80% identical to the amino acid sequence of SEQ ID NO: 114, and a heavy chain comprising an amino acid sequence at least 80% identical to the amino acid sequence of SEQ ID NO: 129; or
xxxii. a light chain comprising an amino acid sequence at least 80% identical to the amino acid sequence of SEQ ID NO: 124, and a heavy chain comprising an amino acid sequence at least 80% identical to the amino acid sequence of SEQ ID NO: 129; or
xxxiii. a light chain comprising an amino acid sequence at least 80% identical to the amino acid sequence of SEQ ID NO: 154, and a heavy chain comprising an amino acid sequence at least 80% identical to the amino acid sequence of SEQ ID NO: 129; or
xxxiv. a light chain comprising an amino acid sequence at least 80% identical to the amino acid sequence of SEQ ID NO: 164, and a heavy chain comprising an amino acid sequence at least 80% identical to the amino acid sequence of SEQ ID NO: 129; or
xxxv. a light chain comprising an amino acid sequence at least 80% identical to the amino acid sequence of SEQ ID NO: 174, and a heavy chain comprising an amino acid sequence at least 80% identical to the amino acid sequence of SEQ ID NO: 129; or
xxxvi. a light chain comprising an amino acid sequence at least 80% identical to the amino acid sequence of SEQ ID NO: 184, and a heavy chain comprising an amino acid sequence at least 80% identical to the amino acid sequence of SEQ ID NO: 129; or
xxxvii. a light chain comprising an amino acid sequence at least 80% identical to the amino acid sequence of SEQ ID NO: 194, and a heavy chain comprising an amino acid sequence at least 80% identical to the amino acid sequence of SEQ ID NO: 129; or
xxxviii. a light chain comprising an amino acid sequence at least 80% identical to the amino acid sequence of SEQ ID NO: 204, and a heavy chain comprising an amino acid sequence at least 80% identical to the amino acid sequence of SEQ ID NO: 129; or
xxxix. a light chain comprising an amino acid sequence at least 80% identical to the amino acid sequence of SEQ ID NO: 214, and a heavy chain comprising an amino acid sequence at least 80% identical to the amino acid sequence of SEQ ID NO: 129; or
xl. a light chain comprising an amino acid sequence at least 80% identical to the amino acid sequence of SEQ ID NO: 224, and a heavy chain comprising an amino acid sequence at least 80% identical to the amino acid sequence of SEQ ID NO: 129; or
xli. a light chain comprising an amino acid sequence at least 80% identical to the amino acid sequence of SEQ ID NO: 234, and a heavy chain comprising an amino acid sequence at least 80% identical to the amino acid sequence of SEQ ID NO: 129; or
xlii. a light chain comprising an amino acid sequence at least 80% identical to the amino acid sequence of SEQ ID NO: 244, and a heavy chain comprising an amino acid sequence at least 80% identical to the amino acid sequence of SEQ ID NO: 129; or
xliii. a light chain comprising an amino acid sequence at least 80% identical to the amino acid sequence of SEQ ID NO: 254, and a heavy chain comprising an amino acid sequence at least 80% identical to the amino acid sequence of SEQ ID NO: 129; or
xliv. a light chain comprising an amino acid sequence at least 80% identical to the amino acid sequence of SEQ ID NO: 104, and a heavy chain comprising an amino acid sequence at least 80% identical to the amino acid sequence of SEQ ID NO: 139; or
xlv. a light chain comprising an amino acid sequence at least 80% identical to the amino acid sequence of SEQ ID NO: 114, and a heavy chain comprising an amino acid sequence at least 80% identical to the amino acid sequence of SEQ ID NO: 139; or
xlvi. a light chain comprising an amino acid sequence at least 80% identical to the amino acid sequence of SEQ ID NO: 124, and a heavy chain comprising an amino acid sequence at least 80% identical to the amino acid sequence of SEQ ID NO: 139; or
xlvii. a light chain comprising an amino acid sequence at least 80% identical to the amino acid sequence of SEQ ID NO: 154, and a heavy chain comprising an amino acid sequence at least 80% identical to the amino acid sequence of SEQ ID NO: 139; or
xlviii. a light chain comprising an amino acid sequence at least 80% identical to the amino acid sequence of SEQ ID NO: 164, and a heavy chain comprising an amino acid sequence at least 80% identical to the amino acid sequence of SEQ ID NO: 139; or
xlix. a light chain comprising an amino acid sequence at least 80% identical to the amino acid sequence of SEQ ID NO: 174, and a heavy chain comprising an amino acid sequence at least 80% identical to the amino acid sequence of SEQ ID NO: 139; or
l. a light chain comprising an amino acid sequence at least 80% identical to the amino acid sequence of SEQ ID NO: 184, and a heavy chain comprising an amino acid sequence at least 80% identical to the amino acid sequence of SEQ ID NO: 139; or
li. a light chain comprising an amino acid sequence at least 80% identical to the amino acid sequence of SEQ ID NO: 194, and a heavy chain comprising an amino acid sequence at least 80% identical to the amino acid sequence of SEQ ID NO: 139; or
lii. a light chain comprising an amino acid sequence at least 80% identical to the amino acid sequence of SEQ ID NO: 204, and a heavy chain comprising an amino acid sequence at least 80% identical to the amino acid sequence of SEQ ID NO: 139; or
liii. a light chain comprising an amino acid sequence at least 80% identical to the amino acid sequence of SEQ ID NO: 214, and a heavy chain comprising an amino acid sequence at least 80% identical to the amino acid sequence of SEQ ID NO: 139; or
liv. a light chain comprising an amino acid sequence at least 80% identical to the amino acid sequence of SEQ ID NO: 224, and a heavy chain comprising an amino acid sequence at least 80% identical to the amino acid sequence of SEQ ID NO: 139; or
lv. a light chain comprising an amino acid sequence at least 80% identical to the amino acid sequence of SEQ ID NO: 234, and a heavy chain comprising an amino acid sequence at least 80% identical to the amino acid sequence of SEQ ID NO: 139; or
lvi. a light chain comprising an amino acid sequence at least 80% identical to the amino acid sequence of SEQ ID NO: 244, and a heavy chain comprising an amino acid sequence at least 80% identical to the amino acid sequence of SEQ ID NO: 139; or
lvii. a light chain comprising an amino acid sequence at least 80% identical to the amino acid sequence of SEQ ID NO: 254, and a heavy chain comprising an amino acid sequence at least 80% identical to the amino acid sequence of SEQ ID NO: 139; or
lviii. a light chain comprising an amino acid sequence at least 80% identical to the amino acid sequence of SEQ ID NO: 104, and a heavy chain comprising an amino acid sequence at least 80% identical to the amino acid sequence of SEQ ID NO: 149; or
lix. a light chain comprising an amino acid sequence at least 80% identical to the amino acid sequence of SEQ ID NO: 114, and a heavy chain comprising an amino acid sequence at least 80% identical to the amino acid sequence of SEQ ID NO: 149; or
lx. a light chain comprising an amino acid sequence at least 80% identical to the amino acid sequence of SEQ ID NO: 124, and a heavy chain comprising an amino acid sequence at least 80% identical to the amino acid sequence of SEQ ID NO: 149; or
lxi. a light chain comprising an amino acid sequence at least 80% identical to the amino acid sequence of SEQ ID NO: 164, and a heavy chain comprising an amino acid sequence at least 80% identical to the amino acid sequence of SEQ ID NO: 149; or
lxii. a light chain comprising an amino acid sequence at least 80% identical to the amino acid sequence of SEQ ID NO: 174, and a heavy chain comprising an amino acid sequence at least 80% identical to the amino acid sequence of SEQ ID NO: 149; or
lxiii. a light chain comprising an amino acid sequence at least 80% identical to the amino acid sequence of SEQ ID NO: 184, and a heavy chain comprising an amino acid sequence at least 80% identical to the amino acid sequence of SEQ ID NO: 149; or
lxiv. a light chain comprising an amino acid sequence at least 80% identical to the amino acid sequence of SEQ ID NO: 194, and a heavy chain comprising an amino acid sequence at least 80% identical to the amino acid sequence of SEQ ID NO: 149; or
lxv. a light chain comprising an amino acid sequence at least 80% identical to the amino acid sequence of SEQ ID NO: 204, and a heavy chain comprising an amino acid sequence at least 80% identical to the amino acid sequence of SEQ ID NO: 149; or
lxvi. a light chain comprising an amino acid sequence at least 80% identical to the amino acid sequence of SEQ ID NO: 214, and a heavy chain comprising an amino acid sequence at least 80% identical to the amino acid sequence of SEQ ID NO: 149; or
lxvii. a light chain comprising an amino acid sequence at least 80% identical to the amino acid sequence of SEQ ID NO: 224, and a heavy chain comprising an amino acid sequence at least 80% identical to the amino acid sequence of SEQ ID NO: 149; or
lxviii. a light chain comprising an amino acid sequence at least 80% identical to the amino acid sequence of SEQ ID NO: 234, and a heavy chain comprising an amino acid sequence at least 80% identical to the amino acid sequence of SEQ ID NO: 149; or
lxix. a light chain comprising an amino acid sequence at least 80% identical to the amino acid sequence of SEQ ID NO: 244, and a heavy chain comprising an amino acid sequence at least 80% identical to the amino acid sequence of SEQ ID NO: 149; or
lxx. a light chain comprising an amino acid sequence at least 80% identical to the amino acid sequence of SEQ ID NO: 254, and a heavy chain comprising an amino acid sequence at least 80% identical to the amino acid sequence of SEQ ID NO: 149; or
lxxi. a light chain comprising an amino acid sequence at least 80% identical to the amino acid sequence of SEQ ID NO: 104, and a heavy chain comprising an amino acid sequence at least 80% identical to the amino acid sequence of SEQ ID NO: 159; or
lxxii. a light chain comprising an amino acid sequence at least 80% identical to the amino acid sequence of SEQ ID NO: 114, and a heavy chain comprising an amino acid sequence at least 80% identical to the amino acid sequence of SEQ ID NO: 159; or
lxxiii. a light chain comprising an amino acid sequence at least 80% identical to the amino acid sequence of SEQ ID NO: 124, and a heavy chain comprising an amino acid sequence at least 80% identical to the amino acid sequence of SEQ ID NO: 159; or
lxxiv. a light chain comprising an amino acid sequence at least 80% identical to the amino acid sequence of SEQ ID NO: 154, and a heavy chain comprising an amino acid sequence at least 80% identical to the amino acid sequence of SEQ ID NO: 159; or
lxxv. a light chain comprising an amino acid sequence at least 80% identical to the amino acid sequence of SEQ ID NO: 164, and a heavy chain comprising an amino acid sequence at least 80% identical to the amino acid sequence of SEQ ID NO: 159; or
lxxvi. a light chain comprising an amino acid sequence at least 80% identical to the amino acid sequence of SEQ ID NO: 174, and a heavy chain comprising an amino acid sequence at least 80% identical to the amino acid sequence of SEQ ID NO: 159; or
lxxvii. a light chain comprising an amino acid sequence at least 80% identical to the amino acid sequence of SEQ ID NO: 184, and a heavy chain comprising an amino acid sequence at least 80% identical to the amino acid sequence of SEQ ID NO: 159; or
lxxviii. a light chain comprising an amino acid sequence at least 80% identical to the amino acid sequence of SEQ ID NO: 194, and a heavy chain comprising an amino acid sequence at least 80% identical to the amino acid sequence of SEQ ID NO: 159; or
lxxix. a light chain comprising an amino acid sequence at least 80% identical to the amino acid sequence of SEQ ID NO: 214, and a heavy chain comprising an amino acid sequence at least 80% identical to the amino acid sequence of SEQ ID NO: 159; or
lxxx. a light chain comprising an amino acid sequence at least 80% identical to the amino acid sequence of SEQ ID NO: 224, and a heavy chain comprising an amino acid sequence at least 80% identical to the amino acid sequence of SEQ ID NO: 159; or
lxxxi. a light chain comprising an amino acid sequence at least 80% identical to the amino acid sequence of SEQ ID NO: 234, and a heavy chain comprising an amino acid sequence at least 80% identical to the amino acid sequence of SEQ ID NO: 159; or
lxxxii. a light chain comprising an amino acid sequence at least 80% identical to the amino acid sequence of SEQ ID NO: 244, and a heavy chain comprising an amino acid sequence at least 80% identical to the amino acid sequence of SEQ ID NO: 159; or
lxxxiii. a light chain comprising an amino acid sequence at least 80% identical to the amino acid sequence of SEQ ID NO: 254, and a heavy chain comprising an amino acid sequence at least 80% identical to the amino acid sequence of SEQ ID NO: 159; or
lxxxiv. a light chain comprising an amino acid sequence at least 80% identical to the amino acid sequence of SEQ ID NO: 104, and a heavy chain comprising an amino acid sequence at least 80% identical to the amino acid sequence of SEQ ID NO: 169; or
lxxxv. a light chain comprising an amino acid sequence at least 80% identical to the amino acid sequence of SEQ ID NO: 114, and a heavy chain comprising an amino acid sequence at least 80% identical to the amino acid sequence of SEQ ID NO: 169; or
lxxxvi. a light chain comprising an amino acid sequence at least 80% identical to the amino acid sequence of SEQ ID NO: 124, and a heavy chain comprising an amino acid sequence at least 80% identical to the amino acid sequence of SEQ ID NO: 169; or
lxxxvii. a light chain comprising an amino acid sequence at least 80% identical to the amino acid sequence of SEQ ID NO: 164, and a heavy chain comprising an amino acid sequence at least 80% identical to the amino acid sequence of SEQ ID NO: 169; or
lxxxviii. a light chain comprising an amino acid sequence at least 80% identical to the amino acid sequence of SEQ ID NO: 174, and a heavy chain comprising an amino acid sequence at least 80% identical to the amino acid sequence of SEQ ID NO: 169; or
lxxxix. a light chain comprising an amino acid sequence at least 80% identical to the amino acid sequence of SEQ ID NO: 184, and a heavy chain comprising an amino acid sequence at least 80% identical to the amino acid sequence of SEQ ID NO: 169; or
xc. a light chain comprising an amino acid sequence at least 80% identical to the amino acid sequence of SEQ ID NO: 194, and a heavy chain comprising an amino acid sequence at least 80% identical to the amino acid sequence of SEQ ID NO: 169; or
xci. a light chain comprising an amino acid sequence at least 80% identical to the amino acid sequence of SEQ ID NO: 204, and a heavy chain comprising an amino acid sequence at least 80% identical to the amino acid sequence of SEQ ID NO: 169; or
xcii. a light chain comprising an amino acid sequence at least 80% identical to the amino acid sequence of SEQ ID NO: 214, and a heavy chain comprising an amino acid sequence at least 80% identical to the amino acid sequence of SEQ ID NO: 169; or
xciii. a light chain comprising an amino acid sequence at least 80% identical to the amino acid sequence of SEQ ID NO: 224, and a heavy chain comprising an amino acid sequence at least 80% identical to the amino acid sequence of SEQ ID NO: 169; or
xciv. a light chain comprising an amino acid sequence at least 80% identical to the amino acid sequence of SEQ ID NO: 234, and a heavy chain comprising an amino acid sequence at least 80% identical to the amino acid sequence of SEQ ID NO: 169; or
xcv. a light chain comprising an amino acid sequence at least 80% identical to the amino acid sequence of SEQ ID NO: 244, and a heavy chain comprising an amino acid sequence at least 80% identical to the amino acid sequence of SEQ ID NO: 169; or
xcvi. a light chain comprising an amino acid sequence at least 80% identical to the amino acid sequence of SEQ ID NO: 254, and a heavy chain comprising an amino acid sequence at least 80% identical to the amino acid sequence of SEQ ID NO: 169; or
xcvii. a light chain comprising an amino acid sequence at least 80% identical to the amino acid sequence of SEQ ID NO: 104, and a heavy chain comprising an amino acid sequence at least 80% identical to the amino acid sequence of SEQ ID NO: 179; or
xcviii. a light chain comprising an amino acid sequence at least 80% identical to the amino acid sequence of SEQ ID NO: 114, and a heavy chain comprising an amino acid sequence at least 80% identical to the amino acid sequence of SEQ ID NO: 179; or
xcix. a light chain comprising an amino acid sequence at least 80% identical to the amino acid sequence of SEQ ID NO: 124, and a heavy chain comprising an amino acid sequence at least 80% identical to the amino acid sequence of SEQ ID NO: 179; or
c. a light chain comprising an amino acid sequence at least 80% identical to the amino acid sequence of SEQ ID NO: 154, and a heavy chain comprising an amino acid sequence at least 80% identical to the amino acid sequence of SEQ ID NO: 179; or
ci. a light chain comprising an amino acid sequence at least 80% identical to the amino acid sequence of SEQ ID NO: 164, and a heavy chain comprising an amino acid sequence at least 80% identical to the amino acid sequence of SEQ ID NO: 179; or
cii. a light chain comprising an amino acid sequence at least 80% identical to the amino acid sequence of SEQ ID NO: 174, and a heavy chain comprising an amino acid sequence at least 80% identical to the amino acid sequence of SEQ ID NO: 179; or
ciii. a light chain comprising an amino acid sequence at least 80% identical to the amino acid sequence of SEQ ID NO: 184, and a heavy chain comprising an amino acid sequence at least 80% identical to the amino acid sequence of SEQ ID NO: 179; or
civ. a light chain comprising an amino acid sequence at least 80% identical to the amino acid sequence of SEQ ID NO: 194, and a heavy chain comprising an amino acid sequence at least 80% identical to the amino acid sequence of SEQ ID NO: 179; or
cv. a light chain comprising an amino acid sequence at least 80% identical to the amino acid sequence of SEQ ID NO: 204, and a heavy chain comprising an amino acid sequence at least 80% identical to the amino acid sequence of SEQ ID NO: 179; or
cvi. a light chain comprising an amino acid sequence at least 80% identical to the amino acid sequence of SEQ ID NO: 214, and a heavy chain comprising an amino acid sequence at least 80% identical to the amino acid sequence of SEQ ID NO: 179; or
cvii. a light chain comprising an amino acid sequence at least 80% identical to the amino acid sequence of SEQ ID NO: 224, and a heavy chain comprising an amino acid sequence at least 80% identical to the amino acid sequence of SEQ ID NO: 179; or
cviii. a light chain comprising an amino acid sequence at least 80% identical to the amino acid sequence of SEQ ID NO: 234, and a heavy chain comprising an amino acid sequence at least 80% identical to the amino acid sequence of SEQ ID NO: 179; or
cix. a light chain comprising an amino acid sequence at least 80% identical to the amino acid sequence of SEQ ID NO: 244, and a heavy chain comprising an amino acid sequence at least 80% identical to the amino acid sequence of SEQ ID NO: 179.

9. The method according to claim 1, wherein the anti-CTGF antibody or antigen-binding fragment thereof comprises:
i. a light chain comprising an amino acid sequence at least 85% identical to the amino acid sequence of SEQ ID NO: 224, and a heavy chain comprising an amino acid sequence at least 85% identical to the amino acid sequence of SEQ ID NO: 109; or
ii. a light chain comprising an amino acid sequence at least 85% identical to the amino acid sequence of SEQ ID NO: 104, and a heavy chain comprising an amino acid sequence at least 85% identical to the amino acid sequence of SEQ ID NO: 109; or
iii. a light chain comprising an amino acid sequence at least 85% identical to the amino acid sequence of SEQ ID NO: 114, and a heavy chain comprising an amino acid sequence at least 85% identical to the amino acid sequence of SEQ ID NO: 109; or
iv. a light chain comprising an amino acid sequence at least 85% identical to the amino acid sequence of SEQ ID NO: 124, and a heavy chain comprising an amino acid sequence at least 85% identical to the amino acid sequence of SEQ ID NO: 109; or
v. a light chain comprising an amino acid sequence at least 85% identical to the amino acid sequence of SEQ ID NO: 134, and a heavy chain comprising an amino acid sequence at least 85% identical to the amino acid sequence of SEQ ID NO: 109; or
vi. a light chain comprising an amino acid sequence at least 85% identical to the amino acid sequence of SEQ ID NO: 154, and a heavy chain comprising an amino acid sequence at least 85% identical to the amino acid sequence of SEQ ID NO: 109; or
vii. a light chain comprising an amino acid sequence at least 85% identical to the amino acid sequence of SEQ ID NO: 164, and a heavy chain comprising an amino acid sequence at least 85% identical to the amino acid sequence of SEQ ID NO: 109; or
viii. a light chain comprising an amino acid sequence at least 85% identical to the amino acid sequence of SEQ ID NO: 174, and a heavy chain comprising an amino acid sequence at least 85% identical to the amino acid sequence of SEQ ID NO: 109; or
ix. a light chain comprising an amino acid sequence at least 85% identical to the amino acid sequence of SEQ ID NO: 184, and a heavy chain comprising an amino acid sequence at least 85% identical to the amino acid sequence of SEQ ID NO: 109; or
x. a light chain comprising an amino acid sequence at least 85% identical to the amino acid sequence of SEQ ID NO: 194, and a heavy chain comprising an amino acid sequence at least 85% identical to the amino acid sequence of SEQ ID NO: 109; or
xi. a light chain comprising an amino acid sequence at least 85% identical to the amino acid sequence of SEQ ID NO: 204, and a heavy chain comprising an amino acid sequence at least 85% identical to the amino acid sequence of SEQ ID NO: 109; or
xii. a light chain comprising an amino acid sequence at least 85% identical to the amino acid sequence of SEQ ID NO: 214, and a heavy chain comprising an amino acid sequence at least 85% identical to the amino acid sequence of SEQ ID NO: 109; or
xiii. a light chain comprising an amino acid sequence at least 85% identical to the amino acid sequence of SEQ ID NO: 234, and a heavy chain comprising an amino acid sequence at least 85% identical to the amino acid sequence of SEQ ID NO: 109; or
xiv. a light chain comprising an amino acid sequence at least 85% identical to the amino acid sequence of SEQ ID NO: 244, and a heavy chain comprising an amino acid sequence at least 85% identical to the amino acid sequence of SEQ ID NO: 109; or
xv. a light chain comprising an amino acid sequence at least 85% identical to the amino acid sequence of SEQ ID NO: 254, and a heavy chain comprising an amino acid sequence at least 85% identical to the amino acid sequence of SEQ ID NO: 109; or
xvi. a light chain comprising an amino acid sequence at least 85% identical to the amino acid sequence of SEQ ID NO: 104, and a heavy chain comprising an amino acid sequence at least 85% identical to the amino acid sequence of SEQ ID NO: 119; or
xvii. a light chain comprising an amino acid sequence at least 85% identical to the amino acid sequence of SEQ ID NO: 114, and a heavy chain comprising an amino acid sequence at least 85% identical to the amino acid sequence of SEQ ID NO: 119; or
xviii. a light chain comprising an amino acid sequence at least 85% identical to the amino acid sequence of SEQ ID NO: 124, and a heavy chain comprising an amino acid sequence at least 85% identical to the amino acid sequence of SEQ ID NO: 119; or
xix. a light chain comprising an amino acid sequence at least 85% identical to the amino acid sequence of SEQ ID NO: 154, and a heavy chain comprising an amino acid sequence at least 85% identical to the amino acid sequence of SEQ ID NO: 119; or
xx. a light chain comprising an amino acid sequence at least 85% identical to the amino acid sequence of SEQ ID NO: 164, and a heavy chain comprising an amino acid sequence at least 85% identical to the amino acid sequence of SEQ ID NO: 119; or
xxi. a light chain comprising an amino acid sequence at least 85% identical to the amino acid sequence of SEQ ID NO: 174, and a heavy chain comprising an amino acid sequence at least 85% identical to the amino acid sequence of SEQ ID NO: 119; or
xxii. a light chain comprising an amino acid sequence at least 85% identical to the amino acid sequence of SEQ ID NO: 184, and a heavy chain comprising an amino acid sequence at least 85% identical to the amino acid sequence of SEQ ID NO: 119; or
xxiii. a light chain comprising an amino acid sequence at least 85% identical to the amino acid sequence of SEQ ID NO: 194, and a heavy chain comprising an amino acid sequence at least 85% identical to the amino acid sequence of SEQ ID NO: 119; or
xxiv. a light chain comprising an amino acid sequence at least 85% identical to the amino acid sequence of SEQ ID NO: 204, and a heavy chain comprising an amino acid sequence at least 85% identical to the amino acid sequence of SEQ ID NO: 119; or
xxv. a light chain comprising an amino acid sequence at least 85% identical to the amino acid sequence of SEQ ID NO: 214, and a heavy chain comprising an amino acid sequence at least 85% identical to the amino acid sequence of SEQ ID NO: 119; or
xxvi. a light chain comprising an amino acid sequence at least 85% identical to the amino acid sequence of SEQ ID NO: 224, and a heavy chain comprising an amino acid sequence at least 85% identical to the amino acid sequence of SEQ ID NO: 119; or
xxvii. a light chain comprising an amino acid sequence at least 85% identical to the amino acid sequence of SEQ ID NO: 234, and a heavy chain comprising an amino acid sequence at least 85% identical to the amino acid sequence of SEQ ID NO: 119; or
xxviii. a light chain comprising an amino acid sequence at least 85% identical to the amino acid sequence of SEQ ID NO: 244, and a heavy chain comprising an amino acid sequence at least 85% identical to the amino acid sequence of SEQ ID NO: 119; or
xxix. a light chain comprising an amino acid sequence at least 85% identical to the amino acid sequence of SEQ ID NO: 254, and a heavy chain comprising an amino acid sequence at least 85% identical to the amino acid sequence of SEQ ID NO: 119; or
xxx. a light chain comprising an amino acid sequence at least 85% identical to the amino acid sequence of SEQ ID NO: 104, and a heavy chain comprising an amino acid sequence at least 85% identical to the amino acid sequence of SEQ ID NO: 129; or
xxxi. a light chain comprising an amino acid sequence at least 85% identical to the amino acid sequence of SEQ ID NO: 114, and a heavy chain comprising an amino acid sequence at least 85% identical to the amino acid sequence of SEQ ID NO: 129; or
xxxii. a light chain comprising an amino acid sequence at least 85% identical to the amino acid sequence of SEQ ID NO: 124, and a heavy chain comprising an amino acid sequence at least 85% identical to the amino acid sequence of SEQ ID NO: 129; or
xxxiii. a light chain comprising an amino acid sequence at least 85% identical to the amino acid sequence of SEQ ID NO: 154, and a heavy chain comprising an amino acid sequence at least 85% identical to the amino acid sequence of SEQ ID NO: 129; or
xxxiv. a light chain comprising an amino acid sequence at least 85% identical to the amino acid sequence of SEQ ID NO: 164, and a heavy chain comprising an amino acid sequence at least 85% identical to the amino acid sequence of SEQ ID NO: 129; or
xxxv. a light chain comprising an amino acid sequence at least 85% identical to the amino acid sequence of SEQ ID NO: 174, and a heavy chain comprising an amino acid sequence at least 85% identical to the amino acid sequence of SEQ ID NO: 129; or
xxxvi. a light chain comprising an amino acid sequence at least 85% identical to the amino acid sequence of SEQ ID NO: 184, and a heavy chain comprising an amino acid sequence at least 85% identical to the amino acid sequence of SEQ ID NO: 129; or
xxxvii. a light chain comprising an amino acid sequence at least 85% identical to the amino acid sequence of SEQ ID NO: 194, and a heavy chain comprising an amino acid sequence at least 85% identical to the amino acid sequence of SEQ ID NO: 129; or
xxxviii. a light chain comprising an amino acid sequence at least 85% identical to the amino acid sequence of SEQ ID NO: 204, and a heavy chain comprising an amino acid sequence at least 85% identical to the amino acid sequence of SEQ ID NO: 129; or
xxxix. a light chain comprising an amino acid sequence at least 85% identical to the amino acid sequence of SEQ ID NO: 214, and a heavy chain comprising an amino acid sequence at least 85% identical to the amino acid sequence of SEQ ID NO: 129; or
xl. a light chain comprising an amino acid sequence at least 85% identical to the amino acid sequence of SEQ ID NO: 224, and a heavy chain comprising an amino acid sequence at least 85% identical to the amino acid sequence of SEQ ID NO: 129; or
xli. a light chain comprising an amino acid sequence at least 85% identical to the amino acid sequence of SEQ ID NO: 234, and a heavy chain comprising an amino acid sequence at least 85% identical to the amino acid sequence of SEQ ID NO: 129; or
xlii. a light chain comprising an amino acid sequence at least 85% identical to the amino acid sequence of SEQ ID NO: 244, and a heavy chain comprising an amino acid sequence at least 85% identical to the amino acid sequence of SEQ ID NO: 129; or
xliii. a light chain comprising an amino acid sequence at least 85% identical to the amino acid sequence of SEQ ID NO: 254, and a heavy chain comprising an amino acid sequence at least 85% identical to the amino acid sequence of SEQ ID NO: 129; or
xliv. a light chain comprising an amino acid sequence at least 85% identical to the amino acid sequence of SEQ ID NO: 104, and a heavy chain comprising an amino acid sequence at least 85% identical to the amino acid sequence of SEQ ID NO: 139; or
xlv. a light chain comprising an amino acid sequence at least 85% identical to the amino acid sequence of SEQ ID NO: 114, and a heavy chain comprising an amino acid sequence at least 85% identical to the amino acid sequence of SEQ ID NO: 139; or
xlvi. a light chain comprising an amino acid sequence at least 85% identical to the amino acid sequence of SEQ ID NO: 124, and a heavy chain comprising an amino acid sequence at least 85% identical to the amino acid sequence of SEQ ID NO: 139; or
xlvii. a light chain comprising an amino acid sequence at least 85% identical to the amino acid sequence of SEQ ID NO: 154, and a heavy chain comprising an amino acid sequence at least 85% identical to the amino acid sequence of SEQ ID NO: 139; or
xlviii. a light chain comprising an amino acid sequence at least 85% identical to the amino acid sequence of SEQ ID NO: 164, and a heavy chain comprising an amino acid sequence at least 85% identical to the amino acid sequence of SEQ ID NO: 139; or
xlix. a light chain comprising an amino acid sequence at least 85% identical to the amino acid sequence of SEQ ID NO: 174, and a heavy chain comprising an amino acid sequence at least 85% identical to the amino acid sequence of SEQ ID NO: 139; or
l. a light chain comprising an amino acid sequence at least 85% identical to the amino acid sequence of SEQ ID NO: 184, and a heavy chain comprising an amino acid sequence at least 85% identical to the amino acid sequence of SEQ ID NO: 139; or
li. a light chain comprising an amino acid sequence at least 85% identical to the amino acid sequence of SEQ ID NO: 194, and a heavy chain comprising an amino acid sequence at least 85% identical to the amino acid sequence of SEQ ID NO: 139; or
lii. a light chain comprising an amino acid sequence at least 85% identical to the amino acid sequence of SEQ ID NO: 204, and a heavy chain comprising an amino acid sequence at least 85% identical to the amino acid sequence of SEQ ID NO: 139; or
liii. a light chain comprising an amino acid sequence at least 85% identical to the amino acid sequence of SEQ ID NO: 214, and a heavy chain comprising an amino acid sequence at least 85% identical to the amino acid sequence of SEQ ID NO: 139; or
liv. a light chain comprising an amino acid sequence at least 85% identical to the amino acid sequence of SEQ ID NO: 224, and a heavy chain comprising an amino acid sequence at least 85% identical to the amino acid sequence of SEQ ID NO: 139; or
lv. a light chain comprising an amino acid sequence at least 85% identical to the amino acid sequence of SEQ ID NO: 234, and a heavy chain comprising an amino acid sequence at least 85% identical to the amino acid sequence of SEQ ID NO: 139; or
lvi. a light chain comprising an amino acid sequence at least 85% identical to the amino acid sequence of SEQ ID NO: 244, and a heavy chain comprising an amino acid sequence at least 85% identical to the amino acid sequence of SEQ ID NO: 139; or
lvii. a light chain comprising an amino acid sequence at least 85% identical to the amino acid sequence of SEQ ID NO: 254, and a heavy chain comprising an amino acid sequence at least 85% identical to the amino acid sequence of SEQ ID NO: 139; or
lviii. a light chain comprising an amino acid sequence at least 85% identical to the amino acid sequence of SEQ ID NO: 104, and a heavy chain comprising an amino acid sequence at least 85% identical to the amino acid sequence of SEQ ID NO: 149; or
lix. a light chain comprising an amino acid sequence at least 85% identical to the amino acid sequence of SEQ ID NO: 114, and a heavy chain comprising an amino acid sequence at least 85% identical to the amino acid sequence of SEQ ID NO: 149; or
lx. a light chain comprising an amino acid sequence at least 85% identical to the amino acid sequence of SEQ ID NO: 124, and a heavy chain comprising an amino acid sequence at least 85% identical to the amino acid sequence of SEQ ID NO: 149; or
lxi. a light chain comprising an amino acid sequence at least 85% identical to the amino acid sequence of SEQ ID NO: 164, and a heavy chain comprising an amino acid sequence at least 85% identical to the amino acid sequence of SEQ ID NO: 149; or
lxii. a light chain comprising an amino acid sequence at least 85% identical to the amino acid sequence of SEQ ID NO: 174, and a heavy chain comprising an amino acid sequence at least 85% identical to the amino acid sequence of SEQ ID NO: 149; or
lxiii. a light chain comprising an amino acid sequence at least 85% identical to the amino acid sequence of SEQ ID NO: 184, and a heavy chain comprising an amino acid sequence at least 85% identical to the amino acid sequence of SEQ ID NO: 149; or
lxiv. a light chain comprising an amino acid sequence at least 85% identical to the amino acid sequence of SEQ ID NO: 194, and a heavy chain comprising an amino acid sequence at least 85% identical to the amino acid sequence of SEQ ID NO: 149; or
lxv. a light chain comprising an amino acid sequence at least 85% identical to the amino acid sequence of SEQ ID NO: 204, and a heavy chain comprising an amino acid sequence at least 85% identical to the amino acid sequence of SEQ ID NO: 149; or
lxvi. a light chain comprising an amino acid sequence at least 85% identical to the amino acid sequence of SEQ ID NO: 214, and a heavy chain comprising an amino acid sequence at least 85% identical to the amino acid sequence of SEQ ID NO: 149; or
lxvii. a light chain comprising an amino acid sequence at least 85% identical to the amino acid sequence of SEQ ID NO: 224, and a heavy chain comprising an amino acid sequence at least 85% identical to the amino acid sequence of SEQ ID NO: 149; or
lxviii. a light chain comprising an amino acid sequence at least 85% identical to the amino acid sequence of SEQ ID NO: 234, and a heavy chain comprising an amino acid sequence at least 85% identical to the amino acid sequence of SEQ ID NO: 149; or
lxix. a light chain comprising an amino acid sequence at least 85% identical to the amino acid sequence of SEQ ID NO: 244, and a heavy chain comprising an amino acid sequence at least 85% identical to the amino acid sequence of SEQ ID NO: 149; or
lxx. a light chain comprising an amino acid sequence at least 85% identical to the amino acid sequence of SEQ ID NO: 254, and a heavy chain comprising an amino acid sequence at least 85% identical to the amino acid sequence of SEQ ID NO: 149; or
lxxi. a light chain comprising an amino acid sequence at least 85% identical to the amino acid sequence of SEQ ID NO: 104, and a heavy chain comprising an amino acid sequence at least 85% identical to the amino acid sequence of SEQ ID NO: 159; or
lxxii. a light chain comprising an amino acid sequence at least 85% identical to the amino acid sequence of SEQ ID NO: 114, and a heavy chain comprising an amino acid sequence at least 85% identical to the amino acid sequence of SEQ ID NO: 159; or
lxxiii. a light chain comprising an amino acid sequence at least 85% identical to the amino acid sequence of SEQ ID NO: 124, and a heavy chain comprising an amino acid sequence at least 85% identical to the amino acid sequence of SEQ ID NO: 159; or
lxxiv. a light chain comprising an amino acid sequence at least 85% identical to the amino acid sequence of SEQ ID NO: 154, and a heavy chain comprising an amino acid sequence at least 85% identical to the amino acid sequence of SEQ ID NO: 159; or
lxxv. a light chain comprising an amino acid sequence at least 85% identical to the amino acid sequence of SEQ ID NO: 164, and a heavy chain comprising an amino acid sequence at least 85% identical to the amino acid sequence of SEQ ID NO: 159; or
lxxvi. a light chain comprising an amino acid sequence at least 85% identical to the amino acid sequence of SEQ ID NO: 174, and a heavy chain comprising an amino acid sequence at least 85% identical to the amino acid sequence of SEQ ID NO: 159; or
lxxvii. a light chain comprising an amino acid sequence at least 85% identical to the amino acid sequence of SEQ ID NO: 184, and a heavy chain comprising an amino acid sequence at least 85% identical to the amino acid sequence of SEQ ID NO: 159; or
lxxviii. a light chain comprising an amino acid sequence at least 85% identical to the amino acid sequence of SEQ ID NO: 194, and a heavy chain comprising an amino acid sequence at least 85% identical to the amino acid sequence of SEQ ID NO: 159; or
lxxix. a light chain comprising an amino acid sequence at least 85% identical to the amino acid sequence of SEQ ID NO: 214, and a heavy chain comprising an amino acid sequence at least 85% identical to the amino acid sequence of SEQ ID NO: 159; or
lxxx. a light chain comprising an amino acid sequence at least 85% identical to the amino acid sequence of SEQ ID NO: 224, and a heavy chain comprising an amino acid sequence at least 85% identical to the amino acid sequence of SEQ ID NO: 159; or
lxxxi. a light chain comprising an amino acid sequence at least 85% identical to the amino acid sequence of SEQ ID NO: 234, and a heavy chain comprising an amino acid sequence at least 85% identical to the amino acid sequence of SEQ ID NO: 159; or
lxxxii. a light chain comprising an amino acid sequence at least 85% identical to the amino acid sequence of SEQ ID NO: 244, and a heavy chain comprising an amino acid sequence at least 85% identical to the amino acid sequence of SEQ ID NO: 159; or
lxxxiii. a light chain comprising an amino acid sequence at least 85% identical to the amino acid sequence of SEQ ID NO: 254, and a heavy chain comprising an amino acid sequence at least 85% identical to the amino acid sequence of SEQ ID NO: 159; or
lxxxiv. a light chain comprising an amino acid sequence at least 85% identical to the amino acid sequence of SEQ ID NO: 104, and a heavy chain comprising an amino acid sequence at least 85% identical to the amino acid sequence of SEQ ID NO: 169; or
lxxxv. a light chain comprising an amino acid sequence at least 85% identical to the amino acid sequence of SEQ ID NO: 114, and a heavy chain comprising an amino acid sequence at least 85% identical to the amino acid sequence of SEQ ID NO: 169; or
lxxxvi. a light chain comprising an amino acid sequence at least 85% identical to the amino acid sequence of SEQ ID NO: 124, and a heavy chain comprising an amino acid sequence at least 85% identical to the amino acid sequence of SEQ ID NO: 169; or
lxxxvii. a light chain comprising an amino acid sequence at least 85% identical to the amino acid sequence of SEQ ID NO: 164, and a heavy chain comprising an amino acid sequence at least 85% identical to the amino acid sequence of SEQ ID NO: 169; or
lxxxviii. a light chain comprising an amino acid sequence at least 85% identical to the amino acid sequence of SEQ ID NO: 174, and a heavy chain comprising an amino acid sequence at least 85% identical to the amino acid sequence of SEQ ID NO: 169; or
lxxxix. a light chain comprising an amino acid sequence at least 85% identical to the amino acid sequence of SEQ ID NO: 184, and a heavy chain comprising an amino acid sequence at least 85% identical to the amino acid sequence of SEQ ID NO: 169; or
xc. a light chain comprising an amino acid sequence at least 85% identical to the amino acid sequence of SEQ ID NO: 194, and a heavy chain comprising an amino acid sequence at least 85% identical to the amino acid sequence of SEQ ID NO: 169; or
xci. a light chain comprising an amino acid sequence at least 85% identical to the amino acid sequence of SEQ ID NO: 204, and a heavy chain comprising an amino acid sequence at least 85% identical to the amino acid sequence of SEQ ID NO: 169; or
xcii. a light chain comprising an amino acid sequence at least 85% identical to the amino acid sequence of SEQ ID NO: 214, and a heavy chain comprising an amino acid sequence at least 85% identical to the amino acid sequence of SEQ ID NO: 169; or
xciii. a light chain comprising an amino acid sequence at least 85% identical to the amino acid sequence of SEQ ID NO: 224, and a heavy chain comprising an amino acid sequence at least 85% identical to the amino acid sequence of SEQ ID NO: 169; or
xciv. a light chain comprising an amino acid sequence at least 85% identical to the amino acid sequence of SEQ ID NO: 234, and a heavy chain comprising an amino acid sequence at least 85% identical to the amino acid sequence of SEQ ID NO: 169; or
xcv. a light chain comprising an amino acid sequence at least 85% identical to the amino acid sequence of SEQ ID NO: 244, and a heavy chain comprising an amino acid sequence at least 85% identical to the amino acid sequence of SEQ ID NO: 169; or
xcvi. a light chain comprising an amino acid sequence at least 85% identical to the amino acid sequence of SEQ ID NO: 254, and a heavy chain comprising an amino acid sequence at least 85% identical to the amino acid sequence of SEQ ID NO: 169; or
xcvii. a light chain comprising an amino acid sequence at least 85% identical to the amino acid sequence of SEQ ID NO: 104, and a heavy chain comprising an amino acid sequence at least 85% identical to the amino acid sequence of SEQ ID NO: 179; or
xcviii. a light chain comprising an amino acid sequence at least 85% identical to the amino acid sequence of SEQ ID NO: 114, and a heavy chain comprising an amino acid sequence at least 85% identical to the amino acid sequence of SEQ ID NO: 179; or
xcix. a light chain comprising an amino acid sequence at least 85% identical to the amino acid sequence of SEQ ID NO: 124, and a heavy chain comprising an amino acid sequence at least 85% identical to the amino acid sequence of SEQ ID NO: 179; or
c. a light chain comprising an amino acid sequence at least 85% identical to the amino acid sequence of SEQ ID NO: 154, and a heavy chain comprising an amino acid sequence at least 85% identical to the amino acid sequence of SEQ ID NO: 179; or
ci. a light chain comprising an amino acid sequence at least 85% identical to the amino acid sequence of SEQ ID NO: 164, and a heavy chain comprising an amino acid sequence at least 85% identical to the amino acid sequence of SEQ ID NO: 179; or
cii. a light chain comprising an amino acid sequence at least 85% identical to the amino acid sequence of SEQ ID NO: 174, and a heavy chain comprising an amino acid sequence at least 85% identical to the amino acid sequence of SEQ ID NO: 179; or
ciii. a light chain comprising an amino acid sequence at least 85% identical to the amino acid sequence of SEQ ID NO: 184, and a heavy chain comprising an amino acid sequence at least 85% identical to the amino acid sequence of SEQ ID NO: 179; or
civ. a light chain comprising an amino acid sequence at least 85% identical to the amino acid sequence of SEQ ID NO: 194, and a heavy chain comprising an amino acid sequence at least 85% identical to the amino acid sequence of SEQ ID NO: 179; or
cv. a light chain comprising an amino acid sequence at least 85% identical to the amino acid sequence of SEQ ID NO: 204, and a heavy chain comprising an amino acid sequence at least 85% identical to the amino acid sequence of SEQ ID NO: 179; or
cvi. a light chain comprising an amino acid sequence at least 85% identical to the amino acid sequence of SEQ ID NO: 214, and a heavy chain comprising an amino acid sequence at least 85% identical to the amino acid sequence of SEQ ID NO: 179; or
cvii. a light chain comprising an amino acid sequence at least 85% identical to the amino acid sequence of SEQ ID NO: 224, and a heavy chain comprising an amino acid sequence at least 85% identical to the amino acid sequence of SEQ ID NO: 179; or
cviii. a light chain comprising an amino acid sequence at least 85% identical to the amino acid sequence of SEQ ID NO: 234, and a heavy chain comprising an amino acid sequence at least 85% identical to the amino acid sequence of SEQ ID NO: 179; or
cix. a light chain comprising an amino acid sequence at least 85% identical to the amino acid sequence of SEQ ID NO: 244, and a heavy chain comprising an amino acid sequence at least 85% identical to the amino acid sequence of SEQ ID NO: 179.

10. The method according to claim 1, wherein the anti-CTGF antibody or antigen-binding fragment thereof comprises:
i. a light chain comprising an amino acid sequence at least 90% identical to the amino acid sequence of SEQ ID NO: 224, and a heavy chain comprising an amino acid sequence at least 90% identical to the amino acid sequence of SEQ ID NO: 109; or
ii. a light chain comprising an amino acid sequence at least 90% identical to the amino acid sequence of SEQ ID NO: 104, and a heavy chain comprising an amino acid sequence at least 90% identical to the amino acid sequence of SEQ ID NO: 109; or
iii. a light chain comprising an amino acid sequence at least 90% identical to the amino acid sequence of SEQ ID NO: 114, and a heavy chain comprising an amino acid sequence at least 90% identical to the amino acid sequence of SEQ ID NO: 109; or
iv. a light chain comprising an amino acid sequence at least 90% identical to the amino acid sequence of SEQ ID NO: 124, and a heavy chain comprising an amino acid sequence at least 90% identical to the amino acid sequence of SEQ ID NO: 109; or
v. a light chain comprising an amino acid sequence at least 90% identical to the amino acid sequence of SEQ ID NO: 134, and a heavy chain comprising an amino acid sequence at least 90% identical to the amino acid sequence of SEQ ID NO: 109; or
vi. a light chain comprising an amino acid sequence at least 90% identical to the amino acid sequence of SEQ ID NO: 154, and a heavy chain comprising an amino acid sequence at least 90% identical to the amino acid sequence of SEQ ID NO: 109; or
vii. a light chain comprising an amino acid sequence at least 90% identical to the amino acid sequence of SEQ ID NO: 164, and a heavy chain comprising an amino acid sequence at least 90% identical to the amino acid sequence of SEQ ID NO: 109; or
viii. a light chain comprising an amino acid sequence at least 90% identical to the amino acid sequence of SEQ ID NO: 174, and a heavy chain comprising an amino acid sequence at least 90% identical to the amino acid sequence of SEQ ID NO: 109; or
ix. a light chain comprising an amino acid sequence at least 90% identical to the amino acid sequence of SEQ ID NO: 184, and a heavy chain comprising an amino acid sequence at least 90% identical to the amino acid sequence of SEQ ID NO: 109; or
x. a light chain comprising an amino acid sequence at least 90% identical to the amino acid sequence of SEQ ID NO: 194, and a heavy chain comprising an amino acid sequence at least 90% identical to the amino acid sequence of SEQ ID NO: 109; or
xi. a light chain comprising an amino acid sequence at least 90% identical to the amino acid sequence of SEQ ID NO: 204, and a heavy chain comprising an amino acid sequence at least 90% identical to the amino acid sequence of SEQ ID NO: 109; or
xii. a light chain comprising an amino acid sequence at least 90% identical to the amino acid sequence of SEQ ID NO: 214, and a heavy chain comprising an amino acid sequence at least 90% identical to the amino acid sequence of SEQ ID NO: 109; or
xiii. a light chain comprising an amino acid sequence at least 90% identical to the amino acid sequence of SEQ ID NO: 234, and a heavy chain comprising an amino acid sequence at least 90% identical to the amino acid sequence of SEQ ID NO: 109; or
xiv. a light chain comprising an amino acid sequence at least 90% identical to the amino acid sequence of SEQ ID NO: 244, and a heavy chain comprising an amino acid sequence at least 90% identical to the amino acid sequence of SEQ ID NO: 109; or
xv. a light chain comprising an amino acid sequence at least 90% identical to the amino acid sequence of SEQ ID NO: 254, and a heavy chain comprising an amino acid sequence at least 90% identical to the amino acid sequence of SEQ ID NO: 109; or
xvi. a light chain comprising an amino acid sequence at least 90% identical to the amino acid sequence of SEQ ID NO: 104, and a heavy chain comprising an amino acid sequence at least 90% identical to the amino acid sequence of SEQ ID NO: 119; or
xvii. a light chain comprising an amino acid sequence at least 90% identical to the amino acid sequence of SEQ ID NO: 114, and a heavy chain comprising an amino acid sequence at least 90% identical to the amino acid sequence of SEQ ID NO: 119; or
xviii. a light chain comprising an amino acid sequence at least 90% identical to the amino acid sequence of SEQ ID NO: 124, and a heavy chain comprising an amino acid sequence at least 90% identical to the amino acid sequence of SEQ ID NO: 119; or
xix. a light chain comprising an amino acid sequence at least 90% identical to the amino acid sequence of SEQ ID NO: 154, and a heavy chain comprising an amino acid sequence at least 90% identical to the amino acid sequence of SEQ ID NO: 119; or
xx. a light chain comprising an amino acid sequence at least 90% identical to the amino acid sequence of SEQ ID NO: 164, and a heavy chain comprising an amino acid sequence at least 90% identical to the amino acid sequence of SEQ ID NO: 119; or
xxi. a light chain comprising an amino acid sequence at least 90% identical to the amino acid sequence of SEQ ID NO: 174, and a heavy chain comprising an amino acid sequence at least 90% identical to the amino acid sequence of SEQ ID NO: 119; or
xxii. a light chain comprising an amino acid sequence at least 90% identical to the amino acid sequence of SEQ ID NO: 184, and a heavy chain comprising an amino acid sequence at least 90% identical to the amino acid sequence of SEQ ID NO: 119; or
xxiii. a light chain comprising an amino acid sequence at least 90% identical to the amino acid sequence of SEQ ID NO: 194, and a heavy chain comprising an amino acid sequence at least 90% identical to the amino acid sequence of SEQ ID NO: 119; or
xxiv. a light chain comprising an amino acid sequence at least 90% identical to the amino acid sequence of SEQ ID NO: 204, and a heavy chain comprising an amino acid sequence at least 90% identical to the amino acid sequence of SEQ ID NO: 119; or
xxv. a light chain comprising an amino acid sequence at least 90% identical to the amino acid sequence of SEQ ID NO: 214, and a heavy chain comprising an amino acid sequence at least 90% identical to the amino acid sequence of SEQ ID NO: 119; or
xxvi. a light chain comprising an amino acid sequence at least 90% identical to the amino acid sequence of SEQ ID NO: 224, and a heavy chain comprising an amino acid sequence at least 90% identical to the amino acid sequence of SEQ ID NO: 119; or
xxvii. a light chain comprising an amino acid sequence at least 90% identical to the amino acid sequence of SEQ ID NO: 234, and a heavy chain comprising an amino acid sequence at least 90% identical to the amino acid sequence of SEQ ID NO: 119; or
xxviii. a light chain comprising an amino acid sequence at least 90% identical to the amino acid sequence of SEQ ID NO: 244, and a heavy chain comprising an amino acid sequence at least 90% identical to the amino acid sequence of SEQ ID NO: 119; or
xxix. a light chain comprising an amino acid sequence at least 90% identical to the amino acid sequence of SEQ ID NO: 254, and a heavy chain comprising an amino acid sequence at least 90% identical to the amino acid sequence of SEQ ID NO: 119; or
xxx. a light chain comprising an amino acid sequence at least 90% identical to the amino acid sequence of SEQ ID NO: 104, and a heavy chain comprising an amino acid sequence at least 90% identical to the amino acid sequence of SEQ ID NO: 129; or
xxxi. a light chain comprising an amino acid sequence at least 90% identical to the amino acid sequence of SEQ ID NO: 114, and a heavy chain comprising an amino acid sequence at least 90% identical to the amino acid sequence of SEQ ID NO: 129; or
xxxii. a light chain comprising an amino acid sequence at least 90% identical to the amino acid sequence of SEQ ID NO: 124, and a heavy chain comprising an amino acid sequence at least 90% identical to the amino acid sequence of SEQ ID NO: 129; or
xxxiii. a light chain comprising an amino acid sequence at least 90% identical to the amino acid sequence of SEQ ID NO: 154, and a heavy chain comprising an amino acid sequence at least 90% identical to the amino acid sequence of SEQ ID NO: 129; or
xxxiv. a light chain comprising an amino acid sequence at least 90% identical to the amino acid sequence of SEQ ID NO: 164, and a heavy chain comprising an amino acid sequence at least 90% identical to the amino acid sequence of SEQ ID NO: 129; or
xxxv. a light chain comprising an amino acid sequence at least 90% identical to the amino acid sequence of SEQ ID NO: 174, and a heavy chain comprising an amino acid sequence at least 90% identical to the amino acid sequence of SEQ ID NO: 129; or
xxxvi. a light chain comprising an amino acid sequence at least 90% identical to the amino acid sequence of SEQ ID NO: 184, and a heavy chain comprising an amino acid sequence at least 90% identical to the amino acid sequence of SEQ ID NO: 129; or
xxxvii. a light chain comprising an amino acid sequence at least 90% identical to the amino acid sequence of SEQ ID NO: 194, and a heavy chain comprising an amino acid sequence at least 90% identical to the amino acid sequence of SEQ ID NO: 129; or
xxxviii. a light chain comprising an amino acid sequence at least 90% identical to the amino acid sequence of SEQ ID NO: 204, and a heavy chain comprising an amino acid sequence at least 90% identical to the amino acid sequence of SEQ ID NO: 129; or
xxxix. a light chain comprising an amino acid sequence at least 90% identical to the amino acid sequence of SEQ ID NO: 214, and a heavy chain comprising an amino acid sequence at least 90% identical to the amino acid sequence of SEQ ID NO: 129; or
xl. a light chain comprising an amino acid sequence at least 90% identical to the amino acid sequence of SEQ ID NO: 224, and a heavy chain comprising an amino acid sequence at least 90% identical to the amino acid sequence of SEQ ID NO: 129; or
xli. a light chain comprising an amino acid sequence at least 90% identical to the amino acid sequence of SEQ ID NO: 234, and a heavy chain comprising an amino acid sequence at least 90% identical to the amino acid sequence of SEQ ID NO: 129; or
xlii. a light chain comprising an amino acid sequence at least 90% identical to the amino acid sequence of SEQ ID NO: 244, and a heavy chain comprising an amino acid sequence at least 90% identical to the amino acid sequence of SEQ ID NO: 129; or
xliii. a light chain comprising an amino acid sequence at least 90% identical to the amino acid sequence of SEQ ID NO: 254, and a heavy chain comprising an amino acid sequence at least 90% identical to the amino acid sequence of SEQ ID NO: 129; or
xliv. a light chain comprising an amino acid sequence at least 90% identical to the amino acid sequence of SEQ ID NO: 104, and a heavy chain comprising an amino acid sequence at least 90% identical to the amino acid sequence of SEQ ID NO: 139; or
xlv. a light chain comprising an amino acid sequence at least 90% identical to the amino acid sequence of SEQ ID NO: 114, and a heavy chain comprising an amino acid sequence at least 90% identical to the amino acid sequence of SEQ ID NO: 139; or
xlvi. a light chain comprising an amino acid sequence at least 90% identical to the amino acid sequence of SEQ ID NO: 124, and a heavy chain comprising an amino acid sequence at least 90% identical to the amino acid sequence of SEQ ID NO: 139; or
xlvii. a light chain comprising an amino acid sequence at least 90% identical to the amino acid sequence of SEQ ID NO: 154, and a heavy chain comprising an amino acid sequence at least 90% identical to the amino acid sequence of SEQ ID NO: 139; or
xlviii. a light chain comprising an amino acid sequence at least 90% identical to the amino acid sequence of SEQ ID NO: 164, and a heavy chain comprising an amino acid sequence at least 90% identical to the amino acid sequence of SEQ ID NO: 139; or
xlix. a light chain comprising an amino acid sequence at least 90% identical to the amino acid sequence of SEQ ID NO: 174, and a heavy chain comprising an amino acid sequence at least 90% identical to the amino acid sequence of SEQ ID NO: 139; or
l. a light chain comprising an amino acid sequence at least 90% identical to the amino acid sequence of SEQ ID NO: 184, and a heavy chain comprising an amino acid sequence at least 90% identical to the amino acid sequence of SEQ ID NO: 139; or
li. a light chain comprising an amino acid sequence at least 90% identical to the amino acid sequence of SEQ ID NO: 194, and a heavy chain comprising an amino acid sequence at least 90% identical to the amino acid sequence of SEQ ID NO: 139; or
lii. a light chain comprising an amino acid sequence at least 90% identical to the amino acid sequence of SEQ ID NO: 204, and a heavy chain comprising an amino acid sequence at least 90% identical to the amino acid sequence of SEQ ID NO: 139; or
liii. a light chain comprising an amino acid sequence at least 90% identical to the amino acid sequence of SEQ ID NO: 214, and a heavy chain comprising an amino acid sequence at least 90% identical to the amino acid sequence of SEQ ID NO: 139; or
liv. a light chain comprising an amino acid sequence at least 90% identical to the amino acid sequence of SEQ ID NO: 224, and a heavy chain comprising an amino acid sequence at least 90% identical to the amino acid sequence of SEQ ID NO: 139; or
lv. a light chain comprising an amino acid sequence at least 90% identical to the amino acid sequence of SEQ ID NO: 234, and a heavy chain comprising an amino acid sequence at least 90% identical to the amino acid sequence of SEQ ID NO: 139; or
lvi. a light chain comprising an amino acid sequence at least 90% identical to the amino acid sequence of SEQ ID NO: 244, and a heavy chain comprising an amino acid sequence at least 90% identical to the amino acid sequence of SEQ ID NO: 139; or
lvii. a light chain comprising an amino acid sequence at least 90% identical to the amino acid sequence of SEQ ID NO: 254, and a heavy chain comprising an amino acid sequence at least 90% identical to the amino acid sequence of SEQ ID NO: 139; or
lviii. a light chain comprising an amino acid sequence at least 90% identical to the amino acid sequence of SEQ ID NO: 104, and a heavy chain comprising an amino acid sequence at least 90% identical to the amino acid sequence of SEQ ID NO: 149; or
lix. a light chain comprising an amino acid sequence at least 90% identical to the amino acid sequence of SEQ ID NO: 114, and a heavy chain comprising an amino acid sequence at least 90% identical to the amino acid sequence of SEQ ID NO: 149; or
lx. a light chain comprising an amino acid sequence at least 90% identical to the amino acid sequence of SEQ ID NO: 124, and a heavy chain comprising an amino acid sequence at least 90% identical to the amino acid sequence of SEQ ID NO: 149; or
lxi. a light chain comprising an amino acid sequence at least 90% identical to the amino acid sequence of SEQ ID NO: 164, and a heavy chain comprising an amino acid sequence at least 90% identical to the amino acid sequence of SEQ ID NO: 149; or
lxii. a light chain comprising an amino acid sequence at least 90% identical to the amino acid sequence of SEQ ID NO: 174, and a heavy chain comprising an amino acid sequence at least 90% identical to the amino acid sequence of SEQ ID NO: 149; or
lxiii. a light chain comprising an amino acid sequence at least 90% identical to the amino acid sequence of SEQ ID NO: 184, and a heavy chain comprising an amino acid sequence at least 90% identical to the amino acid sequence of SEQ ID NO: 149; or
lxiv. a light chain comprising an amino acid sequence at least 90% identical to the amino acid sequence of SEQ ID NO: 194, and a heavy chain comprising an amino acid sequence at least 90% identical to the amino acid sequence of SEQ ID NO: 149; or
lxv. a light chain comprising an amino acid sequence at least 90% identical to the amino acid sequence of SEQ ID NO: 204, and a heavy chain comprising an amino acid sequence at least 90% identical to the amino acid sequence of SEQ ID NO: 149; or
lxvi. a light chain comprising an amino acid sequence at least 90% identical to the amino acid sequence of SEQ ID NO: 214, and a heavy chain comprising an amino acid sequence at least 90% identical to the amino acid sequence of SEQ ID NO: 149; or
lxvii. a light chain comprising an amino acid sequence at least 90% identical to the amino acid sequence of SEQ ID NO: 224, and a heavy chain comprising an amino acid sequence at least 90% identical to the amino acid sequence of SEQ ID NO: 149; or
lxviii. a light chain comprising an amino acid sequence at least 90% identical to the amino acid sequence of SEQ ID NO: 234, and a heavy chain comprising an amino acid sequence at least 90% identical to the amino acid sequence of SEQ ID NO: 149; or
lxix. a light chain comprising an amino acid sequence at least 90% identical to the amino acid sequence of SEQ ID NO: 244, and a heavy chain comprising an amino acid sequence at least 90% identical to the amino acid sequence of SEQ ID NO: 149; or
lxx. a light chain comprising an amino acid sequence at least 90% identical to the amino acid sequence of SEQ ID NO: 254, and a heavy chain comprising an amino acid sequence at least 90% identical to the amino acid sequence of SEQ ID NO: 149; or
lxxi. a light chain comprising an amino acid sequence at least 90% identical to the amino acid sequence of SEQ ID NO: 104, and a heavy chain comprising an amino acid sequence at least 90% identical to the amino acid sequence of SEQ ID NO: 159; or
lxxii. a light chain comprising an amino acid sequence at least 90% identical to the amino acid sequence of SEQ ID NO: 114, and a heavy chain comprising an amino acid sequence at least 90% identical to the amino acid sequence of SEQ ID NO: 159; or
lxxiii. a light chain comprising an amino acid sequence at least 90% identical to the amino acid sequence of SEQ ID NO: 124, and a heavy chain comprising an amino acid sequence at least 90% identical to the amino acid sequence of SEQ ID NO: 159; or
lxxiv. a light chain comprising an amino acid sequence at least 90% identical to the amino acid sequence of SEQ ID NO: 154, and a heavy chain comprising an amino acid sequence at least 90% identical to the amino acid sequence of SEQ ID NO: 159; or
lxxv. a light chain comprising an amino acid sequence at least 90% identical to the amino acid sequence of SEQ ID NO: 164, and a heavy chain comprising an amino acid sequence at least 90% identical to the amino acid sequence of SEQ ID NO: 159; or
lxxvi. a light chain comprising an amino acid sequence at least 90% identical to the amino acid sequence of SEQ ID NO: 174, and a heavy chain comprising an amino acid sequence at least 90% identical to the amino acid sequence of SEQ ID NO: 159; or
lxxvii. a light chain comprising an amino acid sequence at least 90% identical to the amino acid sequence of SEQ ID NO: 184, and a heavy chain comprising an amino acid sequence at least 90% identical to the amino acid sequence of SEQ ID NO: 159; or
lxxviii. a light chain comprising an amino acid sequence at least 90% identical to the amino acid sequence of SEQ ID NO: 194, and a heavy chain comprising an amino acid sequence at least 90% identical to the amino acid sequence of SEQ ID NO: 159; or
lxxix. a light chain comprising an amino acid sequence at least 90% identical to the amino acid sequence of SEQ ID NO: 214, and a heavy chain comprising an amino acid sequence at least 90% identical to the amino acid sequence of SEQ ID NO: 159; or
lxxx. a light chain comprising an amino acid sequence at least 90% identical to the amino acid sequence of SEQ ID NO: 224, and a heavy chain comprising an amino acid sequence at least 90% identical to the amino acid sequence of SEQ ID NO: 159; or
lxxxi. a light chain comprising an amino acid sequence at least 90% identical to the amino acid sequence of SEQ ID NO: 234, and a heavy chain comprising an amino acid sequence at least 90% identical to the amino acid sequence of SEQ ID NO: 159; or
lxxxii. a light chain comprising an amino acid sequence at least 90% identical to the amino acid sequence of SEQ ID NO: 244, and a heavy chain comprising an amino acid sequence at least 90% identical to the amino acid sequence of SEQ ID NO: 159; or
lxxxiii. a light chain comprising an amino acid sequence at least 90% identical to the amino acid sequence of SEQ ID NO: 254, and a heavy chain comprising an amino acid sequence at least 90% identical to the amino acid sequence of SEQ ID NO: 159; or
lxxxiv. a light chain comprising an amino acid sequence at least 90% identical to the amino acid sequence of SEQ ID NO: 104, and a heavy chain comprising an amino acid sequence at least 90% identical to the amino acid sequence of SEQ ID NO: 169; or
lxxxv. a light chain comprising an amino acid sequence at least 90% identical to the amino acid sequence of SEQ ID NO: 114, and a heavy chain comprising an amino acid sequence at least 90% identical to the amino acid sequence of SEQ ID NO: 169; or
lxxxvi. a light chain comprising an amino acid sequence at least 90% identical to the amino acid sequence of SEQ ID NO: 124, and a heavy chain comprising an amino acid sequence at least 90% identical to the amino acid sequence of SEQ ID NO: 169; or
lxxxvii. a light chain comprising an amino acid sequence at least 90% identical to the amino acid sequence of SEQ ID NO: 164, and a heavy chain comprising an amino acid sequence at least 90% identical to the amino acid sequence of SEQ ID NO: 169; or
lxxxviii. a light chain comprising an amino acid sequence at least 90% identical to the amino acid sequence of SEQ ID NO: 174, and a heavy chain comprising an amino acid sequence at least 90% identical to the amino acid sequence of SEQ ID NO: 169; or
lxxxix. a light chain comprising an amino acid sequence at least 90% identical to the amino acid sequence of SEQ ID NO: 184, and a heavy chain comprising an amino acid sequence at least 90% identical to the amino acid sequence of SEQ ID NO: 169; or
xc. a light chain comprising an amino acid sequence at least 90% identical to the amino acid sequence of SEQ ID NO: 194, and a heavy chain comprising an amino acid sequence at least 90% identical to the amino acid sequence of SEQ ID NO: 169; or
xci. a light chain comprising an amino acid sequence at least 90% identical to the amino acid sequence of SEQ ID NO: 204, and a heavy chain comprising an amino acid sequence at least 90% identical to the amino acid sequence of SEQ ID NO: 169; or
xcii. a light chain comprising an amino acid sequence at least 90% identical to the amino acid sequence of SEQ ID NO: 214, and a heavy chain comprising an amino acid sequence at least 90% identical to the amino acid sequence of SEQ ID NO: 169; or
xciii. a light chain comprising an amino acid sequence at least 90% identical to the amino acid sequence of SEQ ID NO: 224, and a heavy chain comprising an amino acid sequence at least 90% identical to the amino acid sequence of SEQ ID NO: 169; or
xciv. a light chain comprising an amino acid sequence at least 90% identical to the amino acid sequence of SEQ ID NO: 234, and a heavy chain comprising an amino acid sequence at least 90% identical to the amino acid sequence of SEQ ID NO: 169; or
xcv. a light chain comprising an amino acid sequence at least 90% identical to the amino acid sequence of SEQ ID NO: 244, and a heavy chain comprising an amino acid sequence at least 90% identical to the amino acid sequence of SEQ ID NO: 169; or
xcvi. a light chain comprising an amino acid sequence at least 90% identical to the amino acid sequence of SEQ ID NO: 254, and a heavy chain comprising an amino acid sequence at least 90% identical to the amino acid sequence of SEQ ID NO: 169; or
xcvii. a light chain comprising an amino acid sequence at least 90% identical to the amino acid sequence of SEQ ID NO: 104, and a heavy chain comprising an amino acid sequence at least 90% identical to the amino acid sequence of SEQ ID NO: 179; or
xcviii. a light chain comprising an amino acid sequence at least 90% identical to the amino acid sequence of SEQ ID NO: 114, and a heavy chain comprising an amino acid sequence at least 90% identical to the amino acid sequence of SEQ ID NO: 179; or
xcix. a light chain comprising an amino acid sequence at least 90% identical to the amino acid sequence of SEQ ID NO: 124, and a heavy chain comprising an amino acid sequence at least 90% identical to the amino acid sequence of SEQ ID NO: 179; or
c. a light chain comprising an amino acid sequence at least 90% identical to the amino acid sequence of SEQ ID NO: 154, and a heavy chain comprising an amino acid sequence at least 90% identical to the amino acid sequence of SEQ ID NO: 179; or
ci. a light chain comprising an amino acid sequence at least 90% identical to the amino acid sequence of SEQ ID NO: 164, and a heavy chain comprising an amino acid sequence at least 90% identical to the amino acid sequence of SEQ ID NO: 179; or
cii. a light chain comprising an amino acid sequence at least 90% identical to the amino acid sequence of SEQ ID NO: 174, and a heavy chain comprising an amino acid sequence at least 90% identical to the amino acid sequence of SEQ ID NO: 179; or
ciii. a light chain comprising an amino acid sequence at least 90% identical to the amino acid sequence of SEQ ID NO: 184, and a heavy chain comprising an amino acid sequence at least 90% identical to the amino acid sequence of SEQ ID NO: 179; or
civ. a light chain comprising an amino acid sequence at least 90% identical to the amino acid sequence of SEQ ID NO: 194, and a heavy chain comprising an amino acid sequence at least 90% identical to the amino acid sequence of SEQ ID NO: 179; or
cv. a light chain comprising an amino acid sequence at least 90% identical to the amino acid sequence of SEQ ID NO: 204, and a heavy chain comprising an amino acid sequence at least 90% identical to the amino acid sequence of SEQ ID NO: 179; or
cvi. a light chain comprising an amino acid sequence at least 90% identical to the amino acid sequence of SEQ ID NO: 214, and a heavy chain comprising an amino acid sequence at least 90% identical to the amino acid sequence of SEQ ID NO: 179; or
cvii. a light chain comprising an amino acid sequence at least 90% identical to the amino acid sequence of SEQ ID NO: 224, and a heavy chain comprising an amino acid sequence at least 90% identical to the amino acid sequence of SEQ ID NO: 179; or
cviii. a light chain comprising an amino acid sequence at least 90% identical to the amino acid sequence of SEQ ID NO: 234, and a heavy chain comprising an amino acid sequence at least 90% identical to the amino acid sequence of SEQ ID NO: 179; or
cix. a light chain comprising an amino acid sequence at least 90% identical to the amino acid sequence of SEQ ID NO: 244, and a heavy chain comprising an amino acid sequence at least 90% identical to the amino acid sequence of SEQ ID NO: 179.

11. The method according to claim 1, wherein the anti-CTGF antibody or antigen-binding fragment thereof comprises:
i. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 109; or
ii. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 109; or
iii. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 109; or
iv. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 109; or
v. a light chain comprising an amino acid sequence at least 95% identical to the amino acid sequence of SEQ ID NO: 134, and a heavy chain comprising an amino acid sequence at least 95% identical to the amino acid sequence of SEQ ID NO: 109; or
vi. a light chain comprising an amino acid sequence at least 95% identical to the amino acid sequence of SEQ ID NO: 154, and a heavy chain comprising an amino acid sequence at least 95% identical to the amino acid sequence of SEQ ID NO: 109; or
vii. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 109; or
viii. a light chain comprising an amino acid sequence at least 95% identical to the amino acid sequence of SEQ ID NO: 174, and a heavy chain comprising an amino acid sequence at least 95% identical to the amino acid sequence of SEQ ID NO: 109; or
ix. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 109; or
x. a light chain comprising an amino acid sequence at least 95% identical to the amino acid sequence of SEQ ID NO: 194, and a heavy chain comprising an amino acid sequence at least 95% identical to the amino acid sequence of SEQ ID NO: 109; or
xi. a light chain comprising an amino acid sequence at least 95% identical to the amino acid sequence of SEQ ID NO: 204, and a heavy chain comprising an amino acid sequence at least 95% identical to the amino acid sequence of SEQ ID NO: 109; or
xii. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 109; or
xiii. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 109; or
xiv. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 109; or
xv. a light chain comprising an amino acid sequence at least 95% identical to the amino acid sequence of SEQ ID NO: 254, and a heavy chain comprising an amino acid sequence at least 95% identical to the amino acid sequence of SEQ ID NO: 109; or
xvi. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 119; or
xvii. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 119; or
xviii. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 119; or
xix. a light chain comprising an amino acid sequence at least 95% identical to the amino acid sequence of SEQ ID NO: 154, and a heavy chain comprising an amino acid sequence at least 95% identical to the amino acid sequence of SEQ ID NO: 119; or
xx. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 119; or
xxi. a light chain comprising an amino acid sequence at least 95% identical to the amino acid sequence of SEQ ID NO: 174, and a heavy chain comprising an amino acid sequence at least 95% identical to the amino acid sequence of SEQ ID NO: 119; or
xxii. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 119; or
xxiii. a light chain comprising an amino acid sequence at least 95% identical to the amino acid sequence of SEQ ID NO: 194, and a heavy chain comprising an amino acid sequence at least 95% identical to the amino acid sequence of SEQ ID NO: 119; or
xxiv. a light chain comprising an amino acid sequence at least 95% identical to the amino acid sequence of SEQ ID NO: 204, and a heavy chain comprising an amino acid sequence at least 95% identical to the amino acid sequence of SEQ ID NO: 119; or
xxv. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 119; or
xxvi. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 119; or
xxvii. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 119; or
xxviii. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 119; or
xxix. a light chain comprising an amino acid sequence at least 95% identical to the amino acid sequence of SEQ ID NO: 254, and a heavy chain comprising an amino acid sequence at least 95% identical to the amino acid sequence of SEQ ID NO: 119; or
xxx. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 129; or
xxxi. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 129; or
xxxii. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 129; or
xxxiii. a light chain comprising an amino acid sequence at least 95% identical to the amino acid sequence of SEQ ID NO: 154, and a heavy chain comprising an amino acid sequence at least 95% identical to the amino acid sequence of SEQ ID NO: 129; or
xxxiv. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 129; or
xxxv. a light chain comprising an amino acid sequence at least 95% identical to the amino acid sequence of SEQ ID NO: 174, and a heavy chain comprising an amino acid sequence at least 95% identical to the amino acid sequence of SEQ ID NO: 129; or
xxxvi. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 129; or
xxxvii. a light chain comprising an amino acid sequence at least 95% identical to the amino acid sequence of SEQ ID NO: 194, and a heavy chain comprising an amino acid sequence at least 95% identical to the amino acid sequence of SEQ ID NO: 129; or
xxxviii. a light chain comprising an amino acid sequence at least 95% identical to the amino acid sequence of SEQ ID NO: 204, and a heavy chain comprising an amino acid sequence at least 95% identical to the amino acid sequence of SEQ ID NO: 129; or
xxxix. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 129; or
xl. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 129; or
xli. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 129; or
xlii. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 129; or
xliii. a light chain comprising an amino acid sequence at least 95% identical to the amino acid sequence of SEQ ID NO: 254, and a heavy chain comprising an amino acid sequence at least 95% identical to the amino acid sequence of SEQ ID NO: 129; or
xliv. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 139; or
xlv. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 139; or
xlvi. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 139; or
xlvii. a light chain comprising an amino acid sequence at least 95% identical to the amino acid sequence of SEQ ID NO: 154, and a heavy chain comprising an amino acid sequence at least 95% identical to the amino acid sequence of SEQ ID NO: 139; or
xlviii. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 139; or
xlix. a light chain comprising an amino acid sequence at least 95% identical to the amino acid sequence of SEQ ID NO: 174, and a heavy chain comprising an amino acid sequence at least 95% identical to the amino acid sequence of SEQ ID NO: 139; or
l. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 139; or
li. a light chain comprising an amino acid sequence at least 95% identical to the amino acid sequence of SEQ ID NO: 194, and a heavy chain comprising an amino acid sequence at least 95% identical to the amino acid sequence of SEQ ID NO: 139; or
lii. a light chain comprising an amino acid sequence at least 95% identical to the amino acid sequence of SEQ ID NO: 204, and a heavy chain comprising an amino acid sequence at least 95% identical to the amino acid sequence of SEQ ID NO: 139; or
liii. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 139; or
liv. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 139; or
lv. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 139; or
lvi. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 139; or
lvii. a light chain comprising an amino acid sequence at least 95% identical to the amino acid sequence of SEQ ID NO: 254, and a heavy chain comprising an amino acid sequence at least 95% identical to the amino acid sequence of SEQ ID NO: 139; or
lviii. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 149; or
lix. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 149; or
lx. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 149; or
lxi. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 149; or
lxii. a light chain comprising an amino acid sequence at least 95% identical to the amino acid sequence of SEQ ID NO: 174, and a heavy chain comprising an amino acid sequence at least 95% identical to the amino acid sequence of SEQ ID NO: 149; or
lxiii. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 149; or
lxiv. a light chain comprising an amino acid sequence at least 95% identical to the amino acid sequence of SEQ ID NO: 194, and a heavy chain comprising an amino acid sequence at least 95% identical to the amino acid sequence of SEQ ID NO: 149; or
lxv. a light chain comprising an amino acid sequence at least 95% identical to the amino acid sequence of SEQ ID NO: 204, and a heavy chain comprising an amino acid sequence at least 95% identical to the amino acid sequence of SEQ ID NO: 149; or
lxvi. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 149; or
lxvii. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 149; or
lxviii. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 149; or
lxix. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 149; or
lxx. a light chain comprising an amino acid sequence at least 95% identical to the amino acid sequence of SEQ ID NO: 254, and a heavy chain comprising an amino acid sequence at least 95% identical to the amino acid sequence of SEQ ID NO: 149; or
lxxi. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 159; or
lxxii. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 159; or
lxxiii. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 159; or
lxxiv. a light chain comprising an amino acid sequence at least 95% identical to the amino acid sequence of SEQ ID NO: 154, and a heavy chain comprising an amino acid sequence at least 95% identical to the amino acid sequence of SEQ ID NO: 159; or
lxxv. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 159; or
lxxvi. a light chain comprising an amino acid sequence at least 95% identical to the amino acid sequence of SEQ ID NO: 174, and a heavy chain comprising an amino acid sequence at least 95% identical to the amino acid sequence of SEQ ID NO: 159; or
lxxvii. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 159; or
lxxviii. a light chain comprising an amino acid sequence at least 95% identical to the amino acid sequence of SEQ ID NO: 194, and a heavy chain comprising an amino acid sequence at least 95% identical to the amino acid sequence of SEQ ID NO: 159; or
lxxix. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 159; or
lxxx. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 159; or
lxxxi. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 159; or
lxxxii. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 159; or
lxxxiii. a light chain comprising an amino acid sequence at least 95% identical to the amino acid sequence of SEQ ID NO: 254, and a heavy chain comprising an amino acid sequence at least 95% identical to the amino acid sequence of SEQ ID NO: 159; or
lxxxiv. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 169; or
lxxxv. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 169; or
lxxxvi. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 169; or
lxxxvii. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 169; or
lxxxviii. a light chain comprising an amino acid sequence at least 95% identical to the amino acid sequence of SEQ ID NO: 174, and a heavy chain comprising an amino acid sequence at least 95% identical to the amino acid sequence of SEQ ID NO: 169; or
lxxxix. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 169; or
xc. a light chain comprising an amino acid sequence at least 95% identical to the amino acid sequence of SEQ ID NO: 194, and a heavy chain comprising an amino acid sequence at least 95% identical to the amino acid sequence of SEQ ID NO: 169; or
xci. a light chain comprising an amino acid sequence at least 95% identical to the amino acid sequence of SEQ ID NO: 204, and a heavy chain comprising an amino acid sequence at least 95% identical to the amino acid sequence of SEQ ID NO: 169; or
xcii. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 169; or
xciii. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 169; or
xciv. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 169; or
xcv. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 169; or
xcvi. a light chain comprising an amino acid sequence at least 95% identical to the amino acid sequence of SEQ ID NO: 254, and a heavy chain comprising an amino acid sequence at least 95% identical to the amino acid sequence of SEQ ID NO: 169; or
xcvii. a light chain comprising an amino acid sequence at least 95% identical to the amino acid sequence of SEQ ID NO: 104, and a heavy chain comprising an amino acid sequence at least 95% identical to the amino acid sequence of SEQ ID NO: 179; or
xcviii. a light chain comprising an amino acid sequence at least 95% identical to the amino acid sequence of SEQ ID NO: 114, and a heavy chain comprising an amino acid sequence at least 95% identical to the amino acid sequence of SEQ ID NO: 179; or
xcix. a light chain comprising an amino acid sequence at least 95% identical to the amino acid sequence of SEQ ID NO: 124, and a heavy chain comprising an amino acid sequence at least 95% identical to the amino acid sequence of SEQ ID NO: 179; or
c. a light chain comprising an amino acid sequence at least 95% identical to the amino acid sequence of SEQ ID NO: 154, and a heavy chain comprising an amino acid sequence at least 95% identical to the amino acid sequence of SEQ ID NO: 179; or
ci. a light chain comprising an amino acid sequence at least 95% identical to the amino acid sequence of SEQ ID NO: 164, and a heavy chain comprising an amino acid sequence at least 95% identical to the amino acid sequence of SEQ ID NO: 179; or
cii. a light chain comprising an amino acid sequence at least 95% identical to the amino acid sequence of SEQ ID NO: 174, and a heavy chain comprising an amino acid sequence at least 95% identical to the amino acid sequence of SEQ ID NO: 179; or
ciii. a light chain comprising an amino acid sequence at least 95% identical to the amino acid sequence of SEQ ID NO: 184, and a heavy chain comprising an amino acid sequence at least 95% identical to the amino acid sequence of SEQ ID NO: 179; or
civ. a light chain comprising an amino acid sequence at least 95% identical to the amino acid sequence of SEQ ID NO: 194, and a heavy chain comprising an amino acid sequence at least 95% identical to the amino acid sequence of SEQ ID NO: 179; or
cv. a light chain comprising an amino acid sequence at least 95% identical to the amino acid sequence of SEQ ID NO: 204, and a heavy chain comprising an amino acid sequence at least 95% identical to the amino acid sequence of SEQ ID NO: 179; or
cvi. a light chain comprising an amino acid sequence at least 95% identical to the amino acid sequence of SEQ ID NO: 214, and a heavy chain comprising an amino acid sequence at least 95% identical to the amino acid sequence of SEQ ID NO: 179; or
cvii. a light chain comprising an amino acid sequence at least 95% identical to the amino acid sequence of SEQ ID NO: 224, and a heavy chain comprising an amino acid sequence at least 95% identical to the amino acid sequence of SEQ ID NO: 179; or
cviii. a light chain comprising an amino acid sequence at least 95% identical to the amino acid sequence of SEQ ID NO: 234, and a heavy chain comprising an amino acid sequence at least 95% identical to the amino acid sequence of SEQ ID NO: 179; or
cix. a light chain comprising an amino acid sequence at least 95% identical to the amino acid sequence of SEQ ID NO: 244, and a heavy chain comprising an amino acid sequence at least 95% identical to the amino acid sequence of SEQ ID NO: 179.

12. The method according to claim 1, wherein the anti-CTGF antibody or antigen-binding fragment thereof comprises:
i. a light chain comprising the amino acid sequence of SEQ ID NO: 224, and a heavy chain comprising the amino acid sequence of SEQ ID NO: 109; or
ii. a light chain comprising the amino acid sequence of SEQ ID NO: 104, and a heavy chain comprising the amino acid sequence of SEQ ID NO: 109; or
iii. a light chain comprising the amino acid sequence of SEQ ID NO: 114, and a heavy chain comprising the amino acid sequence of SEQ ID NO: 109; or
iv. a light chain comprising the amino acid sequence of SEQ ID NO: 124, and a heavy chain comprising the amino acid sequence of SEQ ID NO: 109; or
v. a light chain comprising the amino acid sequence of SEQ ID NO: 134, and a heavy chain comprising the amino acid sequence of SEQ ID NO: 109; or
vi. a light chain comprising the amino acid sequence of SEQ ID NO: 154, and a heavy chain comprising the amino acid sequence of SEQ ID NO: 109; or
vii. a light chain comprising the amino acid sequence of SEQ ID NO: 164, and a heavy chain comprising the amino acid sequence of SEQ ID NO: 109; or
viii. a light chain comprising the amino acid sequence of SEQ ID NO: 174, and a heavy chain comprising the amino acid sequence of SEQ ID NO: 109; or
ix. a light chain comprising the amino acid sequence of SEQ ID NO: 184, and a heavy chain comprising the amino acid sequence of SEQ ID NO: 109; or
x. a light chain comprising the amino acid sequence of SEQ ID NO: 194, and a heavy chain comprising the amino acid sequence of SEQ ID NO: 109; or
xi. a light chain comprising the amino acid sequence of SEQ ID NO: 204, and a heavy chain comprising the amino acid sequence of SEQ ID NO: 109; or
xii. a light chain comprising the amino acid sequence of SEQ ID NO: 214, and a heavy chain comprising the amino acid sequence of SEQ ID NO: 109; or
xiii. a light chain comprising the amino acid sequence of SEQ ID NO: 234, and a heavy chain comprising the amino acid sequence of SEQ ID NO: 109; or
xiv. a light chain comprising the amino acid sequence of SEQ ID NO: 244, and a heavy chain comprising the amino acid sequence of SEQ ID NO: 109; or
xv. a light chain comprising the amino acid sequence of SEQ ID NO: 254, and a heavy chain comprising the amino acid sequence of SEQ ID NO: 109; or
xvi. a light chain comprising the amino acid sequence of SEQ ID NO: 104, and a heavy chain comprising the amino acid sequence of SEQ ID NO: 119; or
xvii. a light chain comprising the amino acid sequence of SEQ ID NO: 114, and a heavy chain comprising the amino acid sequence of SEQ ID NO: 119; or
xviii. a light chain comprising the amino acid sequence of SEQ ID NO: 124, and a heavy chain comprising the amino acid sequence of SEQ ID NO: 119; or
xix. a light chain comprising the amino acid sequence of SEQ ID NO: 154, and a heavy chain comprising the amino acid sequence of SEQ ID NO: 119; or
xx. a light chain comprising the amino acid sequence of SEQ ID NO: 164, and a heavy chain comprising the amino acid sequence of SEQ ID NO: 119; or
xxi. a light chain comprising the amino acid sequence of SEQ ID NO: 174, and a heavy chain comprising the amino acid sequence of SEQ ID NO: 119; or
xxii. a light chain comprising the amino acid sequence of SEQ ID NO: 184, and a heavy chain comprising the amino acid sequence of SEQ ID NO: 119; or
xxiii. a light chain comprising the amino acid sequence of SEQ ID NO: 194, and a heavy chain comprising the amino acid sequence of SEQ ID NO: 119; or
xxiv. a light chain comprising the amino acid sequence of SEQ ID NO: 204, and a heavy chain comprising the amino acid sequence of SEQ ID NO: 119; or
xxv. a light chain comprising the amino acid sequence of SEQ ID NO: 214, and a heavy chain comprising the amino acid sequence of SEQ ID NO: 119; or
xxvi. a light chain comprising the amino acid sequence of SEQ ID NO: 224, and a heavy chain comprising the amino acid sequence of SEQ ID NO: 119; or
xxvii. a light chain comprising the amino acid sequence of SEQ ID NO: 234, and a heavy chain comprising the amino acid sequence of SEQ ID NO: 119; or
xxviii. a light chain comprising the amino acid sequence of SEQ ID NO: 244, and a heavy chain comprising the amino acid sequence of SEQ ID NO: 119; or
xxix. a light chain comprising the amino acid sequence of SEQ ID NO: 254, and a heavy chain comprising the amino acid sequence of SEQ ID NO: 119; or
xxx. a light chain comprising the amino acid sequence of SEQ ID NO: 104, and a heavy chain comprising the amino acid sequence of SEQ ID NO: 129; or
xxxi. a light chain comprising the amino acid sequence of SEQ ID NO: 114, and a heavy chain comprising the amino acid sequence of SEQ ID NO: 129; or
xxxii. a light chain comprising the amino acid sequence of SEQ ID NO: 124, and a heavy chain comprising the amino acid sequence of SEQ ID NO: 129; or
xxxiii. a light chain comprising the amino acid sequence of SEQ ID NO: 154, and a heavy chain comprising the amino acid sequence of SEQ ID NO: 129; or
xxxiv. a light chain comprising the amino acid sequence of SEQ ID NO: 164, and a heavy chain comprising the amino acid sequence of SEQ ID NO: 129; or
xxxv. a light chain comprising the amino acid sequence of SEQ ID NO: 174, and a heavy chain comprising the amino acid sequence of SEQ ID NO: 129; or
xxxvi. a light chain comprising the amino acid sequence of SEQ ID NO: 184, and a heavy chain comprising the amino acid sequence of SEQ ID NO: 129; or
xxxvii. a light chain comprising the amino acid sequence of SEQ ID NO: 194, and a heavy chain comprising the amino acid sequence of SEQ ID NO: 129; or
xxxviii. a light chain comprising the amino acid sequence of SEQ ID NO: 204, and a heavy chain comprising the amino acid sequence of SEQ ID NO: 129; or
xxxix. a light chain comprising the amino acid sequence of SEQ ID NO: 214, and a heavy chain comprising the amino acid sequence of SEQ ID NO: 129; or
xl. a light chain comprising the amino acid sequence of SEQ ID NO: 224, and a heavy chain comprising the amino acid sequence of SEQ ID NO: 129; or
xli. a light chain comprising the amino acid sequence of SEQ ID NO: 234, and a heavy chain comprising the amino acid sequence of SEQ ID NO: 129; or
xlii. a light chain comprising the amino acid sequence of SEQ ID NO: 244, and a heavy chain comprising the amino acid sequence of SEQ ID NO: 129; or
xliii. a light chain comprising the amino acid sequence of SEQ ID NO: 254, and a heavy chain comprising the amino acid sequence of SEQ ID NO: 129; or
xliv. a light chain comprising the amino acid sequence of SEQ ID NO: 104, and a heavy chain comprising the amino acid sequence of SEQ ID NO: 139; or
xlv. a light chain comprising the amino acid sequence of SEQ ID NO: 114, and a heavy chain comprising the amino acid sequence of SEQ ID NO: 139; or
xlvi. a light chain comprising the amino acid sequence of SEQ ID NO: 124, and a heavy chain comprising the amino acid sequence of SEQ ID NO: 139; or
xlvii. a light chain comprising the amino acid sequence of SEQ ID NO: 154, and a heavy chain comprising the amino acid sequence of SEQ ID NO: 139; or
xlviii. a light chain comprising the amino acid sequence of SEQ ID NO: 164, and a heavy chain comprising the amino acid sequence of SEQ ID NO: 139; or
xlix. a light chain comprising the amino acid sequence of SEQ ID NO: 174, and a heavy chain comprising the amino acid sequence of SEQ ID NO: 139; or
l. a light chain comprising the amino acid sequence of SEQ ID NO: 184, and a heavy chain comprising the amino acid sequence of SEQ ID NO: 139; or
li. a light chain comprising the amino acid sequence of SEQ ID NO: 194, and a heavy chain comprising the amino acid sequence of SEQ ID NO: 139; or
lii. a light chain comprising the amino acid sequence of SEQ ID NO: 204, and a heavy chain comprising the amino acid sequence of SEQ ID NO: 139; or
liii. a light chain comprising the amino acid sequence of SEQ ID NO: 214, and a heavy chain comprising the amino acid sequence of SEQ ID NO: 139; or
liv. a light chain comprising the amino acid sequence of SEQ ID NO: 224, and a heavy chain comprising the amino acid sequence of SEQ ID NO: 139; or
lv. a light chain comprising the amino acid sequence of SEQ ID NO: 234, and a heavy chain comprising the amino acid sequence of SEQ ID NO: 139; or
lvi. a light chain comprising the amino acid sequence of SEQ ID NO: 244, and a heavy chain comprising the amino acid sequence of SEQ ID NO: 139; or
lvii. a light chain comprising the amino acid sequence of SEQ ID NO: 254, and a heavy chain comprising the amino acid sequence of SEQ ID NO: 139; or
lviii. a light chain comprising the amino acid sequence of SEQ ID NO: 104, and a heavy chain comprising the amino acid sequence of SEQ ID NO: 149; or
lix. a light chain comprising the amino acid sequence of SEQ ID NO: 114, and a heavy chain comprising the amino acid sequence of SEQ ID NO: 149; or
lx. a light chain comprising the amino acid sequence of SEQ ID NO: 124, and a heavy chain comprising the amino acid sequence of SEQ ID NO: 149; or
lxi. a light chain comprising the amino acid sequence of SEQ ID NO: 164, and a heavy chain comprising the amino acid sequence of SEQ ID NO: 149; or
lxii. a light chain comprising the amino acid sequence of SEQ ID NO: 174, and a heavy chain comprising the amino acid sequence of SEQ ID NO: 149; or
lxiii. a light chain comprising the amino acid sequence of SEQ ID NO: 184, and a heavy chain comprising the amino acid sequence of SEQ ID NO: 149; or
lxiv. a light chain comprising the amino acid sequence of SEQ ID NO: 194, and a heavy chain comprising the amino acid sequence of SEQ ID NO: 149; or
lxv. a light chain comprising the amino acid sequence of SEQ ID NO: 204, and a heavy chain comprising the amino acid sequence of SEQ ID NO: 149; or
lxvi. a light chain comprising the amino acid sequence of SEQ ID NO: 214, and a heavy chain comprising the amino acid sequence of SEQ ID NO: 149; or
lxvii. a light chain comprising the amino acid sequence of SEQ ID NO: 224, and a heavy chain comprising the amino acid sequence of SEQ ID NO: 149; or
lxviii. a light chain comprising the amino acid sequence of SEQ ID NO: 234, and a heavy chain comprising the amino acid sequence of SEQ ID NO: 149; or
lxix. a light chain comprising the amino acid sequence of SEQ ID NO: 244, and a heavy chain comprising the amino acid sequence of SEQ ID NO: 149; or
lxx. a light chain comprising the amino acid sequence of SEQ ID NO: 254, and a heavy chain comprising the amino acid sequence of SEQ ID NO: 149; or
lxxi. a light chain comprising the amino acid sequence of SEQ ID NO: 104, and a heavy chain comprising the amino acid sequence of SEQ ID NO: 159; or
lxxii. a light chain comprising the amino acid sequence of SEQ ID NO: 114, and a heavy chain comprising the amino acid sequence of SEQ ID NO: 159; or
lxxiii. a light chain comprising the amino acid sequence of SEQ ID NO: 124, and a heavy chain comprising the amino acid sequence of SEQ ID NO: 159; or
lxxiv. a light chain comprising the amino acid sequence of SEQ ID NO: 154, and a heavy chain comprising the amino acid sequence of SEQ ID NO: 159; or
lxxv. a light chain comprising the amino acid sequence of SEQ ID NO: 164, and a heavy chain comprising the amino acid sequence of SEQ ID NO: 159; or
lxxvi. a light chain comprising the amino acid sequence of SEQ ID NO: 174, and a heavy chain comprising the amino acid sequence of SEQ ID NO: 159; or
lxxvii. a light chain comprising the amino acid sequence of SEQ ID NO: 184, and a heavy chain comprising the amino acid sequence of SEQ ID NO: 159; or
lxxviii. a light chain comprising the amino acid sequence of SEQ ID NO: 194, and a heavy chain comprising the amino acid sequence of SEQ ID NO: 159; or
lxxix. a light chain comprising the amino acid sequence of SEQ ID NO: 214, and a heavy chain comprising the amino acid sequence of SEQ ID NO: 159; or
lxxx. a light chain comprising the amino acid sequence of SEQ ID NO: 224, and a heavy chain comprising the amino acid sequence of SEQ ID NO: 159; or
lxxxi. a light chain comprising the amino acid sequence of SEQ ID NO: 234, and a heavy chain comprising the amino acid sequence of SEQ ID NO: 159; or
lxxxii. a light chain comprising the amino acid sequence of SEQ ID NO: 244, and a heavy chain comprising the amino acid sequence of SEQ ID NO: 159; or
lxxxiii. a light chain comprising the amino acid sequence of SEQ ID NO: 254, and a heavy chain comprising the amino acid sequence of SEQ ID NO: 159; or
lxxxiv. a light chain comprising the amino acid sequence of SEQ ID NO: 104, and a heavy chain comprising the amino acid sequence of SEQ ID NO: 169; or
lxxxv. a light chain comprising the amino acid sequence of SEQ ID NO: 114, and a heavy chain comprising the amino acid sequence of SEQ ID NO: 169; or
lxxxvi. a light chain comprising the amino acid sequence of SEQ ID NO: 124, and a heavy chain comprising the amino acid sequence of SEQ ID NO: 169; or
lxxxvii. a light chain comprising the amino acid sequence of SEQ ID NO: 164, and a heavy chain comprising the amino acid sequence of SEQ ID NO: 169; or
lxxxviii. a light chain comprising the amino acid sequence of SEQ ID NO: 174, and a heavy chain comprising the amino acid sequence of SEQ ID NO: 169; or
lxxxix. a light chain comprising the amino acid sequence of SEQ ID NO: 184, and a heavy chain comprising the amino acid sequence of SEQ ID NO: 169; or
xc. a light chain comprising the amino acid sequence of SEQ ID NO: 194, and a heavy chain comprising the amino acid sequence of SEQ ID NO: 169; or
xci. a light chain comprising the amino acid sequence of SEQ ID NO: 204, and a heavy chain comprising the amino acid sequence of SEQ ID NO: 169; or
xcii. a light chain comprising the amino acid sequence of SEQ ID NO: 214, and a heavy chain comprising the amino acid sequence of SEQ ID NO: 169; or
xciii. a light chain comprising the amino acid sequence of SEQ ID NO: 224, and a heavy chain comprising the amino acid sequence of SEQ ID NO: 169; or
xciv. a light chain comprising the amino acid sequence of SEQ ID NO: 234, and a heavy chain comprising the amino acid sequence of SEQ ID NO: 169; or
xcv. a light chain comprising the amino acid sequence of SEQ ID NO: 244, and a heavy chain comprising the amino acid sequence of SEQ ID NO: 169; or
xcvi. a light chain comprising the amino acid sequence of SEQ ID NO: 254, and a heavy chain comprising the amino acid sequence of SEQ ID NO: 169; or
xcvii. a light chain comprising the amino acid sequence of SEQ ID NO: 104, and a heavy chain comprising the amino acid sequence of SEQ ID NO: 179; or
xcviii. a light chain comprising the amino acid sequence of SEQ ID NO: 114, and a heavy chain comprising the amino acid sequence of SEQ ID NO: 179; or
xcix. a light chain comprising the amino acid sequence of SEQ ID NO: 124, and a heavy chain comprising the amino acid sequence of SEQ ID NO: 179; or
c. a light chain comprising the amino acid sequence of SEQ ID NO: 154, and a heavy chain comprising the amino acid sequence of SEQ ID NO: 179; or
ci. a light chain comprising the amino acid sequence of SEQ ID NO: 164, and a heavy chain comprising the amino acid sequence of SEQ ID NO: 179; or
cii. a light chain comprising the amino acid sequence of SEQ ID NO: 174, and a heavy chain comprising the amino acid sequence of SEQ ID NO: 179; or
ciii. a light chain comprising the amino acid sequence of SEQ ID NO: 184, and a heavy chain comprising the amino acid sequence of SEQ ID NO: 179; or
civ. a light chain comprising the amino acid sequence of SEQ ID NO: 194, and a heavy chain comprising the amino acid sequence of SEQ ID NO: 179; or
cv. a light chain comprising the amino acid sequence of SEQ ID NO: 204, and a heavy chain comprising the amino acid sequence of SEQ ID NO: 179; or
cvi. a light chain comprising the amino acid sequence of SEQ ID NO: 214, and a heavy chain comprising the amino acid sequence of SEQ ID NO: 179; or
cvii. a light chain comprising the amino acid sequence of SEQ ID NO: 224, and a heavy chain comprising the amino acid sequence of SEQ ID NO: 179; or
cviii. a light chain comprising the amino acid sequence of SEQ ID NO: 234, and a heavy chain comprising the amino acid sequence of SEQ ID NO: 179; or
cix. a light chain comprising the amino acid sequence of SEQ ID NO: 244, and a heavy chain comprising the amino acid sequence of SEQ ID NO: 179.

13. The method according to claim 1, wherein the anti-CTGF antibody or antigen-binding fragment thereof comprises:
(a) a light chain variable region comprising an L-CDR1 having the amino acid sequence of SEQ ID NO: 220, an L-CDR2 having the amino acid sequence of SEQ ID NO: 221, and an L-CDR3 having the amino acid sequence of SEQ ID NO: 222; and a heavy chain variable region comprising an H-CDR1 having the amino acid sequence of SEQ ID NO: 105, an H-CDR2 having the amino acid sequence of SEQ ID NO: 106, and an H-CDR3 having the amino acid sequence of SEQ ID NO: 107; or
(b) a light chain variable region comprising an L-CDR1, an L-CDR2, and an L-CDR3, wherein the amino acid sequence of said L-CDR1 consists of the amino acid sequence of SEQ ID NO: 220, the amino acid sequence of said L-CDR2 consists of the amino acid sequence of SEQ ID NO: 221, and the amino acid sequence of said L-CDR3 consists of the amino acid sequence of SEQ ID NO: 222; and further comprising a heavy chain variable region comprising an H-CDR1, an H-CDR2, and an H-CDR3, wherein the amino acid sequence of said H-CDR1 consists of the amino acid sequence of SEQ ID NO: 105, the amino acid sequence of said H-CDR2 consists of the amino acid sequence of SEQ ID NO: 106, and the amino acid sequence of said H-CDR3 consists of the amino acid sequence of SEQ ID NO: 107; or
(c) a light chain variable region comprising the amino acid sequence of SEQ ID NO: 223, and a heavy chain variable region comprising the amino acid sequence of SEQ ID NO: 108; or
(d) a light chain variable region and a heavy chain variable region, wherein the amino acid sequence of said light chain variable region consists of the amino acid sequence of SEQ ID NO: 223, and the amino acid sequence of said heavy chain variable region consists of the amino acid sequence of SEQ ID NO: 108; or
(e) a light chain comprising the amino acid sequence of SEQ ID NO: 224, and a heavy chain comprising the amino acid sequence of SEQ ID NO: 109; or
(f) a light chain and a heavy chain, wherein the amino acid sequence of said light chain consists of the amino acid sequence of SEQ ID NO: 224, and the amino acid sequence of said heavy chain consists of the amino acid sequence of SEQ ID NO: 109.

14-18. (canceled)

19. The method according to claim 1, wherein the anti-CTGF antibody or antigen-binding fragment thereof comprises:
(a) a light chain variable region comprising an L-CDR1 having the amino acid sequence of SEQ ID NO: 110, an L-CDR2 having the amino acid sequence of SEQ ID NO: 111, and an L-CDR3 having the amino acid sequence of SEQ ID NO: 112; and a heavy chain variable region comprising an H-CDR1 having the amino acid sequence of SEQ ID NO: 145, an H-CDR2 having the amino acid sequence of SEQ ID NO: 146, and an H-CDR3 having the amino acid sequence of SEQ ID NO: 147; or
(b) a light chain variable region comprising an L-CDR1, an L-CDR2, and an L-CDR3, wherein the amino acid sequence of said L-CDR1 consists of the amino acid sequence of SEQ ID NO: 110, the amino acid sequence of said L-CDR2 consists of the amino acid sequence of SEQ ID NO: 111, and the amino acid sequence of said L-CDR3 consists of the amino acid sequence of SEQ ID NO: 112; and further comprising a heavy chain variable region comprising an H-CDR1, an H-CDR2, and an H-CDR3, wherein the amino acid sequence of said H-CDR1 consists of the amino acid sequence of SEQ ID NO: 145, the amino acid sequence of said H-CDR2 consists of the amino acid sequence of SEQ ID NO: 146, and the amino acid sequence of said H-CDR3 consists of the amino acid sequence of SEQ ID NO: 147; or
(c) a light chain variable region comprising the amino acid sequence of SEQ ID NO: 113, and a heavy chain variable region comprising the amino acid sequence of SEQ ID NO: 148; or
(d) a light chain variable region and a heavy chain variable region, wherein the amino acid sequence of said light chain variable region consists of the amino acid sequence of SEQ ID NO: 113, and the amino acid sequence of said heavy chain variable region consists of the amino acid sequence of SEQ ID NO: 148; or
(e) a light chain comprising the amino acid sequence of SEQ ID NO: 114, and a heavy chain comprising the amino acid sequence of SEQ ID NO: 149; or
(f) a light chain and a heavy chain, wherein the amino acid sequence of said light chain consists of the amino acid sequence of SEQ ID NO: 114, and the amino acid sequence of said heavy chain consists of the amino acid sequence of SEQ ID NO: 149.

20-24. (canceled)

25. The method according to claim 1, wherein the anti-CTGF antibody or antigen-binding fragment thereof:
(a) is glycosylated; and/or
(b) comprises a heavy chain constant region selected from IgG₁, IgG₂, IgG₃, IgG₄, IgM, IgA and IgE or a variant thereof; and/or
(c) comprises an IgG₁ heavy chain constant region; and/or
(d) comprises a heavy chain constant region wherein the heavy chain constant region is of an IgG₁ with L234A and L235A substitutions; and/or
(e) comprises a heavy chain lacking a C-terminal lysine residue; and/or
(f) comprises a light chain constant region selected from the group consisting of a kappa and a lambda light chain; and/or
(g) is a monoclonal antibody; and/or
(h) is a monospecific antibody; and/or
(i) is a human, humanized, or chimeric antibody, preferably a humanized antibody; and/or
(j) binds the full length human CTGF of SEQ ID NO: 2 with a binding affinity of 50 pM or lower, 25 pM or lower, 15 pM or lower, 10 pM or lower, 5 pM or lower, or 2 pM or lower; and/or
(k) binds the human CTGF-NTF of SEQ ID NO: 3 with a binding affinity of 10 nM or lower, 5 nM or lower, 2.5 nM or lower, 1 nM or lower, 0.5 nM or lower, or 0.2 nM or lower; and/or
(l) binds human CTGF, preferably of SEQ ID NO: 2, with a K_{D} of 5 pM or lower; and/or
(m) binds the N-terminal fragment of human CTGF with a K_{D} of 0.5 nM or lower.

26-117. (canceled)
